(19) United States
(12) Patent Application Publication
(10) Pub. No.: US 2014/0315889 A1
(43) Pub. Date: Oct. 23, 2014

(54) HYDROXAMATE DERIVATIVES FOR HDAC INHIBITOR, AND THE PHARMACEUTICAL COMPOSITION COMPRISING THEREOF

(71) Applicant: CHONG KUN DANG PHARMACEUTICAL CORP., Seoul (KR)

(72) Inventors: Yuntae Kim, Yongin-si (KR); Changsik Lee, Yongin-si (KR); Hyun-mo Yang, Yongin-si (KR); Hojin Choi, Yongin-si (KR); Jaeki Min, Yongin-si (KR); Soyoung Kim, Yongin-si (KR); Dal-Hyun Kim, Yongin-si (KR); Nina Ha, Yongin-si (KR); Jung-Min Kim, Yongin-si (KR); Hyojin Lim, Yongin-si (KR); Eunhee Ko, Yongin-si (KR)

(73) Assignee: CHONG KUN DANG PHARMACEUTICAL CORP., Seoul (KR)

(21) Appl. No.: 14/354,206

(22) PCT Filed: Oct. 26, 2012
(86) PCT No.: PCT/KR2012/008840

§ 371 Date: Apr. 25, 2014

(30) Foreign Application Priority Data
Oct. 28, 2011 (KR) 10-2011-0111431

(51) Int. Cl. C07D 487/04; C07D 401/04; C07D 471/04; C07D 231/56; C07D 403/06; C07D 209/08; C07D 209/88; C07D 403/04

(52) U.S. Cl. CPC C07D 487/04; C07D 209/88; C07D 401/04; C07D 403/04; C07D 231/56; C07D 403/06; C07D 209/08; C07D 471/04
USPC 514/210.21; 548/449; 514/411; 546/200; 514/323; 544/142; 514/232.8; 548/360.1; 514/406; 544/372; 514/254.08; 548/311.4; 514/397; 548/516; 514/412; 544/250; 514/267; 544/280; 514/265.1; 546/111; 514/300; 544/126; 540/575; 514/218

(57) ABSTRACT

The present invention relates to a novel hydroxamate derivatives, more specifically, to novel hydroxamate derivatives having inhibitory activity against Histone Deacetylase (HDAC), isomers thereof, pharmaceutically acceptable salts thereof, hydrates or solvates thereof, use for preparing pharmaceutical compositions, pharmaceutical compositions comprising the same, treatment method using said composition, and a preparing method of novel hydroxamate derivatives. The novel selective hydroxamate derivatives having inhibitory activity against Histone Deacetylase (HDAC) compositions can be used for treatment of inflammatory disease, rheumatoid arthritis, or degenerative disease.

### TECHNICAL FIELD

The present invention relates to a novel hydroxamate derivatives, more specifically, to novel hydroxamate derivatives having inhibitory activity against Histone Deacetylase (HDAC), isomers thereof, pharmaceutically acceptable salts thereof, hydrates or solvates thereof, use for preparing pharmaceutical compositions, pharmaceutical compositions comprising the same, treatment method using said composition, and a preparing method of novel hydroxamate derivatives.

### BACKGROUND ART

The compounds according to the present invention are used to inhibit or treat HDAC mediated diseases. Examples of those diseases are cell proliferative diseases such as cancers, autosomal dominant disorders such as Huntington's disease, gene-related metabolic diseases including fibrosis such as cystic fibrosis, hepatic fibrosis, renal fibrosis, pulmonary fibrosis and dermatofibrosis, autoimmune disease such as rheumatoid arthritis, acute or chronic neurologic diseases such as diabetes and stroke, hypertrophy such as cardiac hypertrophy, hemorrhagic heart failure, amyotrophic lateral sclerosis, glaucoma, eye diseases (related with the neovascularization) and Alzheimer's disease, but the present invention is not limited thereto.

Control for transcription of cells is complicated biological process. One basic principle is control by deformation after the translation of histone proteins H2A/B, H3 and H4 forming histone octamer core complex. Such a complicated N-terminus deformation of lysine residue by an acetylation or methylation, and of serine residue by phosphorylation forms a portion of so-called “histone code”(Strahl & Ellis, Nature 403, 41-45, 2000).

As a simple model, acetylation of positive-charged lysine residue decreases affinity to negative-charged DNA, thereby, into which transcription factor can enter easily.

Histone acetylation and deacetylation are promoted by histone acetyl transferase (HAT) and histone deacetylase (HDAC) respectively. HDAC associates with transcription inhibitor complex, and changes the same into silent structure, which is inactive for chromatin transcription (Mark etc., Nature cancer Rev. 1, 189-202, 2001). HAT associating with transcription activation factor complex is the opposite. There are three different HDACs, namely, group I (HDAC 1-3, 8; Mr=42-55 kDa) which is located in nucleus, and sensitive to inhibition by trichostatin A (TSA), group II (HDAC 4-7, 9, 10; Mr=120-130 kDa) showing TSA sensitivity, and group III (Sir2) which is differentiated by NAB+ dependency and TSA insensibility.

Histone deacetylase (HDAC) inhibitors become a new trend of anticancer drugs doing cell differentiation and apoptosis. Influencing to histone (protein) acetylation and chromatin structure deacetylation by targeting histone deacetylation induces the reprogramming of complicated transcription, for example, reactivation of tumor suppressor gene and suppression of oncogene. There are important non-histone targets for the cancer biology such as heat shock protein (HSP90), tubulin or p53 tumour suppressor protein other than bringing about acetylation of N-terminus lysine residue in core histone protein. Therefore, medical use of HDAC inhibitor is not restricted for anti-cancer therapy since HDAC inhibitor shows effectiveness for inflammatory diseases, rheumatoid arthritis and neurodegenerative disease in an animal model.

HDAC inhibitors known up to now can be classified by four kinds according to their structure, namely, 1) short chain fatty acid (butyric acid, valproic acid), 2) hydroxamic acids (trichostatin A, SAHA, LBH-589), 3) cyclic peptides (desipeptide) and 4) benzamide (MS-275, MGCD-0103) (International Journal of Onocology 33, 637-646, 2008). These many of HDAC inhibitors (SAHA, LBH-589 and MS-275) induce inhibition of growth, differenciation and apoptosis for various transformed cells in culture medium as well as in animal models (Marks, P. A et. al., Curr Opin Oncol. 2001. 13. 477-483), and some HDAC inhibitors such as SAHA, LBH-589 and MS-275 are appraised for the purpose of treatment of various cancers (Johnstone, R. W Nat. Rev. Drug Discov. 2002 1. 287-299). Presently, representative compounds of HDAC inhibitors are, SAHA (US771760, Zolinza, Vorinostat), PXD101 (WO 02/30879, Belinostat) and LBH-589 (WO 02/22577, Panobinostat), which are hydroxamate compounds, and MS-275 (EP8799) and MGCD0103 (WO 04/69823), which are benzamide compounds. Among them, SAHA was approved on October 2006 and has been used for the treatment of CTCL (cutaneous T-cell lymphoma). Diseases for which medicine is efficacious have been additionally expanded, but are known for its lack of effectiveness and side effects (Cancer Res 2006, 66, 5781-5789).

Accordingly, in spite that many HDAC inhibitors have been reported up to now, a novel HDAC inhibitor that is more selective, less side effects and effective is required in the art in order to overcome the lack of effectiveness and side effects (Mol Cancer Res, 5, 981, 2007)

### DISCLOSURE

### Technical Problem

The object of this invention is to provide a novel hydroxamate derivatives, more specifically, to novel hydroxamate derivatives having inhibitory activity against Histone Deacetylase (HDAC), isomers thereof, pharmaceutically acceptable salts thereof, hydrates or solvates thereof, use thereof for preparing pharmaceutical compositions, pharmaceutical compositions comprising the same, treatment method using said composition, and a preparing method of the novel hydroxamate derivatives.

The other object of this invention is to provide a method for preparing a novel hydroxamate derivative.

### Technical Solution

Accordingly, the present inventors have conducted many studies and, as a result, have developed a novel hydroxamate derivative, isomers thereof, pharmaceutically acceptable salts thereof, hydrates or solvates thereof of the following formula 1:

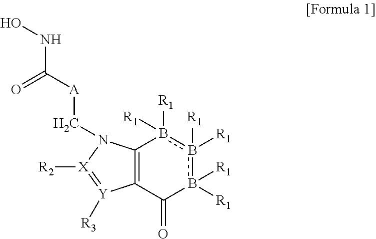

wherein

B are independently C or N;

R₁ are independently absent, -hydrogen, —C₁₋₆ alkyl, —C₂₋₆ alkenyl, —C₃₋₆ cycloalkyl or

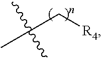

wherein n is 1, 2, 3 or 4, and R₄ is -halogen, —NH(C₁-C₆alkyl), —N(C₁-C₆alkyl)₂, —OH, —O(C₁-C₆alkyl), —S(C₁-C₆alkyl), —N[(C₁-C₆alkyl)(C₁-C₆alcohol)] or 4, 5 or 6 membered heteroaryl or hetero cyclo alkyl compound having 1 to 3 hetero atoms selected independently from the group consisting of O, N and S (wherein heteroaryl or hetero cyclo alkyl compound optionally substituted with -hydrogen, -halogen,

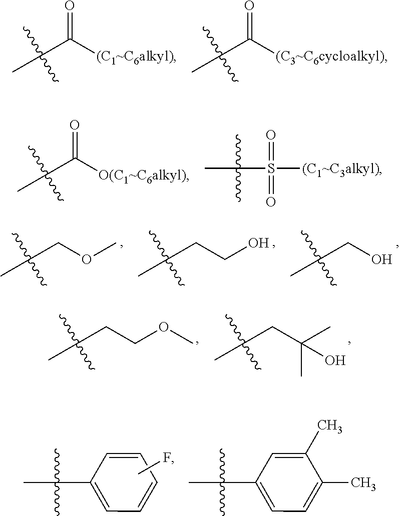

or —C₁-C₆ alkyl);

X and Y are independently C or N;

R₂ and R₃ are independently absent, -hydrogen, -halogen, —CF₃, —CHF₂, —CH₂F, -cyano, -nitro, —C₁-C₆ alkyl, —O(C₁-C₆ alkyl), —NH(C₁-C₆ alkyl), —N(C₁-C₆ alkyl)₂ or —(C₃-C₆ cycloalkyl), or

R₂ and R₃ together with X and Y to which they are bonded may form a 5 or 6 membered aryl or heteroaryl (wherein aryl or heteroaryl has substituent selected independently from -hydrogen, —CF₃, —C₁-C₆ alkyl, —O(C₁-C₆ alkyl), -halogen, —OH, —NH(C₁-C₆ alkyl), —N(C₁-C₆ alkyl)₂ or -nitro); and

A is —C₁-C₅ alkyl, -cycloalkyl, -aryl or -heteroaryl (wherein, alkyl, cycloalkyl, aryl and heteroaryl has substituent selected from -hydrogen, —CF₃, —C₁-C₆ alkyl, —O(C₁-C₆ alkyl), -halogen, —OH, —NH(C₁-C₆ alkyl), —N(C₁-C₆ alkyl)₂ or -nitro).

Preferably, A is

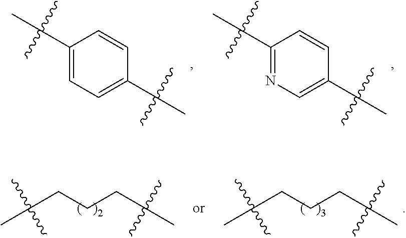

Preferably, B are independently C,

R₁ are independently -hydrogen, methyl or

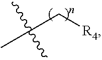

wherein n is 1, and R₄ is -halogen, —NH(C₁-C₆ alkyl), —N(C₁-C₆ alkyl)₂, —OH, —O(C₁-C₆ alkyl), —S(C₁-C₆ alkyl), —N[(C₁-C₆ alkyl)(C₁-C₆ alcohol)] or 4, 5 or 6 membered heteroaryl or hetero cyclo alkyl compound having 1 to 3 atoms selected independently from the group consisting of 0, N and S (wherein heteroaryl or hetero cyclo alkyl compound optionally substituted with -hydrogen, -halogen,

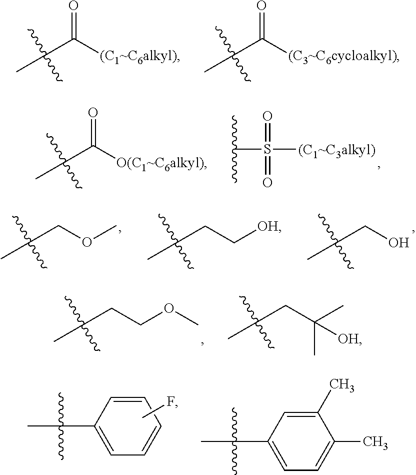

or —C₁-C₆ alkyl); and

A is

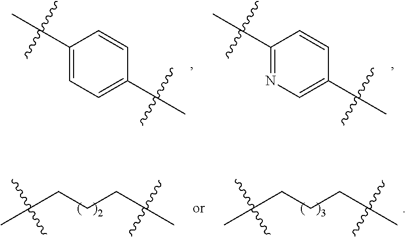

Specific examples of preferred compounds of formula 1 according to the present invention includes:

### Compound 18:

6-(2,2-dimethyl-4-oxo-1,2,3,4-tetrahydrocarbazol-9-yl)-N-hydroxyhexanamide;

### Compound 19:

4-((2,2-dimethyl-4-oxo-1,2,3,4-tetrahydrocarbazol-9-yl)methyl)-N-hydroxybenzamide;

### Compound 20:

7-(2,2-dimethyl-4-oxo-1,2,3,4-tetrahydrocarbazol-9-yl)-N-hydroxyheptanamide;

### Compound 40:

6-(6-bromo-2,2-dimethyl-4-oxo-1,2,3,4-tetrahydrocarbazol-9-yl)-N-hydroxyhexanamide;

### Compound 41:

6-(7-bromo-2,2-dimethyl-4-oxo-1,2,3,4-tetrahydrocarbazol-9-yl)-N-hydroxyhexanamide;

### Compound 45:

4-((6-bromo-2,2-dimethyl-4-oxo-1,2,3,4-tetrahydrocarbazol-9-yl)methyl)-N-hydroxybenzamide;

### Compound 46:

6-(1-bromo-7,7-dimethyl-5-oxo-5,6,7,8-tetrahydrocarbazol-9-yl)-N-hydroxyhexanamide;

### Compound 47:

6-(1,3-dichloro-7,7-dimethyl-5-oxo-5,6,7,8-tetrahydrocarbazol-9-yl)-N-hydroxyhexanamide;

### Compound 48:

6-(1,4-dichloro-7,7-dimethyl-5-oxo-5,6,7,8-tetrahydrocarbazol-9-yl)-N-hydroxyhexanamide;

### Compound 49:

4-((1,4-dichloro-7,7-dimethyl-5-oxo-5,6,7,8-tetrahydrocarbazol-9-yl)methyl)-N-hydroxybenzamide;

### Compound 50:

7-(6-bromo-2,2-dimethyl-4-oxo-1,2,3,4-tetrahydrocarbazol-9-yl)-N-hydroxyheptanamide;

### Compound 51:

7-(1,3-dichloro-7,7-dimethyl-5-oxo-5,6,7,8-tetrahydrocarbazol-9-yl)-N-hydroxyheptanamide;

### Compound 52:

7-(1-bromo-7,7-dimethyl-5-oxo-5,6,7,8-tetrahydrocarbazol-9-yl)-N-hydroxyheptanamide;

### Compound 53:

4-((5-bromo-2,2-dimethyl-4-oxo-1,2,3,4-tetrahydrocarbazol-9-yl)methyl)-N-hydroxybenzamide;

### Compound 54:

4-((7-bromo-2,2-dimethyl-4-oxo-1,2,3,4-tetrahydrocarbazol-9-yl)methyl)-N-hydroxybenzamide;

### Compound 55:

6-(6-fluoro-2,2-dimethyl-4-oxo-1,2,3,4-tetrahydrocarbazol-9-yl)-N-hydroxyhexanamide;

### Compound 56:

7-(6-fluoro-2,2-dimethyl-4-oxo-1,2,3,4-tetrahydrocarbazol-9-yl)-N-hydroxyheptanamide;

### Compound 57:

4-((6-fluoro-2,2-dimethyl-4-oxo-1,2,3,4-tetrahydrocarbazol-9-yl)methyl)-N-hydroxybenzamide;

### Compound 60:

6-(5-fluoro-2,2-dimethyl-4-oxo-2,3,4,4a-tetrahydro-1H-carbazol-9(9aH)-yl)-N-hydroxyhexanamide;

### Compound 71:

4-((6,7-difluoro-2,2-dimethyl-4-oxo-1,2,3,4-tetrahydrocarbazol-9-yl)methyl)-N-hydroxybenzamide;

### Compound 72:

6-(6,7-difluoro-2,2-dimethyl-4-oxo-1,2,3,4-tetrahydrocarbazol-9-yl)-N-hydroxyhexanamide;

### Compound 73:

4-((5,6-difluoro-2,2-dimethyl-4-oxo-1,2,3,4-tetrahydrocarbazol-9-yl)methyl)-N-hydroxybenzamide;

### Compound 74:

6-(5,6-difluoro-2,2-dimethyl-4-oxo-1,2,3,4-tetrahydrocarbazol-9-yl)-N-hydroxyhexanamide;

### Compound 76:

6-(7-fluoro-2,2-dimethyl-4-oxo-1,2,3,4-tetrahydrocarbazol-9-yl)-N-hydroxyhexanamide;

### Compound 85:

N-hydroxy-4-((3-((2-methyl-1H-imidazol-1-yl)methyl)-4-oxo-1,2,3,4-tetrahydrocarbazol-9-yl)methyl)benzamide;

### Compound 86:

N-hydroxy-4-((4-oxo-3-(piperidin-1-ylmethyl)-1,2,3,4-tetrahydrocarbazol-9-yl)methyl)benzamide;

### Compound 87:

N-hydroxy-4-((3-(morpholinomethyl)-4-oxo-1,2,3,4-tetrahydrocarbazol-9-yl)methyl)benzamide;

### Compound 88:

N-hydroxy-4-((4-oxo-3-(pyrrolidin-1-ylmethyl)-1,2,3,4-tetrahydrocarbazol-9-yl)methyl)benzamide;

### Compound 99:

N-hydroxy-4-((4-oxo-1,2,3,4-tetrahydrocarbazol-9-yl)methyl)benzamide;

### Compound 101:

N-hydroxy-4-((3,6,6-trimethyl-4-oxo-4,5,6,7-tetrahydroindazol-1-yl)methyl)benzamide;

### Compound 110:

4-((3-((2,6-dimethylmorpholino)methyl)-4-oxo-1,2,3,4-tetrahydrocarbazol-9-yl)methyl)-N-hydroxybenzamide;

### Compound 111:

N-hydroxy-4-((3-((4-methylpiperazin-1-yl)methyl)-4-oxo-1,2,3,4-tetrahydrocarbazol-9-yl)methyl)benzamide;

### Compound 112:

4-((3-((4-ethylpiperazin-1-yl)methyl)-4-oxo-1,2,3,4-tetrahydrocarbazol-9-yl)methyl)-N-hydroxybenzamide;

### Compound 113:

N-hydroxy-4-((3-((4-isopropylpiperazin-1-yl)methyl)-4-oxo-1,2,3,4-tetrahydrocarbazol-9-yl)methyl)benzamide;

### Compound 114:

N-hydroxy-4-((3-((4-(2-methoxyethyl)piperazin-1-yl)methyl)-4-oxo-1,2,3,4-tetrahydrocarbazol-9-yl)methyl)benzamide;

### Compound 121:

4-((3-((3,3-difluoroazetidin-1-yl)methyl)-4-oxo-1,2,3,4-tetrahydrocarbazol-9-yl)methyl)-N-hydroxybenzamide;

### Compound 122:

4-((2,2-dimethyl-3-(morpholinomethyl)-4-oxo-1,2,3,4-tetrahydrocarbazol-9-yl)methyl)-N-hydroxybenzamide;

### Compound 123:

4-((3-((3,3-difluoropyrrolidin-1-yl)methyl)-4-oxo-1,2,3,4-tetrahydrocarbazol-9-yl)methyl)-N-hydroxybenzamide;

### Compound 126:

4-((2,2-dimethyl-3-((2-methyl-1H-imidazol-1-yl)methyl)-4-oxo-1,2,3,4-tetrahydrocarbazol-9-yl)methyl)-N-hydroxybenzamide;

### Compound 127:

4-((3-((4-(4-fluorophenyl)piperazin-1-yl)methyl)-4-oxo-1,2,3,4-tetrahydrocarbazol-9-yl)methyl)-N-hydroxybenzamide;

### Compound 128:

4-((3-((4-(3,4-dimethylphenyl)piperazin-1-yl)methyl)-4-oxo-1,2,3,4-tetrahydrocarbazol-9-yl)methyl)-N-hydroxybenzamide;

### Compound 129:

4-((3-((dimethylamino)methyl)-2,2-dimethyl-4-oxo-1,2,3,4-tetrahydrocarbazol-9-yl)methyl)-N-hydroxybenzamide;

### Compound 130:

N-hydroxy-4-((3-((4-(methylsulfonyl)piperazin-1-yl)methyl)-4-oxo-1,2,3,4-tetrahydrocarbazol-9-yl)methyl)benzamide;

### Compound 131:

4-((3,3-dimethyl-4-oxo-1,2,3,4-tetrahydrocarbazol-9-yl)methyl)-N-hydroxybenzamide

### Compound 136:

N-hydroxy-4-((3,6,6-trimethyl-4-oxo-4,5,6,7-tetrahydroindol-1-yl)methyl)benzamide;

### Compound 140:

tert-butyl 4-((9-(4-(hydroxycarbamoyl)benzyl)-4-oxo-2,3,4,9-tetrahydro-1H-carbazol-3-yl)methyl)piperazine-1-carboxylate;

### Compound 141:

4-((6-fluoro-3-(morpholinomethyl)-4-oxo-1,2,3,4-tetrahydrocarbazol-9-yl)methyl)-N-hydroxybenzamide;

### Compound 142:

4-((6-fluoro-3-((4-methylpiperazin-1-yl)methyl)-4-oxo-1,2,3,4-tetrahydrocarbazol-9-yl)methyl)-N-hydroxybenzamide;

### Compound 144:

tert-butyl 4-((6-fluoro-9-(4-(hydroxycarbamoyl)benzyl)-4-oxo-2,3,4,9-tetrahydro-1H-carbazol-3-yl)methyl)piperazine-1-carboxylate;

### Compound 145:

4-((3-((3,3-difluoroazetidin-1-yl)methyl)-6-fluoro-4-oxo-1,2,3,4-tetrahydrocarbazol-9-yl)methyl)-N-hydroxybenzamide;

### Compound 156:

4-((3-((4-(cyclopropanecarbonyl)piperazin-1-yl)methyl)-4-oxo-1,2,3,4-tetrahydrocarbazol-9-yl)methyl)-N-hydroxybenzamide;

### Compound 157:

4-((3-fluoro-5-oxo-5,6,7,8-tetrahydrocarbazol-9-yl)methyl)-N-hydroxybenzamide;

### Compound 158:

4-((6-fluoro-3-((2-methyl-1H-imidazol-1-yl)methyl)-4-oxo-1,2,3,4-tetrahydrocarbazol-9-yl)methyl)-N-hydroxybenzamide;

### Compound 166:

N-hydroxy-4-((3,6,6-trimethyl-4-oxo-4,5,6,7-tetrahydroindol-1-yl)methyl)benzamide;

### Compound 179:

N-hydroxy-4-((4-oxo-3,4-dihydropyrimido[4,5-b]indol-9-yl)methyl)benzamide;

### Compound 188:

4-((6-fluoro-3-((4-isopropylpiperazin-1-yl)methyl)-4-oxo-1,2,3,4-tetrahydrocarbazol-9-yl)methyl)-N-hydroxybenzamide;

### Compound 189:

4-((3-((4-ethylpiperazin-1-yl)methyl)-6-fluoro-4-oxo-1,2,3,4-tetrahydrocarbazol-9-yl)methyl)-N-hydroxybenzamide;

### Compound 190:

4-((3-((4-butylpiperazin-1-yl)methyl)-6-fluoro-4-oxo-1,2,3,4-tetrahydrocarbazol-9-yl)methyl)-N-hydroxybenzamide;

### Compound 191:

4-((6-fluoro-3-((4-(2-hydroxy-2-methylpropyl)piperazin-1-yl)methyl)-4-oxo-1,2,3,4-tetrahydrocarbazol-9-yl)methyl)-N-hydroxybenzamide;

### Compound 192:

4-((6-fluoro-2,2-dimethyl-3-((2-methyl-1H-imidazol-1-yl)methyl)-4-oxo-1,2,3,4-tetrahydrocarbazol-9-yl)methyl)-N-hydroxybenzamide;

### Compound 193:

N-hydroxy-6-(3-(morpholinomethyl)-4-oxo-1,2,3,4-tetrahydrocarbazol-9-yl)hexanamide;

### Compound 194:

N-hydroxy-6-(3-((2-methyl-1H-imidazol-1-yl)methyl)-4-oxo-1,2,3,4-tetrahydrocarbazol-9-yl)hexanamide;

### Compound 203:

N-hydroxy-4-((3-(3-methylbut-2-enyl)-4-oxo-3,4,5,6-tetrahydropyrrolo[2,3-d]pyrimidin-7-yl)methyl)benzamide;

### Compound 204:

6-(3-((3,3-difluoropyrrolidin-1-yl)methyl)-4-oxo-1,2,3,4-tetrahydrocarbazol-9-yl)-N-hydroxyhexanamide;

### Compound 205:

N-hydroxy-6-(3-((4-methylpiperazin-1-yl)methyl)-4-oxo-1,2,3,4-tetrahydrocarbazol-9-yl)hexanamide;

### Compound 206:

6-(3-((4-ethylpiperazin-1-yl)methyl)-4-oxo-1,2,3,4-tetrahydrocarbazol-9-yl)-N-hydroxyhexanamide;

### Compound 207:

4-((6-fluoro-2,2-dimethyl-3-(morpholinomethyl)-4-oxo-1,2,3,4-tetrahydrocarbazol-9-yl)methyl)-N-hydroxybenzamide;

### Compound 208:

6-(3-((4-butylpiperazin-1-yl)methyl)-4-oxo-1,2,3,4-tetrahydrocarbazol-9-yl)-N-hydroxyhexanamide;

### Compound 209:

4-((6-fluoro-2,2-dimethyl-3-((4-methylpiperazin-1-yl)methyl)-4-oxo-1,2,3,4-tetrahydrocarbazol-9-yl)methyl)-N-hydroxybenzamide;

### Compound 220:

4-((2,3-dimethyl-4-oxo-4,5,6,7-tetrahydroindol-1-yl)methyl)-N-hydroxybenzamide;

### Compound 228:

N-hydroxy-4-((1-oxo-1,2,3,4-tetrahydropyrido[4,3-b]indol-5-yl)methyl)benzamide;

### Compound 232:

N-hydroxy-4-((3-methyl-4-oxo-4,5,6,7-tetrahydropyrrolo[3,2-c]pyridin-1-yl)methyl)benzamide;

### Compound 235:

6-(2,3-dimethyl-4-oxo-4,5,6,7-tetrahydroindol-1-yl)-N-hydroxyhexanamide;

### Compound 236:

N-hydroxy-4-((2,3,6,6-tetramethyl-4-oxo-4,5,6,7-tetrahydroindol-1-yl)methyl)benzamide;

### Compound 237:

4-((6,6-dimethyl-4-oxo-3-(trifluoromethyl)-4,5,6,7-tetrahydroindazol-1-yl)methyl)-N-hydroxybenzamide;

### Compound 249:

N-hydroxy-4-((2-(2-morpholinoethyl)-1-oxo-1,2,3,4-tetrahydropyrido[4,3-b]indol-5-yl)methyl)benzamide;

### Compound 250:

N-hydroxy-4-((3-methyl-5-(2-morpholinoethyl)-4-oxo-4,5,6,7-tetrahydropyrrolo[3,2-c]pyridin-1-yl)methyl)benzamide;

### Compound 251:

N-hydroxy-4-((2-(2-(4-methylpiperazin-1-yl)ethyl)-1-oxo-1,2,3,4-tetrahydropyrido[4,3-b]indol-5-yl)methyl)benzamide;

### Compound 266:

4-((8-fluoro-2-(2-morpholinoethyl)-1-oxo-1,2,3,4-tetrahydropyrido[4,3-b]indol-5-yl)methyl)-N-hydroxybenzamide;

### Compound 267:

(S)—N-hydroxy-4-((2-(2-(2-(hydroxymethyl)pyrrolidin-1-yl)ethyl-oxo-1,2,3,4, tetrahydropyrido[4,3-b]indol-5-yl)methyl)benzamide;

### Compound 268:

4-((2,3-dimethyl-5-(morpholinomethyl)-4-oxo-4,5,6,7-tetrahydroindol-1-yl)methyl)-N-hydroxybenzamide;

### Compound 283:

4-((6-fluoro-3-(((R)-3-fluoropyrrolidin-1-yl)methyl)-4-oxo-1,2,3,4-tetrahydrocarbazol-9-yl)methyl)-N-hydroxybenzamide;

### Compound 284:

4-((6-fluoro-3-((4-(2-methoxyethyl)piperazin-1-yl)methyl)-4-oxo-1,2,3,4-tetrahydrocarbazol-9-yl)methyl)-N-hydroxybenzamide;

### Compound 285:

(S)-4-((8-fluoro-2-(2-(2-(methoxymethyl)pyrrolidin-1-yl)ethyl)-1-oxo-1,2,3,4-tetrahydropyrido[4,3-b]indol-5-yl)methyl)-N-hydroxybenzamide;

### Compound 286:

4-((8-fluoro-2-(2-(4-(hydroxymethyl)piperidin-1-yl)ethyl)-1-oxo-1,2,3,4-tetrahydropyrido[4,3-b]indol-5-yl)methyl)-N-hydroxybenzamide;

### Compound 287:

4-((8-fluoro-2-(2-(4-methylpiperazin-1-yl)ethyl)-1-oxo-1,2,3,4-tetrahydropyrido[4,3-b]indol-5-yl)methyl)-N-hydroxybenzamide;

### Compound 288:

4-((8-fluoro-2-(2-(4-(2-hydroxyethyl)piperazin-1-yl)ethyl)-1-oxo-1,2,3,4-tetrahydropyrido[4,3-b]indol-5-yl)methyl)-N-hydroxybenzamide;

### Compound 289:

(R)-4-((8-fluoro-2-(2-(2-(hydroxymethyl)pyrrolidin-1-yl)ethyl)-1-oxo-1,2,3,4-tetrahydropyrido[4,3-b]indol-5-yl)methyl)-N-hydroxybenzamide;

### Compound 290:

(S)-4-((8-fluoro-2-(2-(2-(hydroxymethyl)pyrrolidin-1-yl)ethyl)-1-oxo-1,2,3,4-tetrahydropyrido[4,3-b]indol-5-yl)methyl)-N-hydroxybenzamide;

### Compound 291:

4-((8-fluoro-2-(2-(4-methyl-1,4-diazepan-1-yl)ethyl)-1-oxo-1,2,3,4-tetrahydropyrido[4,3-b]indol-5-yl)methyl)-N-hydroxybenzamide;

### Compound 292:

4-((8-fluoro-2-(2-((2-hydroxyethyl)(methyl)amino)ethyl)-1-oxo-1,2,3,4-tetrahydropyrido[4,3-b]indol-5-yl)methyl)-N-hydroxybenzamide;

### Compound 305:

4-((8-fluoro-1-oxo-1,2-dihydropyrido[4,3-b]indol-5-yl)methyl)-N-hydroxybenzamide;

### Compound 306:

4-((8-fluoro-1-oxo-1,2,3,4-tetrahydropyrido[4,3-b]indol-5-yl)methyl)-N-hydroxybenzamide;

### Compound 321:

6-(6-fluoro-3-(morpholinomethyl)-4-oxo-1,2,3,4-tetrahydrocarbazol-9-yl)-N-hydroxyhexanamide;

### Compound 322:

6-(6-fluoro-3-((4-(4-fluorophenyl)piperazin-1-yl)methyl)-4-oxo-1,2,3,4-tetrahydrocarbazol-9-yl)-N-hydroxyhexanamide;

### Compound 323:

6-(3-((2,6-dimethylmorpholino)methyl)-6-fluoro-4-oxo-1,2,3,4-tetrahydrocarbazol-9-yl)-N-hydroxyhexanamide;

### Compound 324:

tert-butyl 4-((6-fluoro-9-(6-(hydroxyamino)-6-oxohexyl)-4-oxo-2,3,4,9-tetrahydro-1H-carbazol-3-yl)methyl)piperazine-1-carboxylate;

### Compound 325:

7-(3-fluoro-5-oxo-5,6,7,8-tetrahydrocarbazol-9-yl)-N-hydroxyheptanamide;

### Compound 326:

6-(3-((4-butylpiperazin-1-yl)methyl)-6-fluoro-4-oxo-1,2,3,4-tetrahydrocarbazol-9-yl)-N-hydroxyhexanamide;

### Compound 328:

7-(2,3-dimethyl-4-oxo-4,5,6,7-tetrahydroindol-1-yl)-N-hydroxyheptanamide;

### Compound 344:

7-(6-fluoro-3-(morpholinomethyl)-4-oxo-1,2,3,4-tetrahydrocarbazol-9-yl)-N-hydroxyheptanamide;

### Compound 345:

N-hydroxy-6-(2,6,6-trimethyl-4-oxo-4,5,6,7-tetrahydroindol-1-yl)hexanamide;

### Compound 346:

N-hydroxy-7-(2-methyl-4-oxo-4,5,6,7-tetrahydroindol-1-yl)heptanamide;

### Compound 347:

N-hydroxy-7-(2,6,6-trimethyl-4-oxo-4,5,6,7-tetrahydroindol-1-yl)heptanamide;

### Compound 350:

N-hydroxy-6-(2,3,6,6-tetramethyl-4-oxo-4,5,6,7-tetrahydroindol-1-yl)hexanamide;

Specific examples of more preferred compounds of formula 1 according to the present invention includes:

### Compound 87:

N-hydroxy-4-((3-(morpholinomethyl)-4-oxo-1,2,3,4-tetrahydrocarbazol-9-yl)methyl)benzamide;

### Compound 142:

4-((6-fluoro-3-((4-methylpiperazin-1-yl)methyl)-4-oxo-1,2,3,4-tetrahydrocarbazol-9-yl)methyl)-N-hydroxybenzamide;

### Compound 237:

4-((6,6-dimethyl-4-oxo-3-(trifluoromethyl)-4,5,6,7-tetrahydroindazol-1-yl)methyl)-N-hydroxybenzamide; and

### Compound 249:

N-hydroxy-4-((2-(2-morpholinoethyl)-1-oxo-1,2,3,4-tetrahydropyrido[4,3-b]indol-5-yl)methyl)benzamide.

The present invention also provides pharmaceutical composition comprising the hydroxamate derivative of the formula 1, isomers thereof, pharmaceutically acceptable salts thereof, hydrates or solvates thereof; and pharmaceutically acceptable carriers thereof.

Preferably, the composition is used for prevention or treatment of a disease associated with HDAC activity.

Preferably, said disease associated with HDAC activity is inflammatory disease, rheumatoid arthritis or neurodegenerative disease.

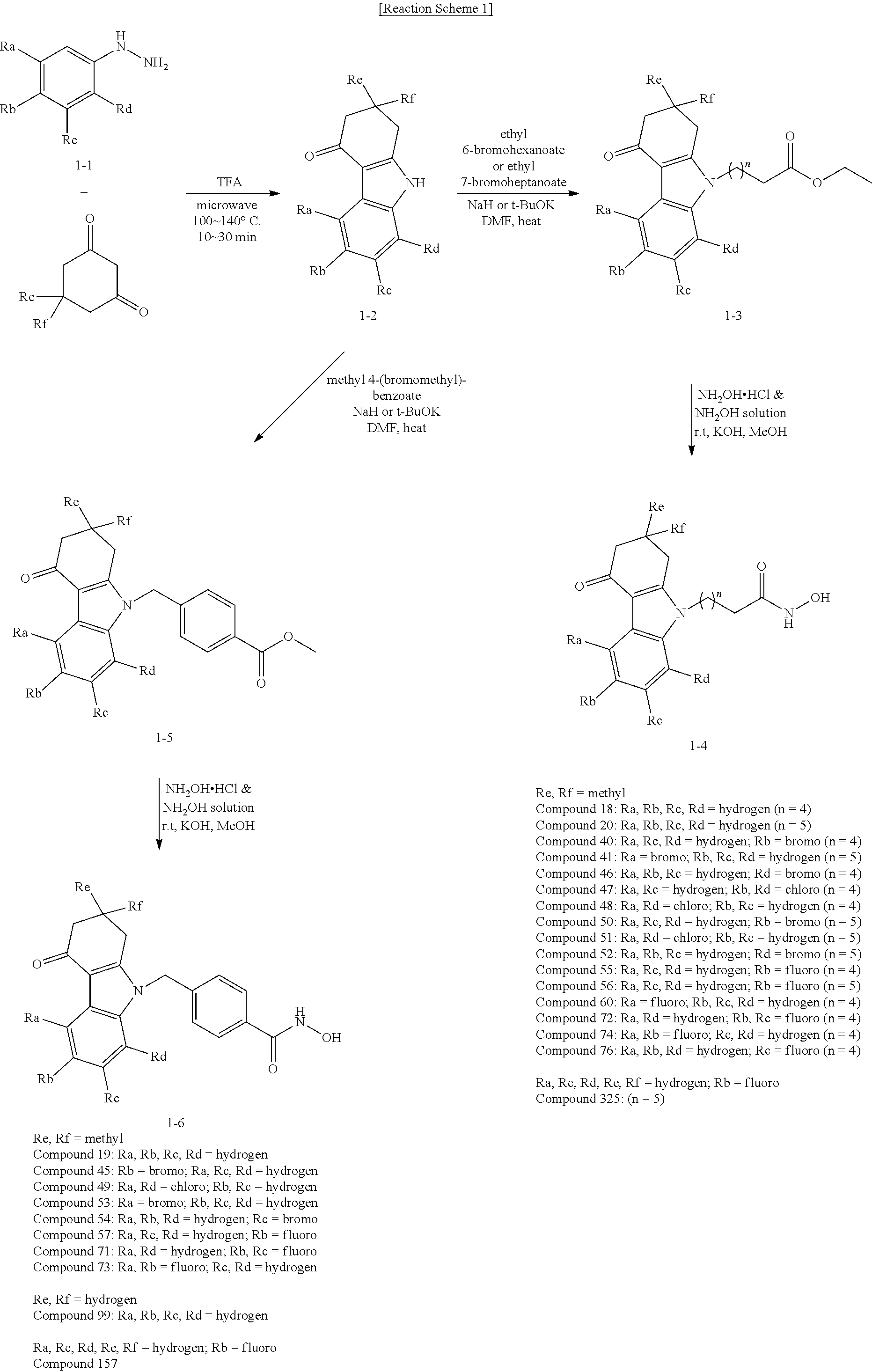

As shown in the reaction scheme 1, the compound of formula 1-1 and various cyclohexanon derivatives are subjected to a Fisher indole synthesis (Bioorganic & Medicinal Chemistry Letters 18, 3517-3521) in microwave reactor at 100 to 140° C. for 10 to 30 minutes to produce the compound of formula 1-2 which is then subjected to a substitution reaction with ethyl 6-bromohexanoate or ethyl 7-bromoheptanoate, thereby synthesizing the compound of formula 1-3. Then, potassium hydroxide (KOH), methanol and hydroxylamine hydrochloride (NH₂OH HCl) are added dropwise to the compound of formula 1-3 and reacted at room temperature, thus systhesizing the desired compounds 18, 20, 40, 41, 46, 47, 48, 50, 51, 52, 55, 56, 60, 72, 74, 76 and 325.

Methyl 4-(bromomethyl)benzoate and NaH or t-BuOK are added to the compound of formular 1-2 and reacted 40 to 60° C. to synthesize the compound of formula 1-5. Then, potassium hydroxide (KOH), methanol, hydroxylamine hydrochloride (NH₂OH HCl) and hydroxylamine aqueous solution are added in order dropwise to the compound of formula 1-5 and reacted at room temperature, thus systhesizing the desired compounds 19, 45, 49, 53, 54, 57, 71, 73, 99 and 157.

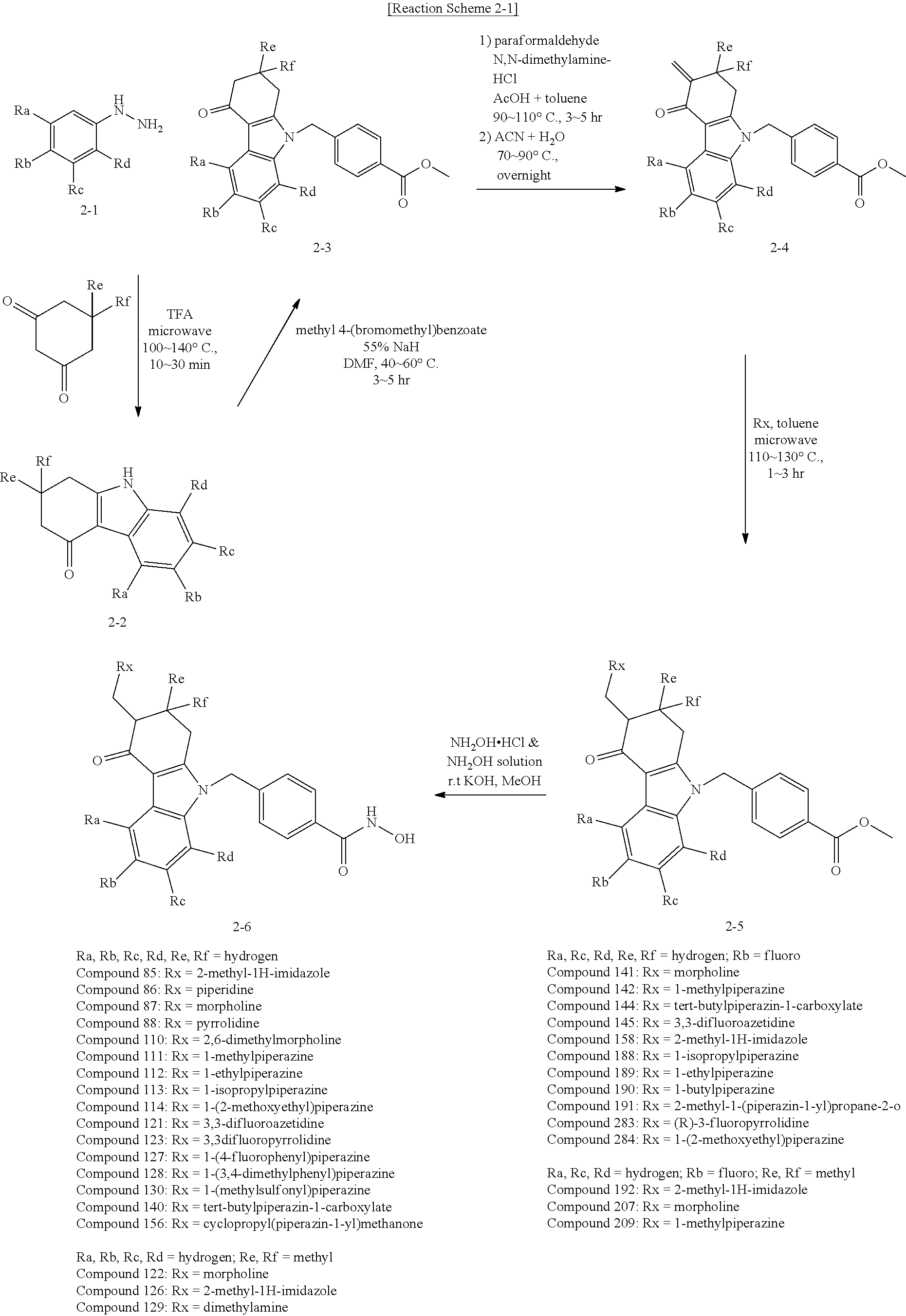

As shown in the reaction scheme 2-1, the compound of formula 2-1 and various cyclohexanon derivatives are subjected to a Fisher indole synthesis in microwave reactor at 100 to 140° C. for 10 to 30 minutes to produce the compound of formula 2-2 which is then allowed to react with methyl 4-(bromomethyl benzoate and NaH at 40 to 60° C. thereby synthesizing the compound of formula 2-3. The obtained compound of formula 2-3 is subjected to a Mannich reaction [U.S. Pat. No. 3,634,430A, U.S. Pat. No. 3,740,404A, U.S. Pat. No. 4,957,609A] with para-formaldehyde and amine compound (Rx) to synthesize the compound of formula 2-5 via the intermediate compound of formula 2-4. Then, potassium hydroxide (KOH), methanol and hydroxylamine hydrochloride (NH₂OH HCl) are added in order dropwise to the compound of formula 2-5 and reacted at room temperature, thus systhesizing the desired compounds 85, 86, 87, 88, 110, 111, 112, 113, 114, 121, 122, 123, 126, 127, 128, 129, 130, 140, 141, 142, 144, 145, 156, 158, 188, 189, 190, 191, 192, 207, 209, 283, and 284.

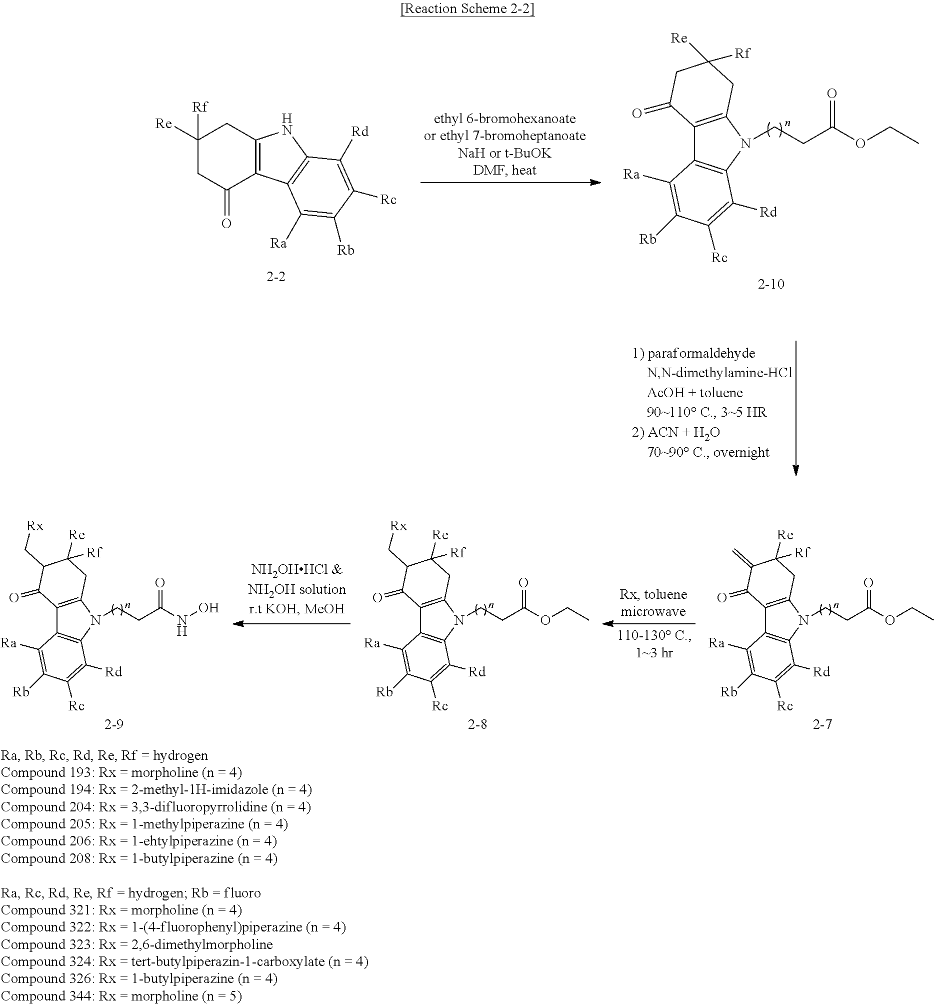

As shown in the reaction scheme 2-2, the compound of formula 2-2 is subjected to a substitution reaction with ethyl 6-bromohexanoate or ethyl 7-bromoheptanoate to synthesize the compound of formula 2-10 (the same as the compound of formula 1-3 in the Reaction Scheme 1) which is then subjected to a Mannich reaction [U.S. Pat. No. 3,634,430A, U.S. Pat. No. 3,740,404A, U.S. Pat. No. 4,957,609A] with para-formaldehyde and amine compound (Rx) to synthesize the compound of formula 2-8 via the intermediate compound of formula 2-7. Then, potassium hydroxide (KOH), methanol and hydroxylamine hydrochloride (NH₂OH HCl) are added in order dropwise to the compound of formula 2-8 and reacted at room temperature, thus systhesizing the desired compounds 193, 194, 204, 205, 206, 208, 321, 322, 323, 324, 326 and 344.

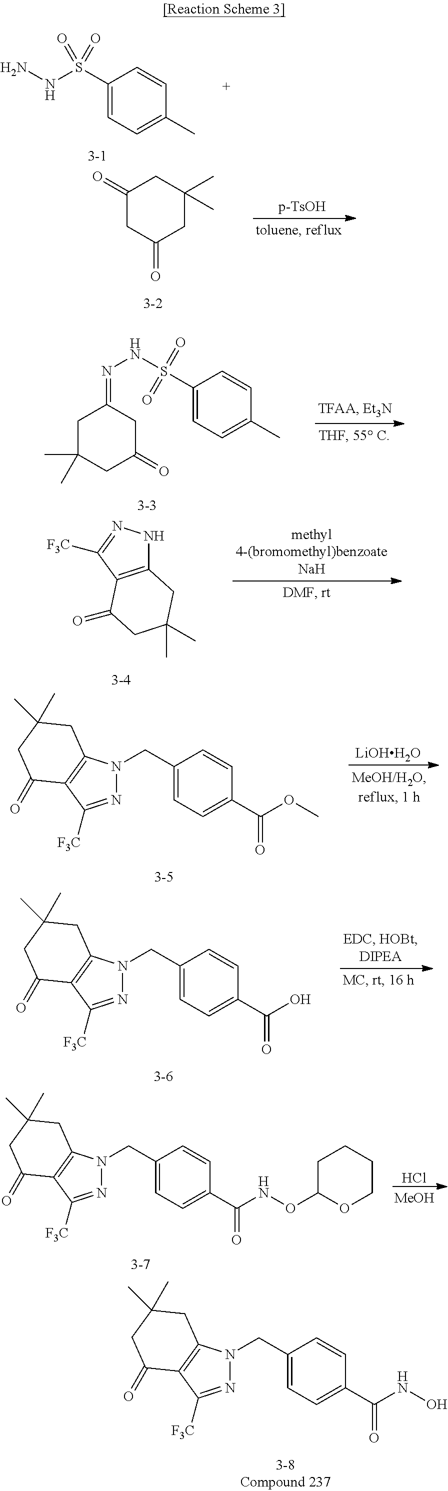

As shown in the reaction scheme 3, p-toluenesulfonylhydrazide, 5,5-dimethyl-1,3-cyclohexandion, and p-toluenesulfonic acid monohydrate are added to toluene, refluxed with stirring, and cooled in room temperature to obtain the compound of formula 3-3. Trifluoroacetic anhydride and triethylamine are added to the obtained compound of formula 3-3 and reacts at 55° C. and is cooled in room temperature thereby synthesizing the compound of formula 3-4, which is allowed to react with methyl 4-(bromomethyl)benzoate and NaH in room temperature to synthesize the compound of formula 3-5, which is then subjected to hydrolyaztion reaction with LiOH to be converted into compound 3-6. The obtained compound of formula 3-6 is subjected to protection and deprotection reaction, thus systhesizing the desired compound 237.

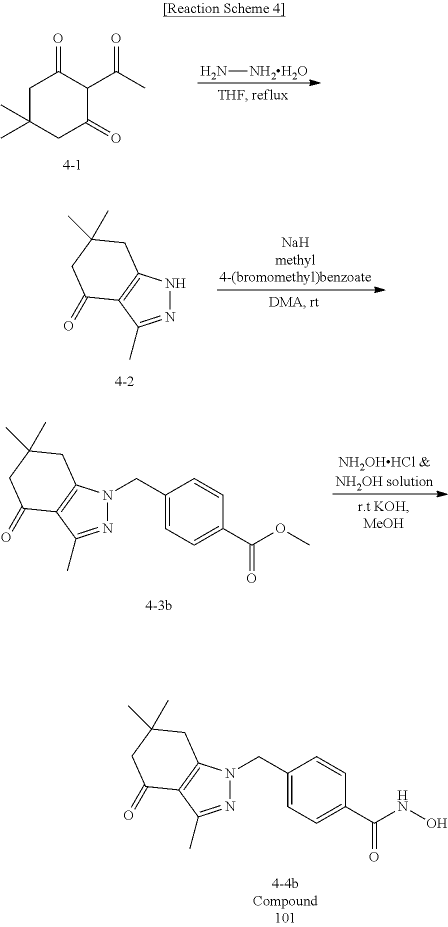

As shown in the reaction scheme 4, 2-acetyldimedone and hydrazine hydrate are allowed to refluxed with stirring for 3 hours to produce the compound of formula 4-2, which is allowed to react with methyl 4-(bromomethyl) benzoate and NaH in room temperature to synthesize the compound of formula 4-3b. Then, potassium hydroxide (KOH), methanol and hydroxylamine hydrochloride (NH₂OH HCl) are added dropwise to the compound of formula 4-3b and reacted at room temperature, thus systhesizing the desired compound 101.

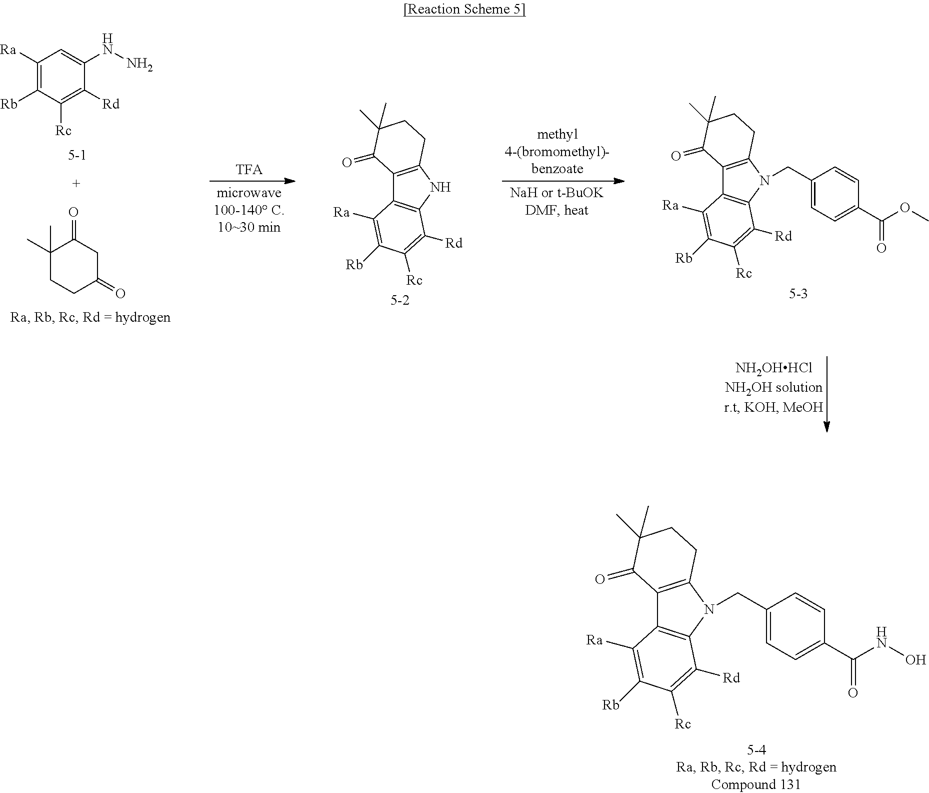

As shown in the reaction scheme 5, 4,4-dimetylcyclohexane-1,3-dion is subjected to a Fisher indole synthesis (Bioorganic & Medicinal Chemistry Letters 18, 3517-3521) in microwave reactor at 100 to 140° C. for 10 to 30 minutes to produce the compound of formula 5-2, which is then subjected to a substitution reaction with methyl 4-(bromomethyl)benzoate and NaH, thereby synthesizing the compound of formula 5-3. Then, potassium hydroxide (KOH), methanol and hydroxylamine hydrochloride (NH₂OH HCl) are added dropwise to the compound of formula 5-3 and reacted at room temperature, thus systhesizing the desired compound 131.

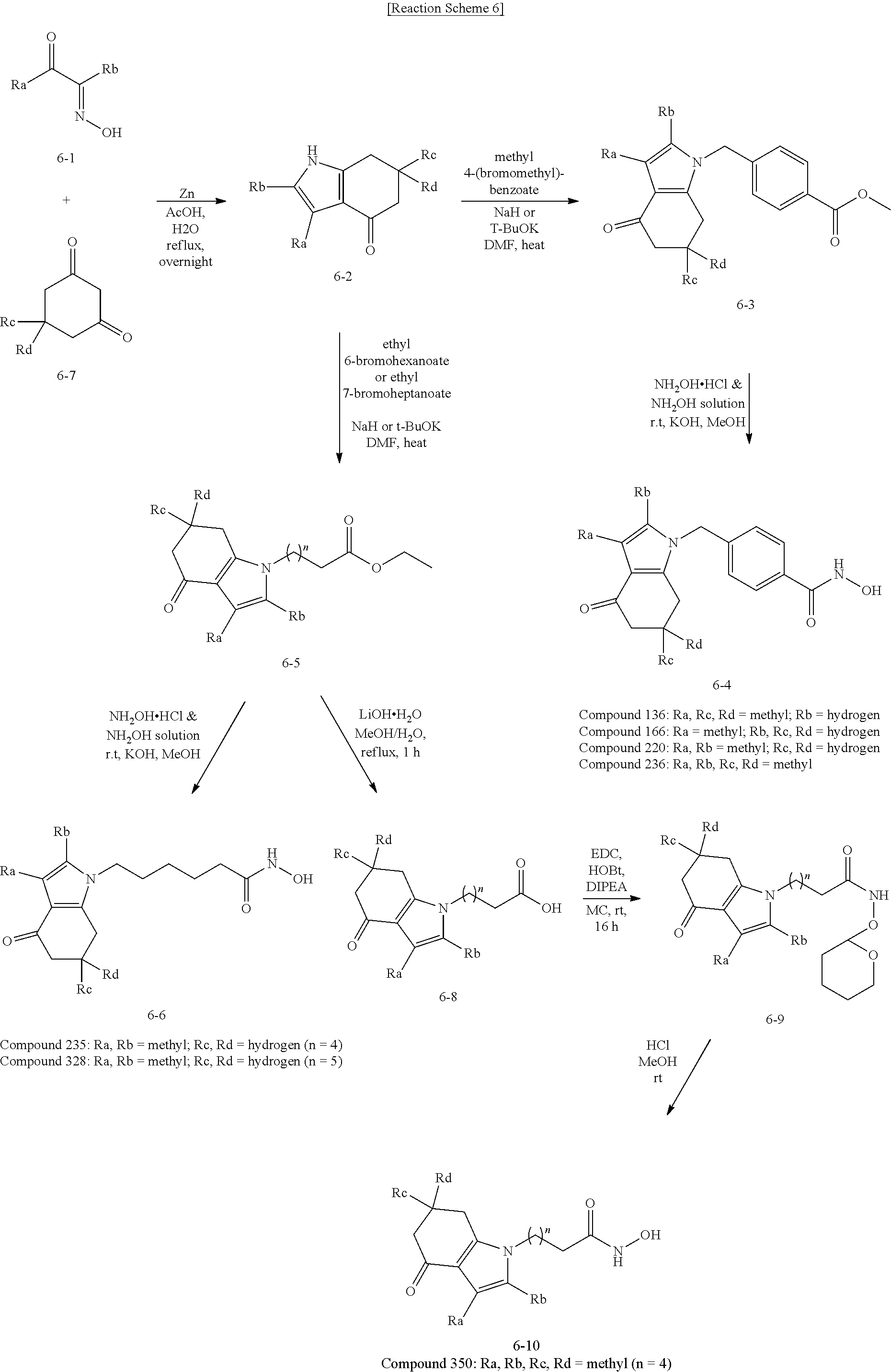

As shown in the reaction scheme 6, the compound of formula 6-7 (cyclohexane-1,3-dion derivative) and the compound of formula 6-1 are subjected to the zinc(Zn) reduction and cyclization reaction to synthesize the indole compound of formula 6-2, which is allowed to react with methyl 4-(bromomethyl) benzoate using NaH or t-BuOK at 40 to 60° C. to synthesize the compound of formula 6-3. Then, potassium hydroxide (KOH), methanol and hydroxylamine hydrochloride (NH₂OH HCl) are added dropwise to the compound of formula 6-3 and reacted at room temperature, thus systhesizing the desired compounds 136, 166, 220 and 236.

The compound of formula 6-2 is subjected to substitution reaction with ethyl 6-bromohexanoate or ethyl 7-bromoheptanoate to synthesize the compound of formula 6-5. Then, potassium hydroxide (KOH), methanol and hydroxylamine hydrochloride (NH₂OH HCl) are added dropwise to the compound of formula 6-5 and reacted at room temperature, thus systhesizing the desired compounds 235 and 328.

The compound of formula 6-2 is allowed to react with ethyl 6-bromohexanoate using NaH in room temperature to synthesize the compound of formula 6-5, which is then subjected to hydrolyaztion reaction with LiOH to be converted into compound of formula 6-8. Then the compound of formula 6-8 is subjected to amide coupling reaction to synthesize the compound of formula 6-9, which is treated with acid (HCl), thus synthesizing the desired compound 350.

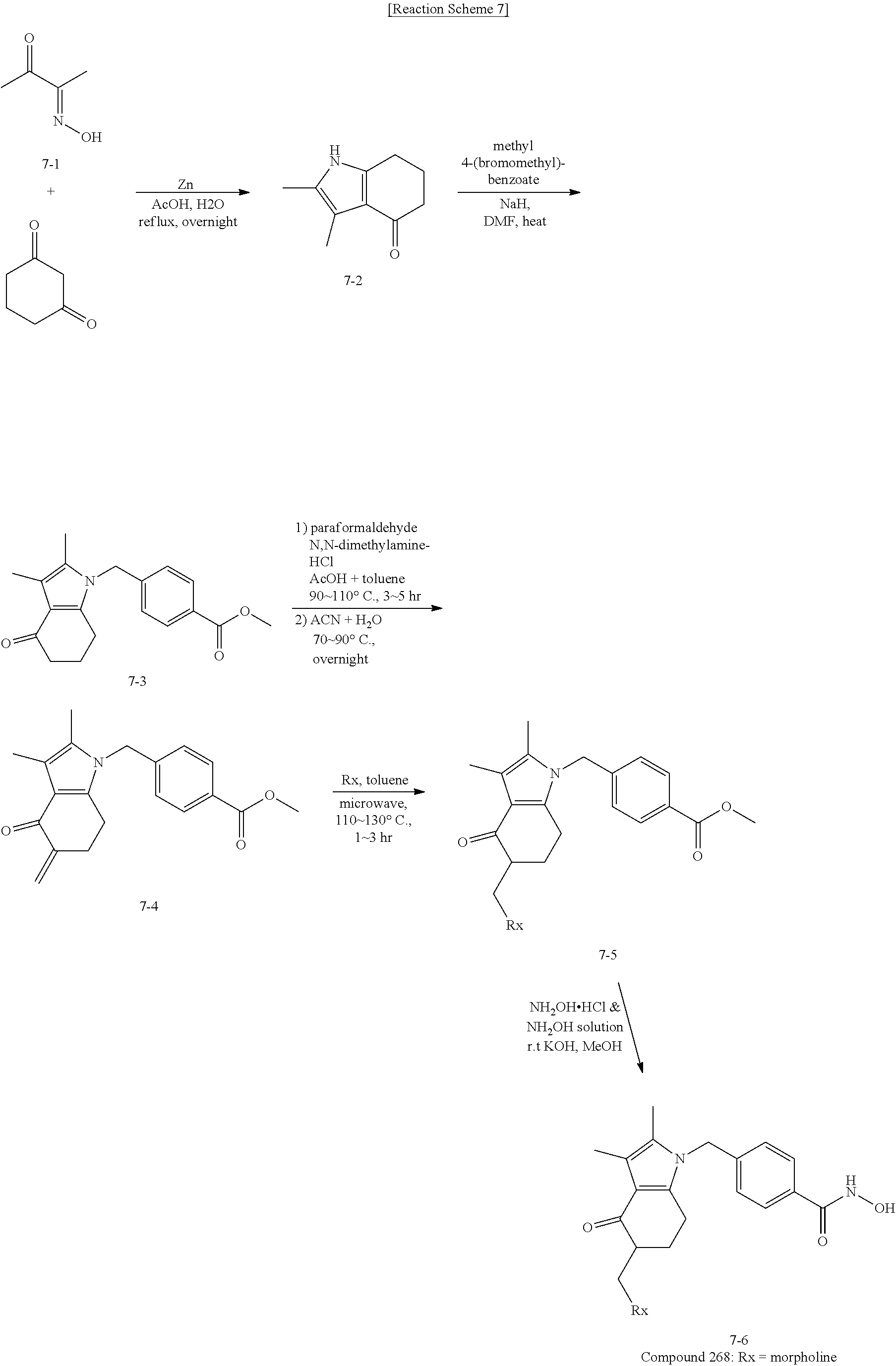

As shown in the reaction scheme 7, cyclohexane-1,3-dion is subjected to the zinc(Zn) reduction and cyclization reaction to produce the indole compound of formula 7-2, which is allowed to react with methyl 4-(bromomethyl) benzoate and NaH at 40 to 60° C. to synthesize the compound of formula 7-3.

The obtained compound of formula 7-3 reacts with para-formaldehyde to synthesize the compound of formula 7-4 which is subjected to a Mannich reaction [U.S. Pat. No. 3,634,430A, U.S. Pat. No. 3,740,404A, U.S. Pat. No. 4,957,609A] with amine compound (Rx) to synthesize the compound of formula 7-5. Then, potassium hydroxide (KOH), methanol and hydroxylamine hydrochloride (NH₂OH HCl) are added dropwise to the compound of formula 7-5 and reacted at room temperature, thus systhesizing the desired compound 268.

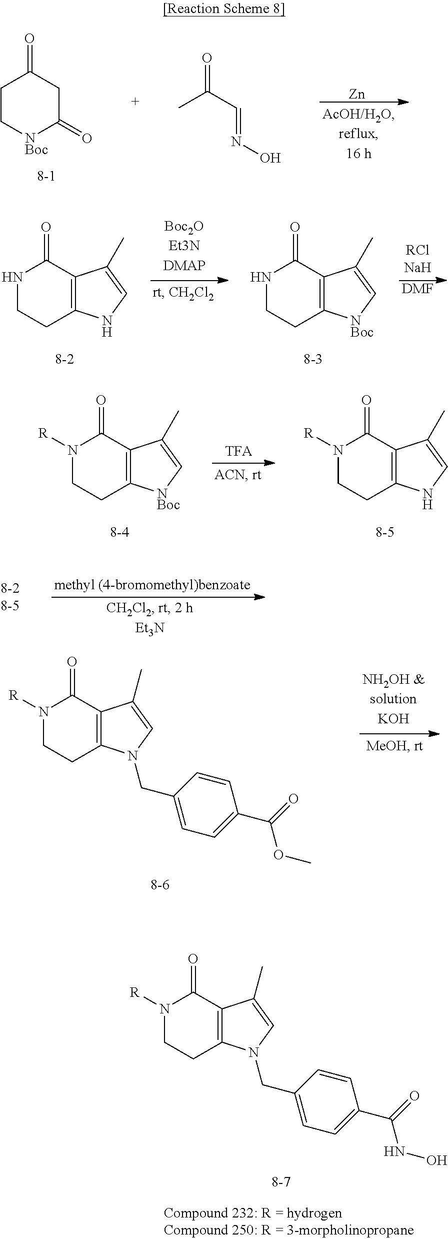

As shown in the reaction scheme 8, the compound of formula 8-1 reacts with anti-pyruvic aldehyde 1-oxime using Zn-dust to carry out reduction and cyclization thereby synthesizing indole compound of formula 8-2, which is reacted with di-tert-butyl dicarbonate to synthesize the compound of formula 8-3 having protecting group. Then, the compound of formula 8-3 is subjected to substitution reaction with N-(2-Chloroethyl)morpholine to synthesize the compound of formula 8-4. The protecting group is deprotected from the compound of formula 8-4. To the compound of formula 8-4, methyl 4-(bromomethyl)benzoate is added, and then potassium hydroxide (KOH), methanol and hydroxylamine hydrochloride (NH₂OH HCl) are added in order dropwise and reacted at room temperature, thus systhesizing the desired compound 250. The desired compound 232, which has no substitution group, is synthesized with the compound of formula 8-2 according to the same process as above.

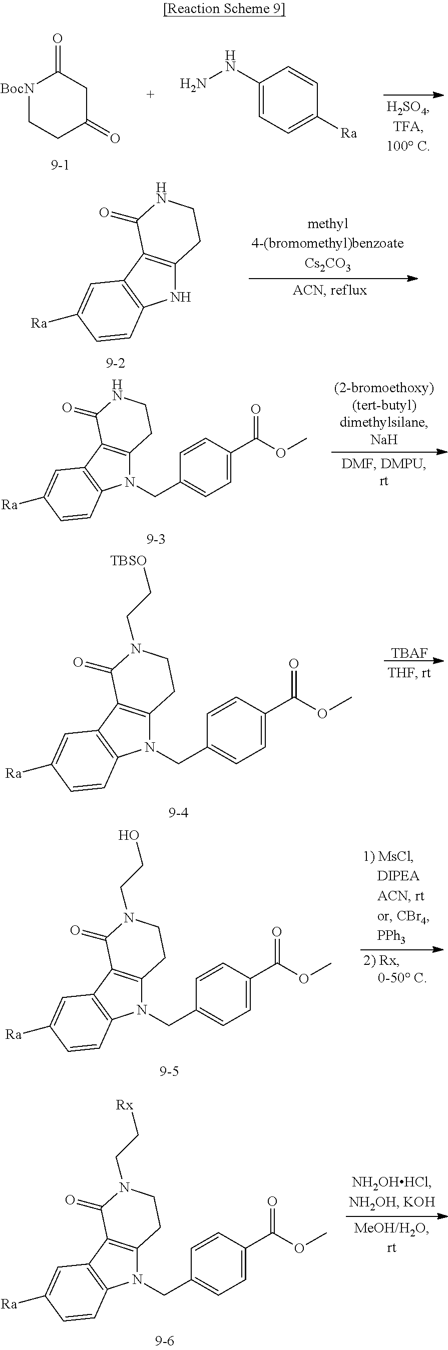

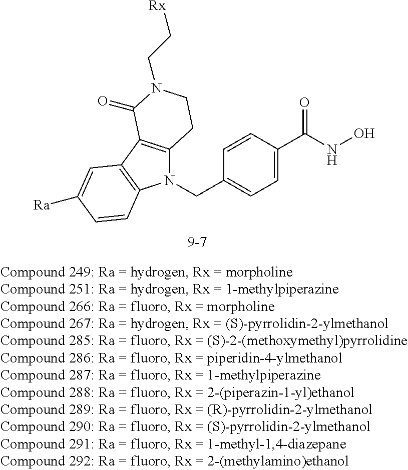

As shown in the reaction scheme 9, the compound of formula 9-1 and phenylhydrazine derivatives are subjected to a Fisher indole synthesis method (Bioorganic & Medicinal Chemistry Letters 18, 3517-3521) at 100° C. for 16 hours to produce the compound of formula 9-2, which is then subjected to a substitution reaction with methyl 4-(bromomethyl)benzoate and Cs₂CO₃, thereby synthesizing the compound of formula 9-3. An etoxy group of which amide is protected by TBS is added to the compound of formula 9-3 to synthesize the compound of formula 9-4. The protecting group is deprotected from the compound of formula 9-4 by TBAF to synthesize the compound of formula 9-5. Various ethyl tertiary amines are introduced to the compound of formula 9-5 to produce the compound of formula 9-6, then, potassium hydroxide (KOH), methanol and hydroxylamine hydrochloride (NH₂OH HCl) are added dropwise to the compound of formula 9-6 and reacted at room temperature, thus systhesizing the desired compounds 249, 251, 266, 267, 285, 286, 287, 288, 289, 290, 291 and 292.

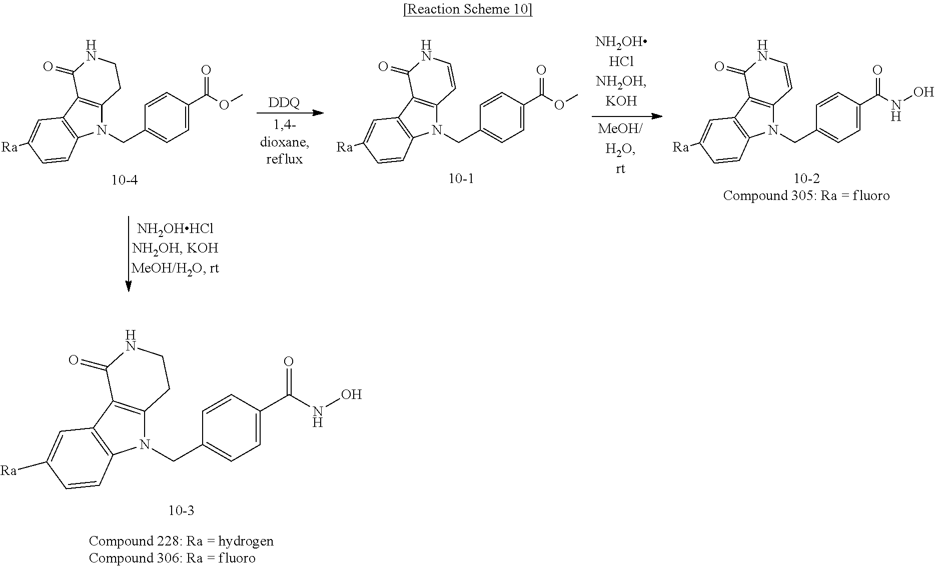

As shown in the reaction scheme 10, the compound of formula 10-4 (the same as the compound of formula 9-3 in the Reaction Scheme 9) is subjected to an oxidation reaction with 2,3-dichloro-5,6-dicyanobenzoquinone to produce the compound of formula 10-1, then, potassium hydroxide (KOH), methanol and hydroxylamine hydrochloride (NH₂OH HCl) are added in order dropwise to the compound of formula 10-1 and reacted at room temperature, thus systhesizing the desired compound 305.

Potassium hydroxide (KOH), methanol and hydroxylamine hydrochloride (NH₂OH HCl) are added in order dropwise to the compound of formula 10-4 (the same as the compound of formula 9-3 in the Reaction Scheme 9) and reacted at room temperature, thus systhesizing the desired compounds 228 and 306.

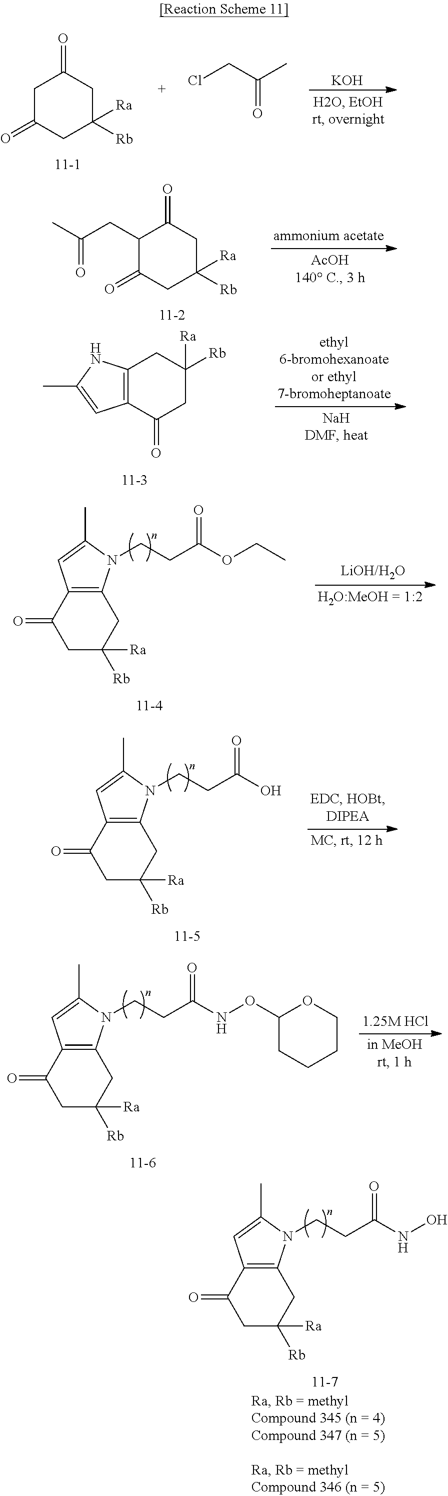

As shown in the reaction scheme 11, 1-chloropropane-2-on is added to the compound of formula 11-1 and stirred for one day to produce the compound of formula 11-2, to which ammonium acetate is added, and refluxed with stirring at 140° C. for 3 hours to synthesize the compound of formula 11-3. Then, the compound of formula 11-3 is subjected to substitution reaction with ethyl 6-bromohexanoate or ethyl 7-bromoheptanoate to synthesize the compound of formula 11-4, which is subjected to hydrolyzation reaction with lithium hydroxide monohydrate, thereby synthesizing the compound of formula 11-5. The compound of formula 11-5 is subjected to EDC imide coupling reaction, thereby synthesizing the compound of formula 11-6, to which 1.25 M HCl in methanol is added to delete THP, thus synthesizing the desired compounds 345, 346 and 347.

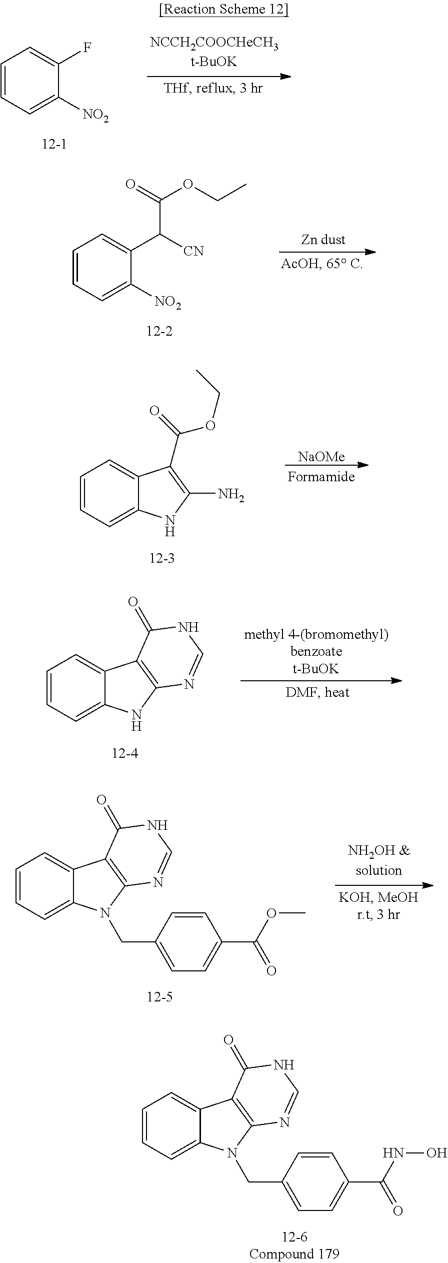

As shown in the reaction scheme 12, the compound of formula 12-1 is subjected to addition reaction with ethylcyanoacetate to produce the compound of formula 12-2, which is then subjected to zinc(Zn) reduction and cyclization reaction to synthesize the indole compound of formula 12-3, which is allowed to react with formamide to synthesize tricyclic compound of formula 12-4. Then, methyl 4-(bromomethyl)benzoate is added to the compound of formula 12-4 to synthesize the compound of formula 12-5. Potassium hydroxide (KOH), methanol and hydroxylamine hydrochloride (NH₂OH HCl) are added dropwise to the compound of formula 12-5 and reacted at room temperature, thus systhesizing the desired compound 179.

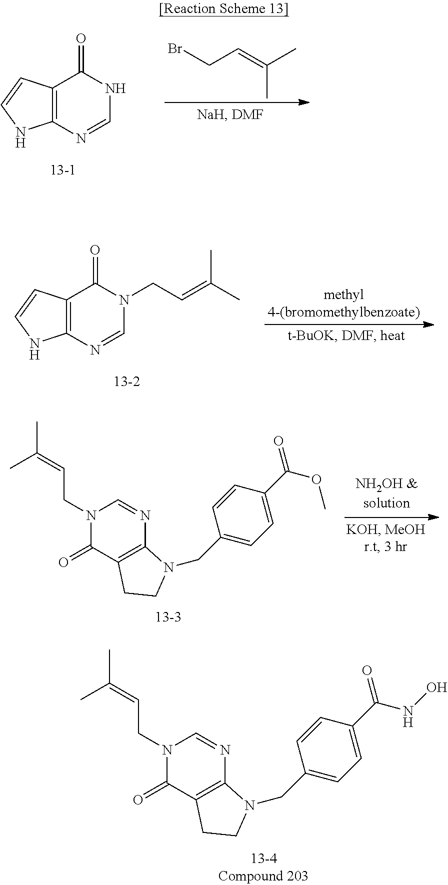

As shown in the reaction scheme 13, the compound of formula 13-1 is subjected to addition reaction with 3,3-dimethylallylbrimide to produce the compound of formula 13-2. Methyl 4-(bromomethyl)benzoate is added to the compound of formula 13-2 to synthesize the compound of formula 13-3, then potassium hydroxide (KOH), methanol and hydroxylamine hydrochloride (NH₂OH HCl) are added dropwise to the compound of formula 13-3 and reacted at room temperature, thus systhesizing the desired compound 203.

### Advantageous Effects

As above, the present invention relates to a hydroxamate derivatives for HDAC inhibitors as a novel selective inhibitor for histone deacetylase (HDAC), which can be used for treatment of inflammatory diseases, rheumatoid arthritis and degenerative diseases.

### DESCRIPTION OF DRAWINGS

FIG. 1 shows the Western blot analysis for compound 237;

FIG. 2 shows the test result of effectiveness of compound 87 in a collagen-induced arthritis model; and

FIG. 3 shows the test result of effectiveness of compound 237 in a collagen-induced arthritis model.

### BEST MODE FOR CARRYING OUT THE INVENTION

### Preparation of Compounds and Preparing Method of Compounds

The compound of formula 1 can be prepared by the method known from various references (e.g. WO 2011011186). Hereinafter, the preparing method for compound of formula 1 will be described in further detail with reaction scheme.

### Example 1

### Synthesis of Compound 18

### Step 1. Synthesis of 2,2-dimethyl-2,3-dihydro-1H-carbazol-4(9H)-on [formula 1-2]

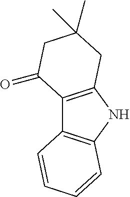 [SMILES: CC1(C)CC(=O)c2c([nH]c3ccccc23)C1]

To a microwave vial were added penylhydrazine [formula 1-1] (0.5 g, 4.62 mmol), 5-5-dimethyl-1,3-cyclohexandion (0.71 g, 5.08 mmol) and TFA (2 mL), and a reaction was carried out in a microwave reactor at 140° C. for 5 minutes. Then, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.43 g, 44%).

### Step 2. Synthesis of ethyl 6-(2,2-dimethyl-4-oxo-1,2,3,4-tetrahydrocarbazol-9-yl)hexanoate [formula 1-3]

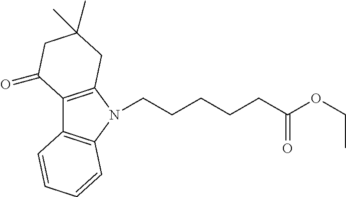 [SMILES: CCOC(=O)CCCCCn1c2c(c3ccccc31)C(=O)CC(C)(C)C2]

2,2-dimethyl-2,3-dihydro-1H-carbazol-4(9H)-on [formula 1-2] (0.1 g, 0.46 mmol) was dissolved in DMF (10 mL), then ethyl 6-bromohexanoate (0.1 g, 0.46 mmol), and NaH (0.014 g, 0.59 mmol) were added and stirred at 60° C. for 6 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.104 g, 64%).

### Step 3. Synthesis of 6-(2,2-dimethyl-4-oxo-1,2,3,4-tetrahydrocarbazol-9-yl)-N-hydroxyhexanamide [formula 1-4]

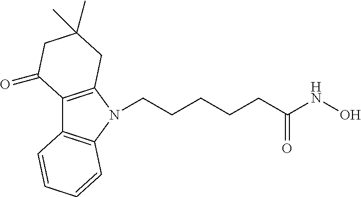 [SMILES: CC1(C)CC(=O)c2c(n(CCCCCC(=O)NO)c3ccccc23)C1]

To a flask were added ethyl 6-(2,2-dimethyl-4-oxo-1,2,3,4-tetrahydrocarbazol-9-yl)hexanoate [formula 1-3] (0.1 g, 0.28 mmol), hydroxylamine hydrochloride (NH₂OH HCl) (0.097 g, 1.4 mmol), potassium hydroxide (0.157 g, 2.8 mmol), and methanol (10 mL), and the mixture was stirred for 10 minutes. Then, hydroxylamine 50% aqueous solution had been added drop-wise slowly until mixed solution in the flask became clear, and the mixture was stirred at room temperature for 3 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; Dichloromethane/methanol, 20/1) to yield the compound 18 as white solid (0.056 g, 58%).

¹H NMR (400 MHz, MeOD-d₃) δ 8.08 (d, 1H, J=7.2 Hz), 7.49 (d, 1H, J=7.8 Hz), 7.27-7.23 (m, 2H), 4.22 (t, 2H, J=7.2 Hz), 2.93 (s, 2H), 2.45 (s, 2H), 2.08 (t, 2H, J=7.2 Hz), 1.84-1.36 (m, 6H), 1.19 (s. 6H). MS (ESI) m/z 343 (M⁺+H).

### Example 2

### Synthesis of Compound 19

### Step 1. Synthesis of methyl 4-((2,2-dimethyl-4-oxo-1,2,3,4-tetrahydrocarbazol-9-yl)methyl)benzoate [formula 1-5]

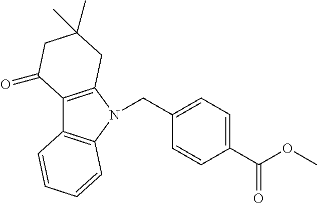 [SMILES: COC(=O)c1ccc(Cn2c3c(c4ccccc42)C(=O)CC(C)(C)C3)cc1]

2,2-dimethyl-2,3-dihydro-1H-carbazol-4(9H)-on [formula 1-2] (0.1 g, 0.46 mmol) was dissolved in DMF (10 mL), then methyl 4-(bromomethyl)benzoate (0.105 g, 0.46 mmol) and NaH (0.014 g, 0.59 mmol) were added and the mixture was stirred at 60° C. for 6 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.13 g, 78%).

### Step 2. Synthesis of 4-((2,2-dimethyl-4-oxo-1,2,3,4-tetrahydrocarbazol-9-yl)methyl)-N-hydroxybenzamide [formula 1-6]

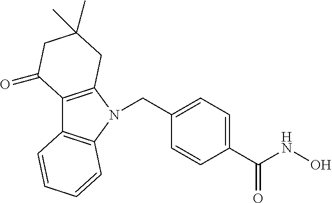 [SMILES: CC1(C)CC(=O)c2c(n(Cc3ccc(C(=O)NO)cc3)c3ccccc23)C1]

To a flask were added methyl 4-((2,2-dimethyl-4-oxo-1,2,3,4-tetrahydrocarbazol-9-yl)methyl)benzoate [formula 1-5] (0.035 g, 0.09 mmol), hydroxylamine hydrochloride (NH₂OH HCl) (0.033 g, 0.48 mmol), potassium hydroxide (0.05 g, 0.9 mmol) and methanol (10 mL), and the mixture was stirred for 10 minutes. Then, hydroxylamine 50% aqueous solution was added drop-wise slowly until the mixed solution in the flask became clear, and the mixture was stirred at room temperature for 3 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified with column chromatography (SiO₂; dichloromethane/methanol, 20/1) and to yield the compound 19 as white solid (0.012 g, 40%).

¹H NMR (400 MHz, MeOD-d₃) δ 8.03-8.00 (m, 1H), 7.68 (d, 2H, J=8.2 Hz), 7.50-7.47 (m, 1H), 7.19-7.11 (m, 4H), 5.55 (s, 2H), 2.88 (s, 2H), 2.37 (s, 2H), 1.08 (s, 6H). MS (ESI) m/z 363 (M⁺+H).

### Example 3

### Synthesis of Compound 20

### Step 1. Synthesis of methyl 7-(2,2-dimethyl-4-oxo-1,2,3,4-tetrahydrocarbazol-9-yl)-heptanoate [formula 1-3]

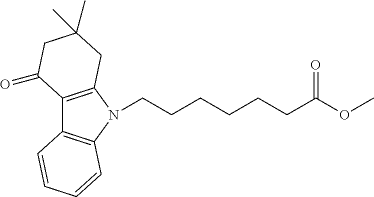 [SMILES: COC(=O)CCCCCCn1c2c(c3ccccc31)C(=O)CC(C)(C)C2]

2,2-dimethyl-2,3-dihydro-1H-carbazol-4(9H)-on [formula 1-2] (0.1 g, 0.46 mmol) was dissolved in DMF (10 mL), then ethyl 7-bromoheptanoate (0.109 g, 0.46 mmol) and NaH (0.014 g, 0.59 mmol) were added and stirred at 60° C. for 6 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.065 g, 38%).

### Step 2. Synthesis of 7-(2,2-dimethyl-4-oxo-1,2,3,4-tetrahydrocarbazol-9-yl)-N-hydroxyheptanamide [formula 1-4]

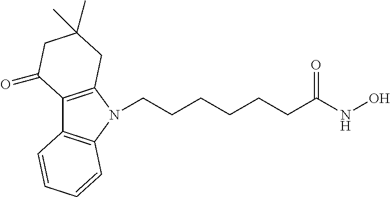 [SMILES: CC1(C)CC(=O)c2c(n(CCCCCCC(=O)NO)c3ccccc23)C1]

To a flask were added methyl 7-(2,2-dimethyl-4-oxo-1,2,3,4-tetrahydrocarbazol-9-yl)heptanoate [formula 1-3] (0.065 g, 0.17 mmol), hydroxylamine hydrochloride (NH₂OH HCl) (0.061 g, 0.87 mmol), potassium hydroxide (0.095 g, 1.7 mmol), and methanol (10 mL), and the mixture was stirred for 10 minutes. Then, hydroxylamine 50% aqueous solution had been added drop-wise slowly until the mixed solution in the flask became clear, and the mixture was stirred at room temperature for 3 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 20/1) to yield the compound 20 as white solid (0.019 g, 32%).

¹H NMR (400 MHz, DMSO-d₆) δ 7.97 (d, 1H, J=7.1 Hz), 7.38 (d, 1H, J=7.9 Hz), 7.15-7.11 (m, 2H), 4.09 (t, 2H, J=7.1 Hz), 2.81 (s, 2H), 2.32 (s, 2H), 1.92 (t, 2H, J=7.2 Hz), 1.73-1.20 (m, 8H), 1.07 (s, 6H). MS (ESI) m/z 357 (M⁺+H).

### Example 4

### Synthesis of Compound 40

### Step 1. Synthesis of 6-bromo-2,2-dimethyl-2,3-dihydro-1H-carbazol-4(9H)-on [formula 1-2]

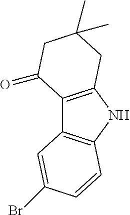 [SMILES: CC1(C)CC(=O)c2c([nH]c3ccc(Br)cc23)C1]

To a microwave vial were added 4-bromophenylhydrazine HCl [formula 1-1] (0.17 g, 0.76 mmol), 5-5-dimethyl-1,3-cyclohexandion (0.12 g, 0.85 mmol) and TFA (1 mL) and a reaction was carried out in a microwave reactor at 140° C. for 10 minutes. Then, the reaction mixture was extracted with ethyl acetate and saturated NaHCO₃ aqueous solution; the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.12 g, 54%).

### Step 2. Synthesis of ethyl 6-(6-bromo-2,2-dimethyl-4-oxo-1,2,3,4-tetrahydrocarbazol-9-yl)hexanoate [formula 1-3]

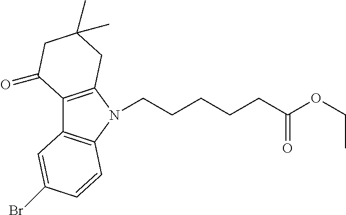 [SMILES: CCOC(=O)CCCCCn1c2c(c3cc(Br)ccc31)C(=O)CC(C)(C)C2]

6-bromo-2,2-dimethyl-2,3-dihydro-1H-carbazol-4(9H)-on) [formula 1-2] (0.05 g, 0.17 mmol) was dissolved in DMF (10 mL), then ethyl 6-bromo hexanoate (0.038 g, 0.17 mmol) and

NaH (0.006 g, 0.22 mmol) were added and stirred at 60° C. for 12 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.03 g, 42%).

### Step 3. Synthesis of 6-(6-bromo-2,2-dimethyl-4-oxo-1,2,3,4-tetrahydrocarbazol-9-yl)-N-hydroxy hexanamide [formula 1-4]

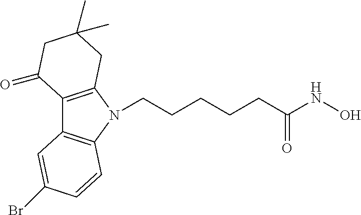 [SMILES: CC1(C)CC(=O)c2c(n(CCCCCC(=O)NO)c3ccc(Br)cc23)C1]

To a flask were added ethyl 6-(6-bromo-2,2-dimethyl-4-oxo-1,2,3,4-tetrahydrocarbazol-9-yl)hexanoate [formula 1-3] (0.03 g, 0.07 mmol), hydroxylamine hydrochloride (NH₂OH HCl) (0.024 g, 0.34 mmol), potassium hydroxide (0.04 g, 0.7 mmol) and methanol (10 mL), and the mixture was stirred for 10 minutes. Then, hydroxylamine 50% aqueous solution had been added drop-wise slowly until mixed solution in the flask became clear, and the mixture was stirred at room temperature for 12 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; Dichloromethane/Methanol, 15/1) to yield the compound 40 as solid (0.016 g, 55%).

¹H NMR (400 MHz, MeOD-d₃) δ 8.18 (d, 1H, J=1.8 Hz), 7.41 (d, 1H, J=8.6 Hz), 7.35 (dd, 1H, J=8.6, 1.9 Hz), 4.20 (t, 2H, J=7.2 Hz), 2.91 (s, 2H), 2.43 (s, 2H), 2.07 (t, 2H, J=7.3 Hz) 1.82-1.77 (m, 2H), 1.68-1.61 (m, 2H), 1.39-1.33 (m, 2H), 1.18 (s, 6H). MS (ESI) m/z 422 (M⁺+H).

### Example 5

### Synthesis of Compound 41

### Step 1. Synthesis of 7-bromo-2,2-dimethyl-2,3-dihydro-1H-carbazol-4(9H)-on) [formula 1-2]

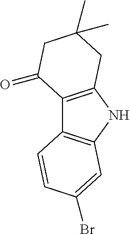 [SMILES: CC1(C)CC(=O)c2c([nH]c3cc(Br)ccc23)C1]

To a microwave vial were added 3-bromophenyl hydrazine [formula 1-1] (0.17 g, 0.76 mmol), 5-5-dimethyl-1,3-cyclohexandion (0.12 g, 0.85 mmol) and TFA (1 mL), and a reaction was carried out in a microwave reactor at 140° C. for 10 minutes. Then, the reaction mixture was extracted with ethyl acetate and saturated NaHCO₃ aqueous solution; the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.04 g, 18%).

### Step 2. Synthesis of ethyl 6-(7-bromo-2,2-dimethyl-4-oxo-1,2,3,4-tetrahydrocarbazol-9-yl)hexanoate [Formula 1-3]

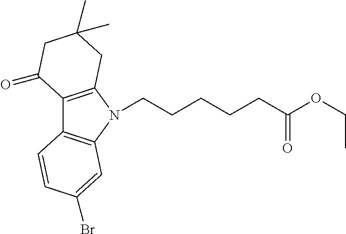 [SMILES: CCOC(=O)CCCCCn1c2c(c3ccc(Br)cc31)C(=O)CC(C)(C)C2]

7-bromo-2,2-dimethyl-2,3-dihydro-1H-carbazol-4(9H)-on [formula 1-2] (0.04 g, 0.13 mmol) was dissolved in DMF (10 mL), then ethyl 6-bromo hexanoate (0.029 g, 0.13 mmol) and NaH (0.005 g, 0.1955 mmol) were added and stirred at 60° C. for 12 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.035 g, 37%).

### Step 3. Synthesis of 6-(7-bromo-2,2-dimethyl-4-oxo-1,2,3,4-tetrahydrocarbazol-9-yl)-N-hydroxy hexanamide [formula 1-4]

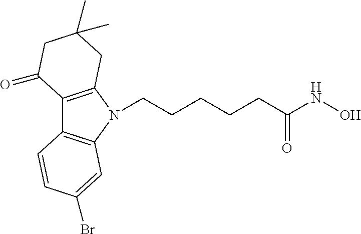 [SMILES: CC1(C)CC(=O)c2c(n(CCCCCC(=O)NO)c3cc(Br)ccc23)C1]

To a flask were added ethyl 6-(7-bromo-2,2-dimethyl-4-oxo-1,2,3,4-tetrahydrocarbazol-9-yl)hexanoate [formula 1-3] (0.03 g, 0.07 mmol), hydroxylamine hydrochloride (NH₂OH HCl) (0.023 g, 0.34 mmol), potassium hydroxide (0.039 g, 0.7 mmol) and methanol (2 mL), and stirred for 10 minutes. Then, hydroxylamine 50% aqueous solution had been added drop-wise slowly until the mixed solution in the flask became clear, and the mixture was stirred at room temperature for 6 hours. After the completion of the reaction, methanol was distilled out under reduced pressure, and extraction was carried out with ethyl acetate only, dried over anhydrous Na₂SO₄, filtered and concentrated under reduced pressure. Residue was purified by column chromatography (SiO₂; Dichloromethane/methanol, 20/1) to yield the compound 41 as solid (0.015 g, 52%).

¹H NMR (400 MHz, MeOD-d₃) δ 7.95 (d, 1H, J=8.4 Hz), 7.68 (d, 1H, J=1.4 Hz), 7.34 (dd, 1H, J=8.4, 1.6 Hz), 4.19 (t, 2H, J=7.2 Hz), 2.90 (s, 2H), 2.44 (s, 2H), 2.09 (t, 2H, J=7.2 Hz), 1.81-1.77 (m, 2H), 1.67-1.63 (m, 2H), 1.37-1.32 (m, 2H), 1.18 (s, 6H). MS (ESI) m/z 422 (M⁺+H).

### Example 6

### Synthesis of Compound 45

### Step 1. Synthesis of methyl 4-((6-bromo-2,2-dimethyl-4-oxo-1,2,3,4-tetrahydro carbazol-9-yl)methyl)benzoate [formula 1-5]

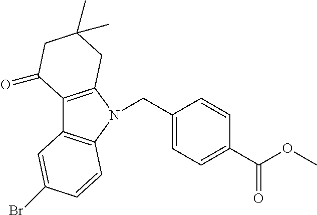 [SMILES: COC(=O)c1ccc(Cn2c3c(c4cc(Br)ccc42)C(=O)CC(C)(C)C3)cc1]

6-bromo-2,2-dimethyl-2,3-dihydro-1H-carbazol-4(9H)-on [formula 1-2] (0.08 g, 0.27 mmol) was dissolved in DMF (10 mL), then methyl 4-(bromomethyl)benzoate (0.063 g, 0.27 mmol) and NaH (0.01 g, 0.41 mmol) were added and stirred at 60° C. for 6 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.099 g, 84%).

### Step 2. Synthesis of 4-((6-bromo-2,2-dimethyl-4-oxo-1,2,3,4-tetrahydrocarbazol-9-yl)methyl)-N-hydroxy benzamide [formula 1-6]

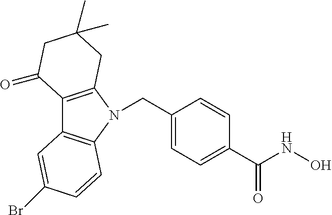 [SMILES: CC1(C)CC(=O)c2c(n(Cc3ccc(C(=O)NO)cc3)c3ccc(Br)cc23)C1]

To a flask were added methyl 4-((6-bromo-2,2-dimethyl-4-oxo-1,2,3,4-tetrahydrocarbazol-9-yl)methyl)benzoate [formula 1-5] (0.099 g, 0.23 mmol), hydroxylamine hydrochloride (NH₂OH HCl) (0.078 g, 1.13 mmol), potassium hydroxide (0.063 g, 11.3 mmol) and methanol (10 mL), and stirred for 10 minutes. Then, hydroxylamine 50% aqueous solution had been added drop-wise slowly until the mixed solution in a flask became clear, and the mixture was stirred at room temperature for 3 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 20/1) to yield the compound 45 as white solid (0.035 g, 34%).

¹H NMR (400 MHz, MeOD-d₃) δ 8.23 (s, 1H), 7.70 (d, 2H, J=8.2 Hz), 7.32 (s, 1H), 7.14 (d, 2H, J=8.2 Hz), 5.54 (s, 2H), 2.87 (s, 2H), 2.47 (s, 2H) 1.14 (s, 6H). MS (ESI) m/z 442 (M⁺+H)

### Example 7

### Synthesis of Compound 46

### Step 1. Synthesis of 8-bromo-2,2-dimethyl-2,3-dihydro-1H-carbazol-4(9H)-on [formula 1-2]

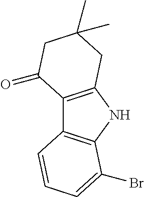 [SMILES: CC1(C)CC(=O)c2c([nH]c3c(Br)cccc23)C1]

To a microwave vial were added 2-bromophenyl hydrazine HCl [formula 1-1] (0.17 g, 0.76 mmol), 5-5-dimethyl-1,3-cyclohexandion (0.12 g, 0.86 mmol) and TFA (1 mL), and a reaction was carried out in a microwave reactor at 140° C. for 10 minutes. Then, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.08 g, 36%).

### Step 2. Synthesis of ethyl 6-(1-bromo-7,7-dimethyl-5-oxo-5,6,7,8-tetrahydrocarbazol-9-yl)hexanoate [Formula 1-3]

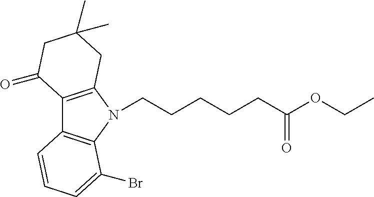 [SMILES: CCOC(=O)CCCCCn1c2c(c3cccc(Br)c31)C(=O)CC(C)(C)C2]

8-bromo-2,2-dimethyl-2,3-dihydro-1H-carbazol-4(9H)-on [formula 1-2] (0.08 g, 0.27 mmol), ethyl 6-bromo hexanoate (0.061 g, 0.27 mmol) and NaH (0.01 g, 0.41 mmol) were dissolved in DMF (10 mL), and stirred at 60° C. for 12 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.064 g, 55%).

### Step 3. Synthesis of 6-(1-bromo-7,7-dimethyl-5-oxo-5,6,7,8-tetrahydrocarbazol-9-yl)-N-hydroxy hexanamide [formula 1-4]

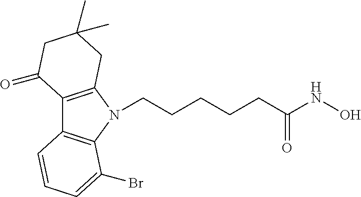 [SMILES: CC1(C)CC(=O)c2c(n(CCCCCC(=O)NO)c3c(Br)cccc23)C1]

To a flask were added ethyl 6-(1-bromo-7,7-dimethyl-5-oxo-5,6,7,8-tetrahydrocarbazol-9-yl)hexanoate [formula 1-3] (0.064 g, 0.15 mmol), hydroxylamine hydrochloride (NH₂OH HCl) (0.051 g, 0.73 mmol), potassium hydroxide (0.84 g, 1.5 mmol) and methanol (10 mL), and stirred for 10 minutes. Then, hydroxylamine 50% aqueous solution had been added drop-wise slowly until the mixed solution in the flask became clear, and the mixture was stirred at room temperature for 3 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 20/1) to yield the compound 46 as solid (0.028 g, 44%).

¹H NMR (400 MHz, MeOD-d₃) δ 8.13 (d, 1H, J=7.8 Hz), 7.42 (d, 1H, J=7.7 Hz), 7.08 (t, 1H, J=7.8 Hz), 4.53 (t, 2H, J=7.8 Hz), 2.91 (s, 2H), 2.44 (s, 2H), 2.12 (t, 2H, J=7.3 Hz), 1.84-1.677 (m, 4H), 1.46-1.42 (m, 2H), 1.19 (s, 6H). MS (ESI) m/z 422 (M⁺+H).

### Example 8

### Synthesis of Compound 47

### Step 1. Synthesis of 6,8-dichloro-2,2-dimethyl-2,3-dihydro-1H-carbazol-4(9H)-on [formula 1-2]

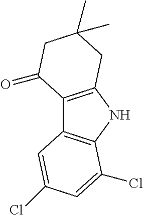 [SMILES: CC1(C)CC(=O)c2c([nH]c3c(Cl)cc(Cl)cc23)C1]

To a microwave vial were added 2,4-dichlorophenyl hydrazine HCl [formula 1-1] (0.2 g, 0.94 mmol), 5-5-dimethyl-1,3-cyclohexandion (0.13 g, 0.94 mmol) and TFA (2 mL), and a reaction was carried out in a microwave reactor at 140° C. for 30 minutes. Then, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.07 g, 26%).

### Step 2. Synthesis of ethyl 6-(1,3-dichloro-7,7-dimethyl-5-oxo-5,6,7,8-tetrahydrocarbazol-9-yl)hexanoate [Formula 1-3]

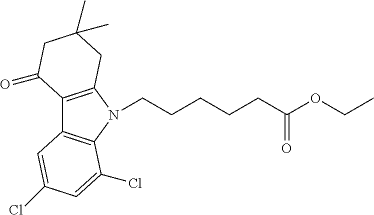 [SMILES: CCOC(=O)CCCCCn1c2c(c3cc(Cl)cc(Cl)c31)C(=O)CC(C)(C)C2]

6,8-dichloro-2,2-dimethyl-2,3-dihydro-1H-carbazol-4(9H)-on [formula 1-2] (0.07 g, 0.25 mmol) was dissolved in DMF (10 mL), then ethyl 6-bromohexanoate (0.055 g, 0.25 mmol) and NaH (0.01 g, 0.38 mmol) were added and stirred at 60° C. for 12 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.042 g, 39%).

### Step 3. Synthesis of 6-(1,3-dichloro-7,7-dimethyl-5-oxo-5,6,7,8-tetrahydrocarbazol-9-yl)-N-hydroxyhexanamide [formula 1-4]

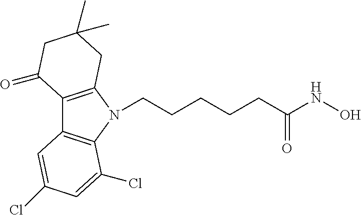 [SMILES: CC1(C)CC(=O)c2c(n(CCCCCC(=O)NO)c3c(Cl)cc(Cl)cc23)C1]

To a flask were added ethyl 6-(1,3-dichloro-7,7-dimethyl-5-oxo-5,6,7,8-tetrahydrocarbazol-9-yl)hexanoate [formula 1-3] (0.042 g, 0.1 mmol), hydroxylamine hydrochloride (NH₂OH HCl) (0.034 g, 0.5 mmol), potassium hydroxide (0.056 g, 1.0 mmol) and methanol (10 mL), and stirred for 10 minutes. Then, hydroxylamine 50% aqueous solution had been added drop-wise slowly until the mixed solution in a flask became clear, and the mixture was stirred at room temperature for 3 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 20/1) to yield the compound 47 as white solid (0.027 g, 65%).

¹H NMR (400 MHz, MeOD-d₃) δ 8.04 (d, 1H, J=2.0 Hz), 7.25 (d, 1H, J=2.0 Hz), 4.49 (t, 2H, J=7.7 Hz), 2.92 (s, 2H), 2.45 (s, 2H), 2.11 (t, 2H, J=7.2 Hz), 1.85-1.66 (m, 4H), 1.44-1.403 (m, 2H), 1.19 (s, 6H). MS (ESI) m/z 411 (M⁺+H).

### Example 9

### Synthesis of Compound 48

### Step 1. Synthesis of 5,8-dichloro-2,2-dimethyl-2,3-dihydro-1H-carbazol-4(9H)-on [formula 1-2]

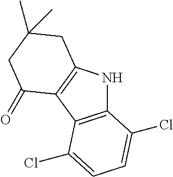 [SMILES: CC1(C)CC(=O)c2c([nH]c3c(Cl)ccc(Cl)c23)C1]

To a microwave vial were added 2,5-dichlorophenyl hydrazine HCl [formula 1-1] (0.2 g, 1.12 mmol), 5-5-dimethyl-1,3-cyclohexandion (0.15 g, 1.12 mmol) and TFA (2 mL), and a reaction was carried out in a microwave reactor at 140° C. for 30 minutes. Then, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.064 g, 20%).

### Step 2. Synthesis of ethyl 6-(1,4-dichloro-7,7-dimethyl-5-oxo-5,6,7,8-tetrahydrocarbazol-9-yl)hexanoate [Formula 1-3]

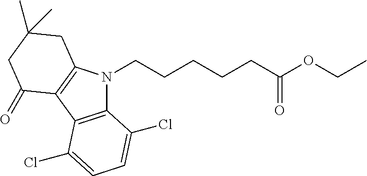 [SMILES: CCOC(=O)CCCCCn1c2c(c3c(Cl)ccc(Cl)c31)C(=O)CC(C)(C)C2]

5,8-dichloro-2,2-dimethyl-2,3-dihydro-1H-carbazol-4(9H)-on [formula 1-2] (0.064 g, 0.23 mmol) was dissolved in DMF (10 mL), then ethyl 6-bromohexanoate (0.05 g, 0.25 mmol) and NaH (0.009 g, 0.35 mmol) were added and stirred at 60° C. for 12 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.05 g, 52%).

### Step 3. Synthesis of 6-(1,4-dichloro-7,7-dimethyl-5-oxo-5,6,7,8-tetrahydrocarbazol-9-yl)-N-hydroxyhexanamide [formula 1-4]

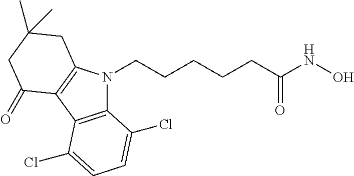 [SMILES: CC1(C)CC(=O)c2c(n(CCCCCC(=O)NO)c3c(Cl)ccc(Cl)c23)C1]

To a flask were added ethyl 6-(1,4-dichloro-7,7-dimethyl-5-oxo-5,6,7,8-tetrahydrocarbazol-9-yl)hexanoate [formula 1-3] (0.05 g, 0.12 mmol), hydroxylamine hydrochloride (NH₂OH HCl) (0.041 g, 0.59 mmol), potassium hydroxide (0.067 g, 1.2 mmol) and methanol (10 mL), and stirred for 10 minutes. Then, hydroxylamine 50% aqueous solution had been added drop-wise slowly until the mixed solution in the flask became clear, and the mixture was stirred at room temperature for 3 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 20/1) to yield the compound 48 as liquid (0.025 g, 51%).

¹H NMR (400 MHz, MeOD-d₃) δ 7.09 (d, 1H, J=8.2 Hz), 7.04 (d, 1H, J=8.2 Hz), 4.49 (t, 2H, J=7.8 Hz), 2.74 (s, 2H), 2.72 (s, 2H), 2.10 (t, 2H, J=7.3 Hz), 1.78-1.65 (m, 4H), 1.42-1.37 (m, 2H), 1.15 (s, 6H). MS (ESI) m/z No result (M⁺+H).

### Example 10

### Synthesis of Compound 49

### Step 1. Synthesis of methyl 4-((1,4-dichloro-7,7-dimethyl-5-oxo-5,6,7,8-tetrahydrocarbazol-9-yl)methyl)benzoate [formula 1-5]

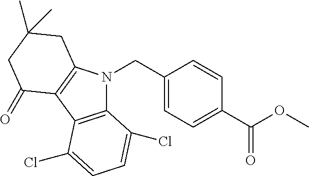 [SMILES: COC(=O)c1ccc(Cn2c3c(c4c(Cl)ccc(Cl)c42)C(=O)CC(C)(C)C3)cc1]

5,8-dichloro-2,2-dimethyl-2,3-dihydro-1H-carbazol-4(9H)-on [formula 1-2] (0.076 g, 0.27 mmol) was dissolved in DMF (10 mL), and t-BuOK (0.045 g, 0.41 mmol) was added and stirred for 10 minutes. Then, methyl 4-(bromomethyl)benzoate (0.074 g, 0.32 mmol) and KI (0.005 g, 0.027 mmol) were added and stirred at 60° C. for 48 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 2/1) to yield the title compound (0.05 g, 43%).

### Step 2. Synthesis of 4-((1,4-dichloro-7,7-dimethyl-5-oxo-5,6,7,8-tetrahydrocarbazol-9-yl)methyl)-N-hydroxybenzamidee [Formula 1-6]

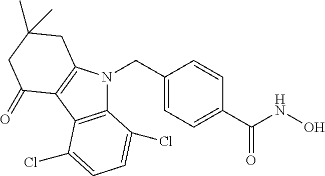 [SMILES: CC1(C)CC(=O)c2c(n(Cc3ccc(C(=O)NO)cc3)c3c(Cl)ccc(Cl)c23)C1]

To a flask were added methyl 4-((1,4-dichloro-7,7-dimethyl-5-oxo-5,6,7,8-tetrahydrocarbazol-9-yl)methyl)benzoate [formula 1-5] (0.05 g, 0.12 mmol), hydroxylamine hydrochloride (NH₂OH HCl) (0.042 g, 0.6 mmol), potassium hydroxide (0.067 g, 1.2 mmol) and methanol (10 mL), and stirred for 10 minutes. Then, hydroxylamine 50% aqueous solution had been added drop-wise slowly until the mixed solution in the flask became clear, and the mixture was stirred at room temperature for 3 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 20/1) to yield the compound 49 as solid (0.007 g, 14%).

¹H NMR (400 MHz, MeOD-d₃) δ 7.68 (d, 2H, J=8.3 Hz), 7.08 (dd, 2H, J=14.0, 8.3 Hz), 6.95 (d, 2H, J=8.2 Hz), 5.91 (s, 2H), 2.73 (s, 2H), 2.63 (s, 2H), 1.08 (s, 6H). MS (ESI) m/z No result (M⁺+H).

### Example 11

### Synthesis of Compound 50

### Step 1. Synthesis of ethyl 7-(6-bromo-2,2-dimethyl-4-oxo-1,2,3,4-tetrahydrocarbazol-9-yl)heptanoate [formula 1-3]

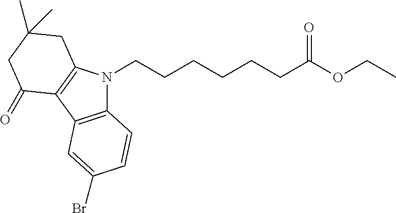 [SMILES: CCOC(=O)CCCCCCn1c2c(c3cc(Br)ccc31)C(=O)CC(C)(C)C2]

6-bromo-2,2-dimethyl-2,3-dihydro-1H-carbazol-4(9H)-on [formula 1-2] (0.03 g, 0.103 mmol) was dissolved in DMF (10 mL), then ethyl 7-bromoheptanoate (0.024 g, 0.103 mol) and NaH (0.037 g, 0.15 mmol) were added and stirred at 80° C. for 12 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.042 g, 91%).

### Step 2. Synthesis of 7-(6-bromo-2,2-dimethyl-4-oxo-1,2,3,4-tetrahydrocarbazol-9-yl)-N-hydroxyheptanamide [Formula 1-4]

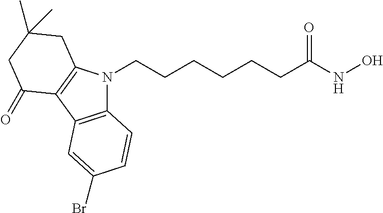 [SMILES: CC1(C)CC(=O)c2c(n(CCCCCCC(=O)NO)c3ccc(Br)cc23)C1]

Ethyl 7-(6-bromo-2,2-dimethyl-4-oxo-1,2,3,4-tetrahydrocarbazol-9-yl)heptanoate [formula 1-3](0.074 g, 0.165 mmol) was dissolved in methanol (5 mL). Thereto, hydroxylamine hydrochloride (NH₂OH HCl) (0.057 g, 0.825 mmol) and potassium hydroxide (0.109 g, 3.301 mmol) were added and stirred. Then, hydroxylamine 50% aqueous solution had been added drop-wise slowly until mixed solution in the flask became clear, and the mixture was stirred at room temperature for 3 hours. After the completion of the reaction, methanol was distilled out under reduced pressure, and the reaction mixture was extracted with ethyl acetate and 2N hydrochloric acid aqueous solution. Residue was purified by column chromatography (SiO₂; dichloromethane/methanol, 15/1) to yield the compound 50 as white solid (0.043 g, 59%).

¹H NMR (400 MHz, MeOD-d₃) δ 8.18 (s, 1H), 7.38 (dd, 2H, J=18.7, 8.7 Hz), 4.18 (t, 2H, J=7.2 Hz), 2.90 (s, 2H), 2.42 (s, 2H), 1.78-1.76 (m, 2H), 1.61-1.58 (m, 2H), 1.37-1.34 (m, 4H), 1.17 (s, 6H). fvMS (ESI) m/z 436 (M⁺+H).

### Example 12

### Synthesis of Compound 51

### Step 1. Synthesis of ethyl 7-(1,3-dichloro-7,7-dimethyl-5-oxo-5,6,7,8-tetrahydrocarbazol-9-yl)heptanoate [formula 1-3]

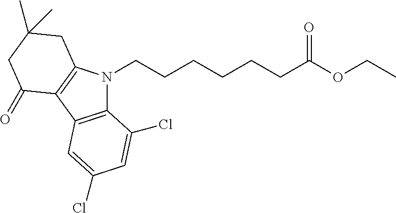 [SMILES: CCOC(=O)CCCCCCn1c2c(c3cc(Cl)cc(Cl)c31)C(=O)CC(C)(C)C2]

6,8-dichloro-2,2-dimethyl-2,3-dihydro-1H-carbazol-4(9H)-on [formula 1-2] (0.05 g, 0.18 mmol) was dissolved in DMF (10 mL), and t-BuOK (0.03 g, 0.27 mmol) was added. Then, ethyl 7-bromoheptanoate (0.05 g, 0.21 mmol) and KI (0.005 g, 0.03 mmol) were added and stirred at 60° C. for 48 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 3/1) to yield the title compound (0.02 g, 25%).

### Step 2. Synthesis of 7-(1,3-dichloro-7,7-dimethyl-5-oxo-5,6,7,8-tetrahydrocarbazol-9-yl)-N-hydroxyheptanamide [Formula 1-4]

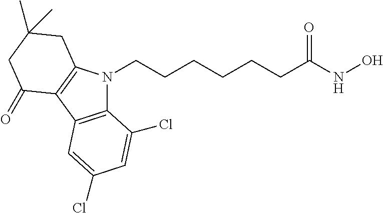 [SMILES: CC1(C)CC(=O)c2c(n(CCCCCCC(=O)NO)c3c(Cl)cc(Cl)cc23)C1]

To a flask were added ethyl 7-(1,3-dichloro-7,7-dimethyl-5-oxo-5,6,7,8-tetrahydrocarbazol-9-yl)heptanoate [formula 1-3] (0.02 g, 0.045 mmol), hydroxylamine hydrochloride (NH₂OH HCl) (0.016 g, 0.23 mmol), potassium hydroxide (0.025 g, 0.45 mmol) and methanol (5 mL), and stirred for 10 minutes. Then, hydroxylamine 50% aqueous solution had been added drop-wise slowly until the mixed solution in the flask became clear, and the mixture was stirred at room temperature for 3 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 20/1) to yield the compound 51 as solid (0.005 g, 26%).

¹H NMR (400 MHz, MeOD-d₃) δ 8.05 (d, 1H, J=2.0 Hz), 7.25 (d, 1H, J=2.0 Hz), 4.49 (t, 2H, J=7.6 Hz), 2.92 (s, 2H), 2.45 (s, 2H), 2.09 (t, 2H, J=7.2 Hz), 1.83-1.79 (m, 2H), 1.65-1.61 (m, 2H) 1.41-1.38 (m, 4H), 1.13 (s, 6H). MS (ESI) m/z No result (M⁺+H).

### Example 13

### Synthesis of Compound 52

### Step 1. Synthesis of ethyl 7-(1-bromo-7,7-dimethyl-5-oxo-5,6,7,8-tetrahydrocarbazol-9-yl)heptanoate [formula 1-3]

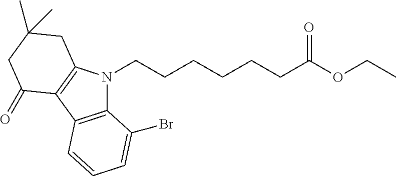 [SMILES: CCOC(=O)CCCCCCn1c2c(c3cccc(Br)c31)C(=O)CC(C)(C)C2]

8-bromo-2,2-dimethyl-2,3-dihydro-1H-carbazol-4(9H)-on [formula 1-2] (0.1 g, 0.34 mmol) was dissolved in DMF (10 mL), and t-BuOK (0.057 g, 0.51 mmol) was added and stirred for 10 minutes. Then, ethyl 7-bromoheptanoate (0.081 g, 0.34 mmol) and KI (0.005 g, 0.034 mmol) were added and stirred at 60° C. for 12 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.083 g, 54%).

### Step 2. Synthesis of 7-(1-bromo-7,7-dimethyl-5-oxo-5,6,7,8-tetrahydrocarbazol-9-yl)-N-hydroxyheptanamide [Formula 1-4]

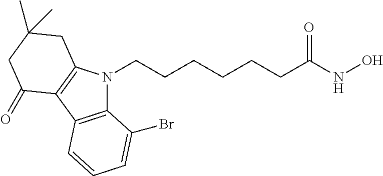 [SMILES: CC1(C)CC(=O)c2c(n(CCCCCCC(=O)NO)c3c(Br)cccc23)C1]

To a flask were added ethyl 7-(1-bromo-7,7-dimethyl-5-oxo-5,6,7,8-tetrahydrocarbazol-9-yl)heptanoate [formula 1-3](0.083 g, 0.18 mmol), hydroxylamine hydrochloride (NH₂OH HCl) (0.064 g, 0.92 mmol), potassium hydroxide (0.1 g, 1.8 mmol) and methanol (20 mL), and stirred for 10 minutes. Then, hydroxylamine 50% aqueous solution had been added drop-wise slowly until the mixed solution in the flask became clear, and the mixture was stirred at room temperature for 6 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 10/1) to yield the compound 52 as solid (0.052 g, 67%).

¹H NMR (400 MHz, DMSO-d₆) δ 10.32 (s, 1H), 8.65 (s, 1H), 8.07 (d, 1H, J=7.8 Hz), 7.41 (d, 1H, J=7.7 Hz), 7.09 (t, 1H, J=7.7 Hz), 4.46 (t, 2H, J=7.5 Hz), 2.92 (s, 2H), 2.36 (s, 2H), 1.92 (t, 2H, J=7.2 Hz), 1.69-1.46 (m, 2H), 1.34-1.32 (m, 2H), 1.32-1.23 (m, 2H), 1.10 (s, 6H). MS (ESI) m/z 436 (M⁺+H).

### Example 14

### Synthesis of Compound 53

### Step 1. Synthesis of 5-bromo-2,2-dimethyl-2,3-dihydro-1H-carbazol-4(9H)-on [formula 1-2]

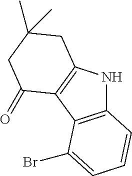 [SMILES: CC1(C)CC(=O)c2c([nH]c3cccc(Br)c23)C1]

To a microwave vial were added 3-bromophenyl hydrazine [formula 1-1] (0.17 g, 0.76 mmol), 5-5-dimethyl-1,3-cyclohexandion (0.12 g, 0.85 mmol) and TFA (1 mL) and a reaction was carried out in a microwave reactor at 140° C. for 10 minutes. Then, the reaction mixture was extracted with ethyl acetate and saturated NaHCO₃ aqueous solution; the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.04 g, 18%).

### Step 2. Synthesis of methyl 4-((5-bromo-2,2-dimethyl-4-oxo-1,2,3,4-tetrahydrocarbazol-9-yl)-methyl)benzoate [formula 1-5]

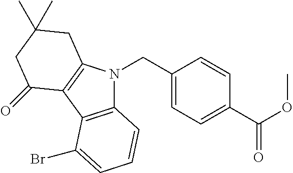 [SMILES: COC(=O)c1ccc(Cn2c3c(c4c(Br)cccc42)C(=O)CC(C)(C)C3)cc1]

5-bromo-2,2-dimethyl-2,3-dihydro-1H-carbazol-4(9H)-on [formula 1-2] (0.12 g, 0.41 mol) was dissolved in DMF (10 mL). Thereto, t-BuOK (0.055 g, 0.49 mmol), methyl 4-(bromomethyl) benzoate (0.074 g, 0.32 mmol) and KI (0.005 g, 0.027 mmol) were added and stirred at 60° C. for 6 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 3/1) to yield the title compound (0.05 g, 27%).

### Step 3. Synthesis of 4-((5-bromo-2,2-dimethyl-4-oxo-1,2,3,4-tetrahydrocarbazol-9-yl) methyl)-N-hydroxy benzamide [Formula 1-6]

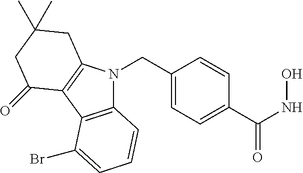 [SMILES: CC1(C)CC(=O)c2c(n(Cc3ccc(C(=O)NO)cc3)c3cccc(Br)c23)C1]

To a flask were added methyl 4-((5-bromo-2,2-dimethyl-4-oxo-1,2,3,4-tetrahydrocarbazol-9-y)methyl)benzoate [formula 1-5] (0.05 g, 0.11 mmol), hydroxylamine hydrochloride (NH₂OH HCl) (0.039 g, 0.55 mmol), potassium hydroxide (0.062 g, 1.1 mmol) and methanol (20 mL), and stirred for 10 minutes. Then, hydroxylamine 50% aqueous solution had been added drop-wise slowly until the mixed solution in the flask became clear, and the mixture was stirred at room temperature for 12 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 20/1) to yield the compound 53 as solid (0.021 g, 44%).

¹H NMR (400 MHz, DMSO-d₆) δ 10.45 (s, 1H), 7.66 (d, 2H, J=8.2 Hz), 7.43 (d, 1H, J=8.2 Hz), 7.30 (d, 1H, J=7.6 Hz), 7.03 (d, 2H, J=8.2 Hz), 6.98 (d, 1H, J=7.9 Hz), 5.49 (s, 2H), 2.66 (s, 2H), 2.59 (s, 2H), 1.01 (s, 6H). MS (ESI) m/z 442 (M⁺+H).

### Example 15

### Synthesis of Compound 54

### Step 1. Synthesis of methyl 4-((7-bromo-2,2-dimethyl-4-oxo-1,2,3,4-tetrahydrocarbazol-9-yl)methyl)benzoate [formula 1-5]

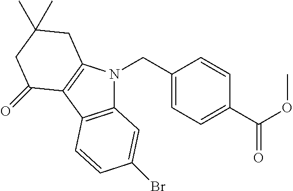 [SMILES: COC(=O)c1ccc(Cn2c3c(c4ccc(Br)cc42)C(=O)CC(C)(C)C3)cc1]

7-bromo-2,2-dimethyl-2,3-dihydro-1H-carbazol-4(9H)-on [formula 1-2] (0.2 g, 0.68 mmol) was dissolved in DMF (20 mL), and t-BuOK (0.092 g, 0.82 mmol) was added and stirred for 10 minutes. Then, methyl 4-(bromomethyl)benzoate (0.17 g, 0.75 mmol) and KI (0.005 g, 0.034 mmol) were added and stirred at 60° C. for 12 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.05 g, 17%).

### Step 2. Synthesis of 4-((7-bromo-2,2-dimethyl-4-oxo-1,2,3,4-tetrahydrocarbazol-9-yl) methyl)-N-hydroxy benzamide [Formula 1-6]

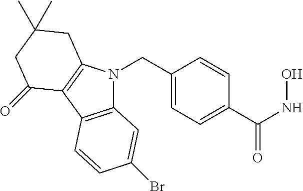 [SMILES: CC1(C)CC(=O)c2c(n(Cc3ccc(C(=O)NO)cc3)c3cc(Br)ccc23)C1]

To a flask were added methyl 4-((7-bromo-2,2-dimethyl-4-oxo-1,2,3,4-tetrahydrocarbazol-9-yl)methyl)benzoate [formula 1-5](0.05 g, 0.11 mmol), hydroxylamine hydrochloride (NH₂OH HCl) (0.039 g, 0.55 mmol), potassium hydroxide (0.062 g, 1.1 mmol) and methanol (30 mL), and stirred for 10 minutes. Then, hydroxylamine 50% aqueous solution had been added drop-wise slowly until the mixed solution in the flask became clear, and the mixture was stirred at room temperature for 6 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 20/1) to yield the compound 54 as solid (0.025 g, 52%).

¹H NMR (400 MHz, DMSO-d₆) δ 7.94 (d, 1H, J=8.3 Hz), 7.79 (s, 1H), 7.69 (d, 2H, J=8.2 Hz), 7.34 (dd, 1H, J=8.3, 1.6 Hz), 7.10 (d, 2H, J=8.1 Hz), 5.57 (s, 2H), 2.85 (s, 2H), 2.38 (s, 2H), 1.06 (s, 6H). MS (ESI) m/z 442 (M⁺+H).

### Example 16

### Synthesis of Compound 55

### Step 1. Synthesis of 6-fluoro-2,2-dimethyl-2,3-dihydro-1H-carbazol-4(9H)-on [formula 1-2]

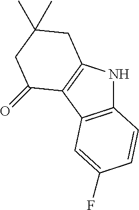 [SMILES: CC1(C)CC(=O)c2c([nH]c3ccc(F)cc23)C1]

To a microwave vial were added 4-fluorophenyl hydrazine HCl [formula 1-1] (0.2 g, 1.23 mmol), 5-5-dimethyl-1,3-cyclohexandion (0.18 g, 1.35 mmol) and TFA (1.5 mL), and a reaction was carried out in a microwave reactor at 140° C. for 30 minutes. Then, the reaction mixture was extracted with ethyl acetate and saturated NaHCO₃ aqueous solution. The organic layer was washed with brine, dried over anhydrous Na₂SO₄, filtered and concentrated under reduced pressure. Residue was purified by column chromatography (SiO₂; hexane/ethylacetate, 3/1) to yield the title compound (0.117 g, 41%).

### Step 2. Synthesis of ethyl 6-(6-fluor-2,2-dimethyl-4-oxo-1,2,3,4-tetrahydrocarbazol-9-yl)hexanoate [Formula 1-3]

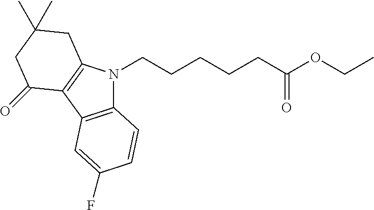 [SMILES: CCOC(=O)CCCCCn1c2c(c3cc(F)ccc31)C(=O)CC(C)(C)C2]

6-fluoro-2,2-dimethyl-2,3-dihydro-1H-carbazol-4(9H)-on [formula 1-2] (0.1 g, 0.43 mmol) was dissolved in DMF (20 mL), and NaH (0.015 g, 0.645 mmol) was added and stirred for 10 minutes. Then, ethyl 6-bromohexanoate (0.096 g, 0.43 mmol) was added and stirred at 60° C. for 12 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 3/1) to yield the title compound (0.18 g, 100%).

### Step 3. Synthesis of 6-(6-fluoro-2,2-dimethyl-4-oxo-1,2,3,4-tetrahydrocarbazol-9-yl)-N-hydroxyhexanamide [formula 1-4]

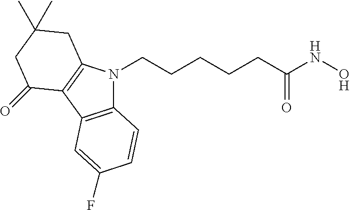 [SMILES: CC1(C)CC(=O)c2c(n(CCCCCC(=O)NO)c3ccc(F)cc23)C1]

To a flask were added ethyl 6-(6-fluoro-2,2-dimethyl-4-oxo-1,2,3,4-tetrahydrocarbazol-9-yl)hexanoate [formula 1-3] (0.18 g, 0.48 mmol), hydroxylamine hydrochloride (NH₂OH HCl) (0.167 g, 2.41 mmol), potassium hydroxide (0.269 g, 4.8 mmol) and methanol (10 mL), and stirred for 10 minutes. Then, hydroxylamine 50% aqueous solution had been added drop-wise slowly until the mixed solution in the flask became clear, and the mixture was stirred at room temperature for 5 hours. After the completion of the reaction, methanol was distilled out under reduced pressure, the reaction mixture was extracted with ethyl acetate only, dried over anhydrous Na₂SO₄, filtered and concentrated under reduced pressure. Residue was purified by column chromatography (SiO₂; dichloromethane/methanol, 20/1) to yield the compound 55 as solid (0.035 g, 20%).

¹H NMR (400 MHz, MeOD-d₃) δ 7.71 (dd, 1H, J=9.3, 2.5 Hz), 7.45 (dd, 1H, J=8.9, 4.2 Hz), 7.02 (td, 1H, J=9.1, 2.6 Hz), 4.21 (t, 2H, J=7.2 Hz), 2.91 (s, 2H), 2.44 (s, 2H), 2.08 (t, 2H, J=7.3 Hz), 1.85-1.78 (m, 2H), 1.69-1.61 (m, 2H), 1.41-1.37 (m, 2H), 1.18 (s, 6H). MS (ESI) m/z 361 (M⁺+H).

### Example 17

### Synthesis of Compound 56

### Step 1. Synthesis of ethyl 7-(6-fluoro-2,2-dimethyl-4-oxo-1,2,3,4-tetrahydrocarbazol-9-yl)heptanoate [formula 1-3]

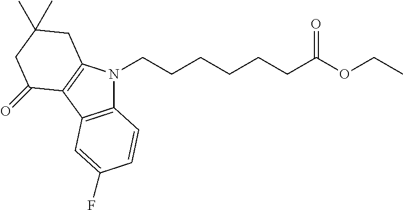 [SMILES: CCOC(=O)CCCCCCn1c2c(c3cc(F)ccc31)C(=O)CC(C)(C)C2]

6-fluoro-2,2-dimethyl-2,3-dihydro-1H-carbazol-4(9H)-on (0.1 g, 0.43 mmol) was dissolved in DMF (20 mL), and NaH (0.015 g, 0.645 mmol) was added and stirred for 10 minutes. Then, ethyl 7-bromoheptanoate (0.102 g, 0.43 mmol) was added and stirred at 60° C. for 12 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.173 g, 100%).

### Step 2. Synthesis of 7-(6-fluoro-2,2-dimethyl-4-oxo-1,2,3,4-tetrahydrocarbazol-9-yl)-N-hydroxyheptanamide [Formula 1-4]

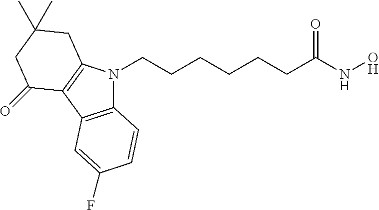 [SMILES: CC1(C)CC(=O)c2c(n(CCCCCCC(=O)NO)c3ccc(F)cc23)C1]

To a flask were added ethyl 7-(6-fluoro-2,2-dimethyl-4-oxo-1,2,3,4-tetrahydrocarbazol-9-yl)heptanoate [formula 1-3](0.173 g, 0.45 mmol), hydroxylamine hydrochloride (NH₂OH HCl) (0.154 g, 2.23 mmol), potassium hydroxide (0.252 g, 4.5 mmol) and methanol (10 mL) and stirred for 10 minutes. Then, hydroxylamine 50% aqueous solution had been added drop-wise slowly until the mixed solution in the flask became clear, and the mixture was stirred at room temperature for 5 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 20/1) to yield the compound 56 as solid (0.028 g, 17%).

¹H NMR (400 MHz, MeOD-d₃) δ 7.72 (dd, 1H, J=9.3, 2.5 Hz), 7.45 (dd, 1H, J=8.9, 4.2 Hz), 7.02 (td, 1H, J=9.1, 2.6 Hz), 4.21 (t, 2H, J=7.3 Hz), 2.91 (s, 2H), 2.44 (s, 2H), 2.07 (t, 2H, J=7.3 Hz), 1.82-1.79 (m, 2H), 1.62-1.58 (m, 2H), 1.39-1.35 (m, 4H), 1.18 (s, 6H). MS (ESI) m/z 375 (M⁺+H).

### Example 18

### Synthesis of Compound 57

### Step 1. Synthesis of methyl 4-((6-fluoro-2,2-dimethyl-4-oxo-1,2,3,4-tetrahydrocarbazol-9-yl)methyl)benzoate [formula 1-3]

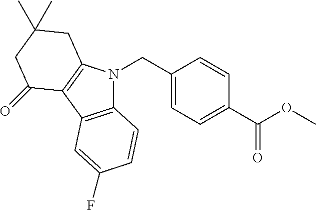 [SMILES: COC(=O)c1ccc(Cn2c3c(c4cc(F)ccc42)C(=O)CC(C)(C)C3)cc1]

6-fluoro-2,2-dimethyl-2,3-dihydro-1H-carbazol-4(9H)-on [formula 1-2] (0.1 g, 0.43 mmol) was dissolved in DMF (20 mL). Thereto, t-BuOK (0.058 g, 0.516 mmol) was added and stirred for 10 minutes. Then, ethyl 7-bromoheptanoate (0.102 g, 0.43 mmol) and KI (0.007 g, 0.043 mmol) were added and stirred at 60° C. for 12 hours. After the completion of the reaction, DMF was distilled out under reduced pressure, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 2/1) to yield the title compound (0.28 g, 100%).

### Step 2. Synthesis of 4-((6-fluoro-2,2-dimethyl-4-oxo-1,2,3,4-tetrahydrocarbazol-9-yl)methyl)-N-hydroxybenzamide [formula 1-4]

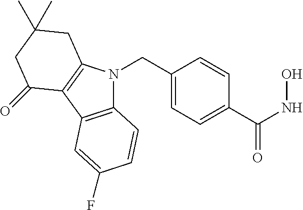 [SMILES: CC1(C)CC(=O)c2c(n(Cc3ccc(C(=O)NO)cc3)c3ccc(F)cc23)C1]

To a flask were added methyl 4-((6-fluoro-2,2-dimethyl-4-oxo-1,2,3,4-tetrahydrocarbazol-9-yl)methyl)benzoate [formula 1-3](0.28 g, 0.74 mmol), hydroxylamine hydrochloride (NH₂OH HCl) (0.256 g, 3.68 mmol), potassium hydroxide (0.415 g, 7.4 mmol) and methanol (20 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only. And, it was dried over anhydrous Na₂SO₄, filtered and concentrated under reduced pressure. Residue was purified by column chromatography (SiO₂; dichloromethane/methanol, 20/1) to yield the compound 57 as solid (0.08 g, 28%).

¹H NMR (400 MHz, MeOD-d₃) δ 7.77 (dd, 1H, J=9.3, 2.5 Hz), 7.70 (d, 2H, J=8.3 Hz), 7.36 (dd, 1H, J=8.9, 4.2 Hz), 7.15 (d, 2H, J=8.3 Hz), 6.98 (td, 1H, J=9.1, 2.6 Hz), 5.55 (s, 2H), 3.36 (s, 3H), 2.87 (s, 2H), 2.47 (s, 2H), 1.15 (s, 6H). MS (ESI) m/z 381 (M⁺+H).

### Example 19

### Synthesis of Compound 60

### Step 1. Synthesis of 5-fluoro-2,2-dimethyl-2,3,9,9a-tetrahydro-1H-carbazol-4(4aH)-on [formula 1-2]

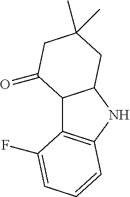 [SMILES: CC1(C)CC(=O)C2c3c(F)cccc3NC2C1]

To a microwave vial were added 3-fluorophenyl hydrazine HCl [formula 1-1] (0.696 g, 4.28 mmol), 5-5-dimethyl-1,3-cyclohexandion (0.5 g, 3.57 mmol) and TFA (4 mL), and a reaction was carried out in a microwave reactor at 140° C. for 10 minutes. Then, solvent (TFA) was concentrated under reduced pressure, the reaction mixture was extracted carried out with ethyl acetate and saturated NaHCO₃ aqueous solution, dried over anhydrous MgSO₄, filtered and concentrated under reduced pressure. Residue was purified by column chromatography (SiO₂; hexane/ethylacetate, 5/5) to yield the title compound (0.102 g, 12.4%).

### Step 2. Synthesis of ethyl 6-(5-fluoro-2,2-dimethyl-4-oxo-2,3,4,4a-tetrahydro-1H-carbazol-9(9aH)-yl)hexanoate [Formula 1-3]

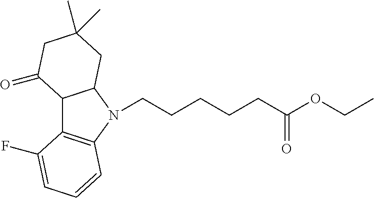 [SMILES: CCOC(=O)CCCCCN1c2cccc(F)c2C2C(=O)CC(C)(C)CC21]

5-fluoro-2,2-dimethyl-2,3,9,9a-tetrahydro-1H-carbazol-4(4aH)-on [formula 1-2] (0.1 g, 0.44 mmol) was dissolved in DMF (5 mL). Thereto, NaH (0.039 g, 0.88 mmol) was added and stirred for 10 minutes. Then, ethyl 6-bromohexanoate (0.12 g, 0.53 mmol) was added and stirred at 60° C. for 5 hours. By adding saturated NaHCO₃, the reaction was completed. The reaction mixture was extracted with ethyl acetate, dried over anhydrous MgSO₄, filtered and concentrated under reduced pressure. Residue was purified by column chromatography (SiO₂; hexane/ethylacetate, 6/4) to yield the title compound (0.102 g, 62%).

### Step 3. Synthesis of 6-(5-fluoro-2,2-dimethyl-4-oxo-2,3,4,4a-tetrahydro-1H-carbazol-9(9aH)-yl)-N-hydroxyhexanamide [formula 1-4]

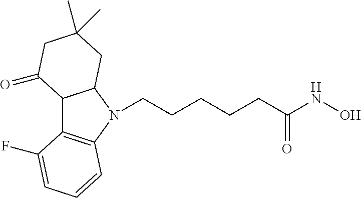 [SMILES: CC1(C)CC(=O)C2c3c(F)cccc3N(CCCCCC(=O)NO)C2C1]

To a flask were added ethyl 6-(5-fluoro-2,2-dimethyl-4-oxo-2,3,4,4a-tetrahydro-1H-carbazol-9(9aH)-yl)hexanoate [formula 1-3] (0.103 g, 0.28 mmol), hydroxylamine hydrochloride (NH₂OH HCl) (0.095 g, 1.37 mmol), potassium hydroxide (0.153 g, 2.74 mmol) and methanol (5 mL), and stirred for 1 hour. Hydroxylamine 50% aqueous solution had been added drop-wise slowly until the mixed solution in the flask became clear and the mixture was stirred at room temperature for overnight. After the completion of the reaction, methanol was distilled out under reduced pressure. And the reaction mixture was extracted with ethyl acetate only, dried over anhydrous Na₂SO₄, filtered and concentrated under reduced pressure. Residue was purified by column chromatography (SiO₂; dichloromethane/methanol, 10/1) to yield the compound 60 (0.057 g, 57.8%).

¹H NMR (400 MHz, DMSO-d₆) δ 10.32 (s, 1H), 8.68 (s, 1H), 7.36 (d, 1H, J=8.2), 7.23-7.18 (m, 1H), 6.92 (dd, 1H, J=10.6 Hz, 7.9 Hz), 4.18 (t, 2H, J=7.3 Hz), 2.91 (s, 2H), 2.36 (s, 2H), 1.92 (t, 2H, J=7.3 Hz), 1.70-1.60 (m, 2H), 1.54-1.47 (m, 2H), 1.29-1.22 (m, 2H), 1.09 (s, 6H); MS (ESI) m/z 361 (M⁺+H).

### Example 20

### Synthesis of Compound 71

### Step 1. Synthesis of 6,7-difluoro-2,2-dimethyl-2,3-dihydro-1H-carbazol-4(9H)-on [formula 1-2]

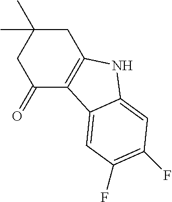 [SMILES: CC1(C)CC(=O)c2c([nH]c3cc(F)c(F)cc23)C1]

To a microwave vial were added 5,5-dimethyl-1,3-cyclohexandion [formula 1-1] (1.0 g, 7.13 mmol), 3,4-difluorophenyl hydrazine HCl (1.55 g, 8.56 mmol) and TFA (9.0 mL), and a reaction was carried out in a microwave reactor at 140° C. for 10 minutes. Then, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 3/1) to yield the title compound (0.580 g, 32%).

### Step 2. Synthesis of methyl 4-((6,7-difluoro-2,2-dimethyl-4-oxo-1,2,3,4-tetrahydrocarbazol-9-yl)methyl)benzoate [Formula 1-3]

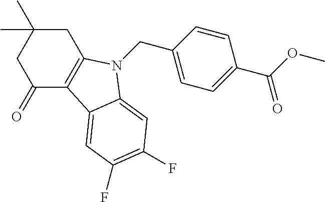

6,7-difluoro-2,2-dimethyl-2,3-dihydro-1H-carbazol-4(9H)-on [formula 1-2] (0.38 g, 1.52 mmol) was dissolved in DMF (10 mL), 55% NaH in paraffin solution (0.133 g, 3.04 mmol) was added and stirred for 10 minutes. Then, methyl-4(bromomethyl)benzoate (0.418 g, 1.82 mmol) was added and stirred at room temperature for 5 hours. After the completion of the reaction, the reaction mixture was washed with ethyl acetate and water, dried over anhydrous MgSO₄, filtered and concentrated under reduced pressure. Residue was purified by column chromatography (SiO₂; hexane/ethylacetate, 6/1) to yield the title compound (0.26 g, 44%).

### Step 3. Synthesis of 4-((6,7-difluoro-2,2-dimethyl-4-oxo-1,2,3,4-tetrahydrocarbazol-9-yl)methyl)-N-hydroxybenzamide [formula 1-4]

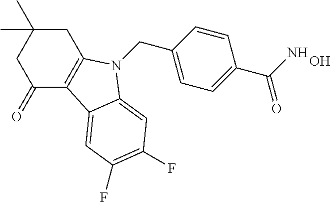

To a flask were added methyl 4-((6,7-difluoro-2,2-dimethyl-4-oxo-1,2,3,4-tetrahydrocarbazol-9-yl)methyl)benzoate [formula 1-3] (0.255 g, 0.64 mmol), hydroxylamine hydrochloride (NH₂OH HCl) (0.222 g, 3.20 mmol), potassium hydroxide (0.358 g, 6.4 mmol) and methanol (10 mL), and stirred for 10 minutes. Then, hydroxylamine 50% aqueous solution had been added drop-wise slowly until the mixed solution in the flask became clear, and the mixture was stirred for one day. After the completion of the reaction, methanol was distilled out under reduced pressure, and residue was purified by column chromatography (SiO₂; dichloromethane/methanol, 10/1) to yield the compound 71 as solid (0.08 g, 31%).

¹H NMR (400 MHz, DMSO-d₆) δ 11.3 (s, 1H), 9.14 (s, 1H), 7.98-7.86 (m, 2H), 7.81 (d, 2H, J=8.2 Hz), 7.25 (d, 2H, J=8.2 Hz), 5.68 (s, 2H), 2.98 (s, 2H), 2.50 (s, 2H), 1.18 (s, 6H). MS (ESI) m/z 399 (M⁺+H).

### Example 21

### Synthesis of Compound 72

### Step 1. Synthesis of ethyl 6-(6,7-difluoro-2,2-dimethyl-4-oxo-1,2,3,4-tetrahydrocarbazol-9-yl)hexanoate [formula 1-3]

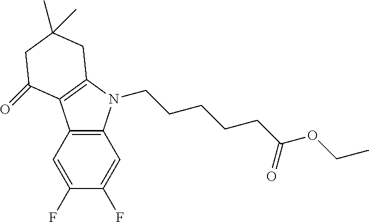 [SMILES: CCOC(=O)CCCCCn1c2c(c3cc(F)c(F)cc31)C(=O)CC(C)(C)C2]

6,7-difluoro-2,2-dimethyl-2,3-dihydro-1H-carbazol-4(9H)-on [formula 1-2] (0.20 g, 0.802 mmol) was dissolved in DMF (10 mL). Thereto, 55% NaH in paraffin solution (0.07 g, 1.60 mmol) was added and stirred for 10 minutes. Then, ethyl 6-bromohexanoate (0.215 g, 0.962 mmol) was added and stirred at room temperature for 5 hours. After the completion of the reaction, the reaction mixture was washed with ethyl acetate and water (H₂O), dried over anhydrous MgSO₄, filtered and concentrated under reduced pressure. Residue was purified by column chromatography (SiO₂; hexane/ethylacetate, 6/1) to yield the title compound (0.073 g, 23%).

### Step 2. Synthesis of 6-(6,7-difluoro-2,2-dimethyl-4-oxo-1,2,3,4-tetrahydrocarbazol-9-yl)-N-hydroxyhexanamide [Formula 1-4]

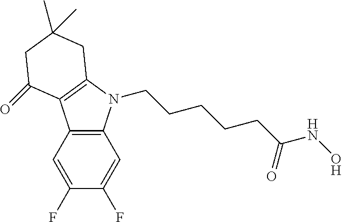 [SMILES: CC1(C)CC(=O)c2c(n(CCCCCC(=O)NO)c3cc(F)c(F)cc23)C1]

To a flask were added ethyl 6-(6,7-difluoro-2,2-dimethyl-4-oxo-1,2,3,4-tetrahydrocarbazol-9-yl)hexanoate [formula 1-3] (0.073 g, 0.186 mmol), hydroxylamine hydrochloride (NH₂OH HCl) (0.065 g, 0.93 mmol), potassium hydroxide (0.104 g, 1.86 mmol) and methanol (10 mL), and stirred for 10 minutes. Then, hydroxylamine 50% aqueous solution had been added drop-wise slowly until the mixed solution in the flask became clear, and the mixture was stirred for one day. After the completion of the reaction, methanol was distilled out under reduced pressure, and residue was purified by column chromatography (SiO₂; dichloromethane/methanol, 10/1) to yield the compound 72 as solid (0.02 g, 27%).

¹H NMR (400 MHz, MeOD-d₃) δ 7.82 (dd, 1H, J=10.8 Hz, J=8.0 Hz), 7.46 (dd, 1H, J=10.8 Hz, J=6.6 Hz), 4.17 (t, 2H, J=7.2 Hz), 2.91 (s, 2H), 2.43 (s, 2H) 2.09-2.06 (m, 2H), 1.81-1.77 (m, 2H), 1.66-1.62 (m, 2H), 1.37-1.35 (m, 2H), 1.17 (s, 6H). MS (ESI) m/z 395 (M⁺+H).

### Example 22

### Synthesis of Compound 73

### Step 1. Synthesis of 5,6-difluoro-2,2-dimethyl-2,3-dihydro-1H-carbazol-4(9H)-on [formula 1-2]

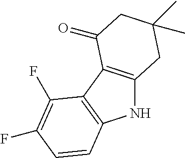 [SMILES: CC1(C)CC(=O)c2c([nH]c3ccc(F)c(F)c23)C1]

To a microwave vial were added 5,5-dimethyl-1,3-cyclohexandion [formula 1-1] (0.5 g, 3.57 mmol), 3,4-difluorophenyl hydrazine HCl (0.773 g, 4.28 mmol) and TFA (4.0 mL), and a reaction was carried out in a microwave reactor at 140° C. for 10 minutes. Then, TFA was distilled out under reduced pressure, the reaction mixture was extracted with ethyl acetate and saturated NaHCO₃ aqueous solution, dried over anhydrous MgSO₄, filtered and concentrated under reduced pressure. Residue was purified by column chromatography (SiO₂; hexane/ethylacetate, 3/1) to yield the title compound (0.10 g, 11%).

### Step 2. Synthesis of methyl 4-((5,6-difluoro-2,2-dimethyl-4-oxo-1,2,3,4-tetrahydrocarbazol-9-yl)methyl)benzoate [Formula 1-3]

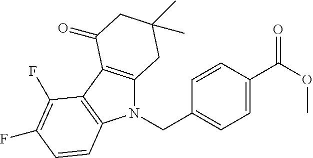 [SMILES: COC(=O)c1ccc(Cn2c3c(c4c(F)c(F)ccc42)C(=O)CC(C)(C)C3)cc1]

5,6-difluoro-2,2-dimethyl-2,3-dihydro-1H-carbazol-4(9H)-on [formula 1-2] (0.217 g, 0.871 mmol) was dissolved in DMF (4.0 mL). Thereto, 55% NaH in paraffin solution (0.076 g, 1.74 mmol) was added and stirred for 10 minutes. Then, methyl-4(bromomethyl)benzoate (0.239 g, 1.045 mmol) was added and stirred at 50° C. for 5 hours. After the completion of the reaction, the reaction mixture was washed with ethyl acetate and water (H₂O), dried over anhydrous MgSO₄, filtered and concentrated under reduced pressure. Residue was purified by column chromatography (SiO₂; hexane/ethylacetate, 6/1) to yield the title compound (0.123 g, 35%).

### Step 3. Synthesis of 4-((5,6,-difluoro-2,2-dimethyl-4-oxo-1,2,3,4-tetrahydrocarbazol-9-yl)methyl)-N-hydroxybenzamide [formula 1-4]

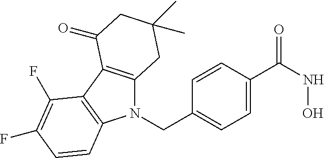 [SMILES: CC1(C)CC(=O)c2c(n(Cc3ccc(C(=O)NO)cc3)c3ccc(F)c(F)c23)C1]

To a flask were added methyl 4-((5,6-difluoro-2,2-dimethyl-4-oxo-1,2,3,4-tetrahydrocarbazol-9-yl)methyl)benzoate [formula 1-3] (0.11 g, 0.277 mmol), hydroxylamine hydrochloride (NH₂OH HCl) (0.096 g, 1.38 mmol), potassium hydroxide (0.155 g, 2.77 mmol) and methanol (5.0 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was purified by column chromatography (SiO₂; dichloromethane/methanol, 10/1) to yield the compound 73 as solid (0.05 g, 45%).

¹H NMR (400 MHz, MeOD-d₃) δ 7.86 (d, 2H, J=8.3 Hz), 7.28-7.26 (m, 4H), 5.70 (s, 2H), 3.02 (s, 2H), 2.64 (s, 2H), 1.38 (s, 6H). MS (ESI) m/z 399 (M⁺+H).

### Example 23

### Synthesis of Compound 74

### Step 1. Synthesis of methyl 6-(5,6-difluoro-2,2-dimethyl-4-oxo-1,2,3,4-tetrahydrocarbazol-9-yl)hexanoate [formula 1-3]

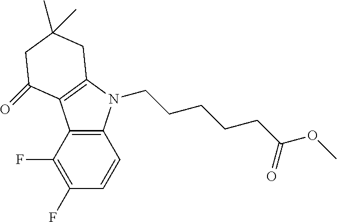 [SMILES: COC(=O)CCCCCn1c2c(c3c(F)c(F)ccc31)C(=O)CC(C)(C)C2]

5,6-difluoro-2,2-dimethyl-2,3-dihydro-1H-carbazol-4(9H)-on [formula 1-2] (0.215 g, 0.863 mmol) was dissolved in DMF (4.0 mL). Thereto, 55% NaH in paraffin solution (0.075 g, 1.72 mmol) was added and stirred for 10 minutes. Then, ethyl 6-bromohexanoate (0.214 g, 0.960 mmol) was added and stirred at 50° C. for 5 hours. After the completion of the reaction, DMF was distilled out under reduced pressure, and the reaction mixture was washed with ethyl acetate and water (H₂O), dried over anhydrous MgSO₄, filtered and concentrated under reduced pressure. Residue was purified by column chromatography (SiO₂; hexane/ethylacetate, 6/1) to yield the title compound (0.202 g, 59%).

### Step 2. Synthesis of 6-(5,6-difluoro-2,2-dimethyl-4-oxo-1,2,3,4-tetrahydrocarbazol-9-yl)-N-hydroxyhexanamide [Formula 1-4]

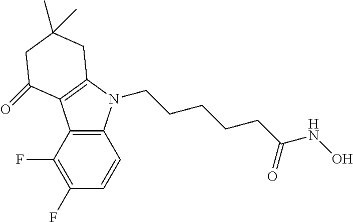 [SMILES: CC1(C)CC(=O)c2c(n(CCCCCC(=O)NO)c3ccc(F)c(F)c23)C1]

To a flask were added methyl 6-(5,6-difluoro-2,2-dimethyl-4-oxo-1,2,3,4-tetrahydrocarbazol-9-yl)hexanoate [formula 1-3] (0.17 g, 0.434 mmol), hydroxylamine hydrochloride (NH₂OH HCl) (0.15 g, 2.171 mmol), potassium hydroxide (0.243 g, 4.34 mmol) and methanol (5.0 mL), and stirred for 10 minutes. Then, hydroxylamine 55% aqueous solution had been added drop-wise slowly until the mixed solution in the flask became clear, and the mixture was stirred for one day. After the completion of the reaction, methanol was distilled out under reduced pressure, and the reaction mixture was extracted with ethyl acetate only, dried over anhydrous MgSO₄, filtered and concentrated under reduced pressure. Residue was purified by column chromatography (SiO₂; dichloromethane/methanol, 10/1) to yield the compound 74 as solid (0.045 g, 27%).

¹H NMR (400 MHz, MeOD-d₃) δ 7.45-7.39 (m, 1H), 7.35-7.22 (m, 1H), 4.36 (t, 2H, J=7.3 Hz), 3.07 (s, 2H), 2.62 (s, 2H), 2.23-2.19 (m, 2H), 1.94-1.92 (m, 2H), 1.81-1.77 (m, 2H), 1.57-1.46 (m, 2H), 1.32 (s, 6H). MS (ESI) m/z 395 (M⁺+H).

### Example 24

### Synthesis of Compound 76

### Step 1. Synthesis of ethyl 6-(7-fluoro-2,2-dimethyl-4-oxo-1,2,3,4-tetrahydrocarbazol-9-yl)hexanoate [formula 1-3]

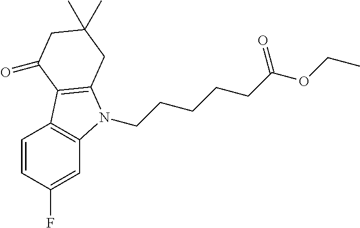 [SMILES: CCOC(=O)CCCCCn1c2c(c3ccc(F)cc31)C(=O)CC(C)(C)C2]

7-fluoro-2,2-dimethyl-2,3-dihydro-1H-carbazol-4(9H)-on [formula 1-2] (0.2 g, 0.87 mmol) was dissolved in DMF (5 mL), and NaH (0.039 g, 0.88 mmol) was added and stirred for 10 minutes. Then, ethyl 6-bromohexanoate (0.23 g, 1.04 mmol) was added and stirred at 60° C. for 5 hours. Reaction was completed by adding saturated NaHCO₃, and the reaction mixture was extracted with ethyl acetate. It was dried over anhydrous MgSO₄, filtered and concentrated under reduced pressure. Residue was purified by column chromatography (SiO₂; hexane/ethylacetate, 6/4) to yield the title compound (0.102 g, 62%).

### Step 2. Synthesis of 6-(7-fluoro-2,2-dimethyl-4-oxo-1,2,3,4-tetrahydrocarbazol-9-yl)-N-hydroxyhexanamide [Formula 1-4]

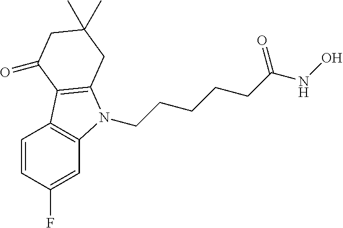 [SMILES: CC1(C)CC(=O)c2c(n(CCCCCC(=O)NO)c3cc(F)ccc23)C1]

To a flask were added ethyl 6-(7-fluoro-2,2-dimethyl-4-oxo-1,2,3,4-tetrahydrocarbazol-9-yl)hexanoate [formula 1-3] (0.18 g, 0.48 mmol), hydroxylamine hydrochloride (NH₂OH HCl) (0.167 g, 2.41 mmol), potassium hydroxide (0.27 g, 4.82 mmol) and methanol (5 mL), and stirred. Then, hydroxylamine 50% aqueous solution had been added drop-wise slowly until the mixed solution in the flask became clear, and the mixture was stirred at room temperature for overnight. After the completion of the reaction, methanol was distilled out under reduced pressure. And, the reaction mixture was diluted with ethyl acetate, washed by brine, dried over anhydrous MgSO₄, filtered and concentrated under reduced pressure. Residue was purified by column chromatography (SiO₂; dichloromethane/methanol, 10/1) to yield the compound 76 (0.077 g, 44%).

¹H NMR (400 MHz, MeOD-d₃) δ 8.01 (dd, 1H, J=8.6 Hz, 5.5 Hz), 7.26 (dd, 1H, J=9.8 Hz, 2.2 Hz), 6.99 (td, 1H, J=8.7, 2.2 Hz), 4.18 (t, 2H, J=7.2), 2.91 (s, 2H), 2.44 (s, 2H), 2.07 (t, 2H, J=7.3 Hz), 1.84-1.76 (m, 2H), 1.69-1.61 (m, 2H), 1.40-1.33 (m, 2H), 1.18 (s, 6H); MS (ESI) m/z 361 (M+1)⁺.

### Example 25

### Synthesis of Compound 85

### Step 1. Synthesis of 2,3-dihydro-1H-carbazol-4(9H)-on [Formula 2-2]

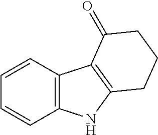 [SMILES: O=C1CCCc2[nH]c3ccccc3c21]

To a microwave vial were added 1,3-cyclohexandion [formula 2-1] (1.5 g, 13.4 mmol), penylhydrazine (1.73 g, 16.1 mmol) and TFA (4.0 mL), and a reaction was carried out in a microwave reactor at 140° C. for 10 minutes. Then, TFA was distilled out under reduced pressure. The reaction mixture was extracted with ethyl acetate and saturated NaHCO₃ aqueous solution, dried over anhydrous MgSO₄, filtered and concentrated under reduced pressure. Residue was purified by column chromatography (SiO₂; hexane/ethylacetate, 4/1) to yield the title compound (1.0 g, 40%).

### Step 2. Synthesis of methyl 4-((4-oxo-1,2,3,4-tetrahydrocarbazol-9-yl)methyl)benzoate [formula 2-3]

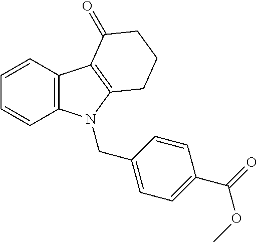 [SMILES: COC(=O)c1ccc(Cn2c3c(c4ccccc42)C(=O)CCC3)cc1]

2,3-dihydro-1H-carbazol-4(9H)-on [formula 2-2] (1.05 g, 5.67 mmol) was dissolved in DMF (4.0 mL), and 55% NaH in paraffin solution (0.495 g, 11.3 mmol) was added and stirred for 10 minutes. Then, methyl 4-(bromomethyl)benzoate (1.55 g, 6.80 mmol) was added and stirred at 50° C. for 5 hours. After the completion of the reaction, the reaction mixture was extracted with ethyl acetate and water (H₂O), dried over anhydrous MgSO₄, filtered and concentrated under reduced pressure. Residue was purified by column chromatography (SiO₂; hexane/ethylacetate, 5/1) to yield the title compound (0.65 g, 34%).

### Step 3. Synthesis of methyl 4-((3-methylen-4-oxo-1,2,3,4-tetrahydrocarbazol-9-yl)methyl)benzoate [formula 2-4]

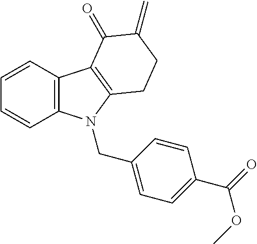 [SMILES: C=C1CCc2c(c3ccccc3n2Cc2ccc(C(=O)OC)cc2)C1=O]

Methyl 4-((4-oxo-1,2,3,4-tetrahydrocarbazol-9-yl)methyl)benzoate [formula 2-3] (0.65 g, 1.95 mmol), N,N-dimethylamine, HCl (0.318 g, 3.90 mmol), paraformaldehyde (0.130 g, 3.90 mmol) and mixed solvent (acetic acid:toluene=4:1, 15 mL) were added, and stirred at 100° C. for 4 hours. After the completion of the reaction, acetic acid was distilled out under reduced pressure, and without purification, the compound was dissolved in a 15 mL of mixed solvent (acetonitrile:H₂O=1:4) and stirred at 80° C. for 12 hours. After the completion of the reaction, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 7/1) to yield the title compound (0.28 g, 41%).

### Step 4. Synthesis of methyl 4-((3-((2-methyl-1H-imidazol-1-yl)methyl)-4-oxo-1,2,3,4-tetrahydrocarbazol-9-yl)methyl)benzoate [Formula 2-5]

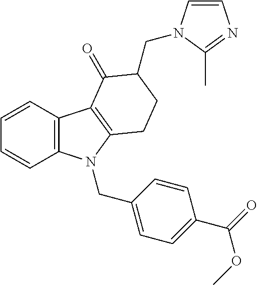 [SMILES: COC(=O)c1ccc(Cn2c3c(c4ccccc42)C(=O)C(Cn2ccnc2C)CC3)cc1]

To a microwave vial were added methyl 4-((3-methylen-4-oxo-1,2,3,4-tetrahydrocarbazol-9-yl)methyl)benzoate [formula 2-4] (0.11 g, 0.318 mmol), 2-methylimidazole (0.078 g, 0.96 mmol) and toluene (3.0 mL), and a reaction was carried out in a microwave reactor at 110° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.07 g, 51%).

### Step 5. Synthesis of N-hydroxy-4-((3-((2-methyl-1H-imidazole-1-yl)methyl)-4-oxo-1,2,3,4-tetrahydrocarbazol-9-yl)methyl)benzamide [formula 2-6]

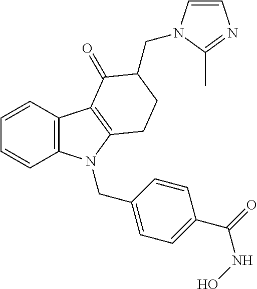 [SMILES: Cc1nccn1CC1CCc2c(c3ccccc3n2Cc2ccc(C(=O)NO)cc2)C1=O]

To a flask were added methyl 4-((3-((2-methyl-1H-imidazole-1-yl)methyl)-4-oxo-1,2,3,4-tetrahydrocarbazol-9-yl)methyl)benzoate [formula 2-5] (0.07 g, 0.164 mmol), hydroxylamine hydrochloride (NH₂OH HCl) (0.06 g, 0.82 mmol), potassium hydroxide (0.092 g, 1.637 mmol) and methanol (5.0 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was purified by column chromatography (SiO₂; dichloromethane/methanol, 10/1) to yield the compound 85 as solid (0.03 g, 42%).

¹H NMR (400 MHz, DMSO-d₆) δ 10.9 (s, 1H), 8.80 (s, 1H), 7.83 (d, 1H, J=6.6 Hz), 7.45 (d, 2H, J=8.0 Hz), 7.28 (d, 1H, J=8.0 Hz), 7.00-6.94 (m, 4H), 6.85 (s, 1H), 6.52 (s, 1H), 5.31 (s, 2H), 4.22-4.27 (m, 1H), 3.83-3.88 (m, 1H), 2.87-2.91 (m, 1H), 2.70-2.74 (m, 2H), 2.11 (s, 3H), 1.73-1.87 (m, 1H), 1.61-1.69 (m, 1H). MS (ESI) m/z 429 (M⁺+H).

### Example 26

### Synthesis of Compound 86

### Step 1. Synthesis of methyl 4-((4-oxo-3-(piperidin-1-ylmethyl)-1,2,3,4-tetrahydrocarbazol-9-yl)methyl)benzoate [formula 2-5]

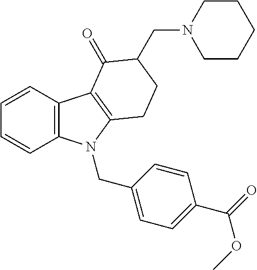 [SMILES: COC(=O)c1ccc(Cn2c3c(c4ccccc42)C(=O)C(CN2CCCCC2)CC3)cc1]

To a microwave vial were added methyl 4-((3-methylen-4-oxo-1,2,3,4-tetrahydrocarbazol-9-yl)methyl)benzoate [formula 2-4] (0.05 g, 0.145 mmol), piperidine (0.015 g, 0.174 mmol) and toluene (3.0 mL), and a reaction was carried out in a microwave reactor at 110° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.057 g, 88%).

### Step 2. Synthesis of N-hydroxy-4-((4-oxo-3-(piperidin-1-ylmethyl)-1,2,3,4-tetrahydrocarbazol-9-yl)methyl)benzamide [formula 2-6]

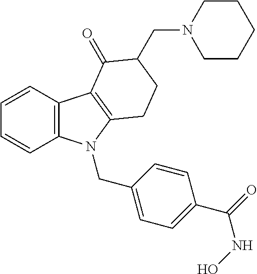 [SMILES: O=C(NO)c1ccc(Cn2c3c(c4ccccc42)C(=O)C(CN2CCCCC2)CC3)cc1]

To a flask were added methyl 4-((4-oxo-3-(piperidin-1-ylmethyl)-1,2,3,4-tetrahydrocarbazol-9-yl)methyl)benzoate [formula 2-5] (0.057 g, 0.132 mmol), hydroxylamine hydrochloride (NH₂OH HCl) (0.046 g, 0.662 mmol), potassium hydroxide (0.074 g, 1.32 mmol) and methanol (10 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 86 as solid (0.02 g, 35%).

¹H NMR (400 MHz, DMSO-d₆) δ 10.9 (s, 1H), 8.80 (s, 1H), 7.83-7.80 (m, 1H), 7.46 (d, 2H, =8.2 Hz), 7.28-7.26 (m, 1H), 6.98-6.86 (m, 4H), 5.33 (s, 2H), 3.11-3.05 (m, 3H), 2.87-2.84 (m, 1H), 2.74-2.72 (m, 1H), 2.69-2.68 (m, 2H), 2.58-2.50 (m, 4H), 2.30-2.27 (m, 2H), 1.80-1.74 (m, 1H), 1.38-1.23 (m, 2H), 1.21-1.17 (m, 1H). (ESI) m/z 432 (M⁺+H).

### Example 27

### Synthesis of Compound 87

### Step 1. Synthesis of methyl 4-((3-(morpholinomethyl)-4-oxo-1,2,3,4-tetrahydrocarbazol-9-yl)methyl)benzoate [formula 2-5]

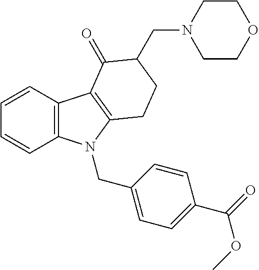 [SMILES: COC(=O)c1ccc(Cn2c3c(c4ccccc42)C(=O)C(CN2CCOCC2)CC3)cc1]

To a microwave vial were added methyl 4-((3-methylen-4-oxo-1,2,3,4-tetrahydrocarbazol-9-yl)methyl)benzoate [formula 2-4] (0.1 g, 0.290 mmol), morpholine (0.076 g, 0.87 mmol) and toluene (3.0 mL), and a reaction was carried out in a microwave reactor at 110° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound as yellow solid (0.07 g, 56%).

### Step 2. Synthesis of N-hydroxy-4-((3-(morpholinomethyl)-4-oxo-1,2,3,4-tetrahydrocarbazol-9-yl)methyl)benzamide [formula 2-6]

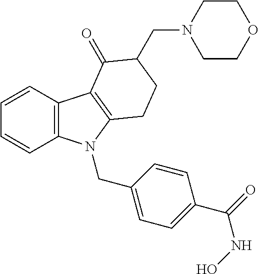 [SMILES: O=C(NO)c1ccc(Cn2c3c(c4ccccc42)C(=O)C(CN2CCOCC2)CC3)cc1]

To a flask were added methyl 4-((3-(morpholinomethyl)-4-oxo-1,2,3,4-tetrahydrocarbazol-9-yl)methyl)benzoate [formula 2-5] (0.07 g, 0.162 mmol), hydroxylamine hydrochloride (NH₂OH HCl) (0.056 g, 0.81 mmol), potassium hydroxide (0.091 g, 1.62 mmol) and methanol (5.0 mL), and stirred for 10 minutes. Then, hydroxylamine 55% aqueous solution had beenadded drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 87 as yellow solid (0.04 g, 57%).

¹H NMR (400 MHz, DMSO-d₆) δ 9.03 (s, 1H), 8.04-8.02 (m, 1H), 7.68 (d, 2H, J=8.2 Hz), 7.50-7.47 (m, 1H), 7.19-7.17 (m, 4H), 5.54 (s, 2H), 3.57 (s, 3H), 3.12-3.06 (m, 1H), 2.99-2.90 (m, 1H), 2.73-2.71 (m, 2H), 2.49-2.30 (m, 3H), 2.06-1.91 (m, 1H). (ESI) m/z 433 (M⁺+H).

### Example 28

### Synthesis of Compound 88

### Step 1. Synthesis of methyl 4-((4-oxo-3-(pyrrolidin-1-ylmethyl)-1,2,3,4-tetrahydrocarbazol-9-yl)methyl)benzoate [formula 2-5]

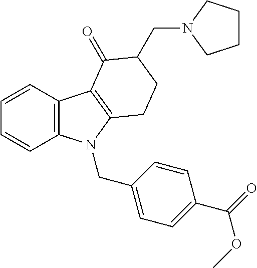 [SMILES: COC(=O)c1ccc(Cn2c3c(c4ccccc42)C(=O)C(CN2CCCC2)CC3)cc1]

To a microwave vial were added methyl 4-((3-methylen-4-oxo-1,2,3,4-tetrahydrocarbazol-9-yl)methyl)benzoate [formula 2-4] (0.05 g, 0.145 mmol), pyrrolidine (0.012 g, 0.17 mmol) and toluene (3.0 mL), and a reaction was carried out in a microwave reactor at 110° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.05 g, 83%).

### Step 2. Synthesis of N-hydroxy-4-((4-oxo-3-(pyrrolidin-1-ylmethyl)-1,2,3,4-tetrahydrocarbazol-9-yl)methyl)benzamide [formula 2-6]

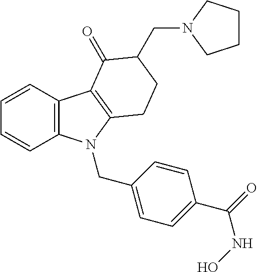 [SMILES: O=C(NO)c1ccc(Cn2c3c(c4ccccc42)C(=O)C(CN2CCCC2)CC3)cc1]

To a flask were added methyl 4-((4-oxo-3-(pyrrolidin-1-ylmethyl)-1,2,3,4-tetrahydrocarbazol-9-yl)methyl)benzoate [formula 2-5] (0.05 g, 0.12 mmol), hydroxylamine hydrochloride (NH₂OH HCl) (0.042 g, 0.60 mmol), potassium hydroxide (0.067 g, 1.20 mmol) and methanol (2.0 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 88 as solid (0.015 g, 30%).

¹H NMR (400 MHz, DMSO-d₆) δ 11.1 (s, 1H), 9.02 (s, 1H), 8.03-8.02 (m, 1H), 7.87-7.66 (m, 2H), 7.47-7.23 (m, 1H), 7.17-7.07 (m, 4H), 5.52 (s, 2H), 3.33-3.21 (m, 4H), 3.07-3.03 (m, 2H), 2.93-2.78 (m, 4H), 2.48-2.31 (m, 1H), 1.98-1.77 (m, 1H), 1.70-1.61 (m, 2H). (ESI) m/z 418 (M⁺+H).

### Example 29

### Synthesis of Compound 99

### Step 1. Synthesis of N-hydroxy-4-((4-oxo-1,2,3,4-tetrahydrocarbazol-9-yl)methyl)benzamide [formula 1-4]

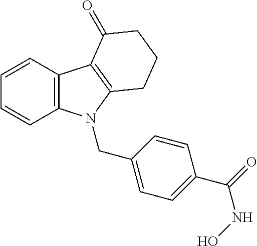 [SMILES: O=C(NO)c1ccc(Cn2c3c(c4ccccc42)C(=O)CCC3)cc1]

To a flask were added methyl 4-((4-oxo-1,2,3,4-tetrahydrocarbazol-9-yl)methyl)benzoate [formula 1-3] (0.1 g, 0.30 mmol), hydroxylamine hydrochloride (NH₂OH HCl) (0.10 g, 1.5 mmol), potassium hydroxide (0.168 g, 3.0 mmol) and methanol (5.0 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 99 as solid (0.04 g, 39%).

¹H NMR (400 MHz, DMSO-d₆) δ 11.1 (s, 1H), 9.05 (s, 1H), 8.02-8.00 (m, 1H), 7.65 (d, 2H, J=8.2 Hz), 7.49-7.48 (m, 1H), 7.18-7.15 (m, 4H), 5.53 (s, 2H), 2.97-2.94 (m, 2H), 2.48-2.42 (m, 2H), 2.13-2.10 (m, 1H). (ESI) m/z 335 (M⁺+H).

### Example 30

### Synthesis of Compound 101

### Step 1. Synthesis of 3,6,6-trimethyl-6,7-dihydro-1H-indazol-4(5H)-on [formula 4-2]

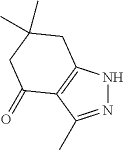

2-acetyldimedone [formula 4-1] (0.5 g, 2.74 mmol) was dissolved in THF (5 mL). Thereto, hydrazine hydrate (0.16 g, 3.16 mmol) was added. The mixture was refluxed with stirring for 3 hours, cooled to room temperature and concentrated under reduced pressure. Residue was purified by column chromatography (SiO₂; hexane/ethylacetate, 1/1) to yield the title compound as white solid (0.45 g, 91.4%).

### Step 2. Synthesis of methyl 4-((3,6,6-trimethyl-4-oxo-4,5,6,7-tetrahydrocarbazol-1-yl) methyl)benzoate [formula 4-3b]

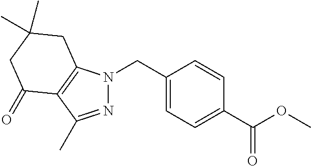

3,6,6-trimethyl-6,7-dihydro-1H-indazol-4(5H)-on [formula 4-2] (0.20 g, 1.12 mmol) was dissolved in DMA (5 mL). Thereto, NaH (0.029 g, 1.23 mmol) was added. 5 minutes later, methyl 4-(bromomethyl)benzoate (0.283 g, 1.23 mmol) was added and a reaction was carried out at room temperature for 4 hours. The reaction mixture was diluted with saturated NH₄Cl and extracted with ethyl acetate, dried over anhydrous MgSO₄, filtered and concentrated under reduced pressure. Residue was purified by column chromatography (SiO₂; hexane/ethylacetate, 6/4) to yield the title compound as white solid (0.069 g, 19%).

### Step 3. Synthesis of N-hydroxy-4-((3,6,6-trimethyl-4-oxo-4,5,6,7-tetrahydroindazol-1-yl)methyl)benzamide [formula 4-4b]

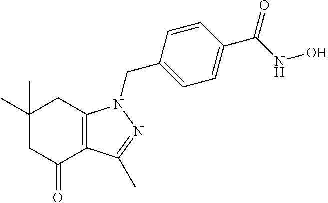 [SMILES: Cc1nn(Cc2ccc(C(=O)NO)cc2)c2c1C(=O)CC(C)(C)C2]

To a flask were added methyl 4-((3,6,6-trimethyl-4-oxo-4,5,6,7-tetrahydroindazol-1-yl)methyl)benzoate [formula 4-3b] (0.069 g, 0.21 mmol), hydroxylamine hydrochloride (NH₂OH HCl) (0.074 g, 1.06 mmol), potassium hydroxide (0.119 g, 2.12 mmol) and methanol (5 mL), and stirred. Then, hydroxylamine 50% aqueous solution had been added drop-wise slowly until the mixed solution in the flask became clear, and the mixture was stirred at room temperature for overnight. After the completion of the reaction, methanol was distilled out under reduced pressure, HCl was added to adjust pH 8-9 to generate white precipitate. The precipitate was filtered and dried to yield the compound 101 as white solid (0.016 g, 22.6%).

¹H NMR (400 MHz, DMSO-d₆) δ 10.49 (s, 1H), 7.68 (d, 2H, J=8.2 Hz), 7.08 (d, 2H, J=8.0 Hz), 5.26 (s, 2H), 2.42 (d, 2H, J=3.8 Hz), 2.38 (s, 3H), 0.95 (s, 6H); MS (ESI) m/z 343 (M+15)⁺.

### Example 31

### Synthesis of Compound 110

### Step 1. Synthesis of 4-((3-((2,6-dimethylmorpholino)methyl)-4-oxo-1,2,3,4-tetrahydrocarbazol-9-yl)methyl)benzoate [formula 2-5]

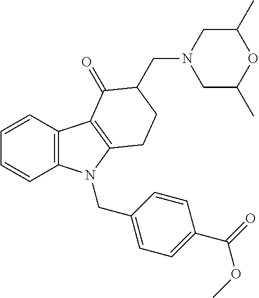 [SMILES: COC(=O)c1ccc(Cn2c3c(c4ccccc42)C(=O)C(CN2CC(C)OC(C)C2)CC3)cc1]

To a microwave vial were added methyl 4-((3-methylen-4-oxo-1,2,3,4-tetrahydrocarbazol-9-yl)methyl)benzoate [formula 2-4] (0.08 g, 0.232 mmol), 2,6-dimethylmorpholine (0.08 g, 0.695 mmol) and toluene (3.0 mL), and a reaction was carried out in a microwave reactor at 110° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.05 g, 47%).

### Step 2. Synthesis of 4-((3-((2,6-dimethylmorpholino)methyl)-4-oxo-1,2,3,4-tetrahydrocarbazol-9-yl)methyl)-N-hydroxybenzamide [formula 2-6]

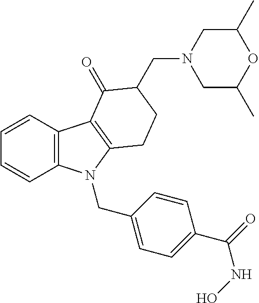 [SMILES: CC1CN(CC2CCc3c(c4ccccc4n3Cc3ccc(C(=O)NO)cc3)C2=O)CC(C)O1]

To a flask were added methyl 4-((3-((2,6-dimethylmorpholino)methyl)-4-oxo-1,2,3,4-tetrahydrocarbazol-9-yl)methyl)benzoate [formula 2-5] (0.05 g, 0.164 mmol), hydroxylamine hydrochloride (NH₂OH HCl) (0.057 g, 0.82 mmol), potassium hydroxide (0.092 g, 1.64 mmol) and methanol (5.0 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 110 as solid (0.02 g, 26%).

¹H NMR (400 MHz, DMSO-d₆) δ 11.2 (s, 1H), 9.05 (s, 1H), 8.00-8.03 (m, 1H), 7.69-7.67 (m, 2H), 7.47-7.48 (m, 1H), 7.18-7.15 (m, 4H), 5.53 (s, 2H), 3.89-3.54 (m, 2H), 3.13-3.08 (m, 1H), 3.05-2.81 (m, 2H), 2.68-2.64 (m, 2H), 2.43-2.21 (m, 2H), 2.03-1.97 (m, 1H), 1.79-1.75 (m, 1H), 1.59-1.43 (m, 1H), 1.24-1.20 (m, 1H), 1.19-1.03 (m, 6H). (ESI) m/z 462 (M⁺+H).

### Example 32

### Synthesis of Compound 111

### Step 1. Synthesis of methyl 4-((3-((4-methylpiperazin-1-yl)methyl)-4-oxo-1,2,3,4-tetrahydrocarbazol-9-yl)methyl)benzoate [formula 2-5]

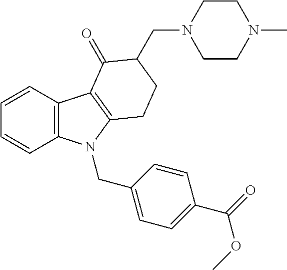 [SMILES: COC(=O)c1ccc(Cn2c3c(c4ccccc42)C(=O)C(CN2CCN(C)CC2)CC3)cc1]

To a microwave vial were added methyl 4-((3-((4-methylen-4-oxo-1,2,3,4-tetrahydrocarbazol-9-yl)methyl)benzoate [formula 2-4] (0.08 g, 0.232 mmol), 1-methylpiperazine (0.07 g, 0.695 mmol) and toluene (3.0 mL), and a reaction was carried out in a microwave reactor at 110° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.06 g, 58%).

### Step 2. Synthesis of N-hydroxy-4-((3-((4-methylpiperazin-1-yl)methyl)-4-oxo-1,2,3,4-tetrahydrocarbazol-9-yl)methyl)benzamide [formula 2-6]

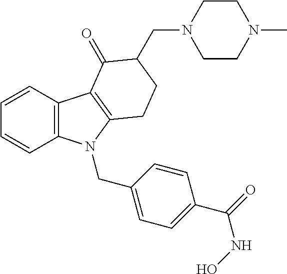 [SMILES: CN1CCN(CC2CCc3c(c4ccccc4n3Cc3ccc(C(=O)NO)cc3)C2=O)CC1]

To a flask were added methyl 4-((3-((4-methylpiperazin-1-yl)methyl)-4-oxo-1,2,3,4-tetrahydrocarbazol-9-yl)methyl)benzoate [formula 2-5] (0.06 g, 0.197 mmol), hydroxylamine hydrochloride (NH₂OH HCl) (0.069 g, 0.986 mmol), potassium hydroxide (0.111 g, 1.97 mmol) and methanol (5.0 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 111 as solid (0.015 g, 17%).

¹H NMR (400 MHz, DMSO-d₆) δ 11.1 (s, 1H), 9.02 (s, 1H), 8.04-8.01 (m, 1H), 7.67 (d, 2H, J=8.2 Hz), 7.48-7.46 (m, 1H), 7.23-7.16 (m, 4H), 5.53 (s, 2H), 3.09-3.03 (m, 1H), 2.96-2.89 (m, 1H), 2.69-2.66 (m, 2H), 2.49-2.43 (m, 4H), 2.32-2.30 (m, 6H), 2.15 (s, 3H), 1.97-1.93 (m, 1H). (ESI) m/z 447 (M⁺+H).

### Example 33

### Synthesis of Compound 112

### Step 1. Synthesis of 4-((3-((4-ethylpiperazin-1-yl)methyl)-4-oxo-1,2,3,4-tetrahydrocarbazol-9-yl)methyl)benzoate [formula 2-5]

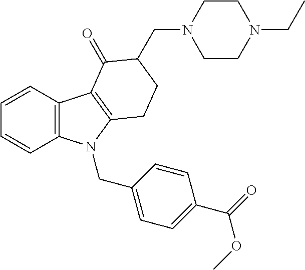 [SMILES: CCN1CCN(CC2CCc3c(c4ccccc4n3Cc3ccc(C(=O)OC)cc3)C2=O)CC1]

To a microwave vial were added methyl 4-((3-methylen-4-oxo-1,2,3,4-tetrahydrocarbazol-9-yl)methyl)benzoate [formula 2-4] (0.05 g, 0.145 mmol), ethylpiperazine (0.038 g, 0.434 mmol) and toluene (3.0 mL), and a reaction was carried out in a microwave reactor at 110° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.043 g, 65%).

### Step 2. Synthesis of 4-((3-((4-ethylpiperazin-1-yl)methyl)-4-oxo-1,2,3,4-tetrahydrocarbazol-9-yl)methyl)-N-hydroxy benzamide [formula 2-6]

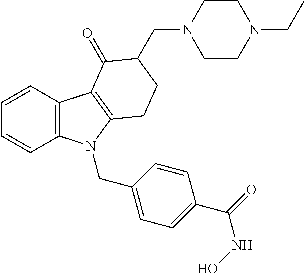 [SMILES: CCN1CCN(CC2CCc3c(c4ccccc4n3Cc3ccc(C(=O)NO)cc3)C2=O)CC1]

To a flask were added methyl 4-((3-((4-ethylpiperazin-1-yl)methyl)-4-oxo-1,2,3,4-tetrahydrocarbazol-9-yl)methyl)benzoate [formula 2-5] (0.043 g, 0.094 mmol), hydroxylamine hydrochloride (NH₂OH HCl) (0.033 g, 0.468 mmol), potassium hydroxide (0.052 g, 0.936 mmol) and methanol (5.0 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 112 as solid (0.021 g, 48%).

¹H NMR (400 MHz, DMSO-d₆) δ 11.3 (s, 1H), 9.01 (s, 1H), 8.03-8.01 (m, 1H), 7.67 (d, 2H, J=8.0 Hz), 7.48-7.46 (m, 1H), 7.18-7.16 (m, 4H), 5.53 (s, 2H), 3.05-3.04 (m, 1H), 2.95-2.82 (m, 1H), 2.68-2.66 (m, 2H), 2.49-2.42 (m, 6H), 2.40-2.25 (m, 2H), 2.02-1.89 (m, 1H), 0.96 (t, 3H, J=7.1 Hz). (ESI) m/z 461 (M⁺+H).

### Example 34

### Synthesis of Compound 113

### Step 1. Synthesis of methyl 4-((3-((4-isopropylpiperazin-1-yl)methyl)-4-oxo-1,2,3,4-tetrahydrocarbazol-9-yl)methyl)benzoate [formula 2-5]

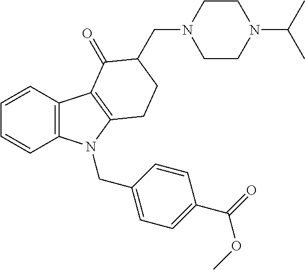 [SMILES: COC(=O)c1ccc(Cn2c3c(c4ccccc42)C(=O)C(CN2CCN(C(C)C)CC2)CC3)cc1]

To a microwave vial were added methyl 4-((3-methylen-4-oxo-1,2,3,4-tetrahydrocarbazol-9-yl)methyl)benzoate [formula 2-4] (0.05 g, 0.145 mmol), isopropylpiperazine (0.038 g, 0.434 mmol) and toluene (3.0 mL), and a reaction was carried out in a microwave reactor at 110° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.029 g, 42%).

### Step 2. Synthesis of N-hydroxy-4-((3-((4-isopropylpiperazin-1-yl)methyl)-4-oxo-1,2,3,4-tetrahydrocarbazol-9-yl)methyl)benzamide [formula 2-6]

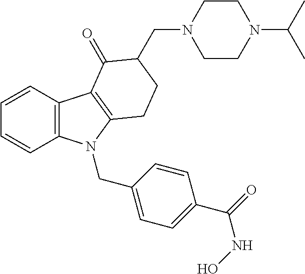 [SMILES: CC(C)N1CCN(CC2CCc3c(c4ccccc4n3Cc3ccc(C(=O)NO)cc3)C2=O)CC1]

To a flask were added methyl 4-((3-((4-isopropylpiperazin-1-yl)methyl)-4-oxo-1,2,3,4-tetrahydrocarbazol-9-yl)methyl)benzoate [formula 2-5] (0.038 g, 0.08 mmol), hydroxylamine hydrochloride (NH₂OH HCl) (0.028 g, 0.401 mmol), potassium hydroxide (0.045 g, 0.802 mmol) and methanol (5.0 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 113 as solid (0.015 g, 39%).

¹H NMR (400 MHz, DMSO-d₆) δ 11.2 (s, 1H), 9.02 (s, 1H), 8.03-8.01 (m, 1H), 7.68 (d, 2H, J=8.0 Hz), 7.48-7.46 (m, 1H), 7.18-7.07 (m, 4H), 5.57 (s, 2H), 3.39-3.26 (m, 5H), 3.16-3.04 (m, 2H), 2.96-2.83 (m, 1H), 2.79-2.66 (m, 3H), 2.32-2.24 (m, 3H), 2.00-1.94 (m, 1H), 1.31-1.22 (m, 1H), 0.95-1.02 (m, 6H). (ESI) m/z 475 (M⁺+H).

### Example 35

### Synthesis of Compound 114

### Step 1. Synthesis of methyl 4-((3-((4-(2-methoxyethyl)piperazin-1-yl)methyl)-4-oxo-1,2,3,4-tetrahydrocarbazol-9-yl)methyl)benzoate [formula 2-5]

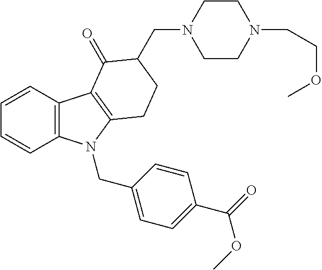 [SMILES: COCCN1CCN(CC2CCc3c(c4ccccc4n3Cc3ccc(C(=O)OC)cc3)C2=O)CC1]

To a microwave vial were added methyl 4-((3-methylen-4-oxo-1,2,3,4-tetrahydrocarbazol-9-yl)methyl)benzoate [formula 2-4] (0.05 g, 0.145 mmol), 1-(2-methoxyethyl)piperazine (0.038 g, 0.434 mmol) and toluene (4.0 mL), and a reaction was carried out at in a microwave reactor 120° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.037 g, 52%).

### Step 2. Synthesis of N-hydroxy-4-((3-((4-(2-methoxyethyl)piperazin-1-yl)-4-oxo-1,2,3,4-tetrahydrocarbazol-9-yl)methyl)benzamide [formula 2-6]

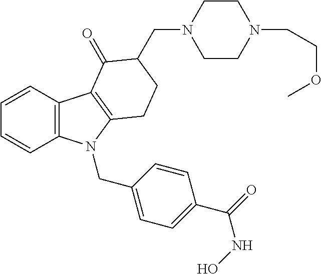 [SMILES: COCCN1CCN(CC2CCc3c(c4ccccc4n3Cc3ccc(C(=O)NO)cc3)C2=O)CC1]

To a flask were added methyl 4-((3-((4-(2-methoxyethyl)piperazin-1-yl)methyl)-4-oxo-1,2,3,4-tetrahydrocarbazol-9-yl)methyl)benzoate [formula 2-5] (0.037 g, 0.076 mmol), hydroxylamine hydrochloride (NH₂OH HCl) (0.026 g, 0.378 mmol), potassium hydroxide (0.042 g, 0.756 mmol) and methanol (10 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 114 as solid (0.016 g, 43%).

¹H NMR (400 MHz, MeOD-d₃) δ 8.13-8.10 (m, 1H), 7.69-7.63 (d, 2H, J=8.0 Hz), 7.36-7.34 (m, 1H), 7.25-7.20 (m, 2H), 7.19-7.14 (m, 2H), 5.50 (s, 2H), 3.53 (t, 2H, J=5.5 Hz), 3.33 (s, 3H), 3.04-3.02 (m, 1H), 2.94-2.86 (m, 2H), 2.76-2.72 (m, 2H), 2.62-2.54 (m, 8H), 2.47-2.40 (m, 3H), 2.18-2.01 (m, 1H). (ESI) m/z 490 (M⁺+H).

### Example 36

### Synthesis of Compound 121

### Step 1. Synthesis of methyl 4-((3-((3,3-difluoroazetidin-1-yl)methyl)-4-oxo-1,2,3,4-tetrahydrocarbazol-9-yl)methyl)benzoate [formula 2-5]

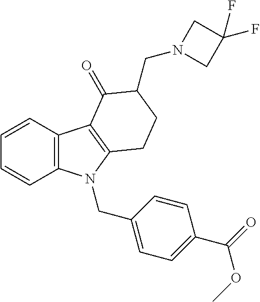 [SMILES: COC(=O)c1ccc(Cn2c3c(c4ccccc42)C(=O)C(CN2CC(F)(F)C2)CC3)cc1]

To a microwave vial were added methyl 4-((3-methylen-4-oxo-1,2,3,4-tetrahydrocarbazol-9-yl)methyl)benzoate [formula 2-4] (0.08 g, 0.232 mmol), 3,3-difluoroazetidin hydrochloride (0.09 g, 0.695 mmol), potassium carbonate (0.16 g, 1.16 mmol) and toluene (4.0 mL), and a reaction was carried out in a microwave reactor at 110° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.042 g, 41%).

### Step 2. Synthesis of 4-((3-((3,3-difluoroazetidin-1-yl)methyl)-4-oxo-1,2,3,4-tetrahydrocarbazol-9-yl)methyl)-N-hydroxybenzamide [formula 2-6]

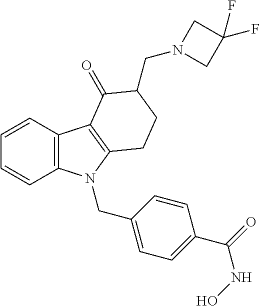 [SMILES: O=C(NO)c1ccc(Cn2c3c(c4ccccc42)C(=O)C(CN2CC(F)(F)C2)CC3)cc1]

To a flask were added methyl 4-((3-((3,3-difluoroazetidin-1-yl)methyl)-4-oxo-1,2,3,4-tetrahydrocarbazol-9-yl)methyl)benzoate [formula 2-5] (0.04 g, 0.091 mmol), hydroxylamine hydrochloride (NH₂OH HCl) (0.032 g, 0.456 mmol), potassium hydroxide (0.051 g, 0.912 mmol) and methanol (5.0 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 121 as solid (0.015 g, 36%).

¹H NMR (400 MHz, DMSO-d₆) δ 11.1 (s, 1H), 9.02 (s, 1H), 8.03-8.01 (m, 1H), 7.67 (d, 2H, J=8.2 Hz), 7.49-7.47 (m, 1H), 7.19-7.16 (m, 4H), 5.53 (s, 2H), 3.64-3.54 (m, 4H), 3.18-3.06 (m, 2H), 2.95-2.78 (m, 2H), 2.36-2.22 (m, 1H), 1.98-1.83 (m, 1H). MS (ESI) m/z 440 (M⁺+H).

### Example 37

### Synthesis of Compound 122

### Step 1. Synthesis of 2,2-dimethyl-2,3-dihydro-1H-carbazol-4(9H)-on [formula 2-2]

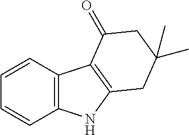 [SMILES: CC1(C)CC(=O)c2c([nH]c3ccccc23)C1]

To a microwave vial were added 5,5-dimethyl-1,3-cyclohexandion (3.0 g, 21.4 mmol), phenylhydrazine (2.78 g, 25.7 mmol) and TFA (4.0 mL), and a reaction was carried out in a microwave reactor at 140° C. for 10 minutes. Then, TFA was distilled out under reduced pressure, the reaction mixture was extracted with ethyl acetate and saturated NaHCO₃ aqueous solution, dried over anhydrous MgSO₄, filtered and concentrated under reduced pressure. Residue was purified by column chromatography (SiO₂; hexane/ethylacetate, 4/1) to yield the title compound (2.2 g, 48%).

### Step 2. Synthesis of methyl 4-((2,2-dimethyl-4-oxo-1,2,3,4-tetrahydrocarbazol-9-yl)methyl)benzoate [formula 2-3]

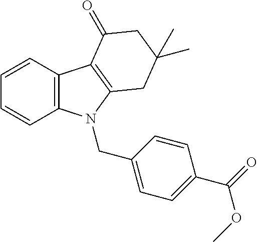 [SMILES: COC(=O)c1ccc(Cn2c3c(c4ccccc42)C(=O)CC(C)(C)C3)cc1]

2,2-dimethyl-2,3-dihydro-1H-carbazol-4(9H)-on) [formula 2-2] (2.2 g, 10.3 mmol) was dissolved in DMF (10 mL). Thereto, 55% NaH in paraffin solution (0.90 g, 20.6 mmol) was added and stirred for 10 minutes. Then, methyl-4(bromomethyl)benzoate (2.83 g, 12.4 mmol) was added and stirred at 50° C. for 5 hours. After the completion of the reaction, the reaction mixture was washed with ethyl acetate and water, dried over anhydrous MgSO₄, filtered and concentrated under reduced pressure. Residue was purified by column chromatography (SiO₂; hexane/ethylacetate, 5/1) to yield the title compound (2.1 g, 56%).

### Step 3. Synthesis of methyl 4-((2,2-dimethyl-3-methylen-4-oxo-1,2,3,4-tetrahydrocarbazol-9-yl)methyl)benzoate [formula 2-4]

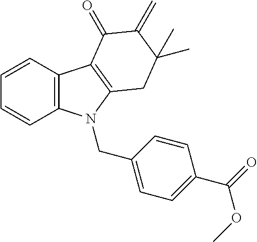 [SMILES: C=C1C(=O)c2c(n(Cc3ccc(C(=O)OC)cc3)c3ccccc23)CC1(C)C]

Methyl 4-((2,2-dimethyl-4-oxo-1,2,3,4-tetrahydrocarbazol-9-yl)methyl)benzoate [formula 2-3] (0.5 g, 1.38 mmol), N,N-dimethylamine HCl (0.226 g, 2.77 mmol), paraformaldehyde (0.092 g, 2.77 mmol) and mixed solvent (acetic acid:toluene=4:1, 15 mL) were added, and stirred at 100° C. for 4 hours. After the completion of the reaction, acetic acid was distilled out under reduced pressure, and, without purification, the compound was dissolved in a 15 mL of mixed solvent (acetonitrile:H₂O=1:4) and stirred at 80° C. for 12 hours. After the completion of the reaction, the reaction mixture was extracted with ethyl acetate and saturated NaHCO₃ aqueous solution, dried over anhydrous MgSO₄, filtered and concentrated under reduced pressure. Residue was purified by column chromatography (SiO₂; hexane/ethylacetate, 7/1) to yield the title compound (0.35 g, 67%).

### Step 4. Synthesis of methyl 4-((2,2-dimethyl-3-(morpholinomethyl)-4-oxo-1,2,3,4-tetrahydrocarbazol-9-yl)methyl)benzoate [formula 2-5]

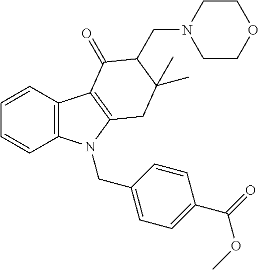 [SMILES: COC(=O)c1ccc(Cn2c3c(c4ccccc42)C(=O)C(CN2CCOCC2)C(C)(C)C3)cc1]

To a microwave vial were added methyl 4-((2,2-dimethyl-3-methylen-4-oxo-1,2,3,4-tetrahydrocarbazol-9-yl)methyl)benzoate [formula 2-4] (0.1 g, 0268 mmol), morpholine (0.07 g, 0.803 mmol) and toluene (4.0 mL), and a reaction was carried out in a microwave reactor at 110° C. for 90 minutes. Then the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.071 g, 57%).

### Step 5. Synthesis of 4-((2,2-dimethyl-3-(morpholinomethyl)-4-oxo-1,2,3,4-tetrahydrocarbazol-9-yl)methyl)-N-hydroxybenzamide [formula 2-6]

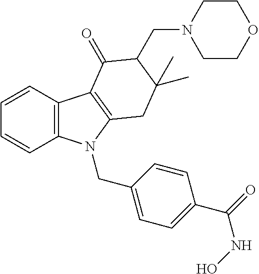 [SMILES: CC1(C)Cc2c(c3ccccc3n2Cc2ccc(C(=O)NO)cc2)C(=O)C1CN1CCOCC1]

To a flask were added methyl 4-((2,2-dimethyl-3-(morpholinomethyl)-4-oxo-1,2,3,4-tetrahydrocarbazol-9-yl)methyl)benzoate [formula 2-5] (0.071 g, 0.154 mmol), hydroxylamine hydrochloride (NH₂OH HCl) (0.0536 g, 0.771 mmol), potassium hydroxide (0.0865 g, 1.542 mmol) and methanol (5.0 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 122 as solid (0.032 g, 45%).

¹H NMR (400 MHz, DMSO-d₆) δ 11.3 (s, 1H), 9.04 (s, 1H), 8.01-7.98 (m, 1H), 7.67 (d, 2H, J=8.2 Hz), 7.47-7.45 (m, 1H), 7.18-7.09 (m, 4H), 5.52 (s, 2H), 3.51-3.48 (m, 4H), 3.00-2.96 (m, 1H), 2.88-2.83 (m, 1H), 2.76-2.61 (m, 1H), 2.47-2.43 (m, 4H), 2.38-2.33 (m, 2H), 1.12 (s, 3H), 1.01 (s, 3H). MS (ESI) m/z 462 (M⁺+H).

### Example 38

### Synthesis of Compound 123

### Step 1. Synthesis of methyl 4-((3-((3,3-difluoropyrrolidin-1-yl)methyl)-4-oxo-1,2,3,4-tetrahydrocarbazol-9-yl)methyl)benzoate [formula 2-5]

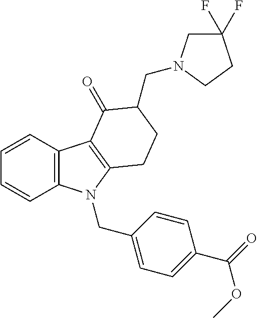 [SMILES: COC(=O)c1ccc(Cn2c3c(c4ccccc42)C(=O)C(CN2CCC(F)(F)C2)CC3)cc1]

To a microwave vial were added methyl 4-((2,2-dimethyl-3-methylen-4-oxo-1,2,3,4-tetrahydrocarbazol-9-yl)methyl)benzoate [formula 2-4] (0.1 g, 0.29 mmol), 3,3-difluoropyrrolidin hydrochloride (0.093 g, 0.869 mmol), potassium carbonate (0.2 g, 1.45 mmol) and toluene (4.0 mL), and a reaction was carried out in a microwave reactor at 110° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.072 g, 55%).

### Step 2. Synthesis of 4-((3-((3,3-difluoropyrrolidin-1-yl)methyl)-4-oxo-1,2,3,4-tetrahydrocarbazol-9-yl)methyl)-N-hydroxybenzamide [formula 2-6]

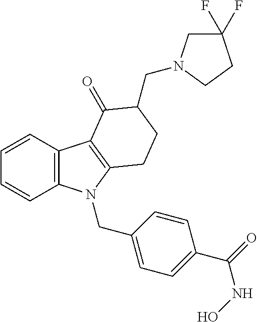 [SMILES: O=C(NO)c1ccc(Cn2c3c(c4ccccc42)C(=O)C(CN2CCC(F)(F)C2)CC3)cc1]

To a flask were added methyl 4-((3-((3,3-difluoropyrrolidin-1-yl)methyl)-4-oxo-1,2,3,4-tetrahydrocarbazol-9-yl)methyl)benzoate [formula 2-5] (0.07 g, 0.16 mmol), hydroxylamine hydrochloride (NH₂OH HCl) (0.055 g, 0.796 mmol), potassium hydroxide (0.089 g, 1.59 mmol) and methanol (5.0 mL), and stirred for 10 minutes. Then, hydroxylamine 55% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous MgSO₄, filtered and concentrated under reduced pressure. Residue was treated with 2N HCl and filtered to yield the compound 123 as solid (0.035 g, 48%).

¹H NMR (400 MHz, DMSO-d₆) δ 7.96-8.01 (m, 1H), 7.67 (d, 2H, J=8.2 Hz), 7.48-7.42 (m, 1H), 7.19-7.15 (m, 4H), 5.52 (s, 2H), 3.08-2.92 (m, 3H), 2.87-2.70 (m, 5H), 2.45-2.41 (m, 1H), 2.36-2.21 (m, 2H), 2.20-2.11 (m, 1H). MS (ESI) m/z 454 (M⁺+H).

### Example 39

### Synthesis of Compound 126

### Step 1. Synthesis of methyl 4-((2,2-dimethyl-3-((2-methyl-1H-imidazol-1-yl)methyl)-4-oxo-1,2,3,4-tetrahydrocarbazol-9-yl)methyl)benzoate [formula 2-5]

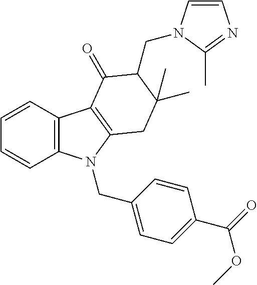 [SMILES: COC(=O)c1ccc(Cn2c3c(c4ccccc42)C(=O)C(Cn2ccnc2C)C(C)(C)C3)cc1]

To a microwave vial were added methyl 4-((2,2-dimethyl-3-methylen-4-oxo-1,2,3,4-tetrahydrocarbazol-9-yl)methyl)benzoate [formula 2-4] (0.09 g, 0.241 mmol), 2-dimethyl imidazole (0.0594 g, 0.723 mmol) and toluene (4.0 mL), and a reaction was carried out in a microwave reactor at 110° C. for 90 minutes. Then, the reaction mixture was extracted with ethyl acetate and saturated NH₄Cl aqueous solution, dried over anhydrous MgSO₄, filtered and concentrated under reduced pressure. Residue was purified by column chromatography (SiO₂; dichloromethane/methanol, 10/1) to yield the title compound (0.055 g, 50%).

### Step 2. Synthesis of 4-((2,2-dimethyl-3-((2-methyl-1H-imidazol-1-yl)methyl)-4-oxo-1,2,3,4-tetrahydrocarbazol-9-yl)methyl)-N-hydroxybenzamide [formula 2-6]

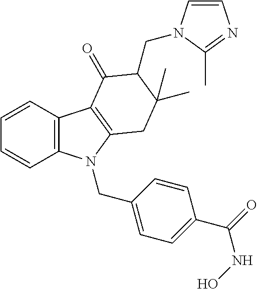 [SMILES: Cc1nccn1CC1C(=O)c2c(n(Cc3ccc(C(=O)NO)cc3)c3ccccc23)CC1(C)C]

To a flask were added methyl 4-((2,2-dimethyl-3-((2-methyl-1H-imidazol-1-yl)methyl)-4-oxo-1,2,3,4-tetrahydrocarbazol-9-yl)methyl)benzoate [formula 2-5] (0.055 g, 0.121 mmol), hydroxylamine hydrochloride (NH₂OH HCl) (0.0419 g, 0.604 mmol), potassium hydroxide (0.0677 g, 1.207 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 10/1) to yield the compound 126 as solid (0.022 g, 40%).

¹H NMR (400 MHz, DMSO-d₆) δ 11.2 (brs, 1H), 9.05 (brs, 1H), 7.96 (t, 1H, J=4.5 Hz), 7.68 (d, 2H, J=8.1 Hz), 7.47 (t, 1H, J=4.5 Hz), 7.20-7.07 (m, 5H), 6.67 (s, 1H), 5.56 (s, 2H), 4.30-4.28 (m, 1H), 4.12-4.08 (m, 1H), 3.00 (s, 2H), 2.79-2.78 (m, 1H), 2.22 (s, 3H), 1.22 (s, 3H), 1.05 (s, 3H); MS (ESI) m/z 457 (M++H).

### Example 40

### Synthesis of Compound 127

### Step 1. Synthesis of methyl 4-((3-((4-(4-fluorophenyl)piperazin-1-yl)methyl)-4-oxo-1,2,3,4-tetrahydrocarbazol-9-yl)methyl)benzoate [formula 2-5]

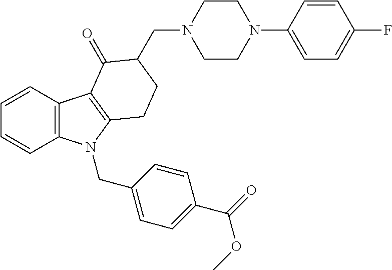 [SMILES: COC(=O)c1ccc(Cn2c3c(c4ccccc42)C(=O)C(CN2CCN(c4ccc(F)cc4)CC2)CC3)cc1]

To a microwave vial were added methyl 4-((3-methylen-4-oxo-1,2,3,4-tetrahydrocarbazol-9-yl)methyl)benzoate [formula 2-4] (0.10 g, 0.290 mmol), 1-(4-fluorophenylpiperazine (0.156 g, 0.869 mmol) and toluene (4.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.072 g, 47%).

### Step 2. Synthesis of 4-((3-((4-(4-fluorophenyl)piperazin-1-yl)methyl)-4-oxo-1,2,3,4-tetrahydrocarbazol-9-yl)methyl)-N-hydroxybenzamide [formula 2-6]

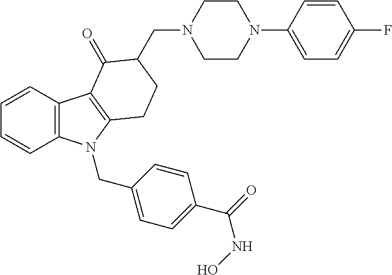 [SMILES: O=C(NO)c1ccc(Cn2c3c(c4ccccc42)C(=O)C(CN2CCN(c4ccc(F)cc4)CC2)CC3)cc1]

To a flask were added methyl 4-((3-((4-(4-fluorophenyl)piperazin-1-yl)methyl)-4-oxo-1,2,3,4-tetrahydrocarbazol-9-yl)methyl)benzoate [formula 2-5] (0.072 g, 0.137 mmol), hydroxylamine hydrochloride (NH₂OH HCl) (0.0476 g, 0.685 mmol), potassium hydroxide (0.0769 g, 1.37 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 10/1) to yield the compound 127 as solid (0.038 g, 53%).

¹H NMR (400 MHz, DMSO-d₆) δ 8.05-8.03 (m, 1H), 7.68 (d, 2H, J=8.1 Hz), 7.50-7.48 (m, 1H), 7.19-7.14 (m, 4H), 7.05-7.00 (m, 2H), 6.94-6.91 (m, 2H), 5.16 (s, 2H), 3.49-3.35 (m, 4H), 2.96-2.90 (m, 2H), 2.75-2.73 (m, 2H), 2.65-2.62 (m, 2H), 2.57-2.50 (m, 1H), 2.49-2.42 (m, 2H), 2.37-2.33 (m, 1H), 1.99-1.97 (m, 1H); MS (ESI) m/z 527 (M++H).

### Example 41

### Synthesis of Compound 128

### Step 1. Synthesis of methyl 4-((3-((4-(3,4-dimethylphenyl)piperazin-1-yl)methyl)-4-oxo-1,2,3,4-tetrahydrocarbazol-9-yl)methyl)benzoate [formula 2-5]

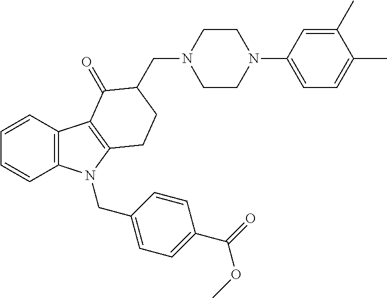 [SMILES: COC(=O)c1ccc(Cn2c3c(c4ccccc42)C(=O)C(CN2CCN(c4ccc(C)c(C)c4)CC2)CC3)cc1]

To a microwave vial were added methyl 4-((3-methylen-4-oxo-1,2,3,4-tetrahydrocarbazol-9-yl)methyl)benzoate [formula 2-4] (0.08 g, 0.232 mmol), 1-(3,4-dimethyl)piperazine (0.1322 g, 0.695 mmol) and toluene (4.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.052 g, 42%).

### Step 2. Synthesis of 4-((3-((4-(3,4-dimethylphenyl)piperazin-1-yl)methyl)-4-oxo-1,2,3,4-tetrahydrocarbazol-9-yl)methyl)-N-hydroxybenzamide [formula 2-6]

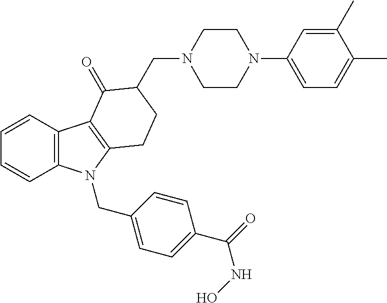 [SMILES: Cc1ccc(N2CCN(CC3CCc4c(c5ccccc5n4Cc4ccc(C(=O)NO)cc4)C3=O)CC2)cc1C]

To a flask were added methyl 4-((3-((4-(3,4-dimethylphenyl)piperazin-1-yl)methyl)-4-oxo-1,2,3,4-tetrahydrocarbazol-9-yl)methyl)benzoate [formula 2-5] (0.052 g, 0.097 mmol), hydroxylamine hydrochloride (NH₂OH HCl) (0.0337 g, 0.485 mmol), potassium hydroxide (0.0545 g, 0.971 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 10/1) to yield the compound 128 as solid (0.021 g, 40%).

¹H NMR (400 MHz, DMSO-d₆) δ 8.05-8.03 (m, 1H), 7.68 (d, 2H, J=8.3 Hz), 7.50-7.47 (m, 1H), 7.21-7.16 (m, 4H), 6.94 (d, 1H, J=8.2 Hz), 6.73 (s, 1H), 6.64-6.61 (m, 1H), 5.54 (s, 2H), 3.11-3.05 (m, 5H), 2.98-2.95 (s, 1H), 2.75-2.73 (m, 2H), 2.64-2.62 (m, 2H), 2.56-2.50 (m, 1H), 2.44-2.34 (m, 3H), 2.15 (s, 3H), 2.09 (s, 3H), 2.00-1.98 (m, 1H); MS (ESI) m/z 537 (M++H).

### Example 42

### Synthesis of Compound 129

### Step 1. Synthesis of methyl 4-((3-((dimethylamino)methyl)-2,2-dimethyl-4-oxo-1,2,3,4-tetrahydrocarbazol-9-yl)methyl)benzoate [formula 2-5]

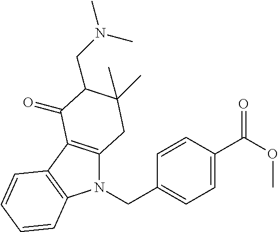 [SMILES: COC(=O)c1ccc(Cn2c3c(c4ccccc42)C(=O)C(CN(C)C)C(C)(C)C3)cc1]

To a microwave vial were added methyl 4-((2,2-dimethyl-4-oxo-1,2,3,4-tetrahydrocarbazol-9-yl)methyl)benzoate [formula 2-3] (0.5 g, 1.38 mmol), N,N-dimethylamine HCl (0.226 g, 2.77 mmol), paraformaldehyde (0.092 g, 2.77 mmol) and mixed solvent (acetic acid:toluene=4:1, 15 mL) were added, and stirred at 100° C. for 4 hours. After the completion of the reaction, acetic acid was distilled out under reduced pressure, and without purification, the compound was dissolved in a 15 mL of mixed solvent (acetonitrile:H₂O=1:4) and stirred at 80° C. for 12 hours. After the completion of the reaction, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 7/1) to yield the title compound (0.067 g, 12%).

### Step 2. Synthesis of 4-((3-((dimethylamino)methyl)-2,2-dimethyl-4-oxo-1,2,3,4-tetrahydrocarbazol-9-yl)methyl)-N-hydroxybenzamide [formula 2-6]

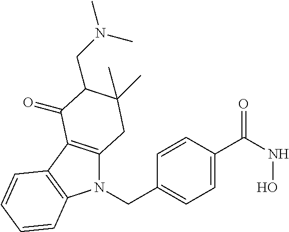 [SMILES: CN(C)CC1C(=O)c2c(n(Cc3ccc(C(=O)NO)cc3)c3ccccc23)CC1(C)C]

To a flask were added methyl 4-((3-((dimethylamino)methyl)-2,2-dimethyl-4-oxo-1,2,3,4-tetrahydrocarbazol-9-yl)methyl)benzoate [formula 2-5] (0.067 g, 0.16 mmol), hydroxylamine hydrochloride (NH₂OH HCl) (0.0556 g, 0.80 mmol), potassium hydroxide (0.0898 g, 1.60 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 10/1) to yield the compound 129 as solid (0.021 g, 31%).

¹H NMR (400 MHz, DMSO-d₆) δ 11.1 (brs, 1H), 9.02 (brs, 1H), 8.01-7.98 (m, 1H), 7.68 (d, 2H, J=8.0 Hz), 7.47-7.44 (m, 1H), 7.19-7.08 (m, 4H), 5.48 (s, 2H), 2.99-2.92 (m, 2H), 2.84-2.79 (m, 1H), 2.49-2.32 (m, 2H), 2.16 (s, 6H), 1.16 (s, 3H), 1.09 (s, 3H); MS (ESI) m/z 420 (M++H).

### Example 43

### Synthesis of Compound 130

### Step 1. Synthesis of methyl 4-((3-((4-(methylsulfonyl)piperazin-1-yl)methyl)-4-oxo-1,2,3,4-tetrahydrocarbazol-9-yl)methyl)benzoate [formula 2-5]

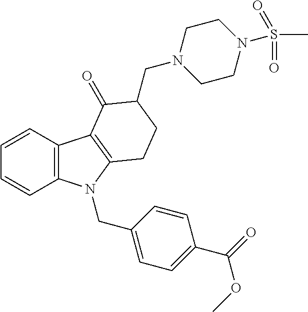 [SMILES: COC(=O)c1ccc(Cn2c3c(c4ccccc42)C(=O)C(CN2CCN(S(C)(=O)=O)CC2)CC3)cc1]

To a microwave vial were added methyl 4-((3-methylen-4-oxo-1,2,3,4-tetrahydrocarbazol-9-yl)methyl)benzoate [formula 2-4] (0.08 g, 0.232 mmol), 1-(methylsulfonyl)piperazine (0.1322 g, 0.695 mmol) and toluene (4.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.032 g, 27%).

### Step 2. Synthesis of N-hydroxy-4-((3-((4-(methylsulfonyl)piperazin-1-yl)methyl)-4-oxo-1,2,3,4-tetrahydrocarbazol-9-yl)methyl)benzamide [formula 2-6]

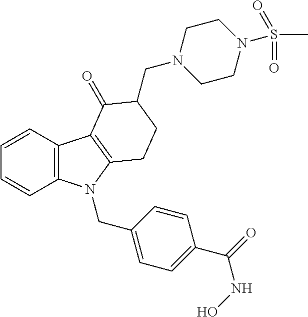 [SMILES: CS(=O)(=O)N1CCN(CC2CCc3c(c4ccccc4n3Cc3ccc(C(=O)NO)cc3)C2=O)CC1]

To a flask were added methyl 4-((3-((4-(methylsulfonyl)piperazin-1-yl)methyl)-4-oxo-1,2,3,4-tetrahydrocarbazol-9-yl)methyl)benzoate [formula 2-5] (0.032 g, 0.063 mmol), hydroxylamine hydrochloride (NH₂OH HCl) (0.0218 g, 0.314 mmol), potassium hydroxide (0.0352 g, 0.628 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate, dried over anhydrous Na₂SO₄, filtered and concentrated under reduced pressure. Residue was purified by column chromatography (SiO₂; dichloromethane/methanol, 10/1) to yield the compound 130 as solid (0.012 g, 37%).

¹H NMR (400 MHz, DMSO-d₆) δ 11.1 (brs, 1H), 9.03 (brs, 1H), 8.04-8.02 (m, 1H), 7.68 (d, 2H, J=8.3 Hz), 7.49-7.47 (m, 1H), 7.19-7.16 (m, 4H), 5.53 (s, 2H), 3.09-2.96 (m, 5H), 2.95-2.93 (m, 1H), 2.87 (s, 3H), 2.78-2.69 (m, 2H), 2.61-2.55 (m, 3H), 2.50-2.32 (s, 3H), 1.98-1.96 (m, 1H); MS (ESI) m/z 511 (M++H).

### Example 44

### Synthesis of Compound 131

### Step 1. Synthesis of 3,3-dimethyl-2,3-dihydro-1H-carbazol-4(9H)-on) [formula 5-2]

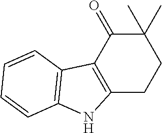 [SMILES: CC1(C)CCc2[nH]c3ccccc3c2C1=O]

To a microwave vial were added phenyl hydrazine [formula 5-1] (1.0 g, 7.13 mmol), 4-4-dimethylcyclohexan-1,3-dion (0.926 g, 8.56 mmol) and TFA (10.0 mL), and a reaction was carried out in a microwave reactor at 140° C. for 10 minutes. Then, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (0.72 g, 47%).

### Step 2. Synthesis of methyl 4-((3,3-dimethyl-4-oxo-1,2,3,4-tetrahydrocarbazol-9-yl)methyl)benzoate [Formula 5-3]

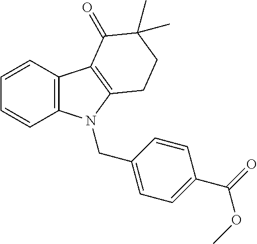 [SMILES: COC(=O)c1ccc(Cn2c3c(c4ccccc42)C(=O)C(C)(C)CC3)cc1]

3,3-dimethyl-2,3-dihydro-1H-carbazol-4(9H)-on [formula 5-2] (0.31 g, 1.453 mmol) was dissolved in DMF (10 mL). Thereto, 55% NaH in paraffin solution (0.127 g, 2.91 mmol) was added and stirred for 10 minutes. Then, methyl-4(bromomethyl)benzoate (0.399 g, 1.744 mmol) was added and stirred at 50° C. for 6 hours. After the completion of the reaction, DMF was distilled out under reduced pressure the reaction mixture was washed with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 5/1) to yield the title compound (0.123 g, 23%)

### Step 3. Synthesis of 4-((3,3-dimethyl-4-oxo-1,2,3,4-tetrahydrocarbazol-9-yl)methyl)-N-hydroxybenzamide [formula 5-4]

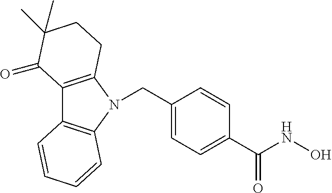 [SMILES: CC1(C)CCc2c(c3ccccc3n2Cc2ccc(C(=O)NO)cc2)C1=O]

To a flask were added methyl 4-((3,3-dimethyl-4-oxo-1,2,3,4-tetrahydrocarbazol-9-yl)methyl)benzoate [formula 5-3] (0.123 g, 0.340 mmol), hydroxylamine hydrochloride (HONH₂, HCl) (0.118 g, 1.702 mmol), potassium hydroxide (0.191 g, 3.40 mmol) and methanol (10 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was purified by column chromatography (SiO₂; dichloromethane/methanol, 10/1) to yield the compound of 131 as solid (0.046 g, 37%).

¹H NMR (400 MHz, DMSO-d₆) δ 8.05-8.03 (m, 1H), 7.67 (d, 2H, J=6.2 Hz), 7.48-7.46 (m, 1H), 7.19-7.16 (m, 2H), 7.13 (d, 2H, J=6.2 Hz), 5.51 (s, 2H), 2.98 (t, 2H, J=4.6 Hz), 2.01 (t, 2H, J=4.6 Hz), 1.12 (s, 6H); MS (ESI) m/z 363 (M++H).

### Example 45

### Synthesis of Compound 136—Scheme 6

### Step 1. Synthesis of 3,6,6-trimethyl-6,7-dihydro-1H-indole-4(5H)-on [formula 6-2]

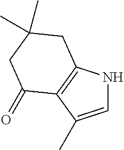

Anti-pyruvic aldehyde-1-oxime [formula 6-1] (0.50 g, 5.74 mmol) and 5,5-dimethyl-1,3-cyclohexandion [formula 6-7] (0.80 g, 5.74 mmol) were dissolved in acetic acid (35 mL) and H₂O (15 mL). Thereto, zinc powder (0.75 g, 11.5 mmol) was added slowly maintaining room temperature. The reaction mixture was refluxed with stirring, concentrated under reduced pressure and extracted with CH₂Cl₂ and brine, of which pH was adjusted to 6 using saturated NaHCO₃. The reaction mixture was extracted with CH₂Cl₂; organic layer was dried over anhydrous MgSO₄ and filtered. Residue was concentrated under reduced pressure and purified by column chromatography (SiO₂; hexane/ethylacetate, 1/3) to yield the title compound as yellow solid (4.9 g, 52%).

### Step 2. Synthesis of methyl 4-((3,6,6-trimethyl-4-oxo-4,5,6,7-tetrahydro-1H-indole-1-yl)methyl)benzoate [formula 6-3]

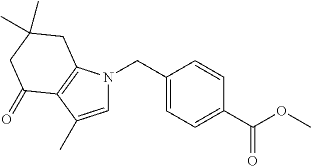

3,6,6-trimethyl-6,7-dihydro-1H-indole-4(5H)-on [formula 6-2] (0.23 g, 1.29 mmol) was dissolved DMF. Thereto, NaH (0.062 g, 2.56 mmol) was added slowly at room temperature. After 5 minutes stirring, methyl 4-(bromethyl)benzoate was added and stirred at room temperature for 4 hours. After the completion of reaction, the reaction mixture was extracted with ethyl acetate and saturated NH₄Cl aqueous solution, the organic layer was dried over anhydrous MgSO₄ and filtered. Filtrate was concentrated under reduced pressure and purified by column chromatography (SiO₂; hexane/ethylacetate, 7/3) to yield the title compound as white solid (0.14 g, 34%).

### Step 3. Synthesis of N-hydroxy-4-((3,6,6-trimethyl-4-oxo-4,5,6,7-tetrahydro-1H-indol-1-yl)methyl)benzamide [formula 6-4]

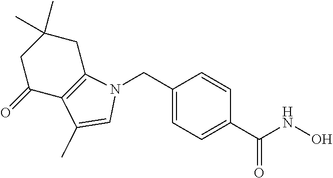 [SMILES: Cc1cn(Cc2ccc(C(=O)NO)cc2)c2c1C(=O)CC(C)(C)C2]

To a flask were added methyl 4-((3,6,6-trimethyl-4-oxo-4,5,6,7-tetrahydro-1H-indol-1-yl)methyl)benzoate [formula 6-3] (0.14 g, 0.44 mmol) was dissolved in methanol (4 mL). Thereto, hydroxylamine hydrochloride (NH₂OH HCl) (0.028 g, 0.40 mmol) and potassium hydroxide (0.090 g, 1.61 mmol) were added and stirred. Hydroxylamine 50% aqueous solution (2 mL) was added slowly until the mixed solution in the flask became clear, and the mixture was stirred at room temperature for one day. After the completion of the reaction, methanol was distilled out under reduced pressure, and solid product was obtained by adding little amount of ethyl acetate. The solid product was filtered, washed with water and dried under reduced pressure to yield the compound 136 as white solid (0.054 g, 38%).

¹H NMR (400 MHz, DMSO-d₆) δ 11.2 (s, 1H), 9.05 (s, 1H), 7.70 (d, 2H, J=8.3 Hz), 6.98 (d, 2H, J=8.2 Hz), 5.15 (s, 2H), 2.54 (s, 2H), 2.18 (s, 2H), 2.12 (s, 3H), 0.98 (s, 6H); MS (ESI) m/z 327 (M⁺+H).

### Example 46

### Synthesis of Compound 140

### Step 1. Synthesis of tert-butyl 4-((9-(4-(methoxycarbonyl)benzyl)-4-oxo-2,3,4,9-tetrahydro-1H-carbazol-3-yl)methyl)piperazine-1-carboxylate [formula 2-5]

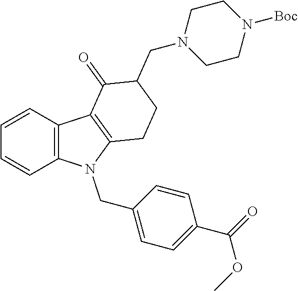 [SMILES: COC(=O)c1ccc(Cn2c3c(c4ccccc42)C(=O)C(CN2CCN(C(=O)OC(C)(C)C)CC2)CC3)cc1]

To a microwave vial were added methyl 4-((3-methylen-4-oxo-1,2,3,4-tetrahydrocarbazol-9-yl)methyl)benzoate [formula 2-4] (0.10 g, 0.290 mmol) and, tert-butyl piperazine-1-carboxylate (0.0757 g, 0.869 mmol) and toluene (3.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.061 g, 40%).

### Step 2. Synthesis of tert-butyl 4-((9-(4-(hydroxycarbamoyl)benzyl)-4-oxo-2,3,4,9-tetrahydro-1H-carbazol-3-yl)methyl)piperazine-1-carboxylate [Formula 2-6]

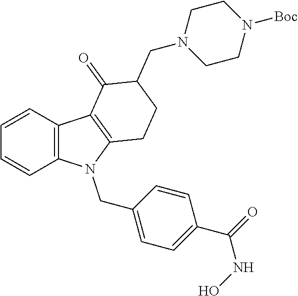 [SMILES: CC(C)(C)OC(=O)N1CCN(CC2CCc3c(c4ccccc4n3Cc3ccc(C(=O)NO)cc3)C2=O)CC1]

To a flask were added tert-butyl 4-((9-(4-(methoxycarbonyl)benzyl)-4-oxo-2,3,4,9-tetrahydro-1H-carbazol-3-yl)methyl)piperazine-1-carboxylate [formula 2-5] (0.061 g, 0.115 mmol), hydroxylamine hydrochloride (NH₂OH HCl) (0.0399 g, 0.574 mmol), potassium hydroxide (0.0644 g, 1.147 mmol) and methanol (5.0 mL) and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 140 as solid (0.025 g, 41%).

¹H NMR (400 MHz, DMSO-d₆) δ 11.2 (brs, 1H), 9.02 (brs, 1H), 8.03-8.01 (m, 1H), 7.68 (d, 2H, J=8.2 Hz), 7.49-7.47 (m, 1H), 7.18-7.16 (m, 4H), 5.53 (s, 2H), 3.30 (s, 5H), 3.10-3.06 (m, 1H), 2.95-2.89 (m, 1H), 2.72-2.69 (m, 2H), 2.44-2.42 (m, 2H), 2.36-2.31 (m, 1H), 2.24-2.22 (m, 2H), 1.97-1.95 (m, 1H), 1.87 (s, 9H); MS (ESI) m/z 533 (M++H).

### Example 47

### Synthesis of Compound 141

### Step 1. Synthesis of methyl 4-((6-fluoro-3-(morpholinomethyl)-4-oxo-1,2,3,4-tetrahydrocarbazol-9-yl)methyl)benzoate [formula 2-5]

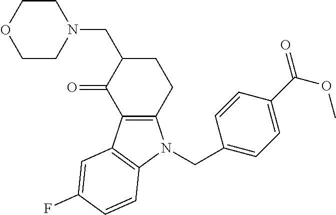 [SMILES: COC(=O)c1ccc(Cn2c3c(c4cc(F)ccc42)C(=O)C(CN2CCOCC2)CC3)cc1]

To a microwave vial were added methyl 4-((6-fluoro-3-methylen-4-oxo-1,2,3,4-tetrahydrocarbazol-9-yl)methyl)benzoate [formula 2-4] (0.10 g, 0.275 mmol), morpholine (0.0719 g, 0.826 mmol) and toluene (3.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.061 g, 49%).

### Step 2. Synthesis of 4-((6-fluoro-3-(morpholinomethyl)-4-oxo-1,2,3,4-tetrahydrocarbazol-9-yl)methyl)-N-hydroxybenzamide [formula 2-6]

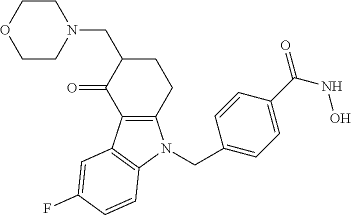 [SMILES: O=C(NO)c1ccc(Cn2c3c(c4cc(F)ccc42)C(=O)C(CN2CCOCC2)CC3)cc1]

To a flask were added methyl 4-((6-fluoro-3-(morpholinomethyl)-4-oxo-1,2,3,4-tetrahydrocarbazol-9-yl)methyl)benzoate [formula 2-5] (0.061 g, 0.135 mmol), hydroxylamine hydrochloride (NH₂OH HCl) (0.0470 g, 0.677 mmol), potassium hydroxide (0.0760 g, 1.354 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 141 as solid (0.027 g, 44%).

¹H NMR (400 MHz, DMSO-d₆) δ 11.1 (brs, 1H), 9.04 (s, 1H), 7.69-7.66 (m, 3H), 7.53-7.49 (m, 1H), 7.18 (d, 2H, J=8.3 Hz), 7.06-7.01 (m, 1H), 5.53 (s, 2H), 3.59-3.33 (brs, 4H), 3.09-3.04 (m, 1H), 2.96-2.88 (m, 1H), 2.74-2.67 (m, 2H), 2.54-2.50 (s, 2H), 2.49-2.27 (m, 4H), 2.01-1.97 (m, 1H); MS (ESI) m/z 452 (M++H).

### Example 48

### Synthesis of Compound 142

### Step 1. Synthesis of methyl 4-((6-fluoro-3-((4-methylpiperazin-1-yl)methyl)-4-oxo-1,2,3,4-tetrahydrocarbazol-9-yl)methyl)benzoate [formula 2-5]

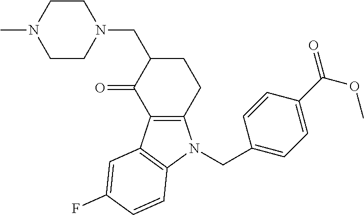

To a microwave vial were added methyl 4-((6-fluoro-3-methylen-4-oxo-1,2,3,4-tetrahydrocarbazol-9-yl)methyl)benzoate [formula 2-4] (0.10 g, 0.275 mmol), 1-methylpiperazine (0.0827 g, 0.826 mmol) and toluene (3.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound as white solid (0.063 g, 49%).

### Step 2. Synthesis of 4-((6-fluoro-3-((4-methylpiperazin-1-yl)methyl)-4-oxo-1,2,3,4-tetrahydrocarbazol-9-yl)methyl)-N-hydroxybenzamide [formula 2-6]

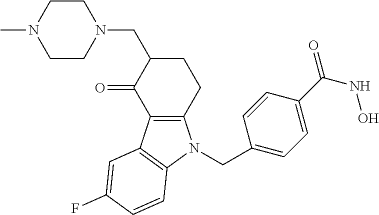 [SMILES: CN1CCN(CC2CCc3c(c4cc(F)ccc4n3Cc3ccc(C(=O)NO)cc3)C2=O)CC1]

To a flask were added methyl 4-((6-fluoro-3-((4-methylpiperazin-1-yl)methyl)-4-oxo-1,2,3,4-tetrahydrocarbazol-9-yl)methyl)benzoate [formula 2-5] (0.063 g, 0.136 mmol), hydroxylamine hydrochloride (NH₂OH HCl) (0.0472 g, 0.680 mmol), potassium hydroxide (0.0763 g, 1.359 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 142 as white solid (0.031 g, 49%).

¹H NMR (400 MHz, DMSO-d₆) δ 11.2 (brs, 1H), 9.03 (brs, 1H), 7.69-7.67 (m, 3H), 7.52-7.49 (m, 1H), 7.18 (d, 2H, J=8.2 Hz), 7.06-7.01 (m, 1H), 5.54 (s, 2H), 3.33 (s, 2H), 3.08-3.04 (m, 1H), 2.96-2.88 (m, 1H), 2.68-2.64 (m, 2H), 2.49-2.42 (m, 2H), 2.33-2.18 (m, 6H), 2.13 (s, 3H), 1.95-1.93 (m, 1H); MS (ESI) m/z 465 (M++H).

### Example 49

### Synthesis of Compound 144

### Step 1. Synthesis of tert-butyl 4-((6-fluoro-9-(4-(hydroxycarbonyl)benzyl)-4-oxo-2,3,4,9-tetrahydro-1H-carbazol-3-yl)methyl)piperazin-1-carboxylate [formula 2-5]

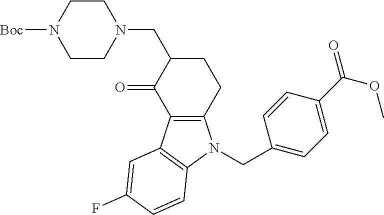 [SMILES: COC(=O)c1ccc(Cn2c3c(c4cc(F)ccc42)C(=O)C(CN2CCN(C(=O)OC(C)(C)C)CC2)CC3)cc1]

To a microwave vial were added methyl 4-((6-fluoro-3-methylen-4-oxo-1,2,3,4-tetrahydrocarbazol-9-yl)methyl)benzoate [formula 2-4] (0.10 g, 0.275 mmol), tert-butylpiperazin-1-carboxylate (0.0719 g, 0.826 mmol) and toluene (3.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.054 g, 36%).

### Step 2. Synthesis of tert-butyl 4-((6-fluoro-9-(4-(hydroxycarbamoyl)benzyl)-4-oxo-2,3,4,9-tetrahydro-1H-carbazol-3-yl)methyl)piperazin-1-carboxylate [formula 2-6]

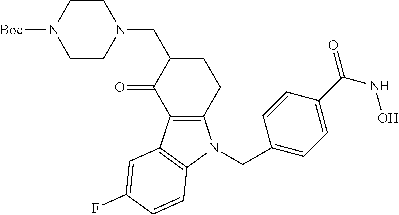 [SMILES: CC(C)(C)OC(=O)N1CCN(CC2CCc3c(c4cc(F)ccc4n3Cc3ccc(C(=O)NO)cc3)C2=O)CC1]

To a flask were added tert-butyl 4-((6-fluoro-9-(4-(methoxycarbonyl)benzyl)-4-oxo-2,3,4,9-tetrahydro-1H-carbazol-3-yl)methyl)piperazin-1-carboxylate [formula 2-5] (0.054 g, 0.098 mmol), hydroxylamine hydrochloride (NH₂OH HCl) (0.0341 g, 0.491 mmol), potassium hydroxide (0.0551 g, 0.982 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 144 as solid (0.032 g, 59%).

¹H NMR (400 MHz, DMSO-d₆) δ 11.1 (brs, 1H), 9.03 (brs, 1H), 7.69-7.66 (m, 3H), 7.53-7.50 (m, 1H), 7.18 (d, 2H, J=8.2 Hz), 7.07-7.02 (m, 1H), 5.54 (s, 2H), 3.30 (brs, 4H), 3.10-3.06 (m, 1H), 2.95-2.92 (m, 1H), 2.72-2.68 (m, 2H), 2.48-2.42 (m, 3H), 2.36-2.32 (m, 1H), 2.25-2.22 (m, 2H), 2.21-1.97 (m, 1H), 1.39 (s, 9H); MS (ESI) m/z 551 (M++H).

### Example 50

### Synthesis of Compound 145

### Step 1. Synthesis of methyl 4-((3-((3,3-difluoroazetidin-1-yl)methyl)-6-fluoro-4-oxo-1,2,3,4-tetrahydrocarbazol-9-yl)benzoate [formula 2-5]

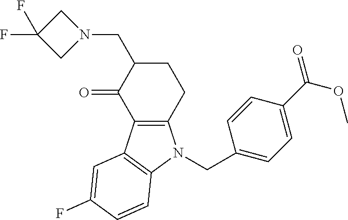 [SMILES: COC(=O)c1ccc(Cn2c3c(c4cc(F)ccc42)C(=O)C(CN2CC(F)(F)C2)CC3)cc1]

To a microwave vial were added methyl 4-((6-fluoro-3-methylen-4-oxo-1,2,3,4-tetrahydrocarbazol-9-yl)methyl)benzoate [formula 2-4] (0.10 g, 0.275 mmol), 3,3-difluoroazetidine HCl (0.1069 g, 0.826 mmol) and toluene (3.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.031 g, 25%).

### Step 2. Synthesis of 4-((3-((3,3-difluoroazetidin-1-yl)methyl)-6-fluoro-4-oxo-1,2,3,4-tetrahydrocarbazol-9-yl)methyl)-N-hydroxybenzamide [formula 2-6]

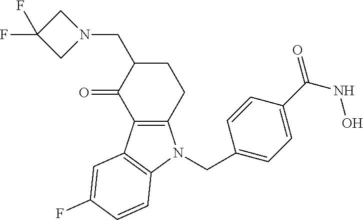 [SMILES: O=C(NO)c1ccc(Cn2c3c(c4cc(F)ccc42)C(=O)C(CN2CC(F)(F)C2)CC3)cc1]

To a flask were added methyl 4-((3-((3,3-difluoroazetidin-1-yl)methyl)-6-fluoro-4-oxo-1,2,3,4-tetrahydrocarbazol-9-yl)benzoate [formula 2-5] (0.031 g, 0.068 mmol), hydroxylamine hydrochloride (NH₂OH HCl) (0.0236 g, 0.340 mmol), potassium hydroxide (0.0381 g, 0.679 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 145 as solid (0.017 g, 55%).

¹H NMR (400 MHz, DMSO-d₆) δ 11.2 (s, 1H), 9.03 (s, 1H), 7.69-7.67 (m, 3H), 7.53-7.50 (m, 1H), 7.17 (d, 2H, J=8.3 Hz), 7.07-7.03 (m, 1H), 5.54 (s, 2H), 3.64-3.54 (m, 4H), 3.18-3.02 (m, 1H), 2.97-2.94 (m, 2H), 2.82-2.80 (m, 1H), 2.60-2.50 (m, 1H), 2.29-2.23 (m, 1H), 2.02-1.97 (m, 1H); MS (ESI) m/z 458 (M++H).

### Example 51

### Synthesis of Compound 156

### Step 1. Synthesis of methyl 4-((3-((4-(cyclopropancarbonyl)piperazin-1-yl)methyl)-4-oxo-1,2,3,4-tetrahydrocarbazol-9-yl)methyl)benzoate [formula 2-5]

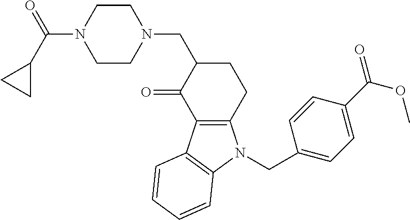 [SMILES: COC(=O)c1ccc(Cn2c3c(c4ccccc42)C(=O)C(CN2CCN(C(=O)C4CC4)CC2)CC3)cc1]

To a microwave vial were added methyl 4-((3-methylen-4-oxo-1,2,3,4-tetrahydrocarbazol-9-yl)methyl)benzoate [formula 2-4] (0.10 g, 0.290 mmol), 1-(cyclopropylcarbonyl)piperazine (0.134 g, 0.869 mmol) and toluene (3.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.056 g, 39%).

### Step 2. Synthesis of 4-((3-((4-(cyclopropancarbonyl)piperazin-1-yl)methyl)-4-oxo-1,2,3,4-tetrahydrocarbazol-9-yl)methyl)-N-hydroxybenzamide [formula 2-6]

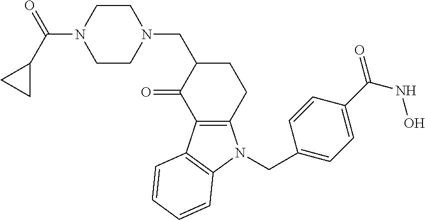 [SMILES: O=C(NO)c1ccc(Cn2c3c(c4ccccc42)C(=O)C(CN2CCN(C(=O)C4CC4)CC2)CC3)cc1]

To a flask were added methyl 4-((3-((4-(cyclopropancarbonyl)piperazin-1-yl)methyl)-4-oxo-1,2,3,4-tetrahydrocarbazol-9-yl)methyl)benzoate [formula 2-5] (0.056 g, 0.112 mmol), hydroxylamine hydrochloride (NH₂OH HCl) (0.0389 g, 0.560 mmol), potassium hydroxide (0.0629 g, 1.121 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 156 as solid (0.031 g, 55%).

¹H NMR (400 MHz, DMSO-d₆) δ 8.03-8.01 (m, 1H), 7.66 (d, 2H, J=8.2 Hz), 7.50-7.49 (m, 1H), 7.20-7.16 (m, 2H), 7.09 (d, 2H, J=8.2 Hz), 5.48 (s, 2H), 3.65 (brs, 2H), 3.44-3.34 (m, 2H), 3.12-3.07 (m, 1H), 2.97-2.94 (m, 1H), 2.72-2.69 (m, 2H), 2.50-2.44 (m, 2H), 2.38-2.32 (m, 2H), 2.22-2.19 (m, 1H), 1.99-1.94 (m, 2H), 0.73-0.69 (m, 4H); MS (ESI) m/z 501 (M++H).

### Example 52

### Synthesis of Compound 157

### Step 1. Synthesis of methyl 4-((3-fluoro-5-oxo-5,6,7,8-tetrahydrocarbazol-9-yl)methyl)benzoate [formula 1-5]

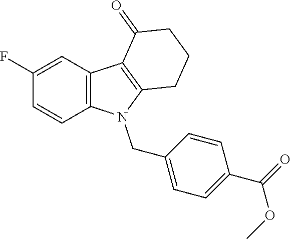 [SMILES: COC(=O)c1ccc(Cn2c3c(c4cc(F)ccc42)C(=O)CCC3)cc1]

6-fluoro-2,3-dihydro-1H-carbazol-4(9H)-on [formula 1-2] (3.50 g, 17.2 mmol) was dissolved in DMF (30 mL), 55% NaH in paraffin solution (1.50 g, 34.4 mmol) was added and stirred for 10 minutes. Then, methyl 4-(bromomethyl)benzoate (4.73 g, 20.7 mmol) was added and stirred at 50° C. for 5 hours. After the completion of the reaction, DMF was distilled out, and the reaction mixture was extracted with ethyl acetate and saturated NaHCO₃, the organic layer was washed with brine, dried over anhydrous Na₂SO₄, filtered and concentrated under reduced pressure. Residue was purified by column chromatography (SiO₂; hexane/ethylacetate, 5/1) to yield the title compound (2.1 g, 35%).

### Step 2. Synthesis of 4-((3-fluoro-5-oxo-5,6,7,8-tetrahydrocarbazol-9-yl)methyl)-N-hydroxybenzamide [formula 1-6]

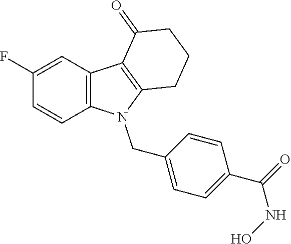 [SMILES: O=C(NO)c1ccc(Cn2c3c(c4cc(F)ccc42)C(=O)CCC3)cc1]

To a flask were added methyl 4-((3-fluoro-5-oxo-5,6,7,8-tetrahydrocarbazol-9-yl)methyl)benzoate [formula 1-5] (0.1 g, 0.285 mmol), hydroxylamine hydrochloride (NH₂OH HCl) (0.0989 g, 1.42 mmol), potassium hydroxide (0.106 g, 2.85 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 157 as solid (0.061 g, 61%). ¹H NMR (400 MHz, DMSO-d₆) δ 11.1 (brs, 1H), 9.04 (brs, 1H), 7.69-7.65 (m, 3H), 7.55-7.51 (m, 1H), 7.18 (d, 2H, J=8.3 Hz), 7.09-7.02 (m, 1H), 5.55 (s, 2H), 2.97 (t, 2H, J=6.0 Hz), 2.46 (m, 2H), 2.13 (t, 2H, J=6.2 Hz); MS (ESI) m/z 353 (M++H).

### Example 53

### Synthesis of Compound 158

### Step 1. Synthesis of methyl 4-((6-fluoro-3-((2-methyl-1H-imidazol-1-yl)methyl)-4-oxo-1,2,3,4-tetrahydrocarbazol-9-yl)methyl)benzoate [formula 2-5]

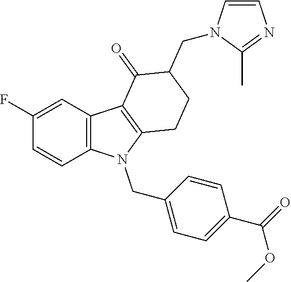 [SMILES: COC(=O)c1ccc(Cn2c3c(c4cc(F)ccc42)C(=O)C(Cn2ccnc2C)CC3)cc1]

To a microwave vial were added methyl 4-((6-fluoro-3-methylen-4-oxo-1,2,3,4-tetrahydrocarbazol-9-yl)methyl)benzoate [formula 2-4] (0.10 g, 0.275 mmol), 2-methyl-1H-imidazole (0.0678 g, 0.826 mmol) and toluene (4.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.057 g, 44%).

### Step 2. Synthesis of 4-((6-fluoro-3-((2-methyl-1H-imidazol-1-yl)methyl)-4-oxo-1,2,3,4-tetrahydrocarbazol-9-yl)methyl)-N-hydroxybenzamide [formula 2-6]

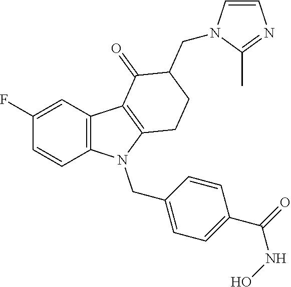 [SMILES: Cc1nccn1CC1CCc2c(c3cc(F)ccc3n2Cc2ccc(C(=O)NO)cc2)C1=O]

To a flask were added methyl 4-((6-fluoro-3-((2-methyl-1H-imidazol-1-yl)methyl)-4-oxo-1,2,3,4-tetrahydrocarbazol-9-yl)methyl)benzoate [formula 2-5] (0.0570 g, 0.120 mmol), hydroxylamine hydrochloride (NH₂OH HCl) (0.0418 g, 0.602 mmol), potassium hydroxide (0.0675 g, 1.204 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 158 as solid (0.031 g, 54%).

¹H NMR (400 MHz, DMSO-d₆) δ 11.2 (brs, 1H), 9.06 (brs, 1H), 7.73-7.66 (m, 3H), 7.54-7.52 (m, 1H), 7.17 (d, 2H, J=7.5 Hz), 7.09-7.04 (m, 2H), 6.72 (s, 1H), 5.53 (s, 2H), 4.45 (d, 1H, J=13.8 Hz), 4.10-4.04 (m, 1H), 3.13-3.08 (m, 1H), 2.99-2.89 (m, 2H), 2.29 (s, 3H), 2.04-1.98 (m, 1H), 1.86-1.81 (m, 1H); MS (ESI) m/z 447 (M++H).

### Example 54

### Synthesis of Compound 166—Scheme 6

### Step 1. Synthesis of 3-methyl-6,7-dihydro-1H-indole-4(5H)-on [formula 6-2]

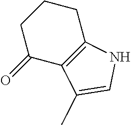

Anti-pyruvic aldehyde-1-oxime [formula 6-1] (2.0 g, 22.9 mmol) and 5,5-dimethyl-1,3-cyclohexandion [formula 6-7] (0.80 g, 5.74 mmol) were dissolved in acetic acid (35 mL) and H₂O (15 mL). Thereto, zinc powder (0.75 g, 11.5 mmol) was added slowly maintaining room temperature. The reaction mixture was refluxed with stirring for one day, concentrated under reduced pressure and extracted with CH₂Cl₂ and brine, of which pH was adjusted to about 6 using saturated NaHCO₃. The reaction mixture was extracted with CH₂Cl₂; organic layer was dried over anhydrous MgSO₄ and filtered. Residue was concentrated under reduced pressure and purified by column chromatography (SiO₂; hexane/ethylacetate, 1/3) to yield the title compound as yellow solid (4.9 g, 52%).

### Step 2. Synthesis of methyl 4-((3,6,6-trimethyl-4-oxo-4,5,6,7-tetrahydro-1H-indole-1-yl)methyl)benzoate [formula 6-3]

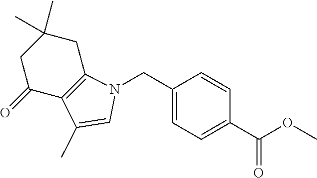 [SMILES: COC(=O)c1ccc(Cn2cc(C)c3c2CC(C)(C)CC3=O)cc1]

3,6,6-trimethyl-6,7-dihydro-1H-indole-4(5H)-on [formula 6-2] (0.23 g, 1.29 mmol) was dissolved in DMF, NaH (0.062 g, 2.56 mmol) was added slowly maintaining room temperature. After 5 minute of stirring, methyl 4-(bromethyl)benzoate was added and stirred at room temperature for 4 hours. After the completion of reaction, the reaction mixture was extracted with ethyl acetate and saturated NH₄Cl aqueous solution; the organic layer was dried over anhydrous MgSO₄ and filtered. Filtrate was concentrated under reduced pressure and purified by column chromatography (SiO₂; hexane/ethylacetate, 7/3) to yield the title compound as white solid (0.14 g, 34%).

### Step 3. Synthesis of N-hydroxy-4-((3,6,6-trimethyl-4-oxo-4,5,6,7-tetrahydro-1H-indol-1-yl)methyl)benzamide [formula 6-4]

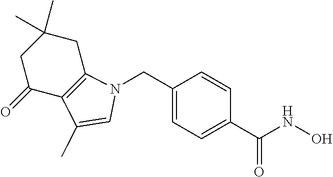 [SMILES: Cc1cn(Cc2ccc(C(=O)NO)cc2)c2c1C(=O)CC(C)(C)C2]

Methyl4-((3,6,6-trimethyl-4-oxo-4,5,6,7-tetrahydro-1H-indole-1-yl)methyl)benzoate [formula 6-3] (0.14 g, 0.44 mmol) was dissolved in methanol (4 mL). Thereto, hydroxylamine 50% aqueous solution (1 mL), hydroxylamine hydrochloride (NH₂OH HCl) (0.028 g, 0.40 mmol) and potassium hydroxide (0.090 g, 1.61 mmol) were added in order and stirred at room temperature for one day. After the completion of the reaction, methanol was distilled out under reduced pressure, and saturated NaHCO₃ (1-2 mL) was added and stirred. Obtained solid product was filtered, washed with water and dried under reduced pressure to yield the compound 166 as white solid (0.054 g, 38%).

¹H NMR (400 MHz, DMSO-d₆) δ 11.2 (s, 1H), 9.06 (s, 1H), 7.72 (d, 2H, J=7.9 Hz), 7.19 (d, 2H, J=7.8 Hz), 6.61 (s, 1H), 5.11 (s, 2H), 2.63 (s, 2H), 2.25 (s, 2H), 2.14 (s, 3H), 1.96 (s, 2H); MS (ESI) m/z 299 (M⁺+H).

### Example 55

### Synthesis of Compound 179—Scheme 12

### Step 1. Synthesis of ethyl-2-cyano-2-(2-nitrophyenyl)acetate [formula 12-2]

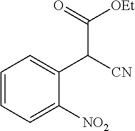 [SMILES: CCOC(=O)C(C#N)c1ccccc1[N+](=O)[O-]]

Potassium tert-Butoxide (17.497 g, 155.918 mmol) was mixed with THF (200 mL) in an ice bath with stirring. Then, ethyl cyanoacetate (15.126 mL, 141.743 mmol) was added drop-wise slowly and obtained white solid. Then, the reaction mixture was stirred for 15 minute, and 1-fluoro-2-nitrobenzen [formula 12-1] (7.463 mL, 70.872 mmol) was added drop-wise and refluxed for 3 hours. After the completion of the reaction, the reaction mixture was extracted with ethyl acetate. 2N HCl was added, and the mixture was purified by column chromatography (Hexane/Dichloromethane=1/4) to yield the compound as yellow oil (16.0 g, 96.4%).

### Step 2. Synthesis of ethyl-2-amino-1H-indol-3-carboxyate [formula 12-3]

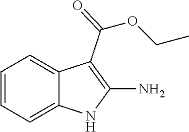

Ethyl-2-cyano-2-(2-nitrophyenyl)acetate [formula 12-2] (16 g, 68.315 mmol) was dissolved in AcOH (200 mL), Zn (17.86 g, 273.25 mmol) was added and stirred at 65° C. for one day. Then Zn was deleted with celite filtering, and AcOH was removed under the reduced pressure to obtain solid product. The obtained solid was dissolved in excess dichloromethane, and then hexane was added to yield the title compound (6 g, 44%).

### Step 3. Synthesis of 3H-pyrimido[4,5-b]indole-4(9H)-on [formula 12-4]

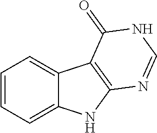 [SMILES: O=c1[nH]cnc2[nH]c3ccccc3c12]

Ethyl-2-amino-1H-indol-3-carboxyate [formula-12-3] (8 g, 39.172 mmol) and sodium methoxide (2.116 g, 39.172 mmol) was dissolved in formamide (40 mL) and stirred at 220° C. for an hour and half. Then the reaction mixture was filtered with adding water at room temperature. The filtered substance was dried to yield the title compound as black solid (0.92 g, 78%).

### Step 4. Synthesis of methyl 4-((4-oxo-3,4-dihydropyrimido[4,5-b]indol-9-yl)methyl)benzoate [formula 12-5]

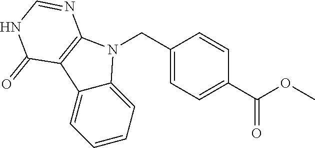 [SMILES: COC(=O)c1ccc(Cn2c3ccccc3c3c(=O)[nH]cnc32)cc1]

3H-pyrimido[4,5-b]indole-4(9H)-on [formula 12-4] was dissolved in DMF, t-BuOK (0.2182 g, 1.944 mmol) was added in an ice bath. Then, methyl(bromomethyl)benzoate (0.4082 g, 1.782 mmol) [formula 2] and small amount of KI (0.027 g, 0.162 mmol) was added and stirred at 50° C. for 12 hours. After the completion of the reaction, solvent was removed under reduced pressure and the reaction mixture was extracted with ethyl acetate to yield the title compound as yellow solid (0.15 g, 27.8%).

### Step 5. Synthesis of N-hydroxy-4-((4-oxo-3,4-dihydropyrimido[4,5-b]indol-9-yl)methyl)benzamide [formula 12-6]

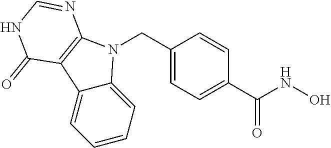 [SMILES: O=C(NO)c1ccc(Cn2c3ccccc3c3c(=O)[nH]cnc32)cc1]

Methyl 4-((4-oxo-3,4-dihydropyrimido[4,5-b]indol-9-yl)methyl)benzoate [formula 12-5] (0.05 g, 0.15 mmol) was dissolved in methanol (20 mL) and THF (20 mL). Then, NH₂OH (0.0521 g, 0.75 mmol) and KOH (0.084 g, 1.5 mmol) was added and stirred for 10 minutes. When the solution became cloudy, hydroxylamine 50 wt % solution in water (0.417 mL, 3.0 mmol) was added drop-wise, and stored overnight. After confirming the completion of the reaction with TLC, Methanol was removed under reduced pressure remaining water only, and a small volume of water (5 mL) was added, and 1N HCL aqueous solution added drop-wise. Then, precipitated white solid was dried to yield the compound of 179. (0.045 g, 89.7%).

¹H NMR (400 MHz, DMSO-d₆) δ 12.28 (s, 1H), 11.21 (s, 1H), 9.05 (s, 1H); 8.66 (s, 1H), 7.97 (d, 1H, J=7.7 Hz), 7.71 (d, 2H, J=8.3 Hz), 7.48 (d, 1H, J=8.1 Hz), 7.40 (d, 2H, J=8.3 Hz), 7.36-7.21 (m, 2H), 5.30 (s, 2H); MS (ESI) m/z 333.1 (M⁺−H).

### Example 56

### Synthesis of Compound 188

### Step 1. Synthesis of methyl 4-((6-fluoro-3-((4-isopropylpiperazin-1-yl)methyl)-4-oxo-1,2,3,4-tetrahydrocarbazol-9-yl)methyl)benzoate [formula 2-5]

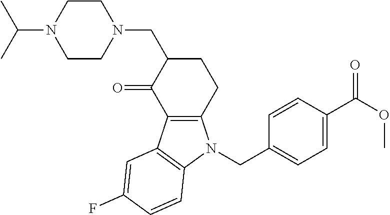 [SMILES: COC(=O)c1ccc(Cn2c3c(c4cc(F)ccc42)C(=O)C(CN2CCN(C(C)C)CC2)CC3)cc1]

To a microwave vial were added methyl 4-((6-fluoro-3-methylen-4-oxo-1,2,3,4-tetrahydrocarbazol-9-yl)methyl)benzoate [formula 2-4] (0.10 g, 0.275 mmol), 1-isopropylpiperazine (0.106 g, 0.826 mmol) and toluene (4.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.051 g, 38%).

### Step 2. Synthesis of 4-((6-fluoro-3-((4-isopropylpiperazin-1-yl)methyl)-4-oxo-1,2,3,4-tetrahydrocarbazol-9-yl)methyl)-N-hydroxybenzamide [formula 2-6]

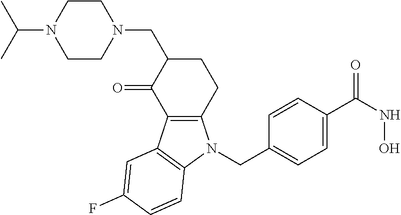 [SMILES: CC(C)N1CCN(CC2CCc3c(c4cc(F)ccc4n3Cc3ccc(C(=O)NO)cc3)C2=O)CC1]

To a flask were added methyl methyl 4-((6-fluoro-3-((4-isopropylpiperazin-1-yl)methyl)-4-oxo-1,2,3,4-tetrahydrocarbazol-9-yl)methyl)benzoate [formula 2-5] (0.051 g, 0.104 mmol), hydroxylamine hydrochloride (NH₂OH HCl) (0.036 g, 0.519 mmol), potassium hydroxide (0.0582 g, 1.037 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 188 as solid (0.034 g, 66%).

¹H NMR (400 MHz, DMSO-d₆) δ 11.2 (brs, 1H), 9.02 (brs, 1H), 7.69-7.66 (m, 3H), 7.52-7.49 (m, 1H), 7.17 (d, 2H, 0.1=8.4 Hz), 7.06-7.01 (m, 1H), 5.53 (s, 2H), 3.09-3.02 (m, 1H), 2.96-2.90 (m, 1H), 2.71-2.62 (m, 2H), 2.60-2.57 (m, 2H), 2.56-2.41 (m, 6H), 2.33-2.29 (m, 3H), 2.01-1.89 (m, 1H), 0.99 (s, 3H), 0.89 (s, 3H); MS (ESI) m/z 493 (M++H).

### Example 57

### Synthesis of Compound 189

### Step 1. Synthesis of methyl 4-((3-((4-ethylpiperazin-1-yl)methyl)-6-fluoro-4-oxo-1,2,3,4-tetrahydrocarbazol-9-yl)methyl)benzoate [formula 2-5]

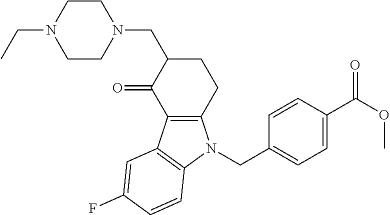 [SMILES: CCN1CCN(CC2CCc3c(c4cc(F)ccc4n3Cc3ccc(C(=O)OC)cc3)C2=O)CC1]

To a microwave vial were added methyl 4-((6-fluoro-3-methylen-4-oxo-1,2,3,4-tetrahydrocarbazol-9-yl)methyl)benzoate [formula 2-4] (0.10 g, 0.275 mmol), 1-ethylpiperazine (0.0943 g, 0.826 mmol) and toluene (4.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.061 g, 46%).

### Step 2. Synthesis of 4-((3-((4-ethylpiperazin-1-yl)methyl)-6-fluoro-4-oxo-1,2,3,4-tetrahydrocarbazol-9-yl)methyl)-N-hydroxybenzamide [formula 2-6]

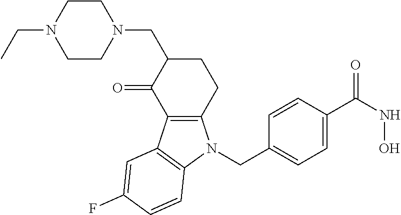 [SMILES: CCN1CCN(CC2CCc3c(c4cc(F)ccc4n3Cc3ccc(C(=O)NO)cc3)C2=O)CC1]

To a flask were added methyl 4-((3-((4-ethylpiperazin-1-yl)methyl)-6-fluoro-4-oxo-1,2,3,4-tetrahydrocarbazol-9-yl)methyl)benzoate [formula 2-5] (0.061 g, 0.128 mmol), hydroxylamine hydrochloride (NH₂OH HCl) (0.044 g, 0.639 mmol), potassium hydroxide (0.0717 g, 1.28 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 189 as solid (0.023 g, 38%).

¹H NMR (400 MHz, DMSO-d₆) δ 11.2 (brs, 1H), 9.03 (brs, 1H), 7.69-7.66 (m, 3H), 7.52-7.49 (m, 1H), 7.17 (d, 2H, J=8.2 Hz), 7.06-7.01 (m, 1H), 5.53 (s, 2H), 3.34-3.04 (m, 1H), 2.96-2.88 (m, 1H), 2.71-2.64 (m, 2H), 2.49-2.42 (m, 2H), 2.33-2.17 (m, 8H), 1.95-1.93 (m, 1H), 0.97 (t, 3H, J=7.1 Hz); MS (ESI) m/z 479 (M++H).

### Example 58

### Synthesis of Compound 190

### Step 1. Synthesis of methyl 4-((3-((4-butylpiperazin-1-yl)methyl)-6-fluoro-4-oxo-1,2,3,4-tetrahydrocarbazol-9-yl)methyl)benzoate [formula 2-5]

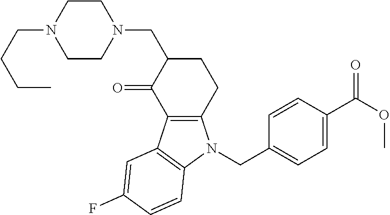 [SMILES: CCCCN1CCN(CC2CCc3c(c4cc(F)ccc4n3Cc3ccc(C(=O)OC)cc3)C2=O)CC1]

To a microwave vial were added methyl 4-((6-fluoro-3-methylen-4-oxo-1,2,3,4-tetrahydrocarbazol-9-yl)methyl)benzoate [formula 2-4] (0.10 g, 0.275 mmol), 1-buthylpiperazine (0.117 g, 0.826 mmol) and toluene (4.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.055 g, 40%).

### Step 2. Synthesis of 4-((3-((4-butylpiperazin-1-yl)methyl)-6-fluoro-4-oxo-1,2,3,4-tetrahydrocarbazol-9-yl)methyl)-N-hydroxybenzamide [formula 2-6]

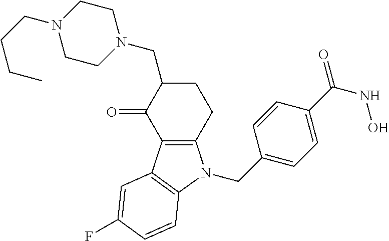 [SMILES: CCCCN1CCN(CC2CCc3c(c4cc(F)ccc4n3Cc3ccc(C(=O)NO)cc3)C2=O)CC1]

Methyl 4-((3-((4-butylpiperazin-1-yl)methyl)-6-fluoro-4-oxo-1,2,3,4-tetrahydrocarbazol-9-yl)methyl)benzoate [formula 2-5] (0.055 g, 0.109 mmol), hydroxylamine hydrochloride (NH₂OH HCl) (0.0378 g, 0.544 mmol), potassium hydroxide (0.061 g, 1.088 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 190 as solid (0.027 g, 49%).

¹H NMR (400 MHz, DMSO-d₆) δ 7.69-7.66 (m, 3H), 7.52-7.49 (m, 1H), 7.17 (d, 2H, J=8.4 Hz), 7.06-7.01 (m, 1H), 5.53 (s, 2H), 3.08-3.04 (m, 1H), 2.96-2.89 (m, 1H), 2.71-2.64 (m, 2H), 2.48-2.41 (m, 2H), 2.39-2.28 (m, 5H), 2.38-2.21 (m, 4H), 2.20-1.96 (m, 1H), 1.41-1.35 (m, 2H), 1.27-1.20 (m, 3H), 0.97 (t, 3H, J=7.1 Hz); MS (ESI) m/z 507 (M++H).

### Example 59

### Synthesis of Compound 191

### Step 1. Synthesis of methyl 4-((6-fluoro-3-((4-(2-hydroxy-2-methylpropyl)piperazin-1-yl)methyl)-4-oxo-1,2,3,4-tetrahydrocarbazol-9-yl)methyl)benzoate [formula 2-5]

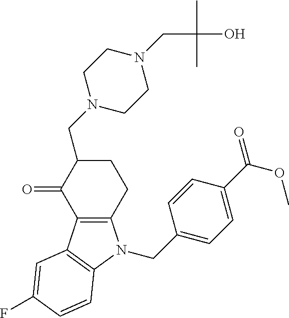 [SMILES: COC(=O)c1ccc(Cn2c3c(c4cc(F)ccc42)C(=O)C(CN2CCN(CC(C)(C)O)CC2)CC3)cc1]

To a microwave vial were added methyl 4-((6-fluoro-3-methylen-4-oxo-1,2,3,4-tetrahydrocarbazol-9-yl)methyl)benzoate [formula 2-4] (0.15 g, 0.413 mmol), piperazine (0.107 g, 1.238 mmol) and toluene (3.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the compound methyl 4-((6-fluoro-4-oxo-3-(piperazin-1-ylmethyl)-1,2,3,4-tetrahydrocarbazol-9-yl)methyl)benzoate (0.090 g, 49%). The obtained compound was dissolved in methanol and added in a microwave vial with 1,2-epoxy-2-methylpropane (0.0144 g, 0.20 mmol), and a reaction was carried out in a microwave reactor at 110° C. for 20 minutes. Then, solvent was concentrated under reduced pressure and extracted with ethyl acetate, and the organic layer was dried. Residue was purified by column chromatography (SiO₂; dichloromethane/methanol, 10/1) to yield the title compound (0.071 g, 68%).

### Step 2. Synthesis of 4-((6-fluoro-3-((4-(2-hydroxy-2-methylpropyl)piperazin-1-yl)methyl)-4-oxo-1,2,3,4-tetrahydrocarbazol-9-yl)methyl)-N-hydroxybenzamide [formula 2-6]

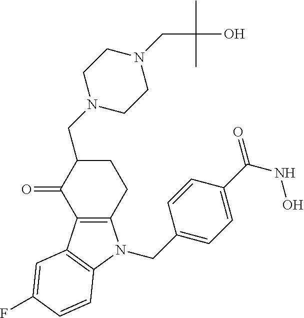 [SMILES: CC(C)(O)CN1CCN(CC2CCc3c(c4cc(F)ccc4n3Cc3ccc(C(=O)NO)cc3)C2=O)CC1]

Methyl 4-((6-fluoro-3-((4-(2-hydroxy-2-methylpropyl)piperazin-1-yl)methyl)-4-oxo-1,2,3,4-tetrahydrocarbazol-9-yl)methyl)benzoate [formula 2-5] (0.071 g, 0.136 mmol), hydroxylamine hydrochloride (NH₂OH HCl) (0.0473 g, 0.681 mmol), potassium hydroxide (0.0764 g, 1.361 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 191 as solid (0.038 g, 53%).

¹H NMR (400 MHz, DMSO-d₆) δ 7.70-7.63 (m, 3H), 7.53-7.50 (m, 1H), 7.18 (d, 2H, J=7.9 Hz), 7.05 (t, 1H, J=9.2 Hz), 5.54 (s, 2H), 4.05 (s, 1H), 3.09-3.05 (m, 1H), 3.01-2.95 (m, 1H), 2.67-2.65 (m, 2H), 2.63-2.40 (m, 6H), 2.39-2.30 (m, 3H), 2.16 (s, 2H), 2.10-1.95 (m, 1H), 1.07 (s, 6H); MS (ESI) ink 523 (M++H).

### Example 60

### Synthesis of Compound 192

### Step 1. Synthesis of methyl 4-((6-fluoro-2,2-dimethyl-3-((2-methyl-1H-imidazol-1-yl)methyl)-4-oxo-1,2,3,4-tetrahydrocarbazol-9-yl)methyl)benzoate [formula 2-5]

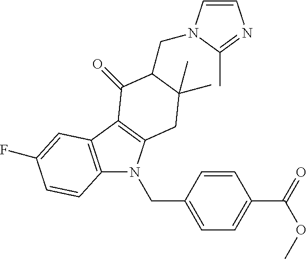 [SMILES: COC(=O)c1ccc(Cn2c3c(c4cc(F)ccc42)C(=O)C(Cn2ccnc2C)C(C)(C)C3)cc1]

To a microwave vial were added methyl 4-((6-fluoro-2,2-dimethyl-3-methylen-4-oxo-1,2,3,4-tetrahydrocarbazol-9-yl)methyl)benzoate [formula 2-4] (0.10 g, 0.255 mmol), 2-methyl-1H-imidazol (0.0629 g, 0.766 mmol) and toluene (4.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.068 g, 56%).

### Step 2. Synthesis of 4-((6-fluoro-2,2-dimethyl-3-((2-methyl-1H-imidazol-1-yl)methyl)-4-oxo-1,2,3,4-tetrahydrocarbazol-9-yl)methyl)-N-hydroxybenzamide [formula 2-6]

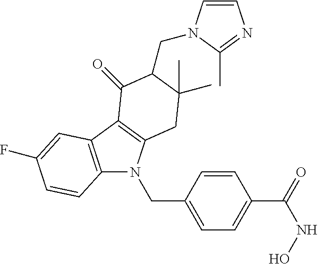 [SMILES: Cc1nccn1CC1C(=O)c2c(n(Cc3ccc(C(=O)NO)cc3)c3ccc(F)cc23)CC1(C)C]

Methyl 4-((6-fluoro-2,2-dimethyl-3-((2-methyl-1H-imidazol-1-yl)methyl)-4-oxo-1,2,3,4-tetrahydrocarbazol-9-yl)methyl)benzoate [formula 2-5] (0.068 g, 0.144 mmol), hydroxylamine hydrochloride (NH₂OH HCl) (0.0499 g, 0.718 mmol), potassium hydroxide (0.0806 g, 1.436 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 192 as solid (0.025 g, 37%).

¹H NMR (400 MHz, DMSO-d₆) δ 11.2 (brs, 1H), 9.02 (brs, 1H), 7.69 (d, 2H, J=8.3 Hz), 7.64-7.61 (m, 1H), 7.53-7.49 (m, 1H), 7.13 (d, 2H, 8.2 Hz), 7.07-7.02 (m, 2H), 6.67 (s, 1H), 5.57 (s, 2H), 4.29-4.25 (m, 1H), 4.13-4.09 (m, 1H), 101 (s, 2H), 2.82-2.80 (m, 1H), 2.24 (s, 3H), 1.22 (s, 3H), 1.05 (s, 3H); MS (ESI) m/z 475 (M++H).

### Example 61

### Synthesis of Compound 193

### Step 1. Synthesis of ethyl 6-(4-oxo-1,2,3,4-tetrahydrocarbazol-9-yl)hexanoate [formula 1-3]

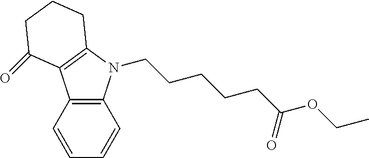 [SMILES: CCOC(=O)CCCCCn1c2c(c3ccccc31)C(=O)CCC2]

2,3-dihydro-1H-carbazol-4(9H)-on) [formula 2-2] (1.0 g, 5.40 mmol) was dissolved in DMF (20 mL), 55% NaH in paraffin solution (0.471 g, 10.8 mmol) was added and stirred for 10 minutes. Then, ethyl 6-bromomhexanoate (1.45 g, 6.48 mmol) was added and stirred at 50° C. for 5 hours. After the completion of the reaction, DMF was distilled out, the reaction mixture was extracted with ethyl acetate and saturated NaHCO₃ aqueous solution, and the organic layer was washed with brine, dried over anhydrous Na₂SO₄, filtered and concentrated under reduced pressure. Residue was purified by column chromatography (SiO₂; hexane/ethylacetate, 6/1) to yield the title compound (0.61 g, 35%).

### Step 2. Synthesis of ethyl 6-(3-methylen-4-oxo-1,2,3,4-tetrahydrocarbazol-9-yl)hexanoate [formula 2-7]

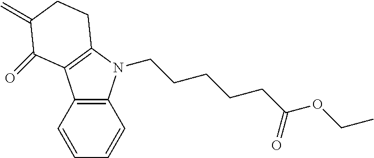 [SMILES: C=C1CCc2c(c3ccccc3n2CCCCCC(=O)OCC)C1=O]

Ethyl 6-(4-oxo-1,2,3,4-tetrahydrocarbazol-9-yl)hexanoate [formula 2-3] (0.61 g, 1.863 mmol), N,N-dimethylamine HCl (0.304 g, 3.726 mmol), paraformaldehyde (0.124 g, 3.73 mmol) and mixed solvent (acetic acid:toluene=4:1, 15 mL) were added, and stirred at 100° C. for 4 hours. After the completion of the reaction, acetic acid was distilled out under reduced pressure, and, without purification, the compound was dissolved in a 15 mL of mixed solvent (acetonitrile:H₂O=1:4) and stirred at 80° C. for 12 hours. After the completion of the reaction, the reaction mixture was extracted with ethyl acetate and saturated NaHCO₃ aqueous solution, dried over anhydrous Na₂SO₄, filtered and concentrated under reduced pressure. Residue was purified by column chromatography (SiO₂; hexane/ethylacetate, 7/1) to yield the title compound (0.35 g, 55%).

### Step 3. Synthesis of 6-(3-(morpholinomethyl)-4-oxo-1,2,3,4-tetrahydrocarbazol-9-yl)methyl) hexanoate [formula 2-8]

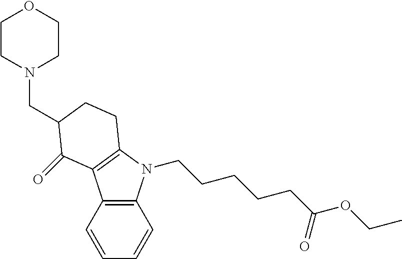 [SMILES: CCOC(=O)CCCCCn1c2c(c3ccccc31)C(=O)C(CN1CCOCC1)CC2]

To a microwave vial were added ethyl 6-(3-methylen-4-oxo-1,2,3,4-tetrahydrocarbazol-9-yl)hexanoate [formula 2-7] (0.1 g, 0.295 mmol), morpholine (0.077 g, 0.884 mmol) and toluene (4.0 mL), and a reaction was carried out in a microwave reactor at 110° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.059 g, 47%).

### Step 4. Synthesis of N-hydroxy-6-(3-(morpholinomethyl)-4-oxo-1,2,3,4-tetrahydrocarbazol-9-yl)hexanamide [formula 2-9]

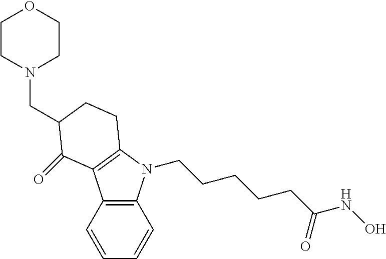 [SMILES: O=C(CCCCCn1c2c(c3ccccc31)C(=O)C(CN1CCOCC1)CC2)NO]

To a flask were added ethyl 6-(3-(morpholinomethyl)-4-oxo-1,2,3,4-tetrahydrocarbazol-9-yl)hexanoate [formula 2-8] (0.059 g, 0.138 mmol), hydroxylamine hydrochloride (NH₂OH HCl) (0.0481 g, 0.692 mmol), potassium hydroxide (0.0776 g, 1.38 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 193 as solid (0.031 g, 54%).

¹H NMR (400 MHz, DMSO-d₆) δ 10.3 (s, 1H), 9.02 (brs, 1H), 8.01 (d, 1H, J=7.1 Hz), 7.53 (d, 1H, J=7.8 Hz), 7.28-7.16 (m, 2H), 4.15 (s, 2H), 3.74-3.37 (m, 4H), 3.17 (s, 1H), 3.08-2.80 (m, 2H), 2.75-2.60 (m, 2H), 2.39-2.21 (m, 3H), 2.05-1.91 (m, 3H), 1.76-1.69 (m, 3H), 1.53-1.44 (m, 2H), 1.38-1.27 (m, 3H); MS (ESI) m/z 414 (M++H).

### Example 62

### Synthesis of Compound 194

### Step 1. Synthesis of ethyl 6-(3-((2-methyl-1H-imidazol-1-yl)methyl)-4-oxo-1,2,3,4-tetrahydrocarbazol-9-yl)hexanoate [formula 2-8]

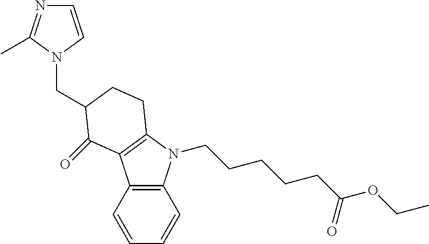 [SMILES: CCOC(=O)CCCCCn1c2c(c3ccccc31)C(=O)C(Cn1ccnc1C)CC2]

To a microwave vial were added ethyl 6-(3-methylen-4-oxo-1,2,3,4-tetrahydrocarbazol-9-yl)hexanoate [formula 2-7] (0.10 g, 0.295 mmol), 2-methyl-1H-imidazol (0.073 g, 0.884 mmol) and toluene (4.0 mL), and a reaction was carried out in a microwave reactor at 110° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.066 g, 53%).

### Step 2. Synthesis of N-hydroxy-6-(3-((2-methyl-1H-imidazol-1-yl)methyl)-4-oxo-1,2,3,4-tetrahydrocarbazol-9-yl)hexanamide [formula 2-9]

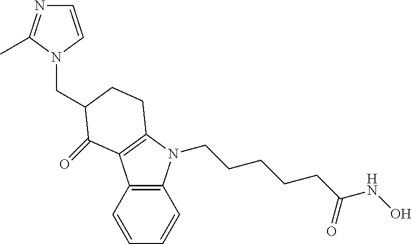 [SMILES: Cc1nccn1CC1CCc2c(c3ccccc3n2CCCCCC(=O)NO)C1=O]

Ethyl 6-(3-((2-methyl-1H-imidazol-1-yl)methyl)-4-oxo-1,2,3,4-tetrahydrocarbazol-9-yl)hexanoate [formula 2-8] (0.066 g, 0.157 mmol), hydroxylamine hydrochloride (NH₂OH HCl) (0.0544 g, 0.783 mmol), potassium hydroxide (0.0879 g, 1.566 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 194 as solid (0.028 g, 44%).

¹H NMR (400 MHz, DMSO-d₆) δ 8.04 (d, 1H, J=8.5 Hz), 7.56 (d, 1H, J=7.3 Hz), 7.26-7.19 (m, 2H), 7.05 (s, 1H), 6.73 (s, 1H), 4.47-4.42 (m, 1H), 4.18-4.14 (m, 2H), 4.10-4.06 (m, 1H), 3.11-3.07 (m, 1H), 2.99-2.89 (m, 2H), 2.30 (s, 3H), 2.03-2.00 (m, 1H), 1.99-1.91 (m, 2H), 1.89-7.80 (m, 1H), 1.79-1.66 (m, 2H), 1.53-1.52 (m, 2H), 1.28-1.21 (m, 2H); MS (ESI) m/z 409 (M++H).

### Example 63

### Synthesis of Compound 203

### Step 1. Synthesis of 3-(3-methylbut-2-enyl)-3H-pyrrolo[2,3-d]pyrimidin-4(7H)-on [formula 13-2]

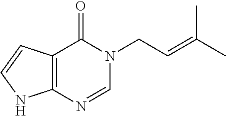

3H-pyrrolo[2,3-d]pyrimidin-4(7H)-on [formula 13-1] (0.81 g, 5.995 mmol) was dissolved in DMF (10 m L), and 55% NaH in paraffin solution (0.288 g, 11.989 mmol) was added and stirred at room temperature for 20 minutes. Then, 3,3-dimethyl allylbrimide (0.698 mL, 5.995 mmol) was added and stored for overnight. After the completion of the reaction, the reaction mixture was extracted with ethyl acetate and water, and purified by column chromatography (SiO₂; hexane/ethylacetate, 1/1) to obtain a compound. The obtained compound was crystallized with dichloromethane and n-hexane, filtered and to yield the title compound as which solid (0.366 g, 30%).

### Step 2. Synthesis of methyl 4-((3-3-methylbut-2-enyl)-4-oxo-3,4,5,6-tetrahydropyrrolo[2,3-d]pyrimidin-7-yl)methyl)benzoate [formula 13-3]

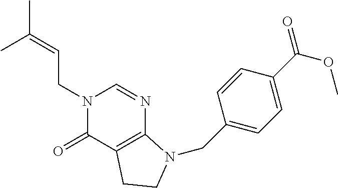 [SMILES: COC(=O)c1ccc(CN2CCc3c2ncn(CC=C(C)C)c3=O)cc1]

3-(3-methylbut-2-enyl)-3H-pyrrolo[2,3-d]pyrimidin-4(7H)-on [formula 13-2] (0.15 g, 0.738 mmol) was dissolved in DMF. Thereto, t-BuOK (0.099 g, 0.886 mmol) was added. Then, methyl 4-(bromomethyl)benzoate (0.186 g, 0.812 mmol) and a small amount of KI (0.012 g, 0.074 mmol) were added, and stirred at 50° C. for 24 hours. After the completion of the reaction, the reaction mixture was extracted with ethyl acetate and water, and purified by column chromatography (SiO₂; hexane/ethylacetate, 1/1) to obtain the title compound (0.1 g, 38.6%).

### Step 3. Synthesis of N-hydroxy-4-((3-(3-methylbut-2-enyl)-4-oxo-3,4,5,6-tetrahydropyrrolo[2,3-d]pyrimidin-7-yl)methyl)benzamide [formula 13-4]

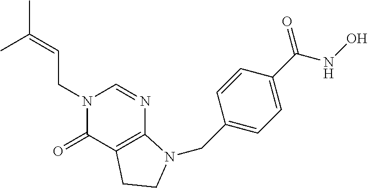 [SMILES: CC(C)=CCn1cnc2c(c1=O)CCN2Cc1ccc(C(=O)NO)cc1]

Methyl 4-((3-3-methylbut-2-enyl)-4-oxo-3,4,5,6-tetrahydropyrrolo[2,3-d]pyrimidin-7-yl)methyl)benzoate [formula 13-3] (0.1 g, 0.285 mmol) was dissolved in methanol (10 mL) and THF (10 mL). Then, NH₂OH (0.098 g, 1.423 mmol) and KOH (0.1597 g, 2.846 mmol) was added and stirred for 10 minutes. When the solution became cloud, hydroxylamine 50 wt % solution in water (0.79 mL, 5.692 mmol) was added drop-wise, and stirred for 24 hours. After confirming the completion of the reaction with TLC, methanol was removed under reduced pressure remaining water only, and a small volume of water (5 mL) was added, and 1N HCL aqueous solution added drop-wise. Then, precipitated white solid was dried to yield the compound of 203 (0.098 g, 97.7%).

¹H NMR (400 MHz, DMSO-d₆) δ 11.15 (s, 1H), 9.04 (s, 1H), 8.18 (s, 1H), 7.67 (d, 2H, J=8.2 Hz), 7.25-7.23 (m, 3H), 6.51 (d, 1H, J=3.3 Hz), 5.37 (s, 2H), 5.25 (t, 1H, J=6.5 Hz), 4.54 (d, 2H, J=6.9 Hz), 1.76 (s, 3H), 1.67 (s, 3H); MS (ESI) m/z 353.1 (M⁺+H).

### Example 64

### Synthesis of Compound 204

### Step 1. Synthesis of ethyl 6-(3-((3,3-difluoropyrrolidin-1-yl)methyl)-4-oxo-1,2,3,4-tetrahydrocarbazol-9-yl)hexanoate [formula 2-8]

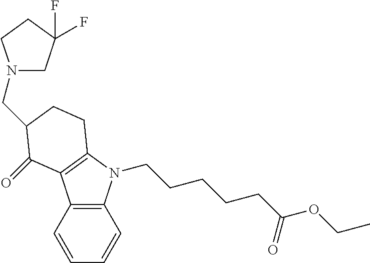 [SMILES: CCOC(=O)CCCCCn1c2c(c3ccccc31)C(=O)C(CN1CCC(F)(F)C1)CC2]

To a microwave vial were added ethyl 6-(3-methylen-4-oxo-1,2,3,4-tetrahydrocarbazol-9-yl)hexanoate [formula 2-7] (0.10 g, 0.295 mmol), 3,3-difluoropyrrolidine, HCl (0.127 g, 0.884 mmol) and toluene (4.0 mL), and a reaction was carried out in a microwave reactor at 110° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.071 g, 54%).

### Step 2. Synthesis of 6-(3-((3,3-difluoropyrrolidin-1-yl)methyl)-4-oxo-1,2,3,4-tetrahydrocarbazol-9-yl)-N-hydroxyhexanamide [formula 2-9]

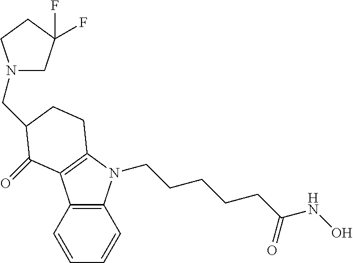 [SMILES: O=C(CCCCCn1c2c(c3ccccc31)C(=O)C(CN1CCC(F)(F)C1)CC2)NO]

Ethyl 6-(3-((3,3-difluoropyrrolidin-1-yl)methyl)-4-oxo-1,2,3,4-tetrahydrocarbazol-9-yl)hexanoate [formula 2-8] (0.071 g, 0.159 mmol), hydroxylamine hydrochloride (NH₂OH HCl) (0.0552 g, 0.795 mmol), potassium hydroxide (0.0892 g, 1.590 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 204 as solid (0.041 g, 60%).

¹H NMR (400 MHz, DMSO-d₆) δ 10.3 (s, 1H), 8.68 (s, 1H), 8.00 (d, 1H, J=8.3 Hz), 7.54 (d, 1H, J=7.9 Hz), 7.22-7.17 (m, 2H), 4.22-4.16 (m, 2H), 3.18-2.98 (m, 3H), 2.78-2.69 (m, 3H), 2.66-2.51 (m, 3H), 2.39-2.20 (m, 3H), 2.01-1.90 (m, 3H), 1.69-1.62 (m, 2H), 1.59-1.51 (m, 2H), 1.27-1.21 (2H); MS (ESI) m/z 434 (M++H).

### Example 65

### Synthesis of Compound 205

### Step 1. Synthesis of ethyl 6-(3-((4-methylpiperazin-1-yl)methyl)-4-oxo-1,2,3,4-tetrahydrocarbazol-9-yl)hexanoate [formula 2-8]

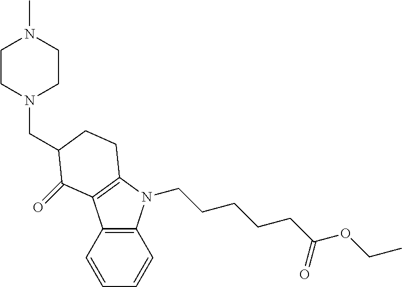 [SMILES: CCOC(=O)CCCCCn1c2c(c3ccccc31)C(=O)C(CN1CCN(C)CC1)CC2]

To a microwave vial were added ethyl 6-(3-methylen-4-oxo-1,2,3,4-tetrahydrocarbazol-9-yl)hexanoate [formula 2-7] (0.10 g, 0.295 mmol), 1-methylpiperazine (0.0885 g, 0.884 mmol) and toluene (4.0 mL), and a reaction was carried out at 110° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.067 g, 52%).

### Step 2. Synthesis of N-hydroxy-6-(3-((4-methylpiperazin-1-yl)methyl)-4-oxo-1,2,3,4-tetrahydrocarbazol-9-yl)hexanamide [formula 2-9]

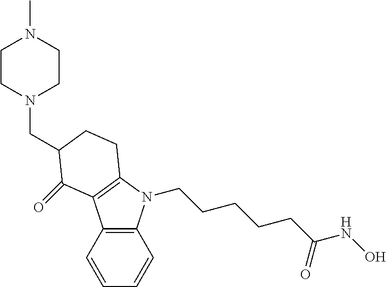 [SMILES: CN1CCN(CC2CCc3c(c4ccccc4n3CCCCCC(=O)NO)C2=O)CC1]

Ethyl 6-(3-((4-methylpiperazin-1-yl)methyl)-4-oxo-1,2,3,4-tetrahydrocarbazol-9-yl)hexanoate [formula 2-8] (0.067 g, 0.152 mmol), hydroxylamine hydrochloride (NH₂OH HCl) (0.0530 g, 0.762 mmol), potassium hydroxide (0.0855 g, 1.524 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 205 as solid (0.045 g, 69%).

¹H NMR (400 MHz, DMSO-d₆) δ 10.3 (s, 1H), 8.68 (s, 1H), 8.00 (d, 1H, J=6.8 Hz), 7.53 (d, 1H, J=7.8 Hz), 7.23-7.15 (m, 2H), 4.16 (t, 2H, J=7.4 Hz), 3.39-3.28 (m, 2H), 3.17-2.96 (m, 2H), 2.67-2.62 (m, 2H), 2.49-2.46 (m, 2H), 2.43-2.29 (m, 6H), 2.15 (s, 3H), 1.96-1.71 (m, 3H), 1.70-1.68 (m, 2H), 1.53-1.49 (m, 2H), 1.31-1.29 (m, 2H); MS (ESI) m/z 427 (M++H).

### Example 66

### Synthesis of Compound 206

### Step 1. Synthesis of ethyl 6-(3-((4-ethylpiperazin-1-yl)methyl)-4-oxo-1,2,3,4-tetrahydrocarbazol-9-yl)hexanoate [formula 2-8]

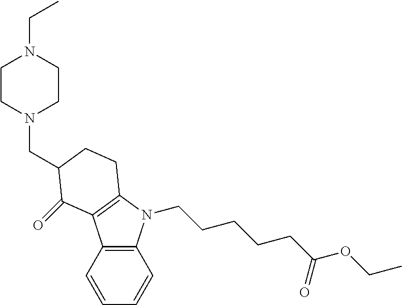 [SMILES: CCOC(=O)CCCCCn1c2c(c3ccccc31)C(=O)C(CN1CCN(CC)CC1)CC2]

To a microwave vial were added ethyl 6-(3-methylen-4-oxo-1,2,3,4-tetrahydrocarbazol-9-yl)hexanoate [formula 2-7] (0.10 g, 0.295 mmol), 1-ethylpiperazine (0.0885 g, 0.884 mmol) and toluene (4.0 mL), and a reaction was carried out in a microwave reactor at 110° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.063 g, 47%).

### Step 2. Synthesis of 6-(3-((4-ethylpiperazin-1-yl)methyl)-4-oxo-1,2,3,4-tetrahydrocarbazol-9-yl)-N-hydroxyhexanamide [formula 2-10]

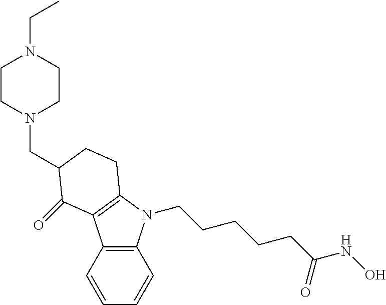 [SMILES: CCN1CCN(CC2CCc3c(c4ccccc4n3CCCCCC(=O)NO)C2=O)CC1]

Ethyl 6-(3-((4-ethylpiperazin-1-yl)methyl)-4-oxo-1,2,3,4-tetrahydrocarbazol-9-yl)hexanoate [formula 2-8] (0.063 g, 0.139 mmol), hydroxylamine hydrochloride (NH₂OH HCl) (0.0483 g, 0.694 mmol), potassium hydroxide (0.0779 g, 1.389 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 206 as solid (0.042 g, 68%).

¹H NMR (400 MHz, DMSO-d₆) δ 10.3 (s, 1H), 8.68 (s, 1H), 8.00 (d, 1H, J=7.0 Hz), 7.53 (d, 1H, J=8.0 Hz), 7.21-7.17 (m, 2H), 4.16 (t, 2H, J=7.2 Hz), 3.34-3.20 (m, 3H), 3.08-2.90 (m, 2H), 2.67-2.60 (m, 2H), 2.58-2.37 (m, 2H), 2.40-2.28 (m, 7H), 1.94-1.90 (m, 3H), 1.78-1.69 (m, 2H), 1.59-1.49 (m, 2H), 1.38-1.21 (m, 2H), 0.98 (t, 3H, J=7.1 Hz); MS (ESI) m/z 441 (M++H).

### Example 67

### Synthesis of Compound 207

### Step 1. Synthesis of methyl 4-((6-fluoro-2,2-dimethyl-3-(morpholinomethyl)-4-oxo-1,2,3,4-tetrahydrocarbazol-9-yl)methyl)benzoate [formula 2-5]

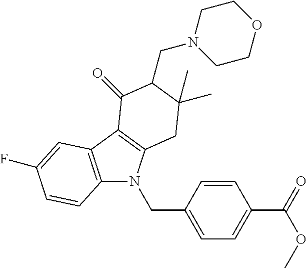 [SMILES: COC(=O)c1ccc(Cn2c3c(c4cc(F)ccc42)C(=O)C(CN2CCOCC2)C(C)(C)C3)cc1]

To a microwave vial were added methyl 4-((6-fluoro-2,2-dimethyl-3-methylen-4-oxo-1,2,3,4-tetrahydrocarbazol-9-yl)methyl)benzoate [formula 2-4] (0.10 g, 0.255 mmol), morpholine (0.0668 g, 0.766 mmol) and toluene (4.0 mL), and a reaction was carried out in a microwave reactor at 110° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.058 g, 47%).

### Step 2. Synthesis of 4-((6-fluoro-2,2-dimethyl-3-(morpholinomethyl)-4-oxo-1,2,3,4-tetrahydrocarbazol-9-yl)methyl)-N-hydroxybenzamide [formula 2-6]

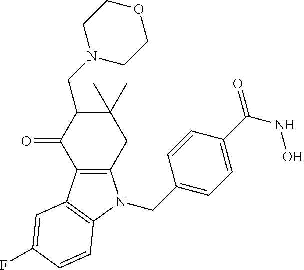 [SMILES: CC1(C)Cc2c(c3cc(F)ccc3n2Cc2ccc(C(=O)NO)cc2)C(=O)C1CN1CCOCC1]

Methyl 4-((6-fluoro-2,2-dimethyl-3-(morpholinomethyl)-4-oxo-1,2,3,4-tetrahydrocarbazol-9-yl)methyl)benzoate [formula 2-5] (0.058 g, 0.121 mmol), hydroxylamine hydrochloride (NH₂OH HCl) (0.0421 g, 0.606 mmol), potassium hydroxide (0.0680 g, 1.212 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 207 as solid (0.032 g, 55%).

¹H NMR (400 MHz, DMSO-d₆) δ 7.69-7.65 (m, 3H), 7.52-7.48 (m, 1H), 7.10 (d, 2H, J=8.3 Hz), 7.06-7.01 (m,H), 5.54 (s, 2H), 3.51-3.48 (brs, 4H), 3.01-2.96 (m, 1H), 2.89-2.84 (m, 1H), 2.71-2.70 (m, 1H), 2.49-2.43 (m, 3H), 2.38-2.33 (m, 3H), 1.12 (s, 3H), 1.01 (s, 3H); MS (ESI) m/z 480 (M++H).

### Example 68

### Synthesis of Compound 208

### Step 1. Synthesis of ethyl 6-(3-((4-butylpiperazin-1-yl)methyl)-4-oxo-1,2,3,4-tetrahydrocarbazol-9-yl)hexanoate [formula 2-8]

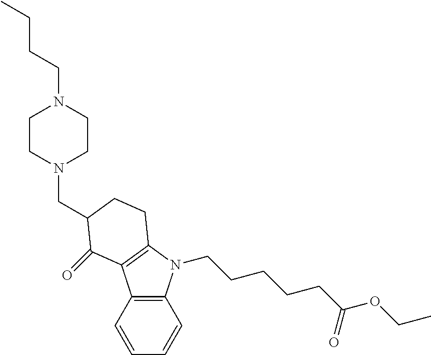 [SMILES: CCCCN1CCN(CC2CCc3c(c4ccccc4n3CCCCCC(=O)OCC)C2=O)CC1]

To a microwave vial were added ethyl 6-(3-methylen-4-oxo-1,2,3,4-tetrahydrocarbazol-9-yl)hexanoate [formula 2-7] (0.10 g, 0.295 mmol), 1-butylpiperazine (0.1257 g, 0.884 mmol) and toluene (4.0 mL), and a reaction was carried out in a microwave reactor at 110° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.059 g, 42%).

### Step 2. Synthesis of 6-(3-((4-butylpiperazin-1-yl)methyl)-4-oxo-1,2,3,4-tetrahydrocarbazol-9-yl)-N-hydroxyhexanamide [formula 2-9]

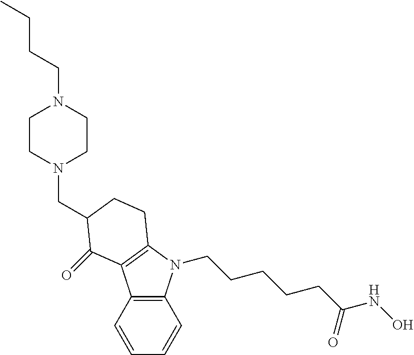 [SMILES: CCCCN1CCN(CC2CCc3c(c4ccccc4n3CCCCCC(=O)NO)C2=O)CC1]

Ethyl 6-(3-((4-butylpiperazin-1-yl)methyl)-4-oxo-1,2,3,4-tetrahydrocarbazol-9-yl)hexanoate [formula 2-8] (0.059 g, 0.122 mmol), hydroxylamine hydrochloride (NH₂OH HCl) (0.0426 g, 0.612 mmol), potassium hydroxide (0.0687 g, 1.225 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 208 as solid (0.031 g, 54%).

¹H NMR (400 MHz, DMSO-d₆) δ 10.3 (s, 1H), 8.73 (s, 1H), 8.00 (d, 1H, J=5.5 Hz), 7.53 (d, 1H, J=5.7 Hz), 7.23-7.15 (m, 2H), 4.19-4.15 (m, 2H), 3.08-2.94 (m, 3H), 2.65-2.63 (m, 3H), 2.30-2.10 (m, 6H), 2.05-1.91 (m, 3H), 1.76-1.69 (m, 3H), 1.60-1.51 (m, 2H), 1.43-4.38 (m, 2H), 1.36-1.19 (m, 6H), 0.89-0.85 (m, 4H); MS (ESI) m/z 469 (M++H).

### Example 69

### Synthesis of Compound 209

### Step 1. Synthesis of methyl 4-((6-fluoro-2,2-dimethyl-3-((4-methylpiperazin-1-yl)methyl)-4-oxo-1,2,3,4-tetrahydrocarbazol-9-yl)methyl)benzoate [formula 2-5]

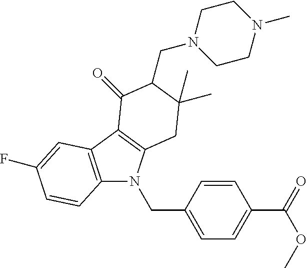 [SMILES: COC(=O)c1ccc(Cn2c3c(c4cc(F)ccc42)C(=O)C(CN2CCN(C)CC2)C(C)(C)C3)cc1]

To a microwave vial were added methyl 4-((6-fluoro-2,2-dimethyl-3-methylen-4-oxo-1,2,3,4-tetrahydrocarbazol-9-yl)methyl)benzoate [formula 2-4] (0.10 g, 0.255 mmol), 1-methylpiperazine (0.0768 g, 0.766 mmol) and toluene (4.0 mL), and a reaction was carried out in a microwave reactor at 110° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.071 g, 57%).

### Step 2. Synthesis of 4-((6-fluoro-2,2-dimethyl-3-((4-methylpiperazin-1-yl)methyl)-4-oxo-1,2,3,4-tetrahydrocarbazol-9-yl)methyl)-N-hydroxybenzamide [formula 2-6]

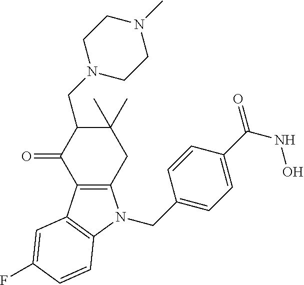 [SMILES: CN1CCN(CC2C(=O)c3c(n(Cc4ccc(C(=O)NO)cc4)c4ccc(F)cc34)CC2(C)C)CC1]

Methyl 4-((6-fluoro-2,2-dimethyl-3-((4-methylpiperazin-1-yl)methyl)-4-oxo-1,2,3,4-tetrahydrocarbazol-9-yl)methyl)benzoate [formula 2-5] (0.071 g, 0.144 mmol), hydroxylamine hydrochloride (NH₂OH HCl) (0.0502 g, 0.722 mmol), potassium hydroxide (0.0810 g, 1.444 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 209 as solid (0.049 g, 69%).

¹H NMR (400 MHz, DMSO-d₆) δ 11.2 (brs, 1H), 9.03 (brs, 1H), 7.70-7.65 (m, 3H), 7.51-7.48 (m, 1H), 7.12 (d, 2H, J=6.2 Hz), 7.06-7.00 (m, 1H), 5.54 (s, 2H), 3.01-2.97 (m, 1H), 2.88-2.84 (m, 1H), 2.72-2.67 (m, 1H), 2.54-2.50 (m, 2H), 2.49-2.39 (m, 5H), 2.37-2.19 (m, 3H), 2.10 (s, 3H), 1.16 (s, 3H), 1.01 (s, 3H); MS (ESI) m/z 493 (M++H).

### Example 70

### Synthesis of Compound 220

### Step 1. Synthesis of 2,3-dimethyl-6,7-dihydro-1H-indole-4(5H)-on [formula 6-2]

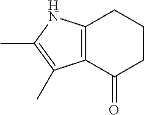

(E)-3-(hydroxyimino)butan-2-on [formula 6-1] (2.0 g, 19.782 mmol), 1,3-cyclohexadione (2.218 g, 19.782 mmol) and mixed solvent (acetic acid:H₂O=7:3)(30.0 mL) were added, and zinc (2.58 g, 39.56 mmol) was slowly added at 0° C., then stirred with heating for 12 hours. After the completion of the reaction, acetic acid was distilled out under reduced pressure. The reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (1.8 g, 56%).

### Step 2. Synthesis of methyl 4-((2,3-dimethyl-4-oxo-4,5,6,7-tetrahydroindol-1-yl)methyl)benzoate [Formula 6-3-]

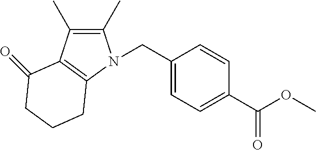 [SMILES: COC(=O)c1ccc(Cn2c(C)c(C)c3c2CCCC3=O)cc1]

2,3-dimethyl-6,7-dihydro-1H-indole-4(5H)-on [formula 6-2] (1.8 g, 11.03 mmol) was dissolved in DMF (10 mL). Thereto, 55% NaH in paraffin solution (0.9624 g, 22.056 mmol) was added and stirred for 10 minutes. Then, methyl-4(bromomethyl)benzoate (3.0316 g, 13.234 mmol) was added and stirred at 50° C. for 5 hours. After the completion of the reaction, DMF was distilled out and the reaction mixture was extracted with ethyl acetate and saturated NaHCO₃, the organic layer was washed with brine, dried over anhydrous Na₂SO₄, filtered and concentrated under reduced pressure. Residue was purified by column chromatography (SiO₂; hexane/ethylacetate, 5/1) to yield the title compound (1.76 g, 51%).

### Step 3. Synthesis of 4-((2,3-dimethyl-4-oxo-4,5,6,7-tetrahydroindol-1-yl)methyl)-N-hydroxybenzamide [formula 6-4]

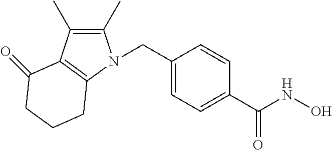 [SMILES: Cc1c2c(n(Cc3ccc(C(=O)NO)cc3)c1C)CCCC2=O]

To a flask were added methyl 4-((2,3-dimethyl-4-oxo-4,5,6,7-tetrahydroindol-1-yl)methyl)benzoate [formula 6-3] (0.150 g, 0.482 mmol), hydroxylamine hydrochloride (NH₂OH HCl) (0.067 g, 0.963 mmol), potassium hydroxide (0.108 g, 1.927 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure. The reaction mixture was extracted with ethyl acetate only, dried over anhydrous Na₂SO₄, filtered and concentrated under reduced pressure. And the reaction mixture was re-crystallized by diethyl ether and ethyl acetate, filtered to yield the compound 220 (0.060 g, 40%).

¹H NMR (400 MHz, DMSO-d₆) δ 9.04 (brs, 1H), 7.70 (d, 2H, J=6.2 Hz), 7.02 (d, 2H, J=6.2 Hz), 5.16 (s, 2H), 2.64 (t, 2H, J=4.5 Hz), 2.27 (d, 2H, J=4.7 Hz), 2.12 (s, 3H), 1.98-1.96 (m, 2H), 1.95 (s, 3H); MS (ESI) m/z 313 (M++H).

### Example 71

### Synthesis of Compound 228

### Synthesis of N-hydroxy-4-((1-oxo-1,2,3,4-tetrahydropyrido[4,3-b]indol-5-yl)methyl)benzamide [formula 10-3]

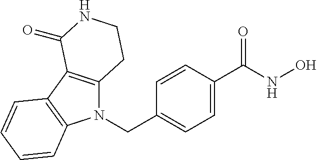 [SMILES: O=C(NO)c1ccc(Cn2c3c(c4ccccc42)C(=O)NCC3)cc1]

Methyl 4-((1-oxo-1,2,3,4-tetrahydropyrido[4,3-b]indol-5-yl)methyl)benzoate (0.06 g, 0.18 mmol) was dissolved in methanol (5 mL) and THF (1 mL). Then, hydroxylamine 50% aqueous solution (2.2 mL), hydroxylamine hydrochloride (NH₂OH HCl) (0.062 g, 0.90 mmol) and potassium hydroxide (0.20 g, 3.59 mmol) were added in order, and stirred at room temperature for 16 hours. After the completion of the reaction, the reaction mixture was concentrated under reduced pressure until the remained solution to be about 2-3 mL, and saturated NaHCO₃ (1-2 mL) was added and stirred. After filtering, the obtained compound was washed with water, dried with vacuum to yield compound 228 as bright brown solid (0.012 g, 20%).

¹H NMR (400 MHz, DMSO-d₆) δ 7.93 (m, 1H), 7.67 (d, 2H, J=8.3 Hz), 7.46 (m, 1H), 7.13 (m, 5H), 5.52 (s, 2H), 3.47 (m, 2H), 2.98 (m, 2H).

### Example 72

### Synthesis of Compound 232

### Step 1. Synthesis of methyl 4-((3-methyl-4-oxo-4,5,6,7-tetrahydropyrrolo[3,2-c]pyridin-1-yl)methyl)benzoate [formula 8-6]

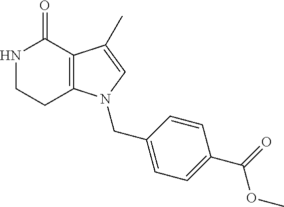 [SMILES: COC(=O)c1ccc(Cn2cc(C)c3c2CCNC3=O)cc1]

Compound of formula 8-2 (3-methyl-6,7-dihydro-1H-pyrrolo[3,2-c]pyridin-4(5H)-one, 0.57 g, 3.80 mmol) was dissolved in acetonitrille (20 mL). Thereto, methyl-4-(bromomethyl)benzoate (1.04 g, 4.56 mmol) and cesium carbonate (1.36 g, 4.18 mmol) were added. After increasing temperature slowly and refluxing with stirring for 3 hours, the reaction was completed. The reaction mixture was washed with brine three times, the organic layer was dried over Na₂SO₄ and filtered, and the filtrate was concentrated under reduced pressure. The concentration was purified by column chromatography (4 g ISCO silica gel cartridge, 0-10% methanol/dichloromethane) to yield the title compound as white solid (0.8 g, 71%).

### Step 2. Synthesis of N-hydroxy-4-((3-methyl-4-oxo-4,5,6,7-tetrahydropyrrolo[3,2-c]pyridin-1-yl)methyl)benzamide [formula 8-7]

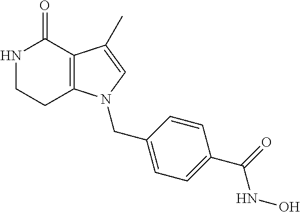 [SMILES: Cc1cn(Cc2ccc(C(=O)NO)cc2)c2c1C(=O)NCC2]

Compound of formula 8-6 (0.080 g, 0.27 mmol) was dissolved in methanol (3 mL), and hydroxylamine hydrochloride (0.093 g, 1.34 mmol) was added slowly. Then, potassium hydroxide (0.15 g, 2.68 mmol) was added and stirred at room temperature for 10 minutes, and hydroxylamine 50% aqueous solution was added and refluxed with stirring for 3 hours. The organic solvent was concentrated under reduced pressure, neutralized by adding 2N HCl, washed with brine for three times. The organic layer was dried over Na₂SO₄ and filtered, and the filtrate was concentrated under reduced pressure to yield the compound 232 as white solid (0.043 g, 54%).

¹H NMR (400 MHz, DMSO-d₆) δ 11.19 (brs, 1H), 9.04 (brs, 1H), 7.70 (d, 2H, J=8.0 Hz), 7.15 (d, 2H, J=7.9 Hz), 6.82 (brs, 1H), 6.54 (s, 1H), 5.07 (s, 2H), 3.31 (t, 2H, J=11.1 Hz), 2.61 (t, 2H, J=6.6 Hz), 2.11 (s, 3H); MS (ESI) m/z 300 (M⁺+H).

### Example 73

### Synthesis of Compound 235

### Step 1. ethyl 6-(2,3-dimethyl-4-oxo-4,5,6,7-tetrahydroindol-1-yl)hexaoate [formula 6-5]

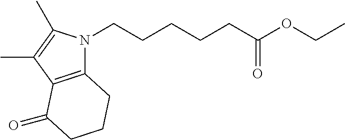 [SMILES: CCOC(=O)CCCCCn1c(C)c(C)c2c1CCCC2=O]

2,3-dimethyl-6,7-dihydro-1H-indol-4(5H)-on [formula 6-2] (0.20 g, 1.225 mmol) was dissolved in DMF (10 mL). Thereto, 55% NaH in paraffin solution (0.107 g, 2.45 mmol) was added and stirred for 10 minutes. Then, 6-bromomethylhexanoate (0.328 g, 1.47 mmol) was added and stirred at room temperature for 5 hours. After the completion of the reaction, DMF was distilled out, and the reaction mixture was extracted with ethyl acetate and saturated NaHCO₃, the organic layer was washed with brine, dried over anhydrous Na₂SO₄, filtered and concentrated under reduced pressure. Residue was purified by column chromatography (SiO₂; hexane/ethylacetate, 5/1) to yield the title compound as solid (0.21 g, 56%).

### Step 2. Synthesis of 6-(2,3-dimethyl-4-oxo-4,5,6,7-tetrahydroindol-1-yl)-N-hydroxyhexanamide [formula 6-6]

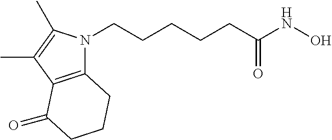 [SMILES: Cc1c2c(n(CCCCCC(=O)NO)c1C)CCCC2=O]

To a flask were added ethyl 6-(2,3-dimethyl-4-oxo-4,5,6,7-tetrahydroindol-1-yl)hexanoate [formula 6-5] (0.21 g, 0.688 mmol), hydroxylamine hydrochloride (NH₂OH HCl) (0.0956 g, 1.375 mmol), potassium hydroxide (0.154 g, 2.75 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. The reaction mixture was re-crystallized by diethyl ether and ethyl acetate, filtered to yield the compound 235 (0.095 g, 47%).

¹H NMR (400 MHz, DMSO-d₆) δ 10.3 (brs, 1H), 8.69 (brs, 1H), 3.75 (t, 2H, J=5.7 Hz), 2.70 (t, 2H, J=4.5 Hz), 2.24 (t, 2H, J=4.7 Hz), 2.08 (s, 3H), 2.06 (s, 3H), 1.98-1.92 (m, 4H), 1.54-1.49 (m, 4H), 1.25-1.23 (m, 2H); MS (ESI) m/z 293 (M++H).

### Example 74

### Synthesis of Compound 236—Scheme 6

### Step 1. Synthesis of 2,3,6,6-tetramethyl-6,7-dihydro-1H-indole-4(5H)-on [formula 6-2]

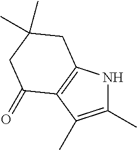

(E)-3-(hydroxyimino)butan-2-on [formula 6-1] (3.0 g, 29.7 mmol) and 5,5-dimethyl-1,3-cyclohexandion (4.16 g, 29.7 mmol) were dissolved in acetic acid (35 mL) and H₂O (15 mL). Thereto, zinc powder (3.88 g, 59.3 mmol) was added slowly maintaining room temperature. The reaction mixture was refluxed with stirring for one day, concentrated under reduced pressure and extracted with CH₂Cl₂ and brine, of which pH was adjusted to about 6 using saturated NaHCO₃. The reaction mixture was extracted with CH₂Cl₂, the organic layer was dried over anhydrous MgSO₄ and filtered. Filtrate was concentrated under reduced pressure and purified by column chromatography (SiO₂; hexane/ethylacetate, 7/3) to yield the title compound as yellow solid (2.59 g, 46%).

### Step 2. Synthesis of methyl 4-((2,3,6,6-tetramethyl-4-oxo-4,5,6,7-tetrahydro-1H-indol-1-yl)methyl)benzbenzoate [formula 6-3]

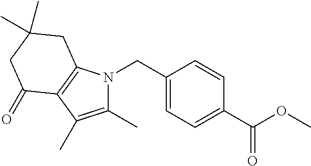

Methyl 4-((2,3,6,6-tetramethyl-6,7-dihydro-1H-indole-4(5H)-on [formula 6-2] (0.25 g, 1.31 mmol) was dissolved DMF. Thereto, NaH (0.035 g, 1.37 mmol) was added slowly at room temperature. After 5 minute of stirring, methyl 4-(bromethyl)benzoate was added and stirred at room temperature for 4 hours. After the completion of the reaction, the reaction mixture was extracted with ethyl acetate and saturated NH₄Cl aqueous solution, the organic layer was dried over MgSO₄ and filtered. Filtrate was concentrated under reduced pressure and purified by column chromatography (SiO₂; hexane/ethylacetate, 6/4) to yield the title compound as white solid (0.28 g, 62%).

### Step 3. Synthesis of N-hydroxy-4-((2,3,6,6-tetramethyl-4-oxo-4,5,6,7-tetrahydro-1H-indol-1-yl)methyl)benzamide [formula 6-4]

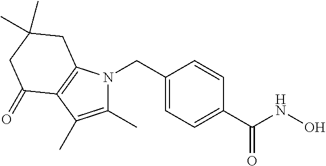 [SMILES: Cc1c2c(n(Cc3ccc(C(=O)NO)cc3)c1C)CC(C)(C)CC2=O]

Methyl 4-((2,3,6,6-tetramethyl-4-oxo-4,5,6,7-tetrahydro-1H-indol-1-yl)methyl)benzoate [formula 6-3] (0.28 g, 0.81 mmol) was dissolved in methanol (5 mL). Thereto, hydroxylamine hydrochloride (NH₂OH HCl) (0.11 g, 1.63 mmol) and potassium hydroxide (0.18 g, 3.25 mmol) were added and stirred. Hydroxylamine 50% aqueous solution (1.5 mL) was added slowly until the reaction solution became clear and stirred at room temperature for 2 hours. After the completion of the reaction, methanol was distilled out under reduced pressure, and the reaction mixture was extracted with methyl acetate and water. The organic layer was dried over MgSO₄ and filtered. Filtrate was concentrated under reduced pressure and concentrated to obtain solid product, which was filtered, washed with ethylacetate and dried under reduced pressure to yield the compound 236 as white solid (0.12 g, 42%).

¹H NMR (400 MHz, DMSO-d₆) δ 11.2 (s, 1H), 9.05 (s, 1H), 7.70 (d, 2H, J=8.3 Hz), 6.98 (d, 2H, J=8.2 Hz), 5.15 (s, 2H), 2.54 (s, 2H), 2.18 (s, 2H), 2.12 (s, 3H), 1.95 (s, 3H), 0.98 (s, 6H); MS (ESI) m/z 341 (M⁺+H).

### Example 75

### Synthesis of Compound 237

### Step 1. Synthesis of (Z)—N′-(3,3-dimethyl-5-oxocyclohexyliden)-4-methylbenzenesulfonohydrazide) [formula 3-3]

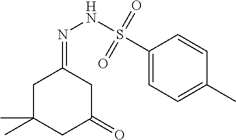

p-toluenesulfonylhydrazide [formula 3-1] (5.0 g, 26.85 mmol), 5-5-dimethyl-1,3-cyclohexandion [formula 3-2] (3.76 g, 26.85 mmol) and p-toluenesulfonic acid monohydrate (0.51 g, 2.68 mmol) were added on toluene (300 mL), refluxed with stirring for 30 minutes and cooled to room temperature. Toluene (50 mL) was added more, refluxed with stirring for 1 hour and cooled to room temperature. Yellow precipitate was filtered and dried to yield the title compound as light yellow solid (7.2 g, 86.9%).

### Step 2. Synthesis of 6,6-dimethyl-3-(trifluoromethyl)-6,7-dihydro-1H-indazol-4(5H)-on [formula 3-4]

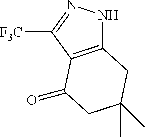 [SMILES: CC1(C)CC(=O)c2c(C(F)(F)F)n[nH]c2C1]

(Z)—N′-(3,3-dimethyl-5-oxocyclohexyliden)-4-methylbenzenesulfonohydrazide) [formula 3-3] (4.0 g, 12.97 mmol) and trifluoroacetic acid anhydride were added to THF (72 mL) and triethylamine (24 mL), and a reaction was carried out at 55° C. for 2 hours and cooled to room temperature. Methanol (16 mL) and a 1:1 solution of water/1M aqueous sodium hydroxide were added. After stirring for 3 h, the reaction mixture was diluted with saturated NH₄Cl aqueous solution (50 mL) and extracted with ethyl acetate. The organic layer was dried over MgSO₄, filtered and concentrated under reduced pressure. Residue was purified by column chromatography (SiO₂; hexane/ethylacetate, 1/1) to yield the title compound as light yellow solid (0.35 g, 11.6%).

### Step 3. Synthesis of methyl 4-((6,6-dimethyl-4-oxo-3-(trifluoromethyl)-4,5,6,7-tetrahydro-1H-indazol-1-yl)methyl)benzoate [formula 3-5]

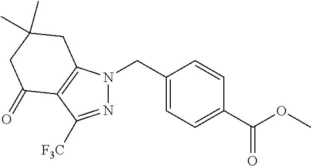

6,6-dimethyl-3-(trifluoromethyl)-6,7-dihydro-1H-indazol-4(5H)-on [formula 3-4] (0.71 g, 3.05 mmol) was dissolved in DMA. Thereto, NaH (0.081 g, 3.20 mmol) was added slowly at room temperature. After 5 minutes stirring, methyl 4-(bromomethyl)benzoate (0.75 g, 3.20 mmol) was added and stirred at room temperature for 4 hours. After the completion of the reaction, the reaction mixture was extracted with ethyl acetate and saturated NH₄Cl aqueous solution, the organic layer was dried over anhydrous MgSO₄ and filtered. Filtrate was concentrated under reduced pressure and purified by column chromatography (SiO₂; hexane/ethylacetate, 6/4) to yield the title compound as yellow solid (0.79 g, 68%).

### Step 4. Synthesis of 4-((6,6-dimethyl-4-oxo-3-(trifluoromethyl)-4,5,6,7-tetrahydro-1H-indazol-1-yl)methyl)benxoic acid [formula 3-6]

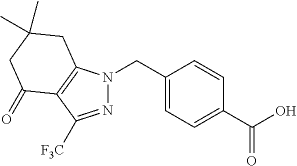 [SMILES: CC1(C)CC(=O)c2c(C(F)(F)F)nn(Cc3ccc(C(=O)O)cc3)c2C1]

Methyl 4-((6,6-dimethyl-4-oxo-3-(trifluoromethyl)-4,5,6,7-tetrahydro-1H-indazol-1-yl)methyl)benzoate [formula 3-5] (0.79 g, 2.08 mmol) was dissolved in methanol (10 mL) and H₂O (5 mL) and LiOH (0.435 g, 10.39 mmol), refluxed with stirring for 1 hour, cooled to room temperature and concentrated under reduced pressure. pH of the reaction mixture was adjusted to 1-2 using 1M HCl to obtain white precipitate. The solid product was filtered, dried under reduced pressure to yield the title compound as light yellow solid (0.73 g, 96%).

### Step 5. Synthesis of 4-((6,6-dimethyl-4-oxo-3-(trifluoromethyl)-4,5,6,7-tetrahydro-1H-indazol-1-yl)methyl)-N-((tetrahydro-2H-pyran-2-yl)oxy)benzamide [formula 3-7]

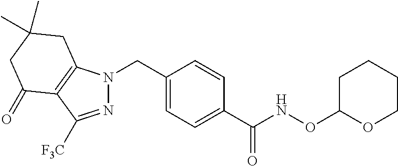 [SMILES: CC1(C)CC(=O)c2c(C(F)(F)F)nn(Cc3ccc(C(=O)NOC4CCCCO4)cc3)c2C1]

4-((6,6-dimethyl-4-oxo-3-(trifluoromethyl)-4,5,6,7-tetrahydro-1H-indazol-1-yl)methyl)benxoic acid [formula 3-6] (0.73 g, 1.99 mmol) and O-(tetrahydro-2H-pyran-2-yl)hydroxylamine (0.28 g, 2.39 mmol) was dissolved in CH₂Cl₂. Thereto, EDC (0.57 g, 2.98 mmol), HOBt (0.40 g, 2.98 mmol) and DIPEA (0.51 g, 3.98 mmol) were added. The reaction was carried out at room temperature for a day and diluted with saturated NaHCO₃. After the completion of the reaction, the reaction mixture was extracted with dichloromethane, the organic layer was dried over anhydrous MgSO₄ and filtered. Filtrate was concentrated under reduced pressure and purified by column chromatography (SiO₂; hexane/ethylacetate, 4/6) to yield the title compound as white solid (0.79 g, 85%).

### Step 6. Synthesis of 4-((6,6-dimethyl-4-oxo-3-(trifluoromethyl)-4,5,6,7-tetrahydro-1H-indazol-1-yl)methyl)-N-hydroxybenzamide [formula 3-8]

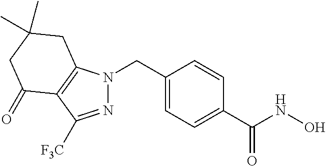 [SMILES: CC1(C)CC(=O)c2c(C(F)(F)F)nn(Cc3ccc(C(=O)NO)cc3)c2C1]

4-((6,6-dimethyl-4-oxo-3-(trifluoromethyl)-4,5,6,7-tetrahydro-1H-indazol-1-yl)methyl)-N-((tetrahydro-2H-pyran-2-yl)oxy)benzamide [formula 3-7] (2.80 g, 6.02 mmol) was dissolved in methanol (10 mL). Thereto, methanolic HCl (14.4 mL, 18.0 mmol) was added, a reaction carried out at room temperature for 1 hour. After the completion of the reaction, the reaction mixture was concentrated under reduced pressure and diluted with brine, extracted with EtOAC, dried over MgSO₄ filtered and concentrated. The residue was purified by column chromatography (SiO₂; dichloromethane/methanol, 9/1) to yield the compound 237 as white solid (1.16 g, 50.6%).

¹H NMR (400 MHz, DMSO-d₆) δ 11.2 (s, 1H), 9.06 (s, 1H), 7.75 (d, 2H, J=8.2 Hz), 7.27 (d, 2H, J=8.2 Hz), 5.51 (s, 2H), 2.86 (s, 2H), 2.39 (s, 2H), 1.03 (s, 6H); MS (ESI) m/z 382 (M⁺+H).

### Example 76

### Synthesis of Compound 249

### Step 1. Synthesis of 3,4-dihydro-2H-pyrido[4,3-b]indol-1(5H)-on [formula 9-2]

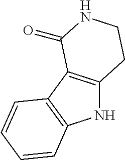 [SMILES: O=C1NCCc2[nH]c3ccccc3c21]

Tert-butyl 2,4-dioxopiperidine-1-carboxylate [formula 9-1] (15.0 g, 70.35 mmol) was dissolved in TFA (30 mL) and stirred at room temperature for 30 minutes. Then, phenyl hydrazine (6.92 mL, 70.35 mmol) and H₂SO₄ (3 mL) were added and stirred at 100° C. for 16 hours. After the completion of the reaction, the reaction mixture was diluted with ethyl acetate (100 mL), stirred and filtered. The filtrate was concentrated under reduced pressure, and residue was extracted with ethyl acetate and saturated NaHCO₃ aqueous solution, the organic layer was dried over anhydrous MgSO₄, filtered, and concentrated under reduced pressure. Residue was purified by column chromatography (SiO₂; THF/dichloromethane, 3/1) to yield the title compound as brown solid (5.24 g, 40%).

### Step 2. Synthesis of methyl-4-((1-oxo-1,2,3,4-tetrahydropyrido[4,3-b]indol-5-yl)methyl)benzoate [formula 9-3]

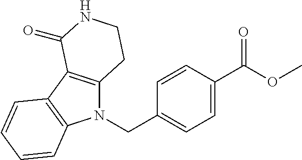 [SMILES: COC(=O)c1ccc(Cn2c3c(c4ccccc42)C(=O)NCC3)cc1]

3,4-dihydro-2H-pyrido[4,3-b]indol-1(5H)-on [formula 9-2] (5.24 g, 28.14 mmol) and methyl 4-(bromomethyl)benzoate (7.09 g, 30.95 mmol) was dissolved in ACN (50 mL). Thereto, cesium carbonate (13.8 g, 42.21 mmol) was added and refluxed with stirring for 1 hour. After the completion of the reaction, the reaction mixture was extracted with ethyl acetate and saturated NaHCO₃ aqueous solution, the organic layer was dried over anhydrous MgSO₄ and filtered. The filtrate was concentrated under reduced pressure, and residue was purified by column chromatography (SiO₂; hexane/ethylacetate, 1/3) to yield the title compound as white solid (4.86 g, 52%).

### Step 3. Synthesis of methyl 4-((2-2-(tert-butyldimethylsilyloxy)ethyl)-1-oxo-1,2,3,4-tetrahydropirido[4,3-b]indol-5-yl)methyl)benzoate [formula 9-4]

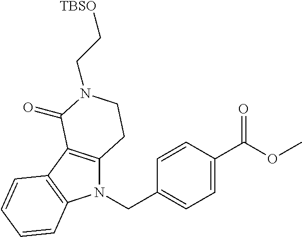 [SMILES: COC(=O)c1ccc(Cn2c3c(c4ccccc42)C(=O)N(CCO[Si](C)(C)C(C)(C)C)CC3)cc1]

Methyl 4-((1-oxo-1,2,3,4-tetrahydropyrido[4,3-b]indol-5-yl)methyl)benzoate [formula 9-3] (0.87 g, 2.60 mmol) was dissolved in DMF (9 mL) and DMPU (3 mL). Thereto, NaH (95%, 0.13 g, 5.20 mmol) and (2-bromoethoxy)(tert-butyl)dimethylsilane (1.1 mL, 5.20 mmol) were slowly added at 0° C. in order, and stirred at room temperature for 16 hours. After the completion of the reaction, the reaction mixture was extracted with ethyl acetate and saturated NaHCO₃ aqueous solution; the organic layer was dried over anhydrous MgSO₄ and filtered. Filtrate was concentrated under reduced pressure and purified by column chromatography (SiO₂; hexane/ethylacetate, 2/1) to yield the title compound as white solid (0.59 g, 45%).

### Step 4. Synthesis of methyl 4-((2-(2-hydroxyethyl)-1-oxo-1,2,3,4-tetrahydropirido[4,3-b]indol-5-yl)methyl)benzoate [formula 9-5]

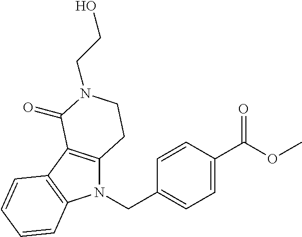 [SMILES: COC(=O)c1ccc(Cn2c3c(c4ccccc42)C(=O)N(CCO)CC3)cc1]

Methyl 4-((2-2-(tert-butyldimethylsilyloxy)ethyl)-1-oxo-1,2,3,4-tetrahydropirido[4,3-b]indol-5-yl)methyl)benzoate [formula 9-4] (0.59 g, 1.20 mmol) was dissolved in THF (10 mL). Thereto, tetrabutyl ammonium fluoride (1.0 M in THF, 1.44 mL, 1.44 mmol) was added and stirred at room temperature for 1 hour. After the completion of the reaction, the reaction mixture was extracted with ethyl acetate and saturated NaHCO₃ aqueous solution, the organic layer was dried over anhydrous MgSO₄ and filtered. Filtrate was concentrated under reduced pressure and purified by column chromatography (SiO₂; ethyl acetate) to yield the title compound as white solid (0.40 g, 88%).

### Step 5. Synthesis of methyl 4-((2-(2-morpholinoethyl)-1-oxo-1,2,3,4-tetrahydropyrido[4,3-b]indol-5-yl)methyl)benzoate [formula 9-6]

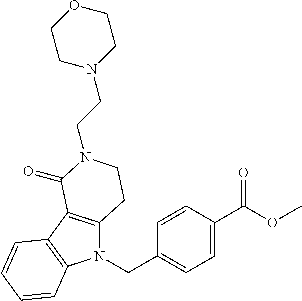 [SMILES: COC(=O)c1ccc(Cn2c3c(c4ccccc42)C(=O)N(CCN2CCOCC2)CC3)cc1]

Methyl 4-((2-(2-hydroxyethyl)-1-oxo-1,2,3,4-tetrahydropirido[4,3-b]indol-5-yl)methyl)benzoate [formula 9-5] (0.050 g, 0.13 mmol) was dissolved in ACN (3 mL). Thereto, DIPEA (0.12 mL, 0.66 mmol) and methanesulfonyl chloride (0.031 mL, 0.40 mmol) were added in order and stirred for 1 minute, morpholine (0.058 mL, 0.66 mmol) was added and stirred at room temperature for 30 minutes. After the completion of the reaction, the reaction mixture was extracted with ethyl acetate and saturated NaHCO₃, the organic layer was dried over anhydrous MgSO₄ and filtered. Filtrate was concentrated under reduced pressure and purified by column chromatography (SiO₂; dichloromethane/methanol, 20/1) to yield the title compound as yellow liquid (0.036 g, 61%).

### Step 6. Synthesis of N-hydroxy-4-((2-(2-morpholinoethyl)-1-oxo-1,2,3,4-tetrahydropyrido[4,3-b]indol-5-yl)methyl)benzamide [formula 9-7]

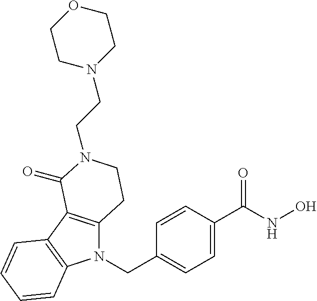 [SMILES: O=C(NO)c1ccc(Cn2c3c(c4ccccc42)C(=O)N(CCN2CCOCC2)CC3)cc1]

Methyl 4-((2-(2-morpholinoethyl)-1-oxo-1,2,3,4-tetrahydropyrido[4,3-b]indol-5-yl)methyl)benzoate [formula 9-6] (0.036 g, 0.080 mmol) was dissolved in methanol (5 mL). Thereto, hydroxylamine 50% aqueous solution (1 mL), hydroxylamine hydrochloride (NH₂OH HCl) (0.028 g, 0.40 mmol) and potassium hydroxide (0.090 g, 1.61 mmol) were added in order, and stirred at room temperature for 1 hour. After the completion of the reaction, the reaction mixture was concentrated under reduced pressure until the remained solution to be about 2-3 mL, and saturated NaHCO₃ (1-2 mL) was added and stirred. The solid product was filtered, washed with water, and dried with vacuum to yield compound 249 as white solid (0.023 g, 64%).

¹H NMR (400 MHz, DMSO-d₆) δ 11.16 (brs, 1H), 9.04 (brs, 1H), 7.95 (m, 1H), 7.67 (d, 2H, J=7.6 Hz), 7.47 (m, 1H), 7.15 (m, 4H), 5.51 (s, 2H), 3.68 (t, 2H, J=6.5 Hz), 3.54 (m, 6H), 3.04 (t, 2H, J=6.1 Hz), 2.42 (m, 4H), 2.17 (m, 2H); MS (ESI) m/z 449 (M⁺+H).

### Example 77

### Synthesis of Compound 250

### Step 1. Synthesis of 3-methyl-6,7-dihydro-1H-pyrrolo[3,2-c]pyridin-4(5H)-on [formula 8-2]

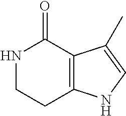

The compound of formula 8-1 (5 g, 23.45 mmol) was dissolved in acetic acid (100 mL). Thereto, anti-pyruvic aldehyde-1-oxime (2.04 g, 23.45 mmol) was slowly added and stirred at room temperature for 10 minutes, and Zn dust (6.13 g, 93.8 mmol) was added. After increasing temperature slowly, the reaction mixture was stirred at 120° C. for 3 hours, the reaction was completed by adding small volume of water. Acetic acid was concentrated under reduced pressure, and the concentration was purified by column chromatography (40 g ISCO silica gel cartridge, 0-100% EtOAc/hexane) to yield the title compound as light yellow solid (0.90 g, 26%).

### Step 2. Synthesis of tert-butyl 3-methyl-4-oxo-4,5,6,7-tetrahydropyrrolo[3,2-c]pyridin-1-carboxylate [formula 8-3]

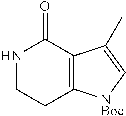 [SMILES: Cc1cn(C(=O)OC(C)(C)C)c2c1C(=O)NCC2]

The compound of formula 8-2 (0.20 g, 1.33 mmol) was dissolved in dichloromethane (10 mL), and triethylamine (0.22 g, 1.60 mmol) was added. Then, di-tert-butyl di carbonate (0.31 g, 1.44 mmol) and a little amount of DMAP were added. After stirring at room temperature for 2 hours, the reaction was completed. The reaction mixture was washed with brine three times, the organic layer was dried over Na₂SO₄ and filtered, and the filtrate was concentrated under reduced pressure. The concentration was purified by column chromatography (40 g ISCO silica gel cartridge, 0-50% EtOAc/hexane) to yield the title compound as yellow solid (0.33 g, 99%).

### Step 3. Synthesis of tert-butyl 3-methyl-5-(2-morpholinoethyl)-4-oxo-4,5,6,7-tetrahydropyrrolo[3,2-c]pyridin-1-carboxylate [formula 8-4]

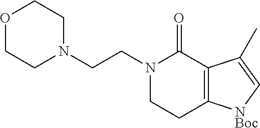 [SMILES: Cc1cn(C(=O)OC(C)(C)C)c2c1C(=O)N(CCN1CCOCC1)CC2]

The compound of formula 8-3 (0.12 g, 0.48 mmol) was dissolved in DMF (2 mL). Thereto, N-(2-chloroethyl)morpholine hydrochloride (0.18 g, 0.96 mmol) and sodium hydride (0.058 g, 2.40 mmol) were slowly added and stirred at room temperature for 5 hours. After the completion of the reaction, the reaction mixture was washed with brine three times, the organic layer was dried over Na₂SO₄ and filtered, and the filtrate was concentrated under reduced pressure. The concentration was purified by column chromatography (40 g ISCO silica gel cartridge, 0-100% EtOAc/hexane) to yield the title compound as white solid (0.053 g, 30%).

### Step 4. Synthesis of 3-methyl-5-(2-morpholinoethyl)-6,7-dehydro-1H-pyrrolo[3,2-c]pyridine-4(5H)-on [formula 8-5]

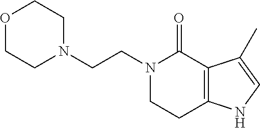 [SMILES: Cc1c[nH]c2c1C(=O)N(CCN1CCOCC1)CC2]

The compound of formula 8-4 (0.05 g, 0.14 mmol) was dissolved in dichloromethane (2 mL). Thereto, a small volume of trifluroacetic acid (0.032 mL, 0.41 mmol) was added, and stirred at room temperature for 1 hour. After the completion of the reaction, the reaction mixture was washed with brine three times, the organic layer was dried over Na₂SO₄ and filtered, and the filtrate was concentrated under reduced pressure. The concentration was refined by column chromatography (4 g ISCO silica gel cartridge, 0-10% methanol/dichloromethane) to yield the title compound as yellow solid (0.020 g, 55%).

### Step 5. Synthesis of methyl 4-((3-methyl-5-(2-morpholinoethyl)-4-oxo-4,5,6,7-tetrahydropyrrolo[3,2-c]pyridin-1-yl)methyl)benzoate [formula 8-6]

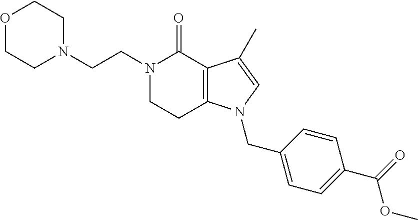 [SMILES: COC(=O)c1ccc(Cn2cc(C)c3c2CCN(CCN2CCOCC2)C3=O)cc1]

The compound of formula 8-5 (0.02 g, 0.076 mmol) was dissolved in DMF (5 mL).

Thereto, small amounts of methyl-4-(bromomethyl)benzoate (0.026 g, 0.11 mmol) and sodium hydride (0.0036 g, 0.15 mmol) were added and stirred at room temperature for 1 hour. After the completion of the reaction, the reaction mixture was washed with brine three times, the organic layer was dried over Na₂SO₄ and filtered, and the filtrate was concentrated under reduced pressure. The concentration was purified by column chromatography (4 g ISCO silica gel cartridge, 0-10% methanol/dichloromethane) to yield the title compound as light yellow oil (0.010 g, 32%).

### Step 6. Synthesis of N-hydroxy-4-((3-methyl-5-(2-morpholinoethyl)-4-oxo-4,5,6,7-tetrahydropyrrolo[3,2-c]pyridin-1-yl)methyl)benzamide [formula 8-7]

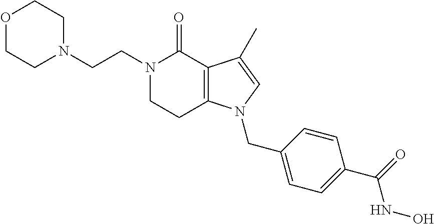 [SMILES: Cc1cn(Cc2ccc(C(=O)NO)cc2)c2c1C(=O)N(CCN1CCOCC1)CC2]

The compound of formula 8-6 (0.007 g, 0.017 mmol) was dissolved in methanol (5 mL), and hydroxylamine hydrochloride (0.006 g, 0.085 mmol) was added slowly. Then potassium hydroxide (0.009 g, 0.17 mmol) was added, stirred at room temperature, and hydroxylamine 50% aqueous solution was added. After stirring at room temperature for one day, organic solvent was concentrated under reduced pressure, washed with brine three times, the organic layer was dried over Na₂SO₄ and filtered, and the filtrate was concentrated under reduced pressure. The concentration was dried to yield the compound 250 as light yellow oil (0.006 g, 86%).

¹H NMR (400 MHz, MeOD-d₃) δ 7.75 (d, 2H, J=8.2 Hz), 7.42 (d, 2H, J=8.2 Hz), 6.56 (t, 1H, J=8.5 Hz), 4.20 (brs, 2H), 3.85 (brs, 4H), 3.57 (t, 2H, J=6.8 Hz), 3.32 (brs, 2H), 3.18 (brs, 4H), 2.90 (t, 2H, J=7.1 Hz), 2.24 (s, 3H); MS (ESI) m/z 413 (M⁺+H).

### Example 78

### Synthesis of Compound 251

### Step 1. Synthesis of methyl 4-((2-(2-(4-methylpiperazin-1-yl)ethyl)-1-oxo-1,2,3,4-tetrahydropyrido[4,3-b]indol-5-yl)methyl)benzoate [formula 9-6]

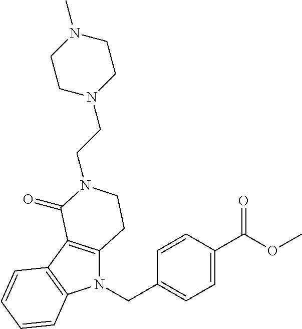 [SMILES: COC(=O)c1ccc(Cn2c3c(c4ccccc42)C(=O)N(CCN2CCN(C)CC2)CC3)cc1]

Methyl 4-((2-(2-hydroxyethyl)-1-oxo-1,2,3,4-tetrahydropirido[4,3-b]indol-5-yl)methyl)benzoate [formula 9-5] (0.15 g, 0.40 mmol) was dissolved in ACN (5 mL). Thereto, carbon tetrabromide (0.17 g, 0.52 mmol) and triphenylphosphine (0.14 g, 0.52 mmol) were added in order and stirred at room temperature for 10 minutes, and 1-methylpiperazine (0.088 g, 0.79 mmol) was added and stirred 50° C. for 1 hour. After the completion of the reaction, the reaction mixture was extracted with ethyl acetate and saturated NaHCO₃, the organic layer was dried over anhydrous MgSO₄ and filtered. Filtrate was concentrated under reduced pressure and purified by column chromatography (SiO₂; dichloromethane/methanol, 10/1) to yield the title compound as yellow liquid (0.086 g, 47%).

### Step 2. Synthesis of N-hydroxy-4-((2-(2-(4-methylpiperazin-1-yl)ethyl)-1-oxo-1,2,3,4-tetrahydropyrido[4,3-b]indol-5-yl)methyl)benzamide [formula 9-7]

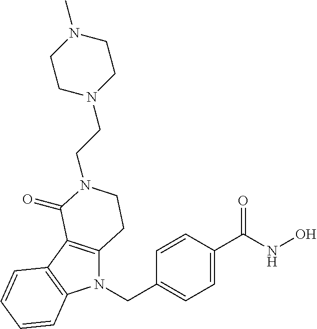 [SMILES: CN1CCN(CCN2CCc3c(c4ccccc4n3Cc3ccc(C(=O)NO)cc3)C2=O)CC1]

Methyl 4-((2-(2-(4-methylpiperazin-1-yl)ethyl)-1-oxo-1,2,3,4-tetrahydropyrido[4,3-b]indol-5-yl)methyl)benzoate [formula 9-6] (0.086 g, 0.21 mmol) was dissolved in methanol (5 mL). Thereto, hydroxylamine 50% aqueous solution (2.6 mL), hydroxylamine hydrochloride (NH₂OH HCl) (0.074 g, 1.06 mmol) and potassium hydroxide (0.24 g, 4.23 mmol) were added in order, and stirred at room temperature for 1 hour. After the completion of the reaction, the reaction mixture was concentrated under reduced pressure until the remained solution to be about 2-3 mL. The reaction mixture was extracted with saturate NaHCO₃, ethyl acetate and THF, the organic layer was dried over anhydrous MgSO₄ and filtered. Filtrate was concentrated under reduced pressure and dried with vacuum to yield the compound 251 as white solid (0.036 g, 37%).

MS (ESI) m/z 462 (M⁺+H).

### Example 79

### Synthesis of Compound 266

### Step 1. Synthesis of 8-fluoro-3,4-dihydro-2H-pyrido[4,3-b]indol-1(5H)-on [formula 9-2]

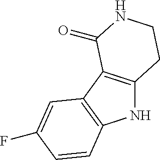 [SMILES: O=C1NCCc2[nH]c3ccc(F)cc3c21]

Tert-butyl 2,4-dioxopiperidine-1-carboxylate [formula 9-1] (9.0 g, 42.21 mmol) was dissolved in TFA (18 mL), and stirred at room temperature for 30 minutes, and (4-fluorophenyl)hydrazine hydrochlororide (6.86 g, 42.21 mmol) and H₂SO₄ (1.8 mL) was added and stirred at 100° C. for 16 hours. Then, the reaction mixture was concentrated under reduced pressure and extracted with ethyl acetate and saturated NaHCO₃ aqueous solution, the organic layer was dried with anhydrous MgSO₄, filtered and concentrated under reduced pressure. Residue was purified by column chromatography (SiO₂; hexane/ethylacetate, 1/3) to yield the title compound as brown solid (4.87 g, 57%)

### Step 2. Synthesis of methyl 4-((8-fluoro-1-oxo-1,2,3,4-tetrahydropyrido[4,3-b]indol-5-yl)methyl)benzoate [formula 9-3]

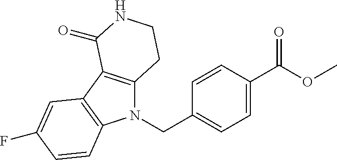 [SMILES: COC(=O)c1ccc(Cn2c3c(c4cc(F)ccc42)C(=O)NCC3)cc1]

8-fluoro-3,4-dihydro-2H-pyrido[4,3-b]indol-1(5H)-on [formula 9-2](6.09 g, 29.82 mmol) and methyl 4-(bromomethyl)benzoate (8.20 g, 35.79 mmol) were dissolved in ACN (50 mL). Thereto, cesium carbonate (14.6 g, 44.74 mmol) was added and refluxed with stirring for 3 hours. After the completion of the reaction, the reaction mixture was extracted with ethyl acetate and NaHCO₃. The organic layer was dried over Na₂SO₄ and filtered, and the filtrate was concentrated under reduced pressure. The filtrate was concentrated under reduced pressure and residue was purified by column chromatography (SiO₂; hexane/ethylacetate, 1/4) to yield the title compound as bright brown solid (5.0 g, 48%).

### Step 3. Synthesis of methyl 4-((2-2-(tert-butyldimethylsilyloxy)ethyl)-8-fluoro-1-oxo-1,2,3,4-tetrahydropirido[4,3-b]indol-5-yl)methyl)benzoate [formula 9-4]

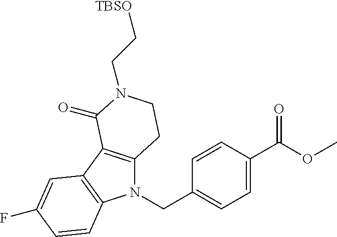 [SMILES: COC(=O)c1ccc(Cn2c3c(c4cc(F)ccc42)C(=O)N(CCO[Si](C)(C)C(C)(C)C)CC3)cc1]

Methyl 4-((8-fluoro-1-oxo-1,2,3,4-tetrahydropyrido[4,3-b]indol-5-yl)methyl)benzoate [formula 9-3] (0.95 g, 2.70 mmol) was dissolved in DMF (9 mL) and DMPU (3 mL). Thereto, NaH (95%, 0.14 g, 5.39 mmol) and (2-bromoethoxy)(tert-butyl)dimethylsilane (1.15 mL, 5.39 mmol) were added in order and stirred at room temperature for 16 hours. After the completion of the reaction, the reaction mixture was extracted with ethyl acetate and saturated NaHCO₃ aqueous solution, the organic layer was dried over anhydrous MgSO₄ and filtered. The filtrate was concentrated under reduced pressure and residue was purified by column chromatography (SiO₂; hexane/ethylacetate, 2/1) to yield the title compound as bright brown solid (0.83 g, 60%).

### Step 4. Synthesis of methyl 4-((8-fluoro2-(2-hydroxyethyl)-1-oxo-1,2,3,4-tetrahydropirido[4,3-b]indol-5-yl)methyl)benzoate [formula 9-5]

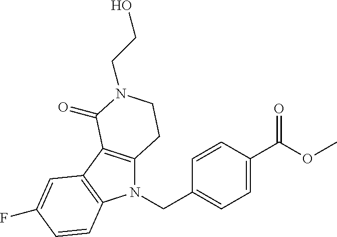 [SMILES: COC(=O)c1ccc(Cn2c3c(c4cc(F)ccc42)C(=O)N(CCO)CC3)cc1]

Methyl 4-((2-2-(tert-butyldimethylsilyloxy)ethyl)-8-fluoro-1-oxo-1,2,3,4-tetrahydropirido[4,3-b]indol-5-yl)methyl)benzoate [formula 9-4] (0.83 g, 1.63 mmol) was dissolved in THF (10 mL). Thereto, tetrabutyl ammonium fluoride (1.0 M in THF, 2.0 mL, 1.95 mmol) was added and stirred at room temperature for 1 hour. After the completion of the reaction, the reaction mixture was extracted with ethyl acetate and saturated NaHCO₃ aqueous solution, the organic layer was dried over anhydrous MgSO₄ and filtered. Filtrate was concentrated under reduced pressure and residue was purified by column chromatography (SiO₂; ethyl acetate) to yield the title compound as white solid (0.50 g, 78%).

### Step 5. Synthesis of methyl 4-((8-fluoro-2-(2-morpholinoethyl)-1-oxo-1,2,3,4-tetrahydropyrido[4,3-b]indol-5-yl)methyl)benzoate [formula 9-6]

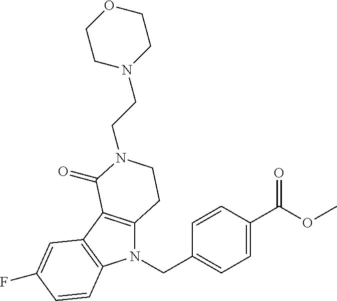 [SMILES: COC(=O)c1ccc(Cn2c3c(c4cc(F)ccc42)C(=O)N(CCN2CCOCC2)CC3)cc1]

Methyl 4-((8-fluoro 2-(2-hydroxyethyl)-1-oxo-1,2,3,4-tetrahydropirido[4,3-b]indol-5-yl)methyl)benzoate [formula 9-5] (0.10 g, 0.25 mmol) was dissolved in ACN (5 mL). Thereto, DIPEA (0.22 g, 1.26 mmol) and methanesulfonyl chloride (0.06 mL, 0.76 mmol) were added in order and stirred at room temperature for 5 minutes, morpholine (0.11 mL, 1.26 mmol) was added and stirred at 50° C. for 30 minutes. After the completion of the reaction, the reaction mixture was extracted with ethyl acetate and saturated NaHCO₃, the organic layer was dried over anhydrous MgSO₄ and filtered. The filtrate was concentrated under reduced pressure and residue was purified by column chromatography (SiO₂; dichloromethane/methanol, 20/1) to yield the title compound as bright yellow solid (0.073 g, 62%).

### Step 6. Synthesis of 4-(8-fluoro-2-(2-morpholinoethyl)-1-oxo-1,2,3,4-tetrahydropyrido[4,3-b]indol-5-yl)methyl)-N-hydroxybenzamide [formula 9-7]

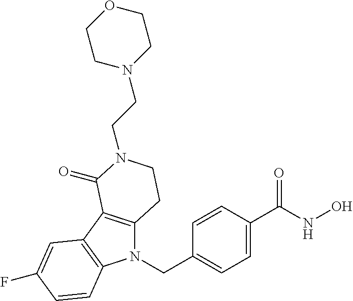 [SMILES: O=C(NO)c1ccc(Cn2c3c(c4cc(F)ccc42)C(=O)N(CCN2CCOCC2)CC3)cc1]

Methyl 4-((8-fluoro-2-(2-morpholinoethyl)-1-oxo-1,2,3,4-tetrahydropyrido[4,3-b]indol-5-yl)methyl)benzoate [formula 9-6] (0.073 g, 0.16 mmol) was dissolved in methanol (5 mL). Thereto, hydroxylamine 50% aqueous solution (2 mL), hydroxylamine hydrochloride (NH₂OH HCl) (0.055 g, 0.78 mmol) and potassium hydroxide (0.18 g, 3.14 mmol) were added in order, and stirred at room temperature for 30 minutes. After the completion of the reaction, the reaction mixture was concentrated under reduced pressure until the remained solution to be about 2-3 mL, and saturated NaHCO₃ (1-2 mL) was added and stirred. The obtained solid product from filtering was washed with water, dried with vacuum to yield compound 266 as white solid (0.062 g, 85%).

¹H NMR (400 MHz, DMSO-d₆) δ 7.67 (d, 2H, J=8.0 Hz), 7.60 (dd, 1H, J=9.6, 2.8 Hz), 7.48 (dd, 1H, J=9.0, 4.2 Hz), 7.14 (d, 2H, J=8.0 Hz), 6.99 (td, 1H, J=9.2, 2.4 Hz), 5.51 (s, 2H), 3.68 (t, 2H, J=6.8 Hz), 3.53 (m, 6H), 3.05 (t, 2H, J=7.0 Hz), 2.49 (m, 2H), 2.41 (s, 4H); MS (ESI) m/z 467 (M⁺+H).

### Example 80

### Synthesis of Compound 267

### Step 1. Synthesis of (S)-methyl 4-((2-(2-(2-(hydroxymethyl)pyrrolidin-1-yl)ethyl)-1-oxo-1,2,3,4-tetrahydropyrido[4,3-b]indol-5-yl)methyl)benzoate [formula 9-6]

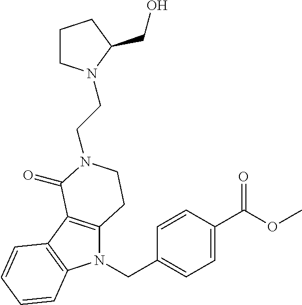 [SMILES: COC(=O)c1ccc(Cn2c3c(c4ccccc42)C(=O)N(CCN2CCC[C@H]2CO)CC3)cc1]

Methyl4-((2-(2-hydroxyethyl)-1-oxo-1,2,3,4-tetrahydropirido[4,3-b]indol-5-yl)methyl)benzoate [formula 9-5] (0.085 g, 0.23 mmol) was dissolved in ACN (5 mL). Thereto, DIPEA (0.20 mL, 1.12 mmol) and methanesulfonyl chloride (0.05 mL, 0.67 mmol) were added in order and stirred at room temperature for 5 minutes, (S)-2-(hydroxymethyl)pyrrolidin (0.11 mL, 1.12 mmol) was added and stirred at 50° C. for 30 minutes. After the completion of the reaction, the reaction mixture was extracted with ethyl acetate and saturated NaHCO₃, the organic layer was dried over anhydrous MgSO₄ and filtered. The filtrate was concentrated under reduced pressure and the residue was purified by column chromatography (SiO₂; dichloromethane/methanol, 10/1) to yield the title compound as bright yellow solid (0.062 g, 60%).

### Step 2. Synthesis of (S)—N-hydroxy-4-((2-(2-(2-(hydroxymethyl)pyrrolidin-1-yl)ethyl)-1-oxo-1,2,3,4-tetrahydropyrido[4,3-b]indol-5-yl)methyl)benzamide [formula 9-7]

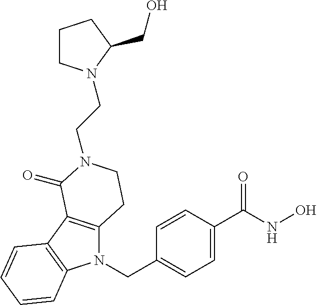 [SMILES: O=C(NO)c1ccc(Cn2c3c(c4ccccc42)C(=O)N(CCN2CCC[C@H]2CO)CC3)cc1]

(S)-methyl 4-((2-(2-(2-(hydroxymethyl)pyrrolidin-1-yl)ethyl)-1-oxo-1,2,3,4-tetrahydropyrido[4,3-b]indol-5-yl)methyl)benzoate [formula 9-6] (0.062 g, 0.13 mmol) was dissolved in methanol (5 mL). Thereto, hydroxylamine 50% aqueous solution (1.6 mL), hydroxylamine hydrochloride (NH₂OH HCl) (0.047 g, 0.67 mmol), and potassium hydroxide (0.15 g, 2.69 mmol) were added in order, and stirred at room temperature for 30 minutes. After the completion of the reaction, the reaction mixture was concentrated under reduced pressure until the remained solution to be about 2-3 mL, and saturated NaHCO₃ (1-2 mL) was added and stirred. The obtained solid product from filtering was washed with water, dried with vacuum to yield compound 267 as white solid (0.048 g, 77%).

¹H NMR (400 MHz, DMSO-d₆) δ 11.17 (brs, 1H), 9.05 (brs, 1H), 7.96 (m, 1H), 7.66 (d, 2H, J=8.4 Hz), 7.45 (m, 1H), 7.15-7.11 (m, 4H), 5.50 (s, 2H), 4.38 (brs, 1H), 3.71-3.63 (m, 2H), 3.54 (m, 1H), 3.49-3.41 (m, 2H), 3.19-3.11 (m, 2H), 3.05-3.00 (m, 3H), 2.47-2.40 (m, 2H), 2.20 (m, 1H), 1.75 (m, 1H); 1.65-1.60 (m, 2H), 1.52 (m, 1H); MS (ESI) m/z 463 (M⁺+H).

### Example 81

### Synthesis of Compound 268

### Step 1. Synthesis of methyl 4-((2,3-dimethyl-5-methylen-4-oxo-4,5,6,7-tetrahydroindol-1-yl)methyl)benzoate [formula 7-4]

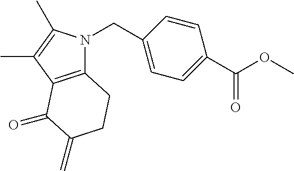

Methyl 4-((2,3-dimethyl-4-oxo-4,5,6,7-tetrahydroindol-1-yl)methyl)benzoate [formula 7-3] (1.0 g, 3.212 mmol), N,N-dimethylamine HCl (0.524 g, 6.423 mmol), paraformaldehyde (0.213 g, 6.423 mmol) and mixed solvent (acetic acid:toluene=4:1, 15 mL) were added, and stirred at 100° C. for 4 hours. After the completion of the reaction, acetic acid was distilled out under reduced pressure, and without purification, the compound was dissolved in a 15 mL of mixed solvent (acetonitrile:H₂O=1:4) and stirred at 80° C. for 12 hours. After the completion of the reaction, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 7/1) to yield the title compound (0.67 g, 65%).

### Step 2. Synthesis of methyl 4-((2,3-dimethyl-5-(morpholinomethyl)-4-oxo-4,5,6,7-tetrahydroindol-1-yl)methyl)benzoate [formula 7-5]

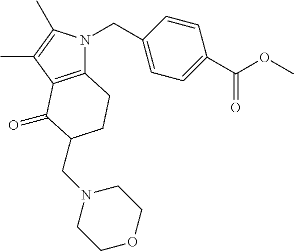

To a microwave vial were added methyl 4-((2,3-dimethyl-5-methylen-4-oxo-4,5,6,7-tetrahydroindol-1-yl)methyl)benzoate [formula 7-4] (0.30 g, 0.928 mmol), morpholine (0.242 g, 2.783 mmol) and toluene (10.0 mL), and a reaction was carried out in a microwave reactor at 110° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.22 g, 58%).

### Step 3. Synthesis of 4-((2,3-dimethyl-5-(morpholinomethyl)-4-oxo-4,5,6,7-tetrahydroindol-1-yl)methyl)-N-hydroxybenzamide [formula 7-6]

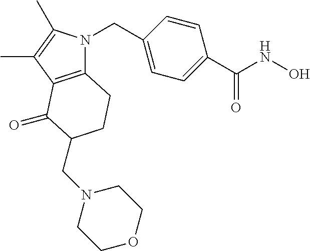 [SMILES: Cc1c2c(n(Cc3ccc(C(=O)NO)cc3)c1C)CCC(CN1CCOCC1)C2=O]

To a flask were added methyl 4-((2,3-dimethyl-5-(morpholinomethyl)-4-oxo-4,5,6,7-tetrahydroindol-1-yl)methyl)benzoate [formula 7-5] (0.10 g, 0.244 mmol), hydroxylamine hydrochloride (NH₂OH HCl) (0.034 g, 0.487 mmol), potassium hydroxide (0.055 g, 0.97 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. And the reaction mixture was re-crystallized by diethyl ether and ethyl acetate, and filtered to yield the compound 268 (0.054 g, 54%).

¹H NMR (400 MHz, CDCl₃) δ 7.73 (d, 2H, J=8.2 Hz), 6.91 (d, 2H, J=8.2 Hz), 5.03 (s, 2H), 3.75-3.72 (m, 4H), 3.02-2.98 (m, 1H), 2.65-2.52 (m, 8H), 2.27 (s, 3H), 2.13-1.99 (m, 4H); MS (ESI) m/z 412 (M++H).

### Example 82

### Synthesis of Compound 283

### Step 1. Synthesis of methyl 4-((6-fluoro-3-(((R)-3-fluoropyrrolidin-1-yl)methyl)-4-oxo-1,2,3,4-tetrahydrocarbazol-9-yl)methyl)benzoate [formula 2-5]

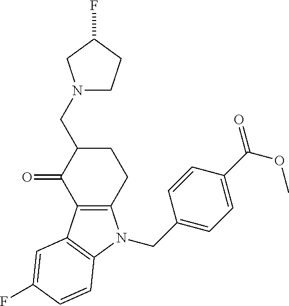 [SMILES: COC(=O)c1ccc(Cn2c3c(c4cc(F)ccc42)C(=O)C(CN2CC[C@@H](F)C2)CC3)cc1]

To a microwave vial were added methyl 4-((6-fluoro-3-methylen-4-oxo-1,2,3,4-tetrahydrocarbazol-9-yl)methyl)benzoate [formula 2-4] (0.1 g, 0.275 mmol), (R)-3-fluoropyrrolidin, HCl (0.0736 g, 0.826 mmol) and toluene (3.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.082 g, 66%).

### Step 2. Synthesis of 4-((6-fluoro-3-(((R)-3-fluoropyrrolidin-1-yl)methyl)-4-oxo-1,2,3,4-tetrahydrocarbazol-9-yl)methyl)-N-hydroxybenzamide [formula 2-6]

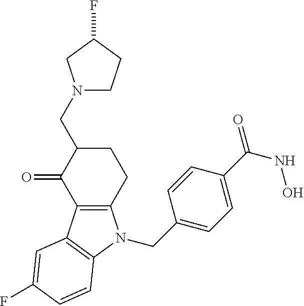 [SMILES: O=C(NO)c1ccc(Cn2c3c(c4cc(F)ccc42)C(=O)C(CN2CC[C@@H](F)C2)CC3)cc1]

To a flask were added methyl 4-((6-fluoro-3-(((R)-3-fluoropyrrolidin-1-yl)methyl)-4-oxo-1,2,3,4-tetrahydrocarbazol-9-yl)methyl)benzoate [formula 2-5] (0.082 g, 0.181 mmol), hydroxylamine hydrochloride (NH₂OH HCl) (0.063 g, 0.906 mmol), potassium hydroxide (0.102 g, 1.812 mmol) and methanol (10 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 283 as solid (0.054 g, 66%).

¹H NMR (400 MHz, DMSO-d₆) δ 7.67 (d, 3H, J=8.2 Hz), 7.53-7.50 (m, 1H), 7.16 (d, 2H, J=7.8 Hz), 7.06-7.01 (m, 1H), 5.54 (s, 2H), 5.23-5.08 (m, 1H), 3.17-3.02 (m, 1H), 3.00-2.82 (m, 2H), 2.78-2.61 (m, 5H), 2.41-2.29 (m, 2H), 2.23-2.03 (m, 2H), 2.00-2.91 (m, 1H); MS (ESI) m/z 454 (M++H).

### Example 83

### Synthesis of Compound 284

### Step 1. Synthesis of methyl 4-((6-fluoro-3-((4-(2-methoxyethyl)piperazin-1-yl)methyl)-4-oxo-1,2,3,4-tetrahydrocarbazol-9-yl)methyl)benzoate [formula 2-5]

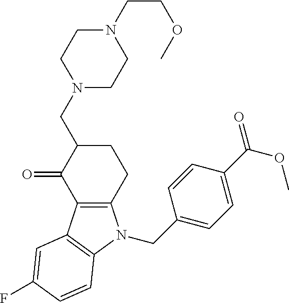 [SMILES: COCCN1CCN(CC2CCc3c(c4cc(F)ccc4n3Cc3ccc(C(=O)OC)cc3)C2=O)CC1]

To a microwave vial were added methyl 4-((6-fluoro-3-methylen-4-oxo-1,2,3,4-tetrahydrocarbazol-9-yl)methyl)benzoate [formula 2-4] (0.10 g, 0.275 mmol), 1-2-methoxyethyl piperazin (0.1191 g, 0.826 mmol) and toluene (3.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.089 g, 64%).

### Step 2. Synthesis of 4-((6-fluoro-3-((4-(2-methoxyethyl)piperazin-1-yl)methyl)-4-oxo-1,2,3,4-tetrahydrocarbazol-9-yl)methyl)-N-hydroxybenzamide [formula 2-6]

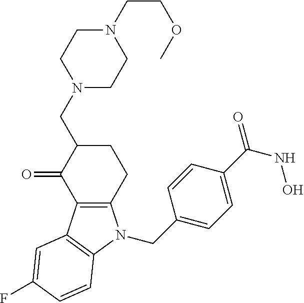 [SMILES: COCCN1CCN(CC2CCc3c(c4cc(F)ccc4n3Cc3ccc(C(=O)NO)cc3)C2=O)CC1]

To a flask were added methyl 4-((6-fluoro-3-((4-(2-methoxyethyl)piperazin-1-yl)methyl)-4-oxo-1,2,3,4-tetrahydrocarbazol-9-yl)methyl)benzoate [formula 2-5] (0.089 g, 0.175 mmol), hydroxylamine hydrochloride (NH₂OH HCl) (0.061 g, 0.877 mmol), potassium hydroxide (0.098 g, 1.753 mmol) and methanol (10 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 284 as solid (0.048 g, 54%).

¹H NMR (400 MHz, DMSO-d₆) δ 7.67-7.50 (m, 4H), 7.30-7.04 (m, 3H), 5.54 (s, 2H), 3.22-3.17 (m, 4H), 3.16-3.01 (m, 2H), 2.96-2.83 (m, 2H), 2.72-2.60 (m, 2H), 2.41-2.19 (m, 5H), 2.03-1.85 (m, 2H), 1.72-1.62 (brs, 1H), 1.24-1.16 (m, 2H); MS (ESI) m/z 509 (M++H).

### Example 84

### Synthesis of Compound 285

### Step 1. Synthesis of (S)-methyl-4-((8-fluoro-2-(2-(2-(methoxymethyl)pyrrolidin-1-yl)ethyl)-1-oxo-1,2,3,4-tetrahydropyrido[4,3-b]indol-5-yl)methyl)benzoate [formula 9-6]

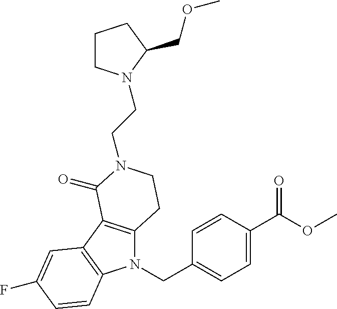 [SMILES: COC[C@@H]1CCCN1CCN1CCc2c(c3cc(F)ccc3n2Cc2ccc(C(=O)OC)cc2)C1=O]

(S)-methyl-4-((8-fluoro-2-(2-hydroxyethyl)-1-oxo-1,2,3,4-tetrahydropyrido[4,3-b]indol-5-yl)methyl)benzoate [formula 9-5] (0.05 g, 0.13 mmol) was dissolved in ACN (1 mL). Thereto, DIPEA (0.11 mL, 0.63 mmol) and methanesulfonyl chloride (0.02 mL, 0.25 mmol) were added in order and stirred at room temperature for 5 minutes. And then, (S)-2-(methoxymethyl)pyrrolidin (0.044 mL, 0.38 mmol) was added and stirred at 50° C. for 30 minutes. After the completion of the reaction, the reaction mixture was purified by column chromatography (SiO₂; dichloromethane/methanol, 20/1) to yield the title compound as yellow liquid (0.021 g, 34%).

### Step 2. Synthesis of (S)-4-((8-fluoro-2-(2-(2-(methoxymethyl)pyrrolidin-1-yl)ethyl)-1-oxo-1,2,3,4-tetrahydropyrido[4,3-b]indol-5-yl)methyl)-N-hydroxybenzamide [formula 9-7]

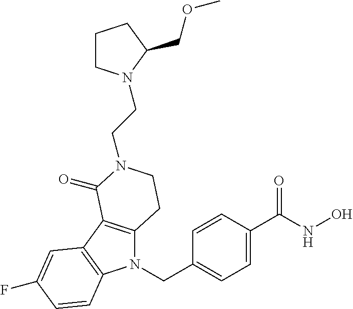 [SMILES: COC[C@@H]1CCCN1CCN1CCc2c(c3cc(F)ccc3n2Cc2ccc(C(=O)NO)cc2)C1=O]

(S)-methyl-4-((8-fluoro-2-(2-(2-(methoxymethyl)pyrrolidin-1-yl)ethyl)-1-oxo-1,2,3,4-tetrahydropyrido[4,3-b]indol-5-yl)methyl)benzoate [formula 9-6] (0.021 g, 0.043 mmol) was dissolved in methanol (5 mL). Thereto, hydroxylamine 50% aqueous solution (2.1 mL) and potassium hydroxide (0.048 g, 0.85 mmol) were added in order, and stirred at room temperature for 1 hour. After the completion of the reaction, the reaction mixture was concentrated under reduced pressure until the remained solution to be about 2-3 mL, and saturated NaHCO₃ (1-2 mL) was added and stirred. The obtained product from filtering was washed with water, dried with vacuum to yield compound 285 as bright brown solid (0.014 g, 67%).

¹H NMR (400 MHz, DMSO-d₆) δ 7.67 (d, 2H, J=8.2 Hz), 7.60 (m, 1H), 7.48 (m, 1H), 7.14 (d, 2H, J=8.2 Hz), 6.98 (m, 1H), 5.51 (s, 2H), 3.66 (m, 2H), 3.49 (m, 2H), 3.17 (m, 2H), 3.11 (m, 6H), 3.03 (m, 2H), 1.58 (m, 4H), 1.29 (m, 2H); MS (ESI) m/z 495 (M⁺+H).

### Example 85

### Synthesis of Compound 286

### Step 1. Synthesis of methyl 4-((8-fluoro-2-(2-(4-(hydroxymethyl)piperidin-1-yl)ethyl)-1-oxo-1,2,3,4-tetrahydropyrido[4,3-b]indol-5-yl)methyl)benzoate [formula 9-6]

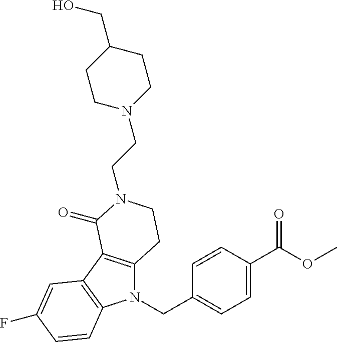 [SMILES: COC(=O)c1ccc(Cn2c3c(c4cc(F)ccc42)C(=O)N(CCN2CCC(CO)CC2)CC3)cc1]

Methyl 4-((8-fluoro-2-(2-hydroxyethyl)-1-oxo-1,2,3,4-tetrahydropyrido[4,3-b]indol-5-yl)methyl)benzoate [formula 9-5] (0.05 g, 0.13 mmol) was dissolved in ACN (1 mL). Thereto, DIPEA (0.11 mL, 0.63 mmol) and methanesulfonyl chloride (0.02 mL, 0.25 mmol) were added in order and stirred at room temperature for 5 minutes, 4-(hydroxymethyl)piperidin (0.044 mL, 0.38 mmol) was added and stirred at 50° C. for 30 minutes. After the completion of the reaction, the reaction mixture was purified by column chromatography (SiO₂; dichloromethane/methanol, 20/1) to yield the title compound as yellow solid (0.02 g, 32%).

### Step 2. Synthesis of 4-((8-fluoro-2-(2-(4-(hydroxymethyl)piperidin-1-yl)ethyl)-1-oxo-1,2,3,4-tetrahydropyrido[4,3-b]indol-5-yl)methyl)-N-hydroxybenzamide [formula 9-7]

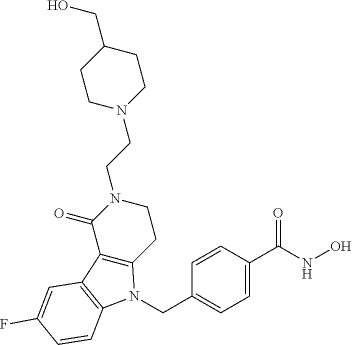 [SMILES: O=C(NO)c1ccc(Cn2c3c(c4cc(F)ccc42)C(=O)N(CCN2CCC(CO)CC2)CC3)cc1]

Methyl 4-((8-fluoro-2-(2-((4-(hydroxymethyl)piperidin-1-yl)ethyl)-1-oxo-1,2,3,4-tetrahydropyrido[4,3-b]indol-5-yl)methyl)benzoate [formula 9-6] (0.02 g, 0.041 mmol) was dissolved in methanol (5 mL). Thereto, hydroxylamine 50% aqueous solution (1.0 mL) and potassium hydroxide (0.046 g, 0.81 mmol) were added in order, and stirred at room temperature for 1 hour. After the completion of the reaction, the reaction mixture was concentrated under reduced pressure until the remained solution to be about 2-3 mL, and saturated NaHCO₃ (1-2 mL) was added and stirred. The obtained solid product from filtering was washed with water dried with vacuum to yield compound 286 as bright brown solid (0.008 g, 40%).

¹H NMR (400 MHz, DMSO-d₆) δ 7.65 (d, 2H, J=8.0 Hz), 7.59 (m, 1H), 7.49 (m, 1H), 7.04 (d, 2H, J=8.0 Hz), 6.98 (m, 1H), 5.45 (s, 2H), 3.68 (t, 2H, J=6.8 Hz), 3.53 (m, 2H), 3.21 (m, 2H), 3.03 (m, 2H), 2.91 (m, 2H), 2.44 (m, 2H), 1.88 (m, 2H), 1.60 (m, 2H), 1.31 (m, 2H), 1.06 (m, 2H); MS (ESI) m/z 495 (M⁺+H).

### Example 86

### Synthesis of Compound 287

### Step 1. Synthesis of methyl 4-((8-fluoro-2-(2-(4-methylpiperazin-1-yl)ethyl)-1-oxo-1,2,3,4-tetrahydropyrido[4,3-b]indol-5-yl)methyl)benzoate [formula 9-6]

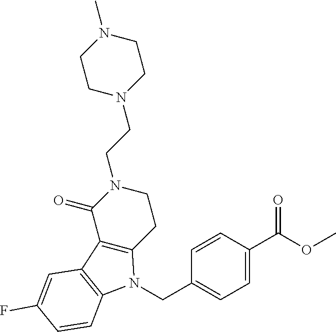 [SMILES: COC(=O)c1ccc(Cn2c3c(c4cc(F)ccc42)C(=O)N(CCN2CCN(C)CC2)CC3)cc1]

Methyl 4-((8-fluoro-2-(2-hydroxyethyl)-1-oxo-1,2,3,4-tetrahydropyrido[4,3-b]indol-5-yl)methyl)benzoate [formula 9-5] (0.05 g, 0.13 mmol) was dissolved in ACN (1 mL). Thereto, DIPEA (0.11 mL, 0.63 mmol) and methanesulfonyl chloride (0.02 mL, 0.25 mmol) were added in order and stirred at room temperature for 5 minutes, 1-methylpiperazin (0.038 g, 0.38 mmol) was added and stirred at 50° C. for 30 minutes. After the completion of the reaction, the reaction mixture was purified by column chromatography (SiO₂; dichloromethane/methanol, 10/1) to yield the title compound as yellow liquid (0.019 g, 32%).

### Step 2. Synthesis of 4-((8-fluoro-2-(2-(4-methylpiperazin-1-yl)ethyl)-1-oxo-1,2,3,4-tetrahydropyrido[4,3-b]indol-5-yl)methyl)-N-hydroxybenzamide [formula 9-7]

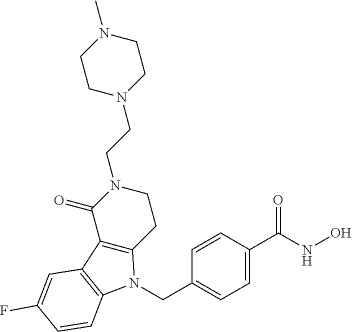 [SMILES: CN1CCN(CCN2CCc3c(c4cc(F)ccc4n3Cc3ccc(C(=O)NO)cc3)C2=O)CC1]

Methyl 4-((8-fluoro-2-(2-(4-methylpiperazin-1-yl)ethyl)-1-oxo-1,2,3,4-tetrahydropyrido[4,3-b]indol-5-yl)methyl)benzoate [formula 9-6] (0.019 g, 0.040 mmol) was dissolved in methanol (5 mL). Thereto, hydroxylamine 50% aqueous solution (1.0 mL) and, potassium hydroxide (0.045 g, 0.79 mmol) were added in order, and stirred at room temperature for 1 hour. After the completion of the reaction, the reaction mixture was concentrated under reduced pressure until the remained solution to be about 2-3 mL, and saturated NaHCO₃ (1-2 mL) was added and stirred. The obtained solid product from filtering was washed with water, dried with vacuum to yield compound 287 as white solid (0.007 g, 37%). MS (ESI) m/z 480 (M⁺+H).

### Example 87

### Synthesis of Compound 288

### Step 1. Synthesis of methyl 4-((8-fluoro-2-(2-(4-(2-hydroxyethyl)piperazin-1-yl)ethyl)-1-oxo-1,2,3,4-tetrahydropyrido[4,3-b]indol-5-yl)methyl)benzoate [formula 9-6]

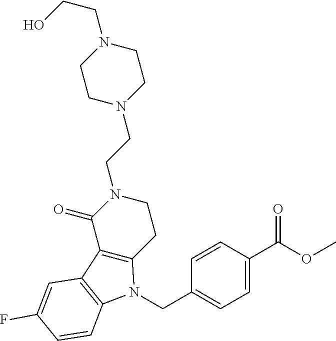 [SMILES: COC(=O)c1ccc(Cn2c3c(c4cc(F)ccc42)C(=O)N(CCN2CCN(CCO)CC2)CC3)cc1]

Methyl 4-((8-fluoro-2-(2-hydroxyethyl)-1-oxo-1,2,3,4-tetrahydropyrido[4,3-b]indol-5-yl)methyl)benzoate [formula 9-5] (0.05 g, 0.13 mmol) was dissolved in ACN (1 mL). Thereto, DIPEA (0.11 mL, 0.63 mmol) and methanesulfonyl chloride (0.02 mL, 0.25 mmol) were added in order and stirred at room temperature for 5 minutes, 1-(2-hydroxyethyl)piperazin (0.049 g, 0.38 mmol) was added and stirred at 50° C. for 30 minutes. After the completion of the reaction, the reaction mixture was purified by column chromatography (SiO₂; dichloromethane/methanol, 10/1) to yield the title compound as yellow solid (0.026 g, 41%).

### Step 2. Synthesis of 4-((8-fluoro-2-(2-(4-(2-hydroxyethyl)piperazin-1-yl)ethyl)-1-oxo-1,2,3,4-tetrahydropyrido[4,3-b]indol-5-yl)methyl)-N-hydroxybenzamide [formula 9-7]

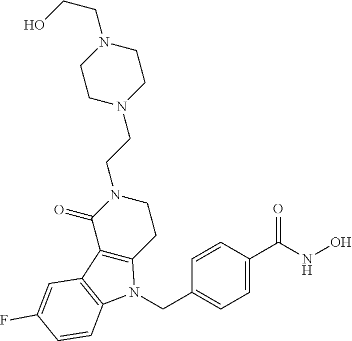 [SMILES: O=C(NO)c1ccc(Cn2c3c(c4cc(F)ccc42)C(=O)N(CCN2CCN(CCO)CC2)CC3)cc1]

Methyl 4-((8-fluoro-2-(2-(4-(2-hydroxyethyl)piperazin-1-yl)ethyl)-1-oxo-1,2,3,4-tetrahydropyrido[4,3-b]indol-5-yl)methyl)benzoate [formula 9-6] (0.026 g, 0.051 mmol) was dissolved in methanol (5 mL). Thereto, hydroxylamine 50% aqueous solution (1.3 mL) and potassium hydroxide (0.057 g, 1.02 mmol) were added in order, and stirred at room temperature for 1 hour. After the completion of the reaction, the reaction mixture was concentrated under reduced pressure until the remained solution to be about 2-3 mL, and saturated NaHCO₃ aqueous solution (1-2 mL) was added and stirred. The obtained solid product from filtering was washed with water, dried with vacuum to yield compound 288 as bright brown solid (0.019 g, 73%)_{.}

¹H NMR (400 MHz, DMSO-d₆) δ 7.67 (d, 2H, J=8.0 Hz), 7.60 (dd, 1H, J=9.6, 2.8 Hz), 7.48 (dd, 1H, J=9.0, 4.2 Hz), 7.14 (d, 2H, J=8.0 Hz), 6.99 (td, 1H, J=9.2, 2.4 Hz), 5.52 (s, 2H), 3.68 (t, 2H, J=6.8 Hz), 3.51 (m, 9H), 3.05 (t, 2H, J=6.8 Hz), 2.49 (m, 8H); MS (ESI) m/z 510 (M⁺+H).

### Example 88

### Synthesis of Compound 289

### Step 1. Synthesis of (R)-methyl 4-((8-fluoro-2-(2-(2-(hydroxymethyl)pyrrolidin-1-yl)ethyl)-1-oxo-1,2,3,4-tetrahydropyrido[4,3-b]indol-5-yl)methyl)benzoate [formula 9-6]

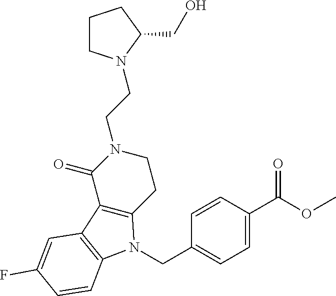 [SMILES: COC(=O)c1ccc(Cn2c3c(c4cc(F)ccc42)C(=O)N(CCN2CCC[C@@H]2CO)CC3)cc1]

Methyl 4-((8-fluoro-2-(2-hydroxyethyl)-1-oxo-1,2,3,4-tetrahydropyrido[4,3-b]indol-5-yl)methyl)benzoate [formula 9-5] (0.05 g, 0.13 mmol) was dissolved in ACN (1 mL). Thereto, DIPEA (0.11 mL, 0.63 mmol) and methanesulfonyl chloride (0.02 mL, 0.25 mmol) were added in order and stirred at room temperature for 1 minute, (R)-2-(hydroxymethyl)pyrrolidin (0.038 g, 0.38 mmol) was added and stirred at 50° C. for 30 minutes. After the completion of the reaction, the reaction mixture was purified by column chromatography (SiO₂; dichloromethane/methanol, 15/1) to yield the title compound as yellow solid (0.02 g, 33%).

### Step 2. Synthesis of (R)-4-((8-fluoro-2-(2-(2-(hydroxymethyl)pyrrolidin-1-yl)ethyl)-1-oxo-1,2,3,4-tetrahydropyrido[4,3-b]indol-5-yl)methyl)-N-hydroxybenzamide [formula 9-7]

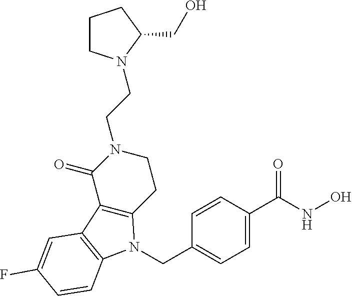 [SMILES: O=C(NO)c1ccc(Cn2c3c(c4cc(F)ccc42)C(=O)N(CCN2CCC[C@@H]2CO)CC3)cc1]

(R)-methyl 4-((8-fluoro-2-(2-(2-(hydroxymethyl)pyrrolidin-1-yl)ethyl)-1-oxo-1,2,3,4-tetrahydropyrido[4,3-b]indol-5-yl)methyl)benzoate [formula 9-6] (0.02 g, 0.042 mmol) was dissolved in methanol (5 mL). Thereto, hydroxylamine 50% aqueous solution (1.0 mL) and potassium hydroxide (0.047 g, 0.83 mmol) were added in order, and stirred at room temperature for 1 hour. After the completion of the reaction, the reaction mixture was concentrated under reduced pressure until the remained solution to be about 2-3 mL. The reaction mixture was extracted with saturated NaHCO₃ aqueous solution, ethyl acetate and THF. The organic layer was dried over MgSO₄, filtered and concentrated under reduced pressure. Residue was dried with vacuum to yield compound 289 as colorless liquid (0.004 g, 20%).

MS (ESI) m/z 481 (M⁺+H).

### Example 89

### Synthesis of Compound 290

### Step 1. Synthesis of (S)-methyl 4-((8-fluoro-2-(2-(2-(hydroxymethyl)pyrrolidin-1-yl)ethyl)-1-oxo-1,2,3,4-tetrahydropyrido[4,3-b]indol-5-yl)methyl)benzoate [formula 9-6]

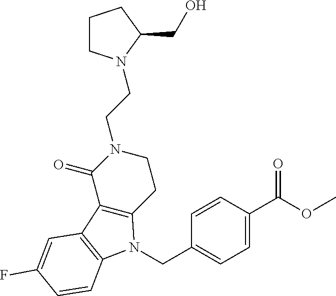 [SMILES: COC(=O)c1ccc(Cn2c3c(c4cc(F)ccc42)C(=O)N(CCN2CCC[C@H]2CO)CC3)cc1]

Methyl 4-((8-fluoro-2-(2-hydroxyethyl)-1-oxo-1,2,3,4-tetrahydropyrido[4,3-b]indol-5-yl)methyl)benzoate [formula 9-5] (0.05 g, 0.13 mmol) was dissolved in ACN (1 mL). Thereto, DIPEA (0.11 mL, 0.63 mmol) and methanesulfonyl chloride (0.02 mL, 0.25 mmol) were added in order and stirred at room temperature for 5 minute, (S)-2-(hydroxymethyl)pyrrolidin (0.038 g, 0.38 mmol) was added and stirred at 50° C. for 30 minutes. After the completion of the reaction, the reaction mixture was purified by column chromatography (SiO₂; dichloromethane/methanol, 15/1) to yield the title compound as yellow solid (0.025 g, 41%).

### Step 2. Synthesis of (S)-4-((8-fluoro-2-(2-(2-(hydroxymethyl)pyrrolidin-1-yl)ethyl)-1-oxo-1,2,3,4-tetrahydropyrido[4,3-b]indol-5-yl)methyl)-N-hydroxybenzamide [formula 9-7]

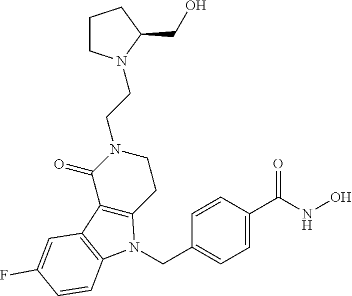 [SMILES: O=C(NO)c1ccc(Cn2c3c(c4cc(F)ccc42)C(=O)N(CCN2CCC[C@H]2CO)CC3)cc1]

(S)-Methyl 4-((8-fluoro-2-(2-(2-hydroxymethyl)pyrrolidin-1-yl)ethyl)-1-oxo-1,2,3,4-tetrahydropyrido[4,3-b]indol-5-yl)methyl)benzoate [formula 9-6] (0.025 g, 0.052 mmol) was dissolved in methanol (5 mL). Thereto, hydroxylamine 50% aqueous solution (1.3 mL) and potassium hydroxide (0.059 g, 1.04 mmol) were added in order, and stirred at room temperature for 1 hour. After the completion of the reaction, the reaction mixture was concentrated under reduced pressure until the remained solution to be about 2-3 mL, and saturated NaHCO₃ aqueous solution (1-2 mL) was added and stirred. The obtained solid product from filtering was washed with water and dried with vacuum to yield compound 290 as bright brown solid (0.014 g, 56%).

¹H NMR (400 MHz, DMSO-d₆) δ 7.67 (d, 2H, J=8.2 Hz), 7.60 (dd, 1H, J=9.6, 2.8 Hz), 7.48 (dd, 1H, J=9.0, 4.2 Hz), 7.14 (d, 2H, J=8.2 Hz), 6.98 (td, 1H, J=9.2, 2.4 Hz), 5.51 (s, 2H), 3.68 (m, 2H), 3.53 (m, 2H), 3.14 (m, 2H), 3.02 (m, 3H), 2.49 (m, 3H), 2.19 (m, 1H), 1.75 (m, 1H); 1.62 (m, 4H); MS (ESI) m/z 481 (M⁺+H).

### Example 90

### Synthesis of Compound 291

### Step 1. Synthesis of methyl 4-((8-fluoro-2-(2-(4-methyl-1,4-diazepan-1-yl)ethyl)-1-oxo-1,2,3,4-tetrahydropyrido[4,3-b]indol-5-yl)methyl)benzoate [formula 9-6]

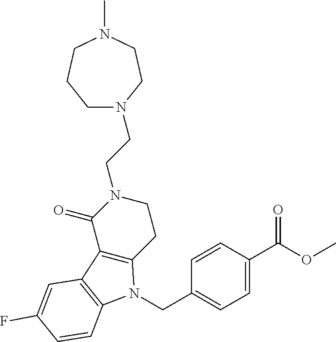 [SMILES: COC(=O)c1ccc(Cn2c3c(c4cc(F)ccc42)C(=O)N(CCN2CCCN(C)CC2)CC3)cc1]

Methyl 4-((8-fluoro-2-(2-hydroxyethyl)-1-oxo-1,2,3,4-tetrahydropyrido[4,3-b]indol-5-yl)methyl)benzoate [formula 9-5] (0.05 g, 0.13 mmol) was dissolved in ACN (1 mL). Thereto, DIPEA (0.11 mL, 0.63 mmol) and methanesulfonyl chloride (0.02 mL, 0.25 mmol) were added in order and stirred at room temperature for 5 minute, 1-methyl-1,4-diazepan (0.043 g, 0.38 mmol) was added and stirred at 50° C. for 30 minutes. After the completion of the reaction, the reaction mixture was purified by column chromatography (SiO₂; dichloromethane/methanol, 10/1) to yield the title compound as yellow liquid (0.018 g, 29%).

### Step 2. Synthesis of 4-((8-fluoro-2-(2-(4-methyl-1,4-diazepan-1-yl)ethyl)-1-oxo-1,2,3,4-tetrahydropyrido[4,3-b]indol-5-yl)methyl)-N-hydroxybenzamide [formula 9-7]

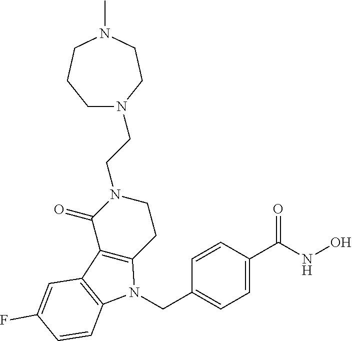 [SMILES: CN1CCCN(CCN2CCc3c(c4cc(F)ccc4n3Cc3ccc(C(=O)NO)cc3)C2=O)CC1]

Methyl 4-((8-fluoro-2-(2-(4-methyl-1,4-diazepan-1-yl)ethyl)-1-oxo-1,2,3,4-tetrahydropyrido[4,3-b]indol-5-yl)methyl)benzoate [formula 9-6] (0.018 g, 0.037 mmol) was dissolved in methanol (5 mL). Thereto, hydroxylamine 50% aqueous solution (0.9 mL), and potassium hydroxide (0.041 g, 0.73 mmol) were added in order, and stirred at room temperature for 1 hour. After the completion of the reaction, the reaction mixture was concentrated under reduced pressure until the remained solution to be about 2 mL. The reaction mixture was extracted with saturated NaHCO₃ aqueous solution, ethyl acetate and THF. The organic layer was dried over MgSO₄, filtered and concentrated under reduced pressure. Residue was dried with vacuum to yield compound 291 as colorless liquid (0.005 g, 28%).

MS (ESI) m/z 494 (M⁺+H).

### Example 91

### Synthesis of Compound 292

### Step 1. Synthesis of methyl 4-((8-fluoro-2-(2-((2-hydroxyethyl)(methyl)amino)ethyl)-1-oxo-1,2,3,4-tetrahydropyrido[4,3-b]indol-5-yl)methyl)benzoate [formula 9-6]

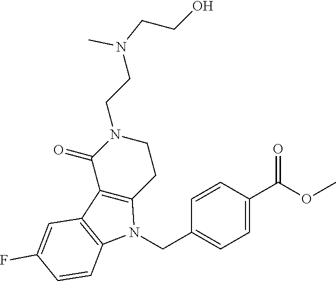 [SMILES: COC(=O)c1ccc(Cn2c3c(c4cc(F)ccc42)C(=O)N(CCN(C)CCO)CC3)cc1]

Methyl 4-((8-fluoro-2-(2-hydroxyethyl)-1-oxo-1,2,3,4-tetrahydropyrido[4,3-b]indol-5-yl)methyl)benzoate [formula 9-5] (0.05 g, 0.13 mmol) was dissolved in ACN (1 mL). Thereto, DIPEA (0.11 mL, 0.63 mmol) and methanesulfonyl chloride (0.02 mL, 0.25 mmol) were added in order and stirred at room temperature for 1 minute, 2-(methylamino)ethanol (0.028 g, 0.38 mmol) was added and stirred at 50° C. for 30 minutes. After the completion of the reaction, the reaction mixture was purified by column chromatography (SiO₂; dichloromethane/methanol, 15/1) to yield the title compound as yellow liquid (0.025 g, 44%).

### Step 2. Synthesis of 4-((8-fluoro-2-(2-((2-hydroxyethyl)(methyl)amino)ethyl)-1-oxo-1,2,3,4-tetrahydropyrido[4,3-b]indol-5-yl)methyl)-N-hydroxybenzamide [formula 9-7]

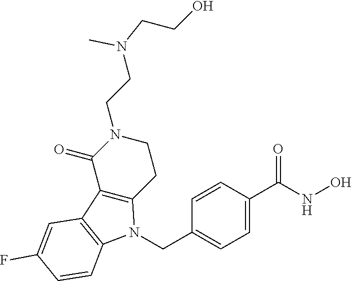 [SMILES: CN(CCO)CCN1CCc2c(c3cc(F)ccc3n2Cc2ccc(C(=O)NO)cc2)C1=O]

Methyl 4-((8-fluoro-2-(2-((2-hydroxyethyl)(methyl)amino)ethyl)-1-oxo-1,2,3,4-tetrahydropyrido[4,3-b]indol-5-yl)methyl)benzoate [formula 9-6] (0.025 g, 0.055 mmol) was dissolved in methanol (5 mL). Thereto, hydroxylamine 50% aqueous solution (1 mL) and potassium hydroxide (0.062 g, 1.10 mmol) were added in order, and stirred at room temperature for 1 hour. After the completion of the reaction, the reaction mixture was concentrated under reduced pressure until the remained solution to be about 2 mL. The reaction mixture was extracted with saturated NaHCO₃ aqueous solution, ethyl acetate and THF. The organic layer was dried over MgSO₄ hydrate, filtered and concentrated under reduced pressure. Residue was dried with vacuum to yield compound 292 as bright yellow liquid (0.006 g, 24%).

MS (ESI) m/z 455 (M⁺+H).

### Example 92

### Synthesis of Compound 305

### Step 1. Synthesis of methyl 4-((8-fluoro-1-oxo-1,2-dihydropyrido[4,3-b]indol-5-yl)methyl)benzoate [formula 10-1]

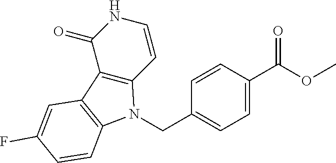 [SMILES: COC(=O)c1ccc(Cn2c3ccc(F)cc3c3c(=O)[nH]ccc32)cc1]

Methyl 4-((8-fluoro-1-oxo-1,2,34-tetrahydropyrido[4,3-b]indol-5-yl)methyl)benzoate [formula 10-4] (0.20 g, 0.57 mmol) was dissolved in 1,4-dioxane (3 mL), and 2,3-dichloro-5,6-dicyanobenzoquinone (0.14 g, 0.62 mmol) was added and refluxed with stirring for 16 hours. After the completion of the reaction, the reaction mixture was extracted with ethyl acetate and saturated NaHCO₃ aqueous solution, the organic layer was washed with MgSO₄ hydrate and filtered. The filtrate was concentrated under reduced pressure, and residue was purified by column chromatography (SiO₂; dichloromethane/methanol, 20/1) to yield the title compound as bright yellow solid (0.12 g, 60%).

### Step 2. Synthesis of 4-((8-fluoro-1-oxo-1,2-dihydropyrido[4,3-b]indol-5-yl)methyl)-N-hydroxybenzamide [formula 10-2]

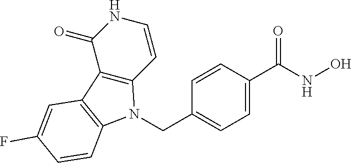 [SMILES: O=C(NO)c1ccc(Cn2c3ccc(F)cc3c3c(=O)[nH]ccc32)cc1]

Methyl 4-((8-fluoro-1-oxo-1,2-dihydropyrido[4,3-b]indol-5-yl)methyl)benzoate [formula 10-1] (0.12 g, 0.34 mmol) was dissolved in methanol (5 mL) and THF (3 mL). Thereto, hydroxylamine 50% aqueous solution (2.1 mL) and potassium hydroxide (0.19 g, 3.43 mmol) were added in order, and stirred at room temperature for 30 minutes. After the completion of the reaction, the reaction mixture was concentrated under reduced pressure until the remained solution to be about 2 mL, and saturated NaHCO₃ aqueous solution (1-2 mL) was added and stirred. The obtained solid product from filtering was washed with water and dried with vacuum to yield compound 305 as white solid (0.10 g, 85%).

¹H NMR (400 MHz, DMSO-d₆) δ 7.79 (m, 1H), 7.73-7.58 (m, 3H), 7.42 (m, 1H), 7.21-7.7.14 (m, 3H), 6.75 (d, 1H, J=7.2 Hz), 5.68 (s, 2H); MS (ESI) m/z 352 (M⁺+H).

### Example 93

### Synthesis of Compound 306

### Synthesis of 4-((8-fluoro-1-oxo-1,2,3,4-tetrahydropyrido[4,3-b]indol-5-yl)methyl)-N-hydroxybenzamide [formula 10-3]

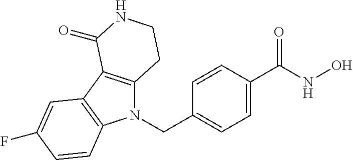 [SMILES: O=C(NO)c1ccc(Cn2c3c(c4cc(F)ccc42)C(=O)NCC3)cc1]

Methyl 4-((8-fluoro-1-oxo-1,2,3,4-tetrahydropyrido[4,3-b]indol-5-yl)methyl)benzoate [formula 10-4] (0.05 g, 0.14 mmol) was dissolved in methanol (5 mL) and THF (3 mL). Thereto, hydroxylamine 50% aqueous solution (1.7 mL) and potassium hydroxide (0.080 g, 1.42 mmol) were added in order, and stirred at room temperature for 30 minutes. After the completion of the reaction, the reaction mixture was concentrated under reduced pressure until the remained solution to be about 2 mL, and saturated NaHCO₃ aqueous solution (1-2 mL) was added and stirred. The obtained solid product from filtering was washed with water and dried with vacuum to yield compound 306 as white solid (0.036 g, 72%).

¹H NMR (400 MHz, DMSO-d₆) δ 7.67 (d, 2H, J=8.4 Hz), 7.58 (dd, 1H, J=9.7, 2.8 Hz), 7.49 (dd, 1H, J=9.2, 4.4 Hz), 7.19 (s, 1H), 7.15 (d, 2H, J=8.4 Hz), 6.99 (td, 1H, J=9.1, 2.7 Hz), 5.52 (s, 2H), 3.47 (m, 2H), 2.98 (t, 2H, J=7.2 Hz).

### Example 94

### Synthesis of Compound 321

### Step 1. Synthesis of ethyl 6-(3-fluoro-5-oxo-5,6,7,8-tetrahydrocarbazol-9-yl)hexanoate [formula 1-3]

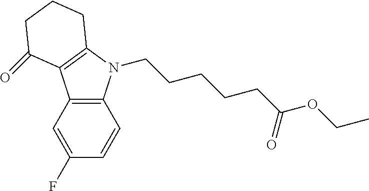 [SMILES: CCOC(=O)CCCCCn1c2c(c3cc(F)ccc31)C(=O)CCC2]

6-Fluoro-2,3-dihydro-1H-carbazol-4(9H)-on [formula 2-2] (1.16 g, 5.708 mmol) was dissolve in ACN (30 mL), and cesium carbonate (Cs₂CO₃)(2.79 g, 8.563 mmol) was added and stirred for 10 minutes. Then, ethyl-6-bromohexanoate (1.528 g, 6.85 mmol) was added and stirred with heating for 3 hours. After the completion of the reaction, the reaction mixture was extracted with ethyl acetate and H₂O, the organic layer was washed with brine, dried over anhydrous Na₂SO₄, filtered and concentrated under reduced pressure. Residue was purified by column chromatography (SiO₂; hexane/ethylacetate, 5/1) to yield the title compound (1.71 g, 87%).

### Step 2. Synthesis of ethyl 6-(6-fluoro-3-methylen-4-oxo-1,2,3,4-tetrahydrocarbazol-9-yl)hexanoate [formula 2-7]

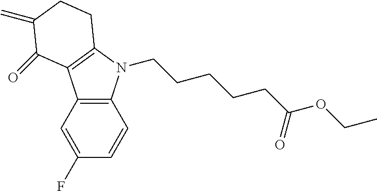 [SMILES: C=C1CCc2c(c3cc(F)ccc3n2CCCCCC(=O)OCC)C1=O]

Ethyl 6-(3-fluoro-5-oxo-5,6,7,8-tetrahydrocarbazol-9-yl)hexanoate [formula 1-3] (1.71 g, 4.951 mmol), N,N-dimethylamine, HCl (0.8074 g, 9.901 mmol), paraformaldehyde (0.329 g, 9.901 mmol) and mixed solvent (acetic acid:toluene=4:1, 15 mL) were added, and stirred at 100° C. for 4 hours. After the completion of the reaction, acetic acid was distilled out under reduced pressure, and without purification, the compound was dissolved in a 15 mL of mixed solvent (acetonitrile:H₂O=1:4) and stirred at 80° C. for 12 hours. After the completion of the reaction, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 7/1) to yield the title compound (1.1 g, 62%).

### Step 3. Synthesis of ethyl 6-(6-(morpholinomethyl)-4-oxo-1,2,3,4-tetrahydrocarbazol-9-yl)hexaoate [formula 2-8]

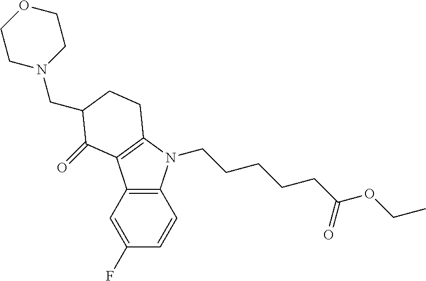 [SMILES: CCOC(=O)CCCCCn1c2c(c3cc(F)ccc31)C(=O)C(CN1CCOCC1)CC2]

To a microwave vial were added ethyl 6-(6-fluoro-3-methylen-4-oxo-1,2,3,4-tetrahydrocarbazol-9-yl)hexanoate [formula 2-7] (0.10 g, 0.295 mmol), morpholine (0.077 g, 0.884 mmol) and toluene (4.0 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.079 g, 62%).

### Step 4. Synthesis of 6-(6-fluoro-3-(morpholinomethyl)-4-oxo-1,2,3,4-tetrahydrocarbazol-9-yl)-N-hydroxyhexanamide [formula 2-9]

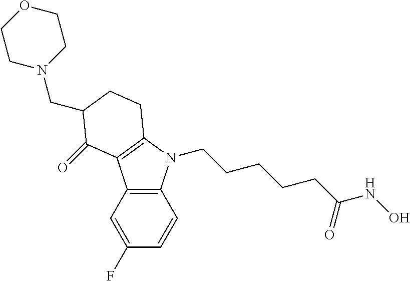 [SMILES: O=C(CCCCCn1c2c(c3cc(F)ccc31)C(=O)C(CN1CCOCC1)CC2)NO]

To a flask were added ethyl 6-(6-fluoro-3-(morpholinomethyl)-4-oxo-1,2,3,4-tetrahydrocarbazol-9-yl)hexaoate [formula 2-8] (0.075 g, 0.169 mmol), hydroxylamine hydrochloride (NH₂OH HCl) (0.0586 g, 0.844 mmol), potassium hydroxide (0.095 g, 1.687 mmol) and methanol (10 mL), and stirred for 10 minutes. Then hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 321 as solid (0.032 g, 44%).

¹H NMR (400 MHz, DMSO-d₆) δ 10.3 (s, 1H), 8.67 (s, 1H), 7.66-7.56 (m, 2H), 7.08-7.04 (m, 1H), 4.16-4.08 (m, 2H), 3.57 (s, 4H), 3.08-2.98 (m, 2H), 2.68-2.66 (m, 2H), 2.45-2.29 (m, 3H), 1.98-1.89 (m, 3H), 1.75-1.68 (m, 4H), 1.59-1.48 (m, 2H), 1.39-1.14 (m, 3H); MS (ESI) m/z 432 (M++H).

### Example 95

### Synthesis of Compound 322

### Step 1. Synthesis of ethyl 6-(6-fluoro-3-((4-(4-fluorophenyl)piperazin-1-yl)methyl)-4-oxo-1,2,3,4-tetrahydrocarbazol-9-yl)hexaoate [formula 2-8]

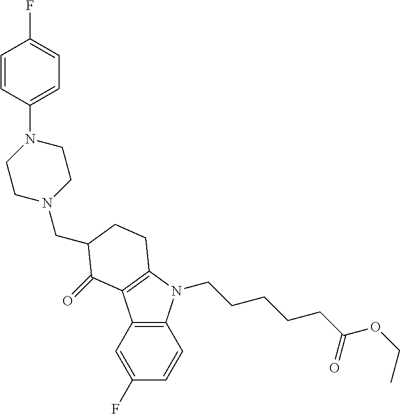 [SMILES: CCOC(=O)CCCCCn1c2c(c3cc(F)ccc31)C(=O)C(CN1CCN(c3ccc(F)cc3)CC1)CC2]

To a microwave vial were added ethyl 6-(6-fluoro-3-methylen-4-oxo-1,2,3,4-tetrahydrocarbazol-9-yl)hexanoate [formula 2-7] (0.10 g, 0.28 mmol), 1-(4-fluoro)piperazine (0.151 g, 0.839 mmol) and toluene (10 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.053 g, 35%).

### Step 2. Synthesis of 6-(6-fluoro-3-((4-(4-fluorophenyl)piperazin-1-yl)methyl)-4-oxo-1,2,3,4-tetrahydrocarbazol-9-yl)-N-hydroxyhexanamide [formula 2-9]

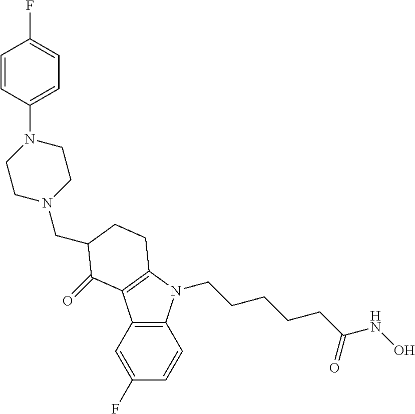 [SMILES: O=C(CCCCCn1c2c(c3cc(F)ccc31)C(=O)C(CN1CCN(c3ccc(F)cc3)CC1)CC2)NO]

To a flask were added ethyl 6-(6-fluoro-3-((4-(4-fluorophenyl)piperazin-1-yl)methyl)-4-oxo-1,2,3,4-tetrahydrocarbazol-9-yl)hexanoate [formula 2-8] (0.255 g, 0.474 mmol), hydroxylamine hydrochloride (NH₂OH HCl) (0.165 g, 2.371 mmol), potassium hydroxide (0.266 g, 4.74 mmol) and methanol (10 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 322 as solid (0.098 g, 39%).

¹H NMR (400 MHz, DMSO-d₆) δ 10.3 (s, 1H), 8.67 (s, 1H), 7.67-7.56 (m, 2H), 7.09-6.94 (m, 5H), 4.17 (s, 2H), 3.07-2.90 (m, 5H), 2.73-2.63 (m, 4H), 2.49-2.46 (m, 3H), 2.42-2.33 (m, 1H), 1.98-1.90 (m, 3H), 1.79-1.68 (m, 2H), 1.59-1.52 (m, 2H), 1.26-1.14 (m, 3H); MS (ESI) m/z 525 (M++H).

### Example 96

### Synthesis of Compound 323

### Step 1. Synthesis of ethyl 6-(3-((2,6-dimethylmorpholino)methyl)-6-fluoro-4-oxo-1,2,3,4-tetrahydrocarbazol-9-yl)hexanoate [formula 2-8]

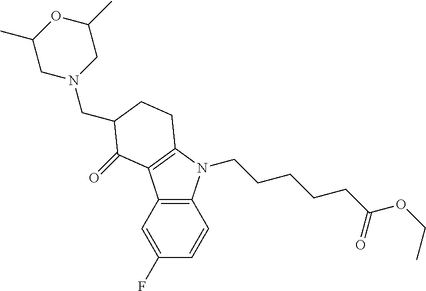 [SMILES: CCOC(=O)CCCCCn1c2c(c3cc(F)ccc31)C(=O)C(CN1CC(C)OC(C)C1)CC2]

To a microwave vial were added ethyl 6-(6-fluoro-3-methylen-4-oxo-1,2,3,4-tetrahydrocarbazol-9-yl)hexanoate [formula 2-7] (0.10 g, 0.28 mmol), 2,6-dimethylmorpholine (0.097 g, 0.839 mmol) and toluene (10 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.049 g, 37%).

### Step 2. Synthesis of 6-(3-((2,6-dimethylmorpholino)methyl)-6-fluoro-4-oxo-1,2,3,4-tetrahydrocarbazol-9-yl)-N-hydroxyhexanamide [formula 2-9]

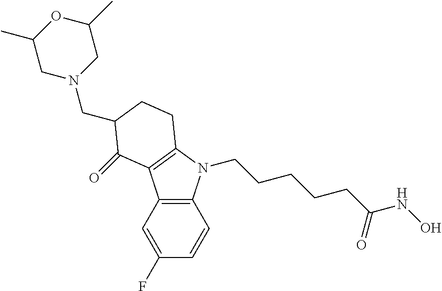 [SMILES: CC1CN(CC2CCc3c(c4cc(F)ccc4n3CCCCCC(=O)NO)C2=O)CC(C)O1]

To a flask were added ethyl 6-(3-((2,6-dimethylmorpholino)methyl)-6-fluoro-4-oxo-1,2,3,4-tetrahydrocarbazol-9-yl)hexanoate [formula 2-8] (0.049 g, 0.104 mmol), hydroxylamine hydrochloride (NH₂OH HCl) (0.036 g, 0.518 mmol), potassium hydroxide (0.58 g, 1.04 mmol) and methanol (10 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 323 as solid (0.084 g, 58%).

¹H NMR (400 MHz, DMSO-d₆) δ 10.3 (s, 1H), 8.68 (s, 1H), 7.66-7.55 (m, 2H), 7.08-7.03 (m, 1H), 4.18-4.01 (m, 2H), 3.55-3.35 (m, 1H), 3.17-2.84 (m, 2H), 2.83-2.80 (m, 1H), 2.68-2.49 (m, 2H), 2.38-2.30 (m, 1H), 1.98-1.89 (m, 2H), 1.75 (s, 3H), 1.72 (s, 3H), 1.49-1.42 (m, 3H), 1.27-1.21 (m, 2H), 1.19-1.16 (m, 2H), 1.14-1.01 (m, 4H); MS (ESI) m/z 460 (M++H).

### Example 97

### Synthesis of Compound 324

### Step 1. Synthesis of tert-butyl 4-((9-(6-etoxy-6-oxohexyl)-6-fluoro-4-oxo-2,3,4,9-tetrahydro-1H-carbazol-3-yl)methyl)piperazin-1-carboxylate [formula 2-8]

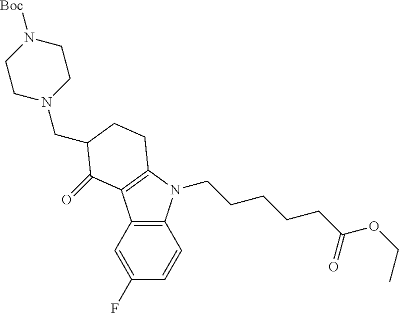 [SMILES: CCOC(=O)CCCCCn1c2c(c3cc(F)ccc31)C(=O)C(CN1CCN(C(=O)OC(C)(C)C)CC1)CC2]

To a microwave vial were added ethyl 6-(6-fluoro-3-methylen-4-oxo-1,2,3,4-tetrahydrocarbazol-9-yl)hexanoate [formula 2-7] (0.10 g, 0.28 mmol), tert-butylpiperazin-1-carboxylate (0.156 g, 0.839 mmol) and toluene (10 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.041 g, 27%).

### Step 2. Synthesis of tert-butyl 4-((6-fluoro-9-(6-(hydroxyamino)-6-oxohexyl)-4-oxo-2,3,4,9-tetrahydro-1H-carbazol-3-yl)methyl)piperazine-1-carboxylate [formula 2-9]

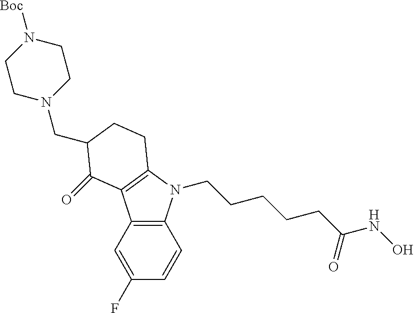 [SMILES: CC(C)(C)OC(=O)N1CCN(CC2CCc3c(c4cc(F)ccc4n3CCCCCC(=O)NO)C2=O)CC1]

To a flask were added tert-butyl 4-((9-(6-etoxy-6-oxohexyl)-6-fluoro-4-oxo-2,3,4,9-tetrahydro-1H-carbazol-3-yl)methyl)piperazine-1-carboxylate [formula 2-8] (0.14 g, 0.265 mmol), hydroxylamine hydrochloride (NH₂OH HCl) (0.092 g, 1.324 mmol), potassium hydroxide (0.149 g, 2.65 mmol) and methanol (10 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 324 as solid (0.099 g, 70%).

¹H NMR (400 MHz, DMSO-d₆) δ 10.3 (s, 1H), 8.68 (s, 1H), 7.66-7.56 (m, 2H), 7.08-7.04 (m, 1H), 4.16 (brs, 2H), 4.02-4.00 (m, 1H), 3.08-2.96 (m, 2H), 2.68-2.66 (m, 2H), 2.39-2.31 (m, 1H), 2.25-2.23 (m, 3H), 1.98-1.89 (m, 4H), 1.68-1.61 (m, 2H), 1.59-1.44 (m, 2H), 1.43-1.38 (m, 11H), 1.27-1.23 (m, 3H), 1.18-1.14 (m, 1H); MS (ESI) m/z 531 (M++H).

### Example 98

### Synthesis of Compound 325

### Step 1. Synthesis of ethyl 7-(3-fluoro-5-oxo-5,6,7,8-tetrahydrocarbazol-9-yl)heptanoate [formula 1-3]

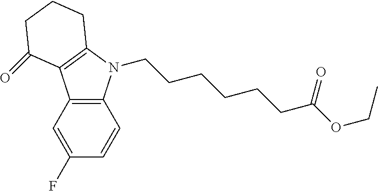 [SMILES: CCOC(=O)CCCCCCn1c2c(c3cc(F)ccc31)C(=O)CCC2]

6-fluoro-2,3-dihydro-1H-carbazol-4(9H)-on [formula 1-2] (2.1 g, 10.33 mmol) was dissolved in ACN (30 mL), and cesium carbonate (Cs₂CO₃)(5.05 g, 15.5 mmol) was added and stirred for 10 minutes. Then, ethyl-7-bromoheptanoate (2.76 g, 12.40 mmol) was added and stirred at 50° C. for 5 hours. After the completion of the reaction, the reaction mixture was extracted with ethyl acetate and H₂O, the organic layer was washed with brine, dried over anhydrous Na₂SO₄, filtered and concentrated under reduced pressure. Residue was purified by column chromatography (SiO₂; hexane/ethylacetate, 5/1) to yield the title compound (1.93 g, 52%).

### Step 2. Synthesis of 7-(3-fluoro-5-oxo-5,6,7,8-tetrahydrocarbazol-9-yl)-N-hydroxyheptanamide [formula 1-4]

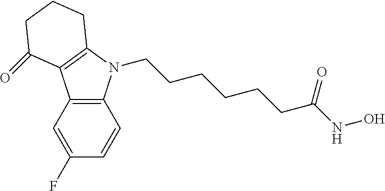 [SMILES: O=C(CCCCCCn1c2c(c3cc(F)ccc31)C(=O)CCC2)NO]

To a flask were added ethyl 7-(3-fluoro-5-oxo-5,6,7,8-tetrahydrocarbazol-9-yl)heptanoate [formula 1-3] (0.20 g, 0.556 mmol), hydroxylamine hydrochloride (NH₂OH HCl) (0.193 g, 2.782 mmol), potassium hydroxide (0.312 g, 5.564 mmol) and methanol (10 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. Residue was treated with 2N HCl and filtered to yield the compound 325 as solid (0.135 g, 70%).

¹H NMR (400 MHz, DMSO-d₆) δ 10.3 (s, 1H), 9.00 (s, 1H), 7.66-7.46 (m, 2H), 7.07 (s, 1H), 4.27 (s, 2H), 3.07-2.92 (m, 2H), 2.45-2.38 (m, 2H), 2.18-2.03 (m, 2H), 1.88-1.83 (m, 2H), 1.79-1.60 (m, 2H), 1.45 (brs, 2H), 1.26-1.17 (m, 4H); MS (ESI) m/z 347 (M++H).

### Example 99

### Synthesis of Compound 326

### Step 1. Synthesis of ethyl 6-(3-((4-butylpiperazin-1-yl)methyl)-6-fluoro-4-oxo-1,2,3,4-tetrahydrocarbazol-9-yl)hexanoate [formula 2-8]

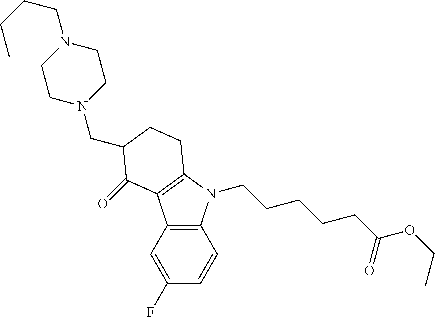 [SMILES: CCCCN1CCN(CC2CCc3c(c4cc(F)ccc4n3CCCCCC(=O)OCC)C2=O)CC1]

To a microwave vial were added ethyl 6-(6-fluoro-3-methylen-4-oxo-1,2,3,4-tetrahydrocarbazol-9-yl)hexanoate [formula 2-7] (0.10 g, 0.28 mmol), 1-butylpiperazin (0.119 g, 0.839 mmol) and toluene (10 mL), and a reaction was carried out in a microwave reactor at 120° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.052 g, 37%).

### Step 2. Synthesis of 6-(3-((4-butylpiperazin-1-yl)methyl)-6-fluoro-4-oxo-1,2,3,4-tetrahydrocarbazol-9-yl)-N-hydroxyhexanamide [formula 2-9]

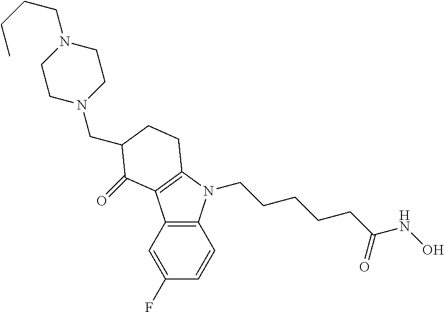 [SMILES: CCCCN1CCN(CC2CCc3c(c4cc(F)ccc4n3CCCCCC(=O)NO)C2=O)CC1]

To a flask were added ethyl 6-(3-((4-butylpiperazin-1-yl)methyl)-6-fluoro-4-oxo-1,2,3,4-tetrahydrocarbazol-9-yl)hexanoate [formula 2-8] (0.052 g, 0,104 mmol), hydroxylamine hydrochloride (NH₂OH HCl) (0.036 g, 0.52 mmol), potassium hydroxide (0.058 g, 1.041 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. And the reaction mixture was re-crystallized by diethyl ether and ethyl acetate, filtered to yield the compound 326 (0.038 g, 75%).

¹H NMR (400 MHz, DMSO-d₆) δ 10.3 (s, 1H), 8.68 (s, 1H), 7.65-7.57 (m, 2H), 7.08-7.04 (m, 1H), 4.16 (s, 2H), 3.07-2.92 (m, 2H), 2.64-2.62 (m, 2H), 2.32-2.22 (m, 8H), 1.93-1.91 (m, 3H), 1.76-1.64 (m, 3H), 1.58-1.42 (m, 2H), 1.41-1.28 (m, 2H), 1.27-1.18 (m, 6H), 0.88-0.84 (m, 4H); MS (ESI) m/z 487 (M++H).

### Example 100

### Synthesis of Compound 328

### Step 1. Synthesis of 2,3-dimethyl-6,7-dihydro-1H-indole-4(5H)-on [formula 6-2]

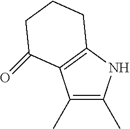

2,3-butanedion 2-oxime [formula 6-1] (5.0 g, 49.5 mmol) and 1,3-cyclohexandion [formula 6-7] (5.55 g, 49.5 mmol) were dissolved in acetic acid (70 mL) and H₂O (30 mL). Thereto, zinc powder (6.60 g, 98.9 mmol) was added slowly maintaining room temperature. The reaction mixture was refluxed with stirring for 1 day, concentrated under reduced pressure and extracted with CH₂Cl₂ and brine, of which pH was adjusted to 6 using saturated NaHCO₃. The reaction mixture was extracted with CH₂Cl₂; organic layer was dried over anhydrous MgSO₄ and filtered. The filtrate was concentrated under reduced pressure and purified by column chromatography (SiO₂; hexane/ethylacetate, 7/3) to yield the title compound as yellow solid (3.26 g, 40%).

### Step 2. Synthesis of ethyl 7-(2,3-dimethyl-4-oxo-4,5,6,7-tetrahydro-1H-indole-1-yl)heptanoate [formula 6-5]

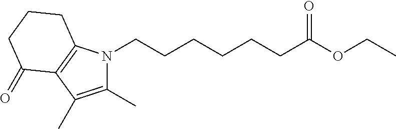 [SMILES: CCOC(=O)CCCCCCn1c(C)c(C)c2c1CCCC2=O]

Ethyl 7-(2,3-dimethyl-4-oxo-4,5,6,7-tetrahydro-1H-indole-1-yl)heptanoate [formula 6-2] (1.0 g, 6.13 mmol) was dissolved DMF, NaH (0.18 g, 7.35 mmol) was added slowly maintaining room temperature, and stirred for 5 minutes. Ethyl 7-bromoheptanoate (1.43 mL, 7.35 mmol) was added and stirred at room temperature for 1 day. After the completion of reaction, the reaction mixture was extracted with ethyl acetate and saturated NH₄Cl aqueous solution, the organic layer was dried over anhydrous MgSO₄ and filtered. The filtrate was concentrated under reduced pressure and purified by column chromatography (SiO₂; hexane/ethylacetate, 7/3) to yield the title compound as transparent oil (1.3 g, 66%).

### Step 3. Synthesis of 7-(2,3-dimethyl-4-oxo-4,5,6,7-tetrahydro-1H-indol-1-yl)-N-hydroxyheptanamide [formula 6-6]

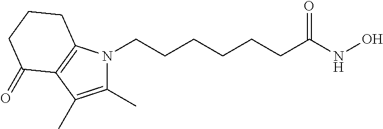 [SMILES: Cc1c2c(n(CCCCCCC(=O)NO)c1C)CCCC2=O]

Ethyl 7-(2,3-dimethyl-4-oxo-4,5,6,7-tetrahydro-1H-indole-1-yl)heptanoate [formula 6-5] (0.2 g, 0.63 mmol) was dissolved in methanol (5 mL), and hydroxylamine hydrochloride (NH₂OH HCl) (0.22 g, 3.13 mmol) and potassium hydroxide (0.53 g, 6.26 mmol) were added and stirred. Hydroxylamine 50% aqueous solution (2 mL) was added and stirred at room temperature for 4 hours. After the completion of the reaction, methanol was distilled out under reduced pressure, water was added, extracted with ethyl acetate, the organic layer was dried over anhydrous MgSO₄ and filtered. The filtrate was concentrated under reduced pressure, ether was added to obtain solid product, the reaction mixture was filtered and dried under reduced pressure to yield the compound 328 as white solid (0.11 g, 57%).

### Example 101

### Synthesis of Compound 344

### Step 1. Synthesis of ethyl 7-(6-fluoro-3-methylen-4-oxo-1,2,3,4-tetrahydrocarbazol-9-yl)heptanoate [formula 2-7]

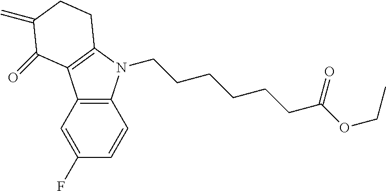 [SMILES: C=C1CCc2c(c3cc(F)ccc3n2CCCCCCC(=O)OCC)C1=O]

To a flask were added ethyl 7-(3-fluoro-5-oxo-5,6,7,8-tetrahydrocarbazol-9-yl)heptanoate [formula 1-3] (1.6 g, 4.451 mmol), N,N-dimethylamine, HCl (0.726 g, 8.903 mmol), paraformaldehyde (0.296 g, 8.903 mmol) and mixed solvent (acetic acid:toluene=4:1, 15 mL) were added, and stirred at 100° C. for 4 hours. After the completion of the reaction, acetic acid was distilled out under reduced pressure, and without purification, the compound was dissolved in a 15 mL of mixed solvent (acetonitrile:H₂O=1:4) and stirred at 80° C. for 12 hours. After the completion of the reaction, the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 7/1) to yield the title compound (1.2 g, 73%).

### Step 2. Synthesis of ethyl 7-(6-fluoro-3-(morpholinomethyl)-4-oxo-1,2,3,4-tetrahydrocarbazol-9-yl)heptanoate [formula 2-8]

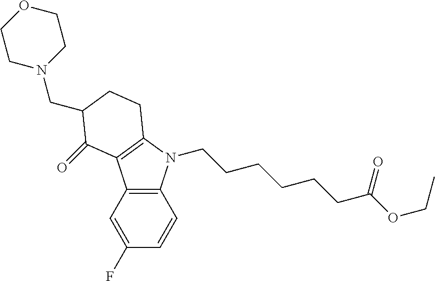 [SMILES: CCOC(=O)CCCCCCn1c2c(c3cc(F)ccc31)C(=O)C(CN1CCOCC1)CC2]

To a microwave vial were added ethyl 7-(6-fluoro-3-methylen-4-oxo-1,2,3,4-tetrahydrocarbazol-9-yl)heptanoate [formula 2-7] (0.1 g, 0.269 mmol), morpholine (0.070 g, 0.808 mmol) and toluene (10 mL), and a reaction was carried out in a microwave reactor at 110° C. for 90 minutes. Then, the reaction mixture was extracted with ethyl acetate and saturated NH₄Cl aqueous solution, the organic layer was washed with brine, dried over anhydrous Na₂SO₄, filtered and concentrated under reduced pressure. Residue was purified by column chromatography (SiO₂; dichloromethane/methanol, 10/1) to yield the title compound (0.072 g, 58%).

### Step 3. Synthesis of 7-(6-fluoro-3-(morpholinomethyl)-4-oxo-1,2,3,4-tetrahydrocarbazol-9-yl)-N-hydroxyheptanamide [formula 2-9]

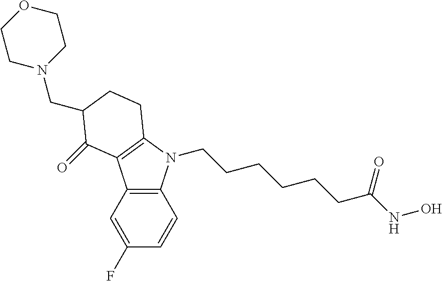 [SMILES: O=C(CCCCCCn1c2c(c3cc(F)ccc31)C(=O)C(CN1CCOCC1)CC2)NO]

To a flask were added ethyl 7-(6-fluoro-3-(morpholinomethyl)-4-oxo-1,2,3,4-tetrahydrocarbazol-9-yl)heptanoate [formula 2-8] (0.072 g, 0.157 mmol), hydroxylamine hydrochloride (NH₂OH HCl) (0.0546 g, 0.785 mmol), potassium hydroxide (0.088 g, 1.57 mmol) and methanol (5.0 mL), and stirred for 10 minutes. Then, hydroxylamine 50% aqueous solution had been added drop-wise slowly until the mixed solution in the flask became clear, and the mixture was stirred at room temperature for 4 hours. After the completion of the reaction, methanol was distilled out under reduced pressure, and the reaction mixture was extracted with ethyl acetate only, dried over anhydrous Na₂SO₄, filtered and concentrated under reduced pressure. And the reaction mixture was re-crystallized by diethyl ether and ethyl acetate, filtered to yield the compound 344 (0.032 g, 44%). MS (ESI) m/z 446 (M++H).

### Example 102

### Synthesis of Compound 345

### Step 1. Synthesis of 5,5-dimethyl-2-(2-oxoprophyl)cyclohexane-1,3-dion [formula 11-2]

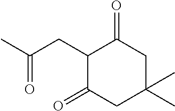

Potassium hydroxide (2.64 g, 47.08 mmol) and H₂O (5.0 mL) were mixed, and stirred for 30 minutes. Then 5,5-dimethyl-2-(2-oxoprophyl)cyclohexane-1,3-dion [formula 11-1] (6.0 g, 42.8 mmol) and ethanol (20.0 mL) were added and stirred for 15 minutes, 1-chloropropan-2-on (13.86 g, 149.81 mmol) were added and stirred at room temperature for 12 hours. After the completion of the reaction, inorganic materials were filtered out; ethanol was distilled out under the reduced pressure. The reaction mixture was extracted with ethyl acetate and saturated NaHCO₄₃ aqueous solution; the organic layer was washed with brine, dried over anhydrous Na₂SO₄, filtered and concentrated under reduced pressure. Without any purification, obtained compound was used in the next step.

### Step 2. Synthesis of 2,6,6-trimethyl-6,7-dihydro-1H-indol-4(H)-on [formula 11-3]

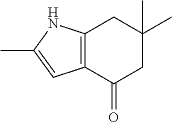

5,5-dimethyl-2-(2-oxoprophyl)cyclohexane-1,3-dion [formula 11-2] (7.0 g, 35.67 mmol), ammonium acetate (16.22 g, 210.46 mmol) and acetic acid (30 mL) were mixed and stirred at 140° C. for 3 hours. After the completion of the reaction, acetic acid was distilled out under the reduced pressure. The reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (2.1 g, 33%)

### Step 3. Synthesis of ethyl 6-(2,6,6-trimethyl-4-oxo-4,5,6,7-tetrahydroindol-1-yl)hexanoate [Formula 11-4]

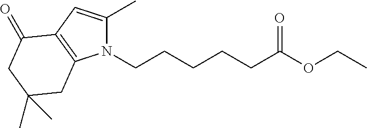 [SMILES: CCOC(=O)CCCCCn1c(C)cc2c1CC(C)(C)CC2=O]

(2,6,6-trimethyl-6,7-dihydro-1H-indol-4(5H)-on [formula 11-3] (0.20 g, 1.128 mmol) was dissolved in DMF (10 mL), 55% NaH in paraffin solution (0.0985 g, 2.257 mmol) was added and stirred for 10 minutes. Then, ethyl 6-bromohexanoate (0.302 g, 1.354 mmol) was added and stirred at room temperature for 3 hours. After the completion of the reaction, DMF was distilled out under reduced pressure, and the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 5/1) to yield the title compound (0.287 g, 80%).

### Step 4. Synthesis of 6-(2,6,6-trimethyl-4-oxo-4,5,6,7-tetrahydroindol-1-yl)hexanoic acid [formula 11-5]

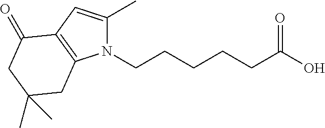 [SMILES: Cc1cc2c(n1CCCCCC(=O)O)CC(C)(C)CC2=O]

Ethyl 6-(2,6,6-trimethyl-4-oxo-4,5,6,7-tetrahydroindol-1-yl)hexanoate [Formula 11-4] (0.287 g, 0.898 mmol) was dissolved in mixed solvent (methanol:H₂O=2:1, 15 mL). Thereto, lithium hydroxide monohydrate (0.377 g, 8.98 mmol) was added and stirred at room temperature for 1 hour. After the completion of the reaction, methanol was distilled out under reduced pressure, acidized with 2N HCl and filtered. Solid product was washed with water sufficiently, and dried with vacuum to yield the title compound (0.189 g, 72%).

### Step 5. Synthesis of N-(tetrahydro-2H-pyran-2-yloxy)-6-(2,6,6-trimethyl-4-oxo-4,5,6,7-tetrahydroindol-1-yl)hexanamide [formula 11-6]

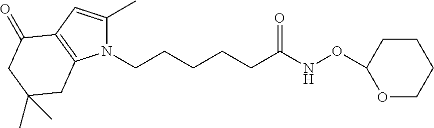 [SMILES: Cc1cc2c(n1CCCCCC(=O)NOC1CCCCO1)CC(C)(C)CC2=O]

6-(2,6,6-trimethyl-4-oxo-4,5,6,7-tetrahydroindol-1-yl)hexanoic acid [formula 11-5] (0.189 g, 0.649 mmol) was dissolved in dichloromethane (10.0 mL). Thereto, o-(tetrahydro-2H-pyran-2-yl)hydroxylamine (0.114, g, 0.973 mmol), EDC (0.2487 g, 0.973 mmol) and HOBt (0.175 g, 1.297 mmol) were added in order, DIPEA (0.4192 g, 3.243 mmol) was added and stirred at room temperature for 12 hours. After the completion of the reaction, the reaction mixture was extracted with dichloromethane and saturated NaHCO₃ aqueous solution, the organic layer was washed with brine, dried over anhydrous MgSO₄, filtered and concentrated under reduced pressure. Residue was purified by column chromatography (SiO₂; hexane/ethylacetate, 4/1) to yield the title compound (0.21 g, 83%)

### Step 6. Synthesis of N-hydroxy-6-(2,6,6-trimethyl-4-oxo-4,5,6,7-tetrahydroindol-1-yl)hexanamide [formula 11-7]

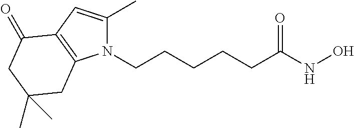 [SMILES: Cc1cc2c(n1CCCCCC(=O)NO)CC(C)(C)CC2=O]

N-(tetrahydro-2H-pyran-2-yloxy)-6-(2,6,6-trimethyl-4-oxo-4,5,6,7-tetrahydroindol-1-yl)hexanamide [formula 11-6] (0.287 g, 0.735 mmol) was dissolved in methanol (3.0 mL). Thereto, 1.25 M HCl in methanol (2.94 mL, 3.675 mmol) was added and stirred at room temperature for 1 hour. After the completion of the reaction, methanol was distilled out under reduced pressure, and the reaction mixture was extracted with ethyl acetate and saturated NaHCO₃ aqueous solution, the organic layer was washed with brine, dried over anhydrous Na₂SO₄, filtered and concentrated under reduced pressure. Residue was re-crystallized by dichloromethane/hexane to yield the compound 345 (0.095 g, 42%).

¹H NMR (400 MHz, DMSO-d₆) δ 10.3 (s, 1H), 8.67 (s, 1H), 6.00 (s, 1H), 3.75 (t, 2H, J=7.4 Hz), 3.33 (s, 3H), 2.61 (s, 2H), 2.16 (s, 4H), 1.92 (t, 2H, J=7.3 Hz), 1.57-1.47 (m, 4H), 1.27-1.23 (m, 2H), 1.02 (s, 6H); MS (ESI) m/z 307 (M++H).

### Example 103

### Synthesis of Compound 346

### Step 1. Synthesis of 2-(2-oxoprophyl)cyclohexan-1,3-dion [formula 11-2]

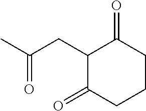

Potassium hydroxide (3.303 g, 58.86 mmol) and H₂O (5.0 mL) were mixed, and stirred for 30 minutes. Then cyclohexane-1,3-dion [formula 11-1] (6.0 g, 53.51 mmol) and ethanol (20.0 mL) were added and stirred for 15 minutes, 1-chloropropan-2-on (17.33 g, 187.28 mmol) was added and stirred at room temperature for 12 hours. After the completion of the reaction, ethanol was distilled out under the reduced pressure. The reaction mixture was extracted with ethyl acetate and saturated NaHCO₃ aqueous solution, the organic layer was washed with brine, dried over anhydrous Na₂SO₄, filtered and concentrated under reduced pressure. Without any purification, obtained compound was used in the next step.

### Step 2. Synthesis of 2-methyl-6,7-dihydro-1H-indol-4(5H)-on [formula 11-3]

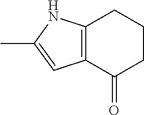

2-(2-oxoprophyl)cyclohexane-1,3-dion [formula 11-2] (7.82 g, 46.49 mmol), ammonium acetate (21.14 g, 274.32 mmol) and acetic acid (20.0 mL) were mixed and stirred at 140° C. for 3 hours. After the completion of the reaction, acetic acid was distilled out under the reduced pressure. The reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 4/1) to yield the title compound (2.26 g, 33%)

### Step 3. Synthesis of ethyl 7-(2-methyl-4-oxo-4,5,6,7-tetrahydroindol-1-yl)heptanoate [Formula 11-4]

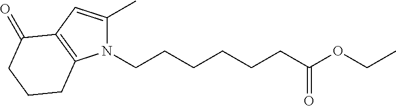 [SMILES: CCOC(=O)CCCCCCn1c(C)cc2c1CCCC2=O]

2-methyl-6,7-dihydro-1H-indol-4(5H)-on [formula 11-3] (0.163 g, 1.093 mmol) was dissolved in DMF (10 mL). Thereto, 55% NaH in paraffin solution (0.0985 g, 2.257 mmol) was added and stirred for 10 minutes. Then, ethyl 7-bromoheptanoate (0.292 g, 1.311 mmol) was added and stirred at 50° C. for 6 hours. After the completion of the reaction, DMF was distilled out under reduced pressure, and the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 5/1) to yield the title compound (0.259 g, 78%).

### Step 4. Synthesis of 7-(2-methyl-4-oxo-4,5,6,7-tetrahydroindol-1-yl)heptanoic acid [formula 11-5]

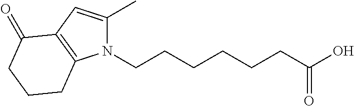 [SMILES: Cc1cc2c(n1CCCCCCC(=O)O)CCCC2=O]

Ethyl 7-(2-methyl-4-oxo-4,5,6,7-tetrahydroindol-1-yl)heptanoate [Formula 11-4] (0.259 g, 0.848 mmol) was dissolved in mixed solvent (methanol:H₂O=2:1, 15 mL). Thereto, lithium hydroxide monohydrate (0.356 g, 8.48 mmol) was added and stirred at room temperature for 1 hour. After the completion of the reaction, methanol was distilled out under reduced pressure, acidized with 2N HCl and filtered. Solid product was washed with water sufficiently, and dried with vacuum to yield the title compound (0.160 g, 68%).

### Step 5. Synthesis of 7-(2-methyl-4-oxo-4,5,6,7-tetrahydroindol-1-yl)-N-(tetrahydro-2H-pyran-2-yloxy)heptanamide [formula 11-6]

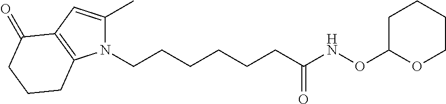 [SMILES: Cc1cc2c(n1CCCCCCC(=O)NOC1CCCCO1)CCCC2=O]

7-(2-methyl-4-oxo-4,5,6,7-tetrahydroindol-1-yl)heptanoic acid [formula 11-5] (0.160 g, 0.577 mmol) was dissolved in dichloromethane (10.0 mL). Thereto, o-(tetrahydro-2H-pyran-2-yl)hydroxylamine (0.101, g, 0.865 mmol), EDC (0.221 g, 13.15 mmol) and HOBt (0.156 g, 1.154 mmol) were added in order, DIPEA (0.373 g, 2.88 mmol) was added and stirred at room temperature for 12 hours. After the completion of the reaction, the reaction mixture was extracted with dichloromethane and saturated NaHCO₃ aqueous solution, the organic layer was washed with brine, dried over anhydrous MgSO₄, filtered and concentrated under reduced pressure. Residue was purified by column chromatography (SiO₂; hexane/ethylacetate, 4/1) to yield the title compound (0.12 g, 55%)

### Step 6. Synthesis of N-hydroxy-7-(2-methyl-4-oxo-4,5,6,7-tetrahydroindol-1-yl)heptanamide [formula 11-7]

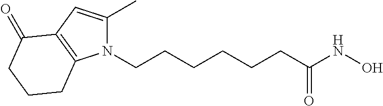 [SMILES: Cc1cc2c(n1CCCCCCC(=O)NO)CCCC2=O]

7-(2-methyl-4-oxo-4,5,6,7-tetrahydroindol-1-yl)-N-(tetrahydro-2H-pyran-2-yloxy)heptanamide [formula 11-6] (0.205 g, 0.545 mmol) was dissolved in methanol (3.0 mL). Thereto, 1.25 M HCl in methanol (2.18 mL, 2.723 mmol) was added and stirred at room temperature for 1 hour. After the completion of the reaction, methanol was distilled out under reduced pressure, and the reaction mixture was extracted with ethyl acetate and saturated NaHCO₃ aqueous solution, the organic layer was washed with brine, dried over anhydrous Na₂SO₄, filtered and concentrated under reduced pressure. Residue was re-crystallized by dichloromethane/hexane to yield the compound 346 (0.080 g, 50%).

¹H NMR (400 MHz, DMSO-d₆) δ 10.3 (s, 1H), 8.67 (s, 1H), 6.00 (s, 1H), 3.77 (s, 2H), 2.71 (s, 2H), 2.25 (brs, 2H), 1.98-1.92 (m, 4H), 1.53-1.48 (m, 4H), 1.26 (s, 3H); MS (ESI) m/z 293 (M++H).

### Example 104

### Synthesis of Compound 347

### Step 1. Synthesis of ethyl 7-(2,6,6-trimethyl-4-oxo-4,5,6,7-tetrahydroindol-1-yl)heptanoate [formula 11-4]

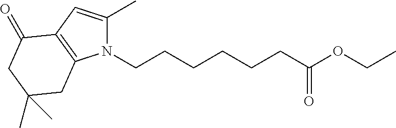 [SMILES: CCOC(=O)CCCCCCn1c(C)cc2c1CC(C)(C)CC2=O]

2-methyl-6,7-dihydro-1H-indol-4(5H)-on [formula 11-3] (0.18 g, 1.016 mmol) was dissolved in DMF (10 mL). Thereto, 55% NaH in paraffin solution (0.0886 g, 2.031 mmol) was added and stirred for 10 minutes. Then, ethyl 7-bromoheptanoate (0.272 g, 1.22 mmol) was added and stirred at room temperature for 3 hours. After the completion of the reaction, DMF was distilled out under reduced pressure, and the reaction mixture was extracted with ethyl acetate and saturated NaHCO₃ aqueous solution, the organic layer was washed with brine, dried over anhydrous Na₂SO₄, filtered and concentrated under reduced pressure. Residue was purified by column chromatography (SiO₂; hexane/ethylacetate, 5/1) to yield the title compound (0.289 g, 85%).

### Step 2. Synthesis of 7-(2,6,6-trimethyl-4-oxo-4,5,6,7-tetrahydroindol-1-yl)heptanoic acid [formula 11-5]

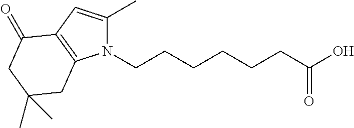 [SMILES: Cc1cc2c(n1CCCCCCC(=O)O)CC(C)(C)CC2=O]

Ethyl 7-(2,6,6-trimethyl-4-oxo-4,5,6,7-tetrahydroindol-1-yl)heptanoate [Formula 11-4] (0.35 g, 1.05 mmol) was dissolved in mixed solvent (methanol:H₂O=2:1, 15 mL). Thereto, lithium hydroxide monohydrate (0.44 g, 10.50 mmol) was added and stirred at room temperature for 1 hour. After the completion of the reaction, methanol was distilled out under reduced pressure, acidized with 2N HCl and filtered. Solid product was washed with water sufficiently, and dried with vacuum to yield the title compound (0.296 g, 92%).

### Step 3. Synthesis of N-(tetrahydro-2H-pyran-2-yloxy)-7-(2,6,6-trimethyl-4-oxo-4,5,6,7-tetrahydroindol-1-yl) heptanamide [formula 11-6]

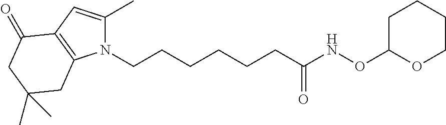 [SMILES: Cc1cc2c(n1CCCCCCC(=O)NOC1CCCCO1)CC(C)(C)CC2=O]

7-(2,6,6-trimethyl-4-oxo-4,5,6,7-tetrahydroindol-1-yl)heptanoic acid [formula 11-5] (0.296 g, 0.969 mmol) was dissolved in dichloromethane (10.0 mL). Thereto, o-(tetrahydro-2H-pyran-2-yl)hydroxylamine (0.170 g, 1.454 mmol), EDC (0.372 g, 1.94 mmol) and HOBt (0.262 g, 1.94 mmol) were added in order, DIPEA (0.626 g, 4.85 mmol) was added and stirred at room temperature for 12 hours. After the completion of the reaction, the reaction mixture was extracted with dichloromethane and saturated NaHCO₃ aqueous solution, the organic layer was washed with brine, dried over anhydrous MgSO₄, filtered and concentrated under reduced pressure. Residue was purified by column chromatography (SiO₂; hexane/ethylacetate, 4/1) to yield the title compound (0.21 g, 54%)

### Step 4. Synthesis of N-hydroxy-7-(2,6,6-trimethyl-4-oxo-4,5,6,7-tetrahydroindol-1-yl)heptanamide [formula 11-7]

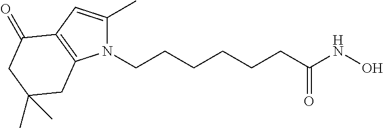 [SMILES: Cc1cc2c(n1CCCCCCC(=O)NO)CC(C)(C)CC2=O]

N-(tetrahydro-2H-pyran-2-yloxy)-7-(2,6,6-trimethyl-4-oxo-4,5,6,7-tetrahydroindol-1-yl)heptanamide [formula 11-6] (0.335 g, 0.828 mmol) was dissolved in methanol (3.0 mL). Thereto, 1.25 M HCl in methanol (3.31 mL, 4.14 mmol) was added and stirred at room temperature for 1 hour. After the completion of the reaction, methanol was distilled out under reduced pressure, and the reaction mixture was extracted with ethyl acetate and saturated NaHCO₃ aqueous solution, the organic layer was washed with brine, dried over anhydrous Na₂SO₄, filtered and concentrated under reduced pressure. Residue was re-crystallized by dichloromethane/hexane to yield the compound 347 (0.105 g, 40%).

¹H NMR (400 MHz, DMSO-d₆) δ 10.3 (s, 1H), 8.66 (s, 1H), 6.00 (s, 1H), 3.76 (s, 2H), 2.61 (s, 2H), 2.16 (brs, 5H), 1.98-1.90 (m, 2H), 1.52-1.46 (m, 4H), 1.24 (s, 4H), 1.02 (s, 6H); MS (ESI) m/z 321 (M++H).

### Example 105

### Synthesis of Compound 350

### Step 1. Synthesis of ethyl 6-(2,3,6,6-tetramethyl-4-oxo-4,5,6,7-tetrahydro-1H-indole-1-yl)hexanoate [formula 6-5]

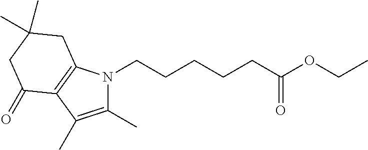 [SMILES: CCOC(=O)CCCCCn1c(C)c(C)c2c1CC(C)(C)CC2=O]

2,3,6,6-tetramethyl-6,7-dihydro-1H-indole-4(5H)-on [formula 6-2] (0.40 g, 2.10 mmol) was dissolved DMF. Thereto, NaH (0.11 g, 2.56 mmol) was added slowly at room temperature, and stirred for 5 minutes. Ethyl 6-bromohexanoate (0.46 mL, 2.52 mmol) was added and stirred at room temperature for 1 day. After the completion of the reaction, the reaction mixture was extracted with ethyl acetate and saturated NH₄Cl aqueous solution, the organic layer was dried over anhydrous MgSO₄ and filtered. The filtrate was concentrated under reduced pressure and purified by column chromatography (SiO₂; hexane/ethylacetate, 7/3) to yield the title compound as transparent oil (0.51 g, 73%).

### Step 2. Synthesis of 6-(2,3,6,6-tetramethyl-4-oxo-4,5,6,7-tetrahydro-1H-indol-1-yl) hexanoic acid [formula 6-8]

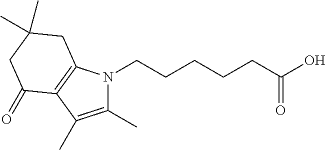 [SMILES: Cc1c2c(n(CCCCCC(=O)O)c1C)CC(C)(C)CC2=O]

Ethyl 6-(2,3,6,6-tetramethyl-4-oxo-4,5,6,7-tetrahydro-1H-indole-1-yl)hexanoate [formula 6-5] (0.51 g, 1.53 mmol) was dissolved in methanol (10 mL) and H₂O (5 mL), and refluxed with stirring for 1 hour. The reaction mixture was cooled to room temperature and concentrated under reduced pressure, and adjusted pH to 1-2 using 1M HCl to obtain white precipitate. The solid product was filtered, washed with water, and dried under reduced pressure to yield the title compound as white solid (0.38 g, 81%).

### Step 3. Synthesis of N-((tetrahydro-2H-pyran-2-yl)oxo)-6-(2,3,6,6-tetramethyl-4-oxo-4,5,6,7-tetrahydro-1H-indol-1-yl) hexanamide [formula 6-9]

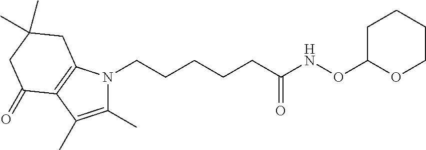 [SMILES: Cc1c2c(n(CCCCCC(=O)NOC3CCCCO3)c1C)CC(C)(C)CC2=O]

6-(2,3,6,6-tetramethyl-4-oxo-4,5,6,7-tetrahydro-1H-indole-1-yl)hexanoic acid [formula 6-8] (0.14 g, 0.46 mmol) and o-(tetrahydro-2H-pyran-2-yl)hydroxylamine (0.064 g, 0.55 mmol) were dissolved in CH₂Cl₂. Thereto, EDC (0.13 g, 0:68 mmol), HOBt (0.092 g, 0.68 mmol) and DIPEA (0.12 g, 0.91 mmol) were added, and reaction was carried out at room temperature for 1 day. After the completion of the reaction, the reaction mixture was extracted with ethyl acetate and saturated NH₄Cl aqueous solution, the organic layer was dried over anhydrous MgSO₄ and filtered. The filtrate was concentrated under reduced pressure, and was purified by column chromatography (SiO₂; hexane/ethylacetate, 0/10) to yield the title compound as transparent oil (0.065 g, 35%)

### Step 4. Synthesis of N-hydroxy-6-(2,3,6,6-tetramethyl-4-oxo-4,5,6,7-tetrahydro-1H-indol-1-yl)hexanamide [formula 6-10]

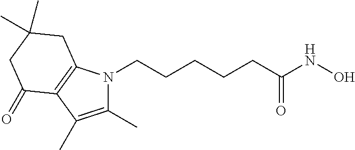 [SMILES: Cc1c2c(n(CCCCCC(=O)NO)c1C)CC(C)(C)CC2=O]

N-((tetrahydro-2H-pyran-2-yl)oxo)-6-(2,3,6,6-tetramethyl-4-oxo-4,5,6,7-tetrahydro-1H-indol-1-yl) hexanamide [formula 6-9] (0.16 g, 0.40 mmol) was dissolved in methanol, and methanlic HCl (1.60 mL, 2.0 mmol) was added. Reaction was carried out at room temperature for 1 hour. After the completion of the reaction, the mixture was concentrated under reduced pressure to obtain white solid product, which is filtered and dried to yield compound 350 as white solid (0.078 g, 61%).

The structural formulae are as following Table 1-10.

TABLE 1

| Com- | |
|---|---|
| pound | Structure |
| 18 | 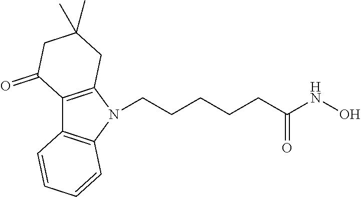 [SMILES: CC1(C)CC(=O)c2c(n(CCCCCC(=O)NO)c3ccccc23)C1] |
| 19 | 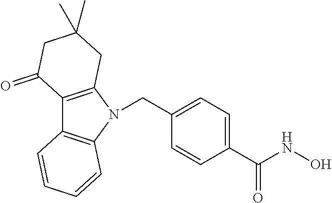 [SMILES: CC1(C)CC(=O)c2c(n(Cc3ccc(C(=O)NO)cc3)c3ccccc23)C1] |
| 20 | 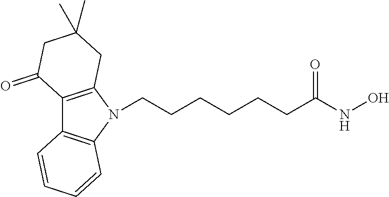 |
| 40 | 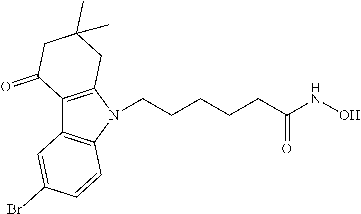 [SMILES: CC1(C)CC(=O)c2c(n(CCCCCC(=O)NO)c3ccc(Br)cc23)C1] |
| 41 | 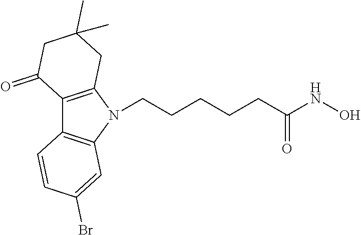 [SMILES: CC1(C)CC(=O)c2c(n(CCCCCC(=O)NO)c3cc(Br)ccc23)C1] |
| 45 | 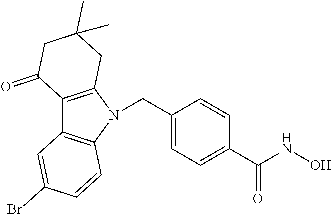 [SMILES: CC1(C)CC(=O)c2c(n(Cc3ccc(C(=O)NO)cc3)c3ccc(Br)cc23)C1] |
| 46 | 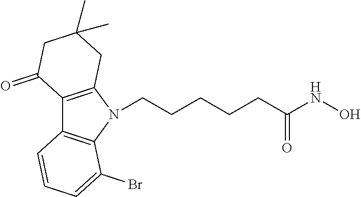 [SMILES: CC1(C)CC(=O)c2c(n(CCCCCC(=O)NO)c3c(Br)cccc23)C1] |
| 47 | 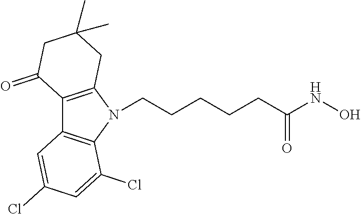 [SMILES: CC1(C)CC(=O)c2c(n(CCCCCC(=O)NO)c3c(Cl)cc(Cl)cc23)C1] |
| 48 | 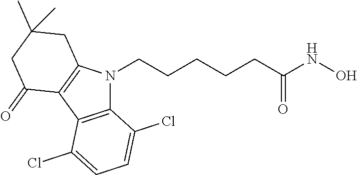 [SMILES: CC1(C)CC(=O)c2c(n(CCCCCC(=O)NO)c3c(Cl)ccc(Cl)c23)C1] |
| 49 | 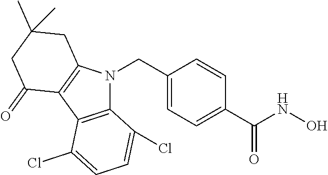 [SMILES: CC1(C)CC(=O)c2c(n(Cc3ccc(C(=O)NO)cc3)c3c(Cl)ccc(Cl)c23)C1] |
| 50 | 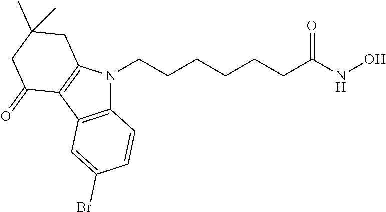 [SMILES: CC1(C)CC(=O)c2c(n(CCCCCCC(=O)NO)c3ccc(Br)cc23)C1] |
| 51 | 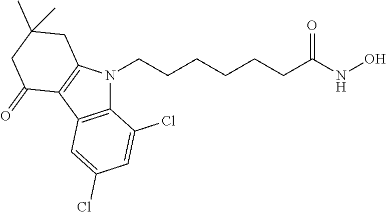 [SMILES: CC1(C)CC(=O)c2c(n(CCCCCCC(=O)NO)c3c(Cl)cc(Cl)cc23)C1] |

TABLE 2

| Com- | |
|---|---|
| pound | Structure |
| 52 | 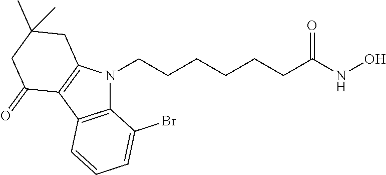 [SMILES: CC1(C)CC(=O)c2c(n(CCCCCCC(=O)NO)c3c(Br)cccc23)C1] |
| 53 | 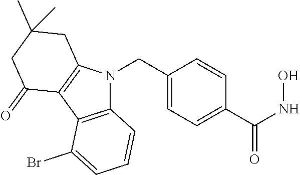 [SMILES: CC1(C)CC(=O)c2c(n(Cc3ccc(C(=O)NO)cc3)c3cccc(Br)c23)C1] |
| 54 | 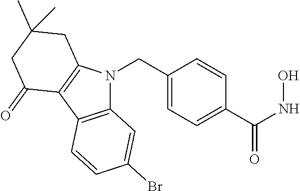 [SMILES: CC1(C)CC(=O)c2c(n(Cc3ccc(C(=O)NO)cc3)c3cc(Br)ccc23)C1] |
| 55 | 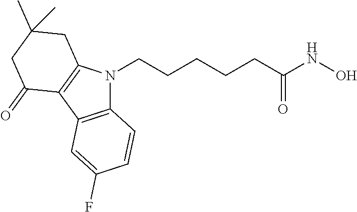 [SMILES: CC1(C)CC(=O)c2c(n(CCCCCC(=O)NO)c3ccc(F)cc23)C1] |
| 56 | 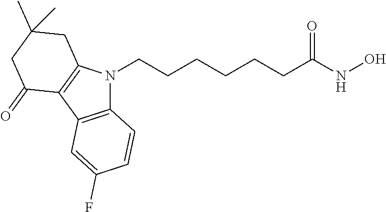 [SMILES: CC1(C)CC(=O)c2c(n(CCCCCCC(=O)NO)c3ccc(F)cc23)C1] |
| 57 | 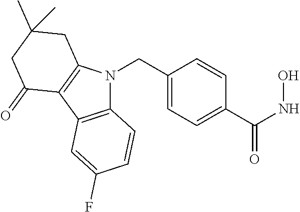 [SMILES: CC1(C)CC(=O)c2c(n(Cc3ccc(C(=O)NO)cc3)c3ccc(F)cc23)C1] |
| 60 | 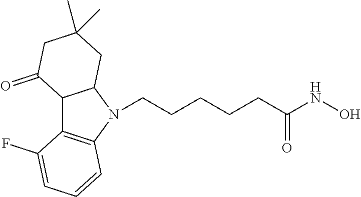 [SMILES: CC1(C)CC(=O)C2c3c(F)cccc3N(CCCCCC(=O)NO)C2C1] |
| 71 | 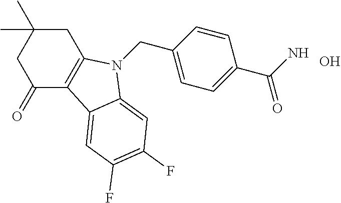 |
| 72 | 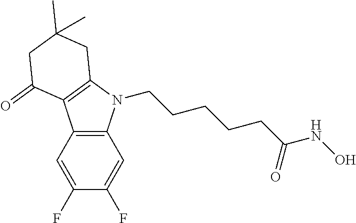 [SMILES: CC1(C)CC(=O)c2c(n(CCCCCC(=O)NO)c3cc(F)c(F)cc23)C1] |
| 73 | 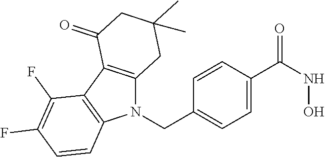 [SMILES: CC1(C)CC(=O)c2c(n(Cc3ccc(C(=O)NO)cc3)c3ccc(F)c(F)c23)C1] |
| 74 | 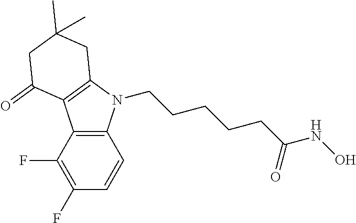 [SMILES: CC1(C)CC(=O)c2c(n(CCCCCC(=O)NO)c3ccc(F)c(F)c23)C1] |
| 76 | 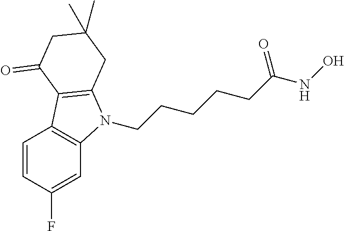 [SMILES: CC1(C)CC(=O)c2c(n(CCCCCC(=O)NO)c3cc(F)ccc23)C1] |

TABLE 3

| Compound | Structure |
|---|---|
| 85 | 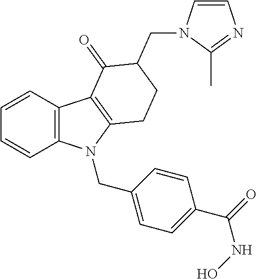 [SMILES: Cc1nccn1CC1CCc2c(c3ccccc3n2Cc2ccc(C(=O)NO)cc2)C1=O] |
| 86 | 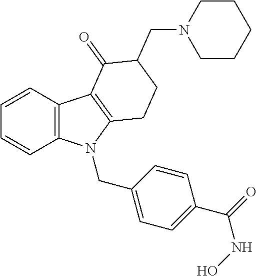 [SMILES: O=C(NO)c1ccc(Cn2c3c(c4ccccc42)C(=O)C(CN2CCCCC2)CC3)cc1] |
| 87 | 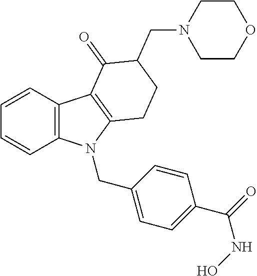 [SMILES: O=C(NO)c1ccc(Cn2c3c(c4ccccc42)C(=O)C(CN2CCOCC2)CC3)cc1] |
| 88 | 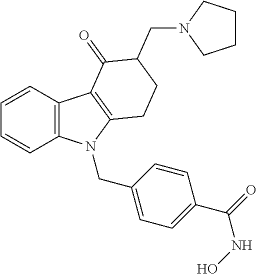 [SMILES: O=C(NO)c1ccc(Cn2c3c(c4ccccc42)C(=O)C(CN2CCCC2)CC3)cc1] |
| 99 | 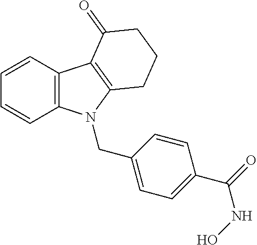 [SMILES: O=C(NO)c1ccc(Cn2c3c(c4ccccc42)C(=O)CCC3)cc1] |
| 101 | 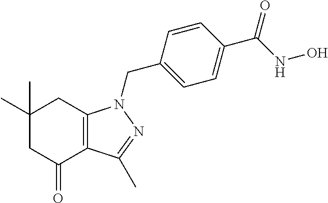 [SMILES: Cc1nn(Cc2ccc(C(=O)NO)cc2)c2c1C(=O)CC(C)(C)C2] |
| 110 | 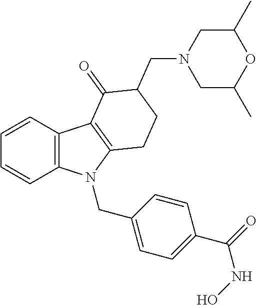 [SMILES: CC1CN(CC2CCc3c(c4ccccc4n3Cc3ccc(C(=O)NO)cc3)C2=O)CC(C)O1] |
| 111 | 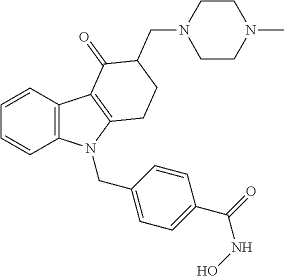 [SMILES: CN1CCN(CC2CCc3c(c4ccccc4n3Cc3ccc(C(=O)NO)cc3)C2=O)CC1] |

TABLE 4

| Com- | |
|---|---|
| pound | Structure |
| 112 | 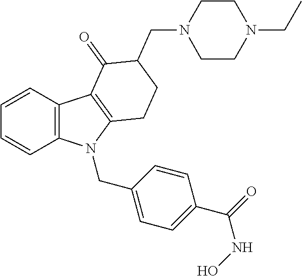 [SMILES: CCN1CCN(CC2CCc3c(c4ccccc4n3Cc3ccc(C(=O)NO)cc3)C2=O)CC1] |
| 113 | 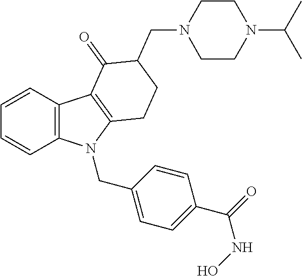 [SMILES: CC(C)N1CCN(CC2CCc3c(c4ccccc4n3Cc3ccc(C(=O)NO)cc3)C2=O)CC1] |
| 114 | 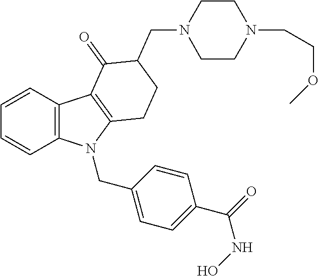 [SMILES: COCCN1CCN(CC2CCc3c(c4ccccc4n3Cc3ccc(C(=O)NO)cc3)C2=O)CC1] |
| 121 | 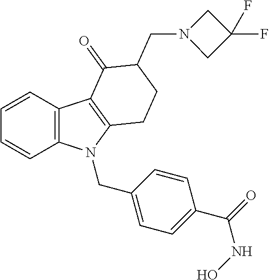 [SMILES: O=C(NO)c1ccc(Cn2c3c(c4ccccc42)C(=O)C(CN2CC(F)(F)C2)CC3)cc1] |
| 122 | 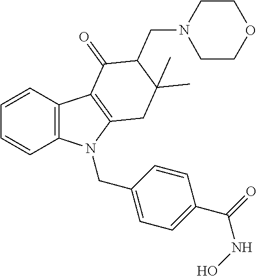 [SMILES: CC1(C)Cc2c(c3ccccc3n2Cc2ccc(C(=O)NO)cc2)C(=O)C1CN1CCOCC1] |
| 123 | 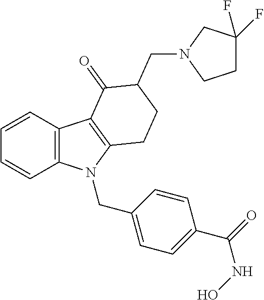 [SMILES: O=C(NO)c1ccc(Cn2c3c(c4ccccc42)C(=O)C(CN2CCC(F)(F)C2)CC3)cc1] |
| 126 | 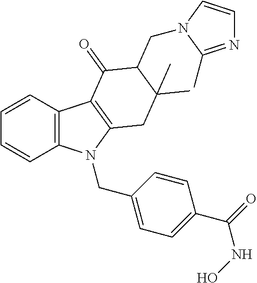 [SMILES: Cc1nccn1CC1C(=O)c2c(n(Cc3ccc(C(=O)NO)cc3)c3ccccc23)CC1(C)C] |
| 127 | 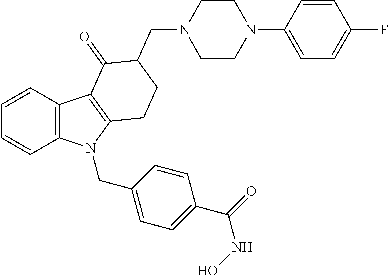 [SMILES: O=C(NO)c1ccc(Cn2c3c(c4ccccc42)C(=O)C(CN2CCN(c4ccc(F)cc4)CC2)CC3)cc1] |
| 128 | 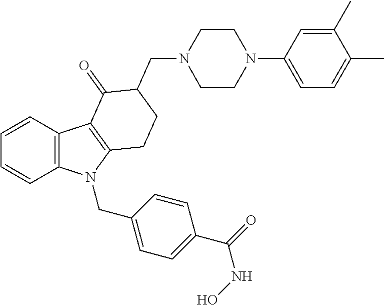 [SMILES: Cc1ccc(N2CCN(CC3CCc4c(c5ccccc5n4Cc4ccc(C(=O)NO)cc4)C3=O)CC2)cc1C] |
| 129 | 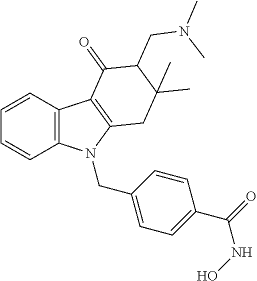 [SMILES: CN(C)CC1C(=O)c2c(n(Cc3ccc(C(=O)NO)cc3)c3ccccc23)CC1(C)C] |

TABLE 5

| Compound | Structure |
|---|---|
| 130 | 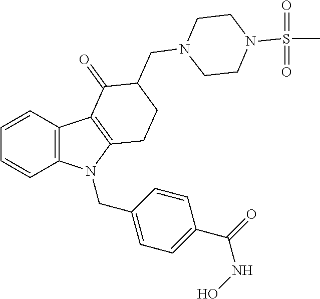 [SMILES: CS(=O)(=O)N1CCN(CC2CCc3c(c4ccccc4n3Cc3ccc(C(=O)NO)cc3)C2=O)CC1] |
| 131 | 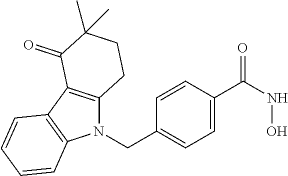 [SMILES: CC1(C)CCc2c(c3ccccc3n2Cc2ccc(C(=O)NO)cc2)C1=O] |
| 136 | 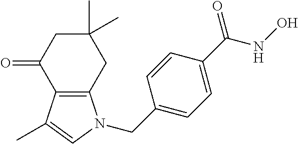 [SMILES: Cc1cn(Cc2ccc(C(=O)NO)cc2)c2c1C(=O)CC(C)(C)C2] |
| 140 | 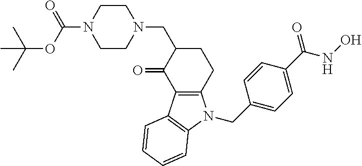 [SMILES: CC(C)(C)OC(=O)N1CCN(CC2CCc3c(c4ccccc4n3Cc3ccc(C(=O)NO)cc3)C2=O)CC1] |
| 141 | 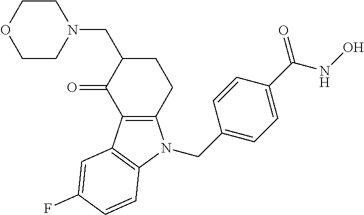 [SMILES: O=C(NO)c1ccc(Cn2c3c(c4cc(F)ccc42)C(=O)C(CN2CCOCC2)CC3)cc1] |
| 142 | 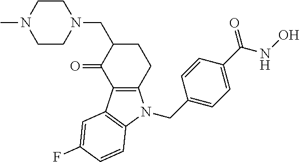 [SMILES: CN1CCN(CC2CCc3c(c4cc(F)ccc4n3Cc3ccc(C(=O)NO)cc3)C2=O)CC1] |
| 144 | 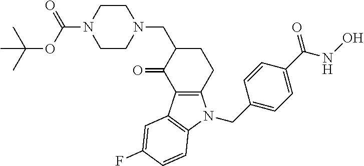 [SMILES: CC(C)(C)OC(=O)N1CCN(CC2CCc3c(c4cc(F)ccc4n3Cc3ccc(C(=O)NO)cc3)C2=O)CC1] |
| 145 | 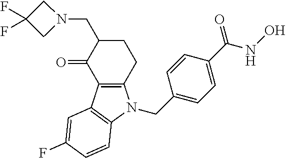 [SMILES: O=C(NO)c1ccc(Cn2c3c(c4cc(F)ccc42)C(=O)C(CN2CC(F)(F)C2)CC3)cc1] |
| 156 | 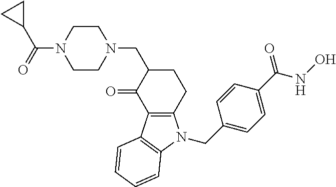 [SMILES: O=C(NO)c1ccc(Cn2c3c(c4ccccc42)C(=O)C(CN2CCN(C(=O)C4CC4)CC2)CC3)cc1] |
| 157 | 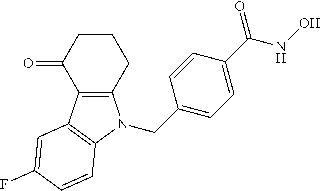 [SMILES: O=C(NO)c1ccc(Cn2c3c(c4cc(F)ccc42)C(=O)CCC3)cc1] |
| 158 | 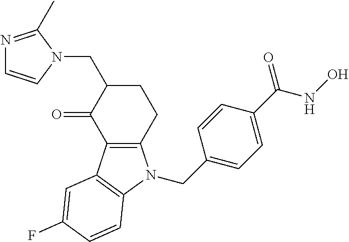 [SMILES: Cc1nccn1CC1CCc2c(c3cc(F)ccc3n2Cc2ccc(C(=O)NO)cc2)C1=O] |
| 166 | 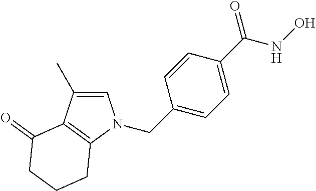 [SMILES: Cc1cn(Cc2ccc(C(=O)NO)cc2)c2c1C(=O)CCC2] |

TABLE 6

| Compound | Structure |
|---|---|
| 179 | 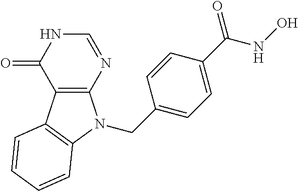 [SMILES: O=C(NO)c1ccc(Cn2c3ccccc3c3c(=O)[nH]cnc32)cc1] |
| 188 | 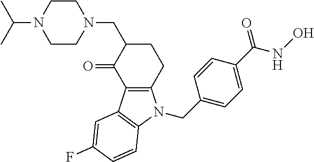 [SMILES: CC(C)N1CCN(CC2CCc3c(c4cc(F)ccc4n3Cc3ccc(C(=O)NO)cc3)C2=O)CC1] |
| 189 | 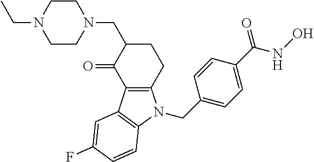 [SMILES: CCN1CCN(CC2CCc3c(c4cc(F)ccc4n3Cc3ccc(C(=O)NO)cc3)C2=O)CC1] |
| 190 | 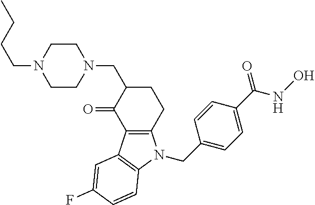 [SMILES: CCCCN1CCN(CC2CCc3c(c4cc(F)ccc4n3Cc3ccc(C(=O)NO)cc3)C2=O)CC1] |
| 191 | 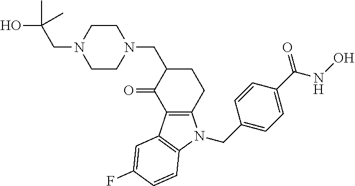 [SMILES: CC(C)(O)CN1CCN(CC2CCc3c(c4cc(F)ccc4n3Cc3ccc(C(=O)NO)cc3)C2=O)CC1] |
| 192 | 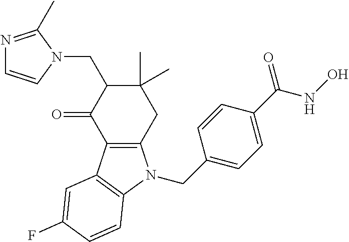 [SMILES: Cc1nccn1CC1C(=O)c2c(n(Cc3ccc(C(=O)NO)cc3)c3ccc(F)cc23)CC1(C)C] |
| 193 | 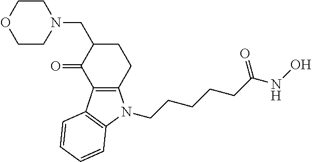 [SMILES: O=C(CCCCCn1c2c(c3ccccc31)C(=O)C(CN1CCOCC1)CC2)NO] |
| 194 | 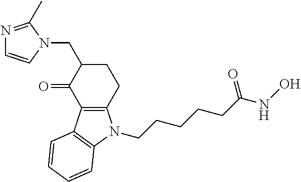 [SMILES: Cc1nccn1CC1CCc2c(c3ccccc3n2CCCCCC(=O)NO)C1=O] |
| 203 | 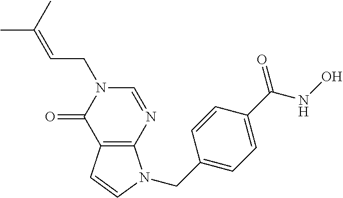 [SMILES: CC(C)=CCn1cnc2c(ccn2Cc2ccc(C(=O)NO)cc2)c1=O] |
| 204 | 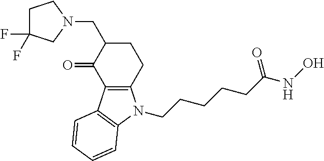 [SMILES: O=C(CCCCCn1c2c(c3ccccc31)C(=O)C(CN1CCC(F)(F)C1)CC2)NO] |
| 205 | 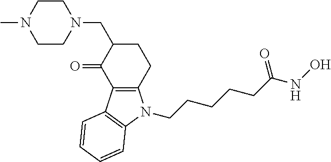 [SMILES: CN1CCN(CC2CCc3c(c4ccccc4n3CCCCCC(=O)NO)C2=O)CC1] |
| 206 | 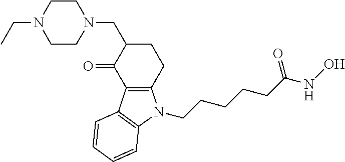 [SMILES: CCN1CCN(CC2CCc3c(c4ccccc4n3CCCCCC(=O)NO)C2=O)CC1] |

TABLE 7

| Com- | |
|---|---|
| pound | Structure |
| 207 | 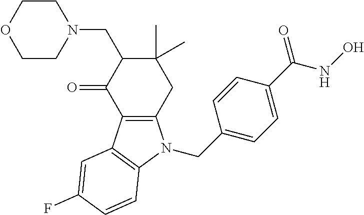 [SMILES: CC1(C)Cc2c(c3cc(F)ccc3n2Cc2ccc(C(=O)NO)cc2)C(=O)C1CN1CCOCC1] |
| 208 | 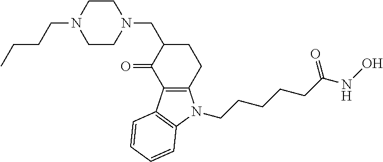 [SMILES: CCCCN1CCN(CC2CCc3c(c4ccccc4n3CCCCCC(=O)NO)C2=O)CC1] |
| 209 | 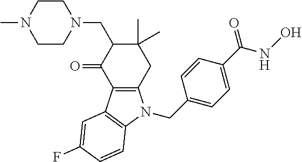 [SMILES: CN1CCN(CC2C(=O)c3c(n(Cc4ccc(C(=O)NO)cc4)c4ccc(F)cc34)CC2(C)C)CC1] |
| 220 | 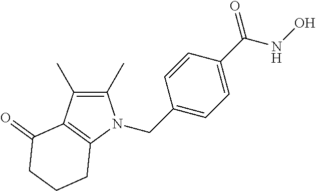 [SMILES: Cc1c2c(n(Cc3ccc(C(=O)NO)cc3)c1C)CCCC2=O] |
| 228 | 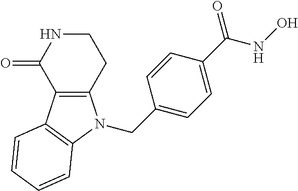 [SMILES: O=C(NO)c1ccc(Cn2c3c(c4ccccc42)C(=O)NCC3)cc1] |
| 232 | 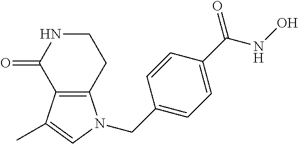 [SMILES: Cc1cn(Cc2ccc(C(=O)NO)cc2)c2c1C(=O)NCC2] |
| 235 | 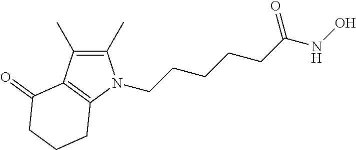 [SMILES: Cc1c2c(n(CCCCCC(=O)NO)c1C)CCCC2=O] |
| 236 | 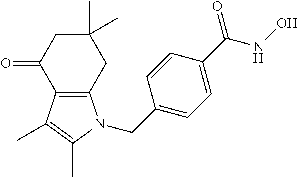 [SMILES: Cc1c2c(n(Cc3ccc(C(=O)NO)cc3)c1C)CC(C)(C)CC2=O] |
| 237 | 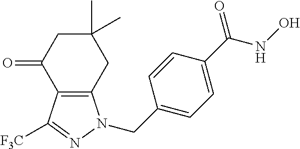 [SMILES: CC1(C)CC(=O)c2c(C(F)(F)F)nn(Cc3ccc(C(=O)NO)cc3)c2C1] |
| 249 | 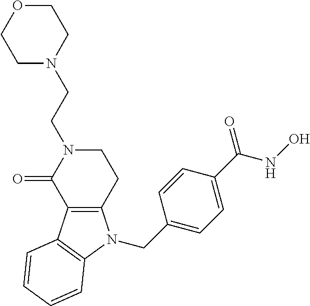 [SMILES: O=C(NO)c1ccc(Cn2c3c(c4ccccc42)C(=O)N(CCN2CCOCC2)CC3)cc1] |

TABLE 8

| Com- | |
|---|---|
| pound | Structure |
| 250 | 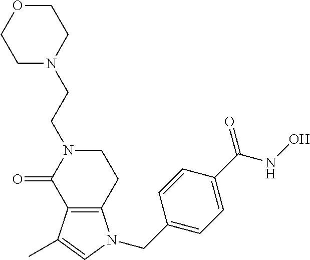 [SMILES: Cc1cn(Cc2ccc(C(=O)NO)cc2)c2c1C(=O)N(CCN1CCOCC1)CC2] |
| 251 | 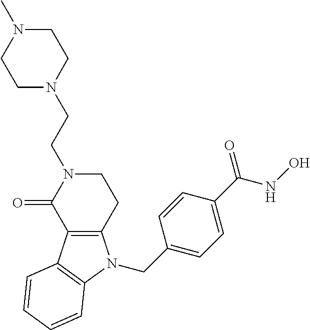 [SMILES: CN1CCN(CCN2CCc3c(c4ccccc4n3Cc3ccc(C(=O)NO)cc3)C2=O)CC1] |
| 266 | 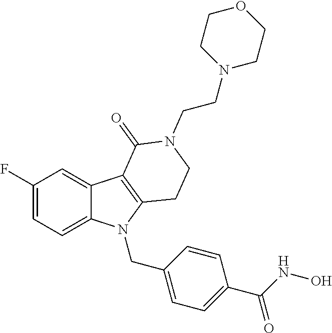 [SMILES: O=C(NO)c1ccc(Cn2c3c(c4cc(F)ccc42)C(=O)N(CCN2CCOCC2)CC3)cc1] |
| 267 | 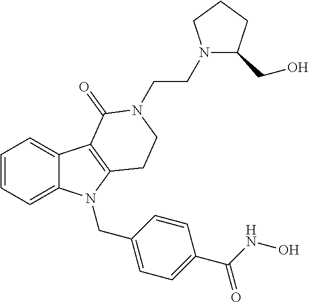 [SMILES: O=C(NO)c1ccc(Cn2c3c(c4ccccc42)C(=O)N(CCN2CCC[C@H]2CO)CC3)cc1] |
| 268 | 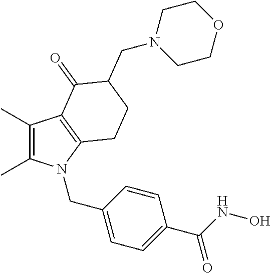 [SMILES: Cc1c2c(n(Cc3ccc(C(=O)NO)cc3)c1C)CCC(CN1CCOCC1)C2=O] |
| 283 | 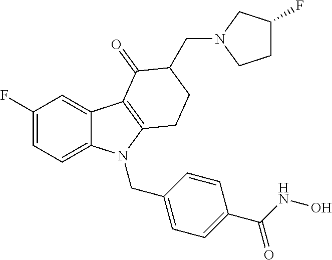 [SMILES: O=C(NO)c1ccc(Cn2c3c(c4cc(F)ccc42)C(=O)C(CN2CC[C@@H](F)C2)CC3)cc1] |
| 284 | 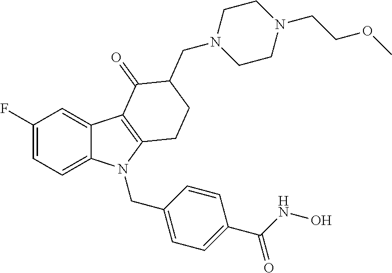 [SMILES: COCCN1CCN(CC2CCc3c(c4cc(F)ccc4n3Cc3ccc(C(=O)NO)cc3)C2=O)CC1] |
| 285 | 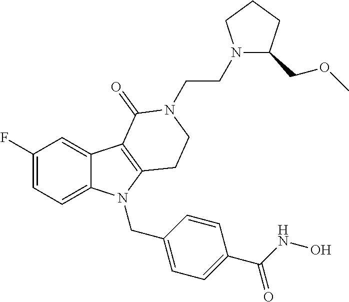 [SMILES: COC[C@@H]1CCCN1CCN1CCc2c(c3cc(F)ccc3n2Cc2ccc(C(=O)NO)cc2)C1=O] |

TABLE 9

| Compound | Structure |
|---|---|
| 286 | 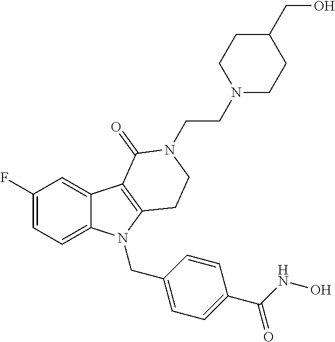 [SMILES: O=C(NO)c1ccc(Cn2c3c(c4cc(F)ccc42)C(=O)N(CCN2CCC(CO)CC2)CC3)cc1] |
| 287 | 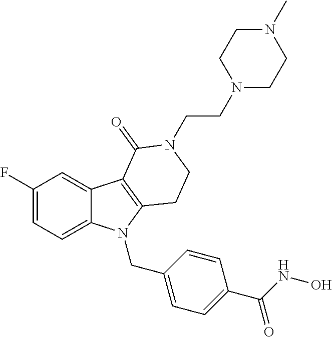 [SMILES: CN1CCN(CCN2CCc3c(c4cc(F)ccc4n3Cc3ccc(C(=O)NO)cc3)C2=O)CC1] |
| 288 | 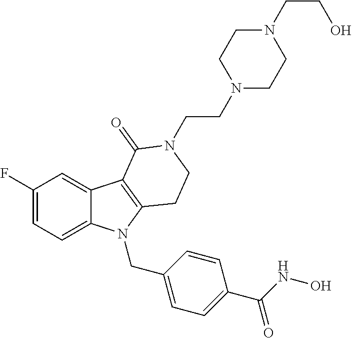 [SMILES: O=C(NO)c1ccc(Cn2c3c(c4cc(F)ccc42)C(=O)N(CCN2CCN(CCO)CC2)CC3)cc1] |
| 289 | 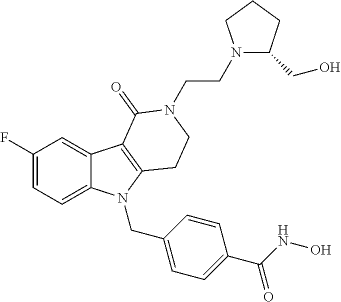 [SMILES: O=C(NO)c1ccc(Cn2c3c(c4cc(F)ccc42)C(=O)N(CCN2CCC[C@@H]2CO)CC3)cc1] |
| 290 | 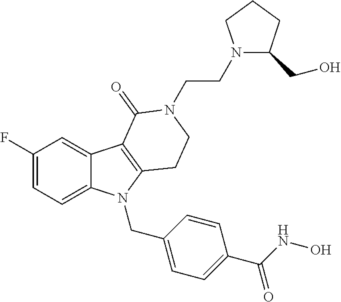 [SMILES: O=C(NO)c1ccc(Cn2c3c(c4cc(F)ccc42)C(=O)N(CCN2CCC[C@H]2CO)CC3)cc1] |
| 291 | 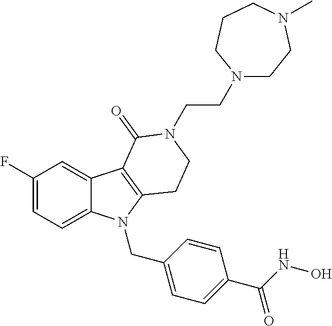 [SMILES: CN1CCCN(CCN2CCc3c(c4cc(F)ccc4n3Cc3ccc(C(=O)NO)cc3)C2=O)CC1] |
| 292 | 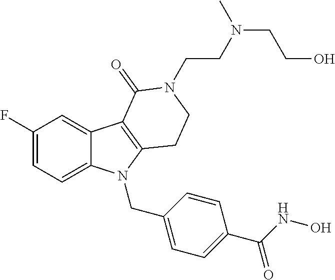 [SMILES: CN(CCO)CCN1CCc2c(c3cc(F)ccc3n2Cc2ccc(C(=O)NO)cc2)C1=O] |
| 305 | 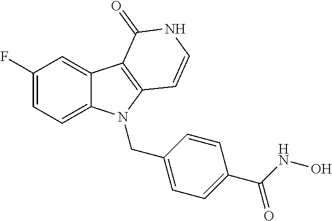 [SMILES: O=C(NO)c1ccc(Cn2c3ccc(F)cc3c3c(=O)[nH]ccc32)cc1] |
| 306 | 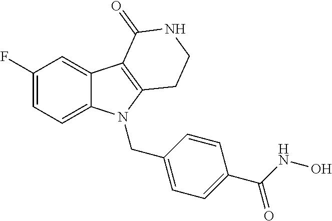 [SMILES: O=C(NO)c1ccc(Cn2c3c(c4cc(F)ccc42)C(=O)NCC3)cc1] |
| 321 | 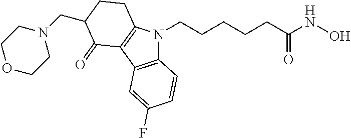 [SMILES: O=C(CCCCCn1c2c(c3cc(F)ccc31)C(=O)C(CN1CCOCC1)CC2)NO] |

TABLE 10

| Com- | |
|---|---|
| pound | Structure |
| 322 | 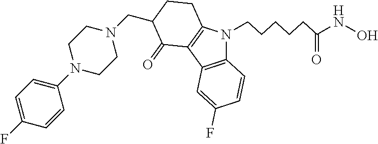 [SMILES: O=C(CCCCCn1c2c(c3cc(F)ccc31)C(=O)C(CN1CCN(c3ccc(F)cc3)CC1)CC2)NO] |
| 323 | 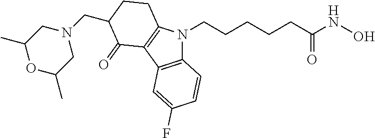 [SMILES: CC1CN(CC2CCc3c(c4cc(F)ccc4n3CCCCCC(=O)NO)C2=O)CC(C)O1] |
| 324 | 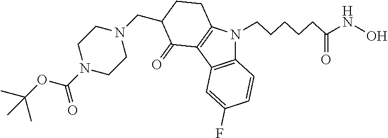 [SMILES: CC(C)(C)OC(=O)N1CCN(CC2CCc3c(c4cc(F)ccc4n3CCCCCC(=O)NO)C2=O)CC1] |
| 325 | 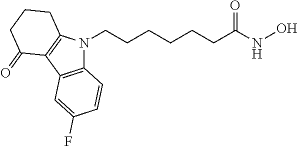 [SMILES: O=C(CCCCCCn1c2c(c3cc(F)ccc31)C(=O)CCC2)NO] |
| 326 | 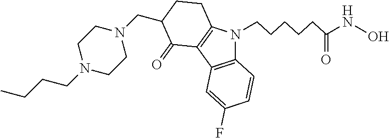 [SMILES: CCCCN1CCN(CC2CCc3c(c4cc(F)ccc4n3CCCCCC(=O)NO)C2=O)CC1] |
| 328 | 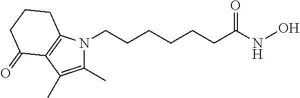 [SMILES: Cc1c2c(n(CCCCCCC(=O)NO)c1C)CCCC2=O] |
| 344 | 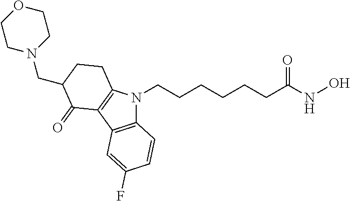 [SMILES: O=C(CCCCCCn1c2c(c3cc(F)ccc31)C(=O)C(CN1CCOCC1)CC2)NO] |
| 345 | 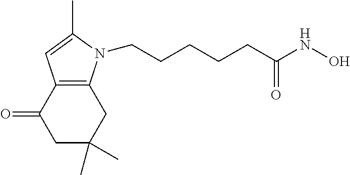 [SMILES: Cc1cc2c(n1CCCCCC(=O)NO)CC(C)(C)CC2=O] |
| 346 | 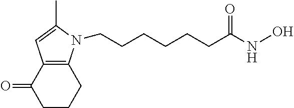 [SMILES: Cc1cc2c(n1CCCCCCC(=O)NO)CCCC2=O] |
| 347 | 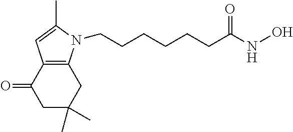 [SMILES: Cc1cc2c(n1CCCCCCC(=O)NO)CC(C)(C)CC2=O] |
| 350 | 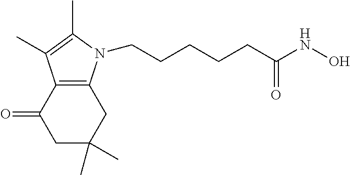 [SMILES: Cc1c2c(n(CCCCCC(=O)NO)c1C)CC(C)(C)CC2=O] |

### Protocol of Experiment: Activity Test for the Compound of the Present Invention

### Experimental Example 1

### Screening of the Inhibition Against HDAC Enzyme Activity (In Vitro)

### 1. HDAC Enzyme Activity Test

HDAC enzyme activity was measured with HDAC Fluorimetric Drug Discovery Kit (BML-AK511, 516) from Enzo Life Science Co. Human recombinant HDAC1 (BML-SE456) was used as a source of enzyme, and Fluor de Lys®-SIRT1 (BML-KI177) was used as a substrate. To 96-wells plate were added the compound, which had been diluted 5 times. Then, 0.3 μg of enzyme and 10 μM of substrate per well were added, reacted at 30° C. for 60 minutes. Thereto, Fluor de Lys® Developer II (BML-KI176) was added and reacted for 30 minutes. After the completion of the reaction, fluorescence level (Ex 360, Em 460) was measured with muti-plate reader (Flexstation 3, Molecular Device). With regard to HDAC6 enzyme, the test was performed using human recombinant HDAC6 (382180) from Calibiochem Co. as a source of enzyme by the same protocol as for the measurement for HDAC1 enzyme activity. From the data, the respective IC₅₀ were calculated by GraphPad Prism 4.0 program.

### 2. Western Blot Analysis

For the Western blot analysis, HH (cutaneous T cell lymphoma) cells were added by 3.0×10⁶ cells per well in RPMI-1640 culture medium (6-well plate), treated with test materials, and cultured in 5% CO₂ incubator at 37° C. for 24 hours. The culture medium was removed, and the cells were washed with PBS, and to each well was added 400 μM of ice-cold lysis buffer (20 nM Tris-HCl, pH 7.5, 1 mM EGTA, 1 mM Na₂ EDTA, 150 mM NaCl, 1% Triton X-100, 1 mM PMSF, 1 μg/mL leupeptin, 2.5 mM sodium pyrophosphate, 1 mM β-glycerophosphate, and 1 mM Na₃VO₄).

Protein concentration of cell lysate was determined with BSA protein assay (Pierce), and 30 μg of protein per lysate was electrophoresed in 4-12% Bis-Tris gel and then, transferred into nitrocellulose membrane. As primary antibody, acetylated α-tubulin (1:1000, Sigma-aldrich), acetylated histon H3 (1:1000, Millipore) and β-actin (1:2000, SantaCruz) were attached to the membrane, and labeled with HRP-conjugated anti-mouse IgG or anti-rabbit IgG (1:5000, SantaCruz) as a secondary antibody, and then detected with ECL (GE Healthcare).

TABLE 11
Inhibition abilities (IC₅₀: nM) against eleven (11) HDAC Isozymes

| (nM) | CKD-581 | LBH-589 | Tubastatin A | ACY-161-89 | Compound 19 |
|---|---|---|---|---|---|
| HDAC1 | 0.6734 | 3.828 | 11010 | 4591 | 18140 |
| HDAC2 | 1.585 | 12.25 | ND | 10580 | ND |
| HDAC3 | 0.6933 | 4.77 | 32100 | 3940 | ND |
| HDAC4 | 1676 | 119 | 4506 | 25470 | ND |
| HDAC5 | 1.09 | 6.33 | 5418 | ND | ND |
| HDAC6 | 2.392 | 3.178 | 20.34 | 11.38 | 35.25 |
| HDAC7 | 10.65 | 12.7 | 9878 | ND | ND |
| HDAC8 | 86.13 | 53 | 2759 | 669.7 | 794.3 |
| HDAC9 | 0.4203 | 8.551 | 4595 | 50000 | 30450 |
| HDAC10 | 2.662 | 8.736 | 9492 | 3399 | ND |
| HDAC11 | 1.516 | 7.578 | 9937 | 3251 | 22190 |
| (nM) | Compound 55 | compound 87 | compound 142 | compound 237 | compound 249 |
| HDAC1 | ND | 11170 | 12230 | ND | 8469 |
| HDAC2 | ND | ND | ND | ND | ND |
| HDAC3 | ND | ND | ND | ND | ND |
| HDAC4 | ND | 933.7 | 2213 | 4990 | 255 |
| HDAC5 | ND | 5079 | 4266 | 15960 | 4623 |
| HDAC6 | 227.8 | 13.05 | 3.546 | 32.23 | 5.09 |
| HDAC7 | ND | 3280 | 4527 | ND | 3417 |
| HDAC8 | 506.8 | 1276 | 1060 | 3930 | 220 |
| HDAC9 | ND | 1752 | 1877 | 16860 | 1033 |
| HDAC10 | ND | 15340 | 37010 | 76130 | 53030 |
| HDAC11 | ND | 37540 | 11130 | 33920 | 5527 |

* “ND” indicates no inhibition or compound activity that could not be fit to an IC50 curve.

TABLE 12
Inhibition abilities (IC₅₀: nM) against HDAC1 and HDAC6, and
Selectivity for HDAC6

| No. | HDAC1 (nM) | HDAC6 (nM) | HD1/HD6 |
|---|---|---|---|
| LBH-589 | 3 | 4 | 1.2 |
| ITF-2357 | 196 | 15 | 13 |
| Compound 18 | 1321 | 71.45 | 18.5 |
| Compound 19 | 4218 | 20.9 | 201.8 |
| Compound 20 | 125.2 | 7.44 | 16.8 |
| Compound 40 | 8876 | 117.5 | 75.5 |
| Compound 41 | 98.8 | 1.95 | 50.7 |
| Compound 45 | 5480 | 130.2 | 42.1 |
| Compound 46 | 2073 | 253 | 8.2 |
| Compound 47 | 2910 | 421 | 6.9 |
| Compound 48 | 4369.5 | 145.15 | 30.1 |
| Compound 49 | 13467 | 93.03 | 144.8 |
| Compound 50 | 158.2 | 8.95 | 17.7 |
| Compound 51 | 1328 | 105.9 | 12.5 |
| Compound 52 | 661.9 | 23.89 | 27.7 |
| Compound 53 | 5762 | 64.93 | 88.7 |
| Compound 54 | 1472 | 59.54 | 24.7 |
| Compound 55 | 4085 | 56.36 | 72.5 |
| Compound 56 | 241.7 | 4.13 | 58.5 |
| Compound 57 | 3772 | 22.41 | 168.3 |
| Compound 60 | 9179 | 72.03 | 127.4 |
| Compound 71 | 4,988 | 133.5 | 37 |
| Compound 72 | 6063 | 215.3 | 28.2 |
| Compound 73 | 3910 | 75.08 | 52.1 |
| Compound 74 | 4719 | 141.5 | 33.3 |
| Compound 76 | 5231 | 170.6 | 30.7 |
| Compound 85 | 2547 | 11.32 | 225 |
| Compound 86 | 2844 | 9.94 | 286.1 |
| Compound 87 | 3068 | 7.45 | 411.8 |
| Compound 88 | 3971 | 11.07 | 358.7 |
| Compound 99 | 2682 | 16.36 | 163.9 |
| Compound 101 | 1652 | 12.23 | 135.1 |
| Compound 110 | 896.4 | 11.2 | 80 |
| Compound 111 | 4140 | 12.24 | 338.2 |
| Compound 112 | 4616 | 14.86 | 310.6 |
| Compound 113 | 2440 | 8.5 | 287.1 |
| Compound 114 | 5790 | 17.7 | 327.1 |
| Compound 121 | 3721 | 8.51 | 437.3 |
| Compound 122 | 6271 | 40.98 | 153 |
| Compound 123 | 3568 | 6.61 | 539.8 |
| Compound 126 | 5802 | 35.53 | 163.3 |
| Compound 127 | 5299 | 8.51 | 622.7 |
| Compound 128 | 8139 | 40.98 | 198.6 |
| Compound 129 | 4248 | 6.61 | 642.7 |
| Compound 130 | 2638 | 4.7 | 564.9 |
| Compound 131 | 2693 | 9.1 | 296.6 |
| Compound 136 | 3263 | 8.58 | 380.3 |
| Compound 140 | 5896 | 18.13 | 325.2 |
| Compound 141 | 4143 | 4.9 | 850.7 |
| Compound 142 | 2654 | 4.7 | 568.3 |
| Compound 144 | 8409 | 27.1 | 310.9 |
| Compound 145 | 7225 | 7.8 | 925.1 |
| Compound 156 | 6398 | 15.06 | 424.8 |
| Compound 157 | 3614 | 11.08 | 326.2 |
| Compound 158 | 5135 | 8.52 | 602.7 |
| Compound 166 | 1131 | 2.08 | 543.8 |
| Compound 179 | 153.8 | 3.52 | 43.693 |
| Compound 188 | 2260 | 45.3 | 49.9 |
| Compound 189 | 2229 | 11.5 | 193.5 |
| Compound 190 | 2249 | 5.8 | 385.1 |
| Compound 191 | 8278 | 7.4 | 1115.6 |
| Compound 192 | 4609 | 1.8 | 2546.4 |
| Compound 193 | 3501 | 7.5 | 464.3 |
| Compound 194 | 3081 | 23.46 | 131.3 |
| Compound 203 | 223.6 | 2.6 | 86 |
| Compound 204 | 1948 | 48.72 | 40 |
| Compound 205 | 3173 | 45.96 | 69 |
| Compound 206 | 1197 | 27.5 | 43.5 |
| Compound 207 | 1823 | 43.91 | 41.5 |
| Compound 208 | 284.3 | 1.9 | 152.8 |
| Compound 209 | 364.5 | 2.5 | 148.8 |
| Compound 220 | 2823 | 4.39 | 643.1 |
| Compound 228 | 3527 | 16.93 | 208.3 |
| Compound 232 | 4838 | 45.04 | 107.4 |
| Compound 235 | 1309 | 37.53 | 34.9 |
| Compound 236 | 5110 | 10.66 | 479.4 |
| Compound 237 | 3906 | 17.81 | 219.3 |
| Compound 249 | 3455 | 21.3 | 162.2 |
| Compound 250 | 2721 | 133 | 20.5 |
| Compound 251 | 1452 | 1.77 | 820.3 |
| Compound 266 | 2908 | 1.02 | 2851 |
| Compound 267 | 2274 | 3.35 | 678.8 |
| Compound 268 | 6054 | 13.5 | 448.4 |
| Compound 283 | 11777 | 8.29 | 1420.6 |
| Compound 284 | 10725 | 7.47 | 1435.7 |
| Compound 285 | 13234 | 22.5 | 588.2 |
| Compound 286 | 10592 | 17.45 | 607 |
| Compound 287 | 8202 | 11.47 | 715.1 |
| Compound 288 | 7507 | 10.59 | 708.9 |
| Compound 289 | 7977 | 11.54 | 691.2 |
| Compound 290 | 6406 | 2.19 | 2925.1 |
| Compound 291 | 7921 | 18.58 | 426.3 |
| Compound 292 | 10000 | 12.8 | 781.3 |
| Compound 305 | 2451 | 2.5 | 980.4 |
| Compound 306 | 3230 | 3.1 | 1041.9 |
| Compound 321 | 13344 | 109.6 | 121.8 |
| Compound 322 | 7443 | 182.3 | 40.8 |
| Compound 323 | 8974 | 87.8 | 102.2 |
| Compound 324 | 11622 | 102.7 | 113.2 |
| Compound 325 | 93.8 | 3 | 31.3 |
| Compound 326 | 10079 | 97.6 | 103.3 |
| Compound 328 | 3207 | 22.1 | 145.1 |
| Compound 344 | 102 | 16.2 | 6.3 |
| Compound 345 | 401.6 | 28.1 | 14.3 |
| Compound 346 | 254.7 | 25 | 10.2 |
| Compound 347 | 453.5 | 27 | 16.8 |
| Compound 350 | 999.7 | 102.5 | 9.8 |

### 3. Results

The object of the present invention is to develop a compound that can inhibit selectively HDAC6 among 11 HDAC isyzymes and shows few adverse effects. Therefore, the compound should exhibits excellent inhibition effect against HDAC6, but not against other HDAC isozymes than HDAC6.

For the test, LBH-589 (Novartis), ITF2357 (Italfarmaco), ACY-161-89 (Acetylon) and CKD-581 (CKD), which are under clinical trial, were used as control compounds. As shown in Table 11, LBH-589 inhibits strongly all of eleven (11) HDAC isozymes, while compound 87 inhibits strongly just HDAC6 (its IC₅₀ was 0.013 μM), but does not inhibit other HDAC isozymes, such as HDAC1 (its IC₅₀ was 11.17 μM), except HDAC6. Among 11 HDAC isozymes, HDAC1 induces the most severe adverse effect. From the data of Table 11, the analysis result of the selectivity for HDAC6 over HDAC1 shows that LBH-589 is only 1.2 times, while compound 87 is more than 800 times (0.013 μM of IC₅₀ against HDAC6; 11.17 μM of IC₅₀ against HDAC1). It is clear that compound 87 has excellent inhibition effect against HDAC6 and the selectivity for HDAC6 over HDAC1.

From the data of Table 12, the analysis result of the selectivity for HDAC6 over HDAC1 shows that compound 237 is more than 219 times (17.81 nM of IC₅₀ against HDAC6; 3,906 nM of IC₅₀ against HDAC1) and compound 290 is more than 2,900 times (2.19 nM of IC₅₀ against HDAC6; 6,406 nM of IC₅₀ against HDAC1). So, it is clear that they have also excellent inhibition effect and low adverse effect.

In order to confirm HDAC6 (Tubulin acetylation) and HDAC1 (Histone acetylation), western blotting was performed in HH (cutaneous T cell lymphoma) cell. As like the results of enzyme assay, HDAC1 (Histone acetylation) was not expressed, but HDAC6 (Tubulin acetylation) was expressed. So, the selectivity is confirmed in cell-based assay also.

### Experimental Example 2

### Effectiveness of Compound 87 in a Collagen-Induced Arthritis Model

1. Experimental Animal

7-week aged male DBA1J mice were used in the experiment. The animal facility was kept at constant temperature and constant humidity with a 12-hr dark/12-hr light cycle, and the animals were allowed to access food and water ad libitum. The animals were acclimated to the cage for 7 days.

2. Administration Group

Each arthritis-onset animals were selected and then promptly used in the experiments. The experimental animal groups consist of:
A. two vehicle-control groups administered with only vehicle via s.c. (subcutaneous injection) and p.o. (per oral),
B. a positive control group administered with ITF2357 of 50 mg/kg, and
C. two compound test groups administered with compound 87 in each doses of 10 and 50 mg/kg.

3. Administration of Drug

A solution of ITF2357 for administration was prepared using 0.5% methylcellulose as a vehicle. It was administered orally to the animals in a volume of 10 ml/kg once a day for 7 days. A solution of compound 87 for administration was prepared using DMSO as a vehicle just before administration. It was administered in a volume of 2 ml/kg via s.c. once a day for 7 days. Two vehicle-control groups were administered with only vehicle once a day for 7 days via s.c. and p.o. separately.

4. Experimental Method

Bovine type 2 collagen (Chondrex) and complete Freund's adjuvant (Chondrex) are mixed in 1:1 to become an emulsion. Each 0.1 ml of the obtained emulsion was injected to the tail of each mouse intradermally. After 21 days, Bovine type 2 collagen (Chondrex) and incomplete Freund's adjuvant (Chondrex) are mixed in 1:1 to become an emulsion. Each 0.1 ml of the obtained emulsion was injected to the hips of each mouse intradermally for boosting. After boosting, feet of the mice were observed once a day. When clinical score was occurred, the drug administration and its evaluation were started. The criterion of clinical score was according to Experimental Example 3.

5. Statistical Processing

All the results were expressed as mean±SEM, and each test group and the control groups (vehicle-control group or positive control group) were compared using one-way ANOVA test (Dunnett's test, p<0.001) with GrapicPad Prism 5.0 program.

6. Experimental Result

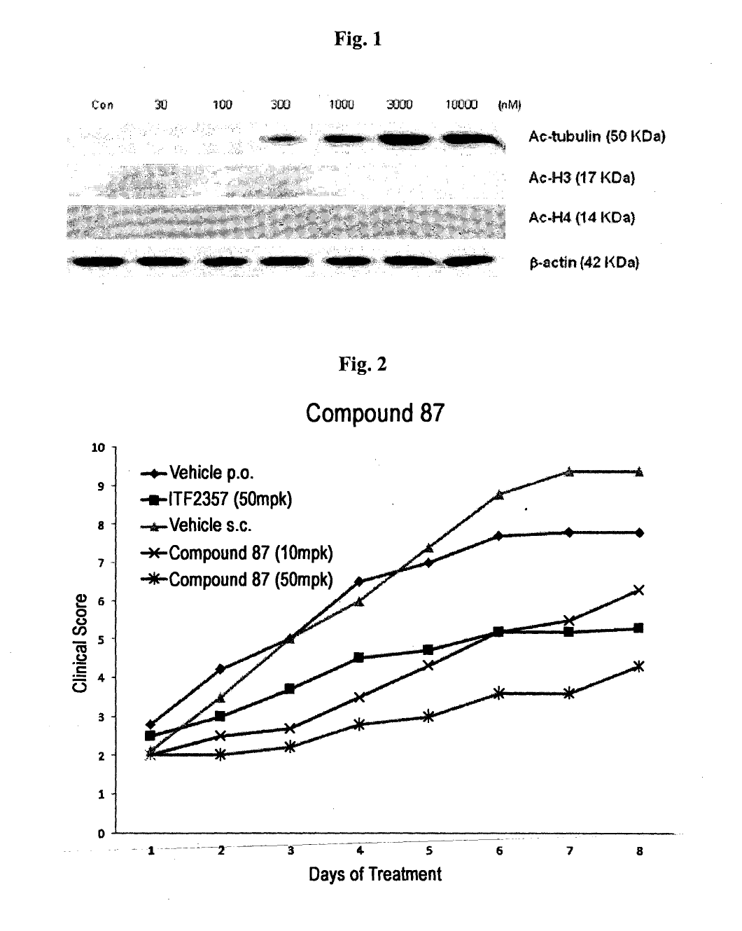

The results are shown in FIG. 2. The effectiveness in a collagen-induced arthritis model was evaluated with clinical score. The clinical score shows the level of arthritis progress. The higher score means more severe level of the arthritis symptom.

In the case of administration of compound 87 of 10 mg/kg (i.e., 10 mpk), the clinical score was decreased by 44%, compared with the positive control group (i.e., the group administered with ITF2357 of 50 mg/kg). In the case of compound 87 of 50 mpk, it was decreased by 68%, compared with the positive control group. So, the excellent anti-arthritis effect of compound 87 was confirmed. Its very low adverse effect was also confirmed from no observation of body-weight loss. The compound shows anti-arthritis effect at low dose of 10 mpk, and the effect is dose-dependant.

### Experimental Example 3

### Effectiveness of Compound 237 in a Collagen-Induced Arthritis Model

### 1. Experimental Animal

6-week aged male DBA1J mice were used in the experiment. The animal facility was kept at constant temperature and constant humidity with a 12-hr dark/12-hr light cycle, and the animals were allowed to access food and water ad libitum. The animals were acclimated to the cage for 7 days.

### 2. Administration Group

Each arthritis-onset animals were selected and then promptly used in the experiments. The experimental animal groups consist of
A. a vehicle-control group administered with only vehicle once a day via s.c. (subcutaneous injection),
B. a compound test group administered with compound 237 in dose of 15 mg/kg q.d. (once a day) via s.c.,
C. a compound test group administered with compound 237 in dose of 15 mg/kg b.i.d. (twice a day) via s.c., and
D. a compound test group administered with compound 237 in dose of 30 mg/kg q.d. via s.c.

### 3. Administration of Drug

A solution of compound 237 for administration was prepared using Cremophore EL:Ethanol:Saline=1.5:1.5:7 as a vehicle just before administration. It was administered in a volume of 5 ml/kg via s.c. with each doses of 15 and 30 mg/kg once or twice a day.

### 4. Experimental Method

Bovine type 2 collagen (Chondrex) and complete Freund's adjuvant (Chondrex) are mixed in 1:1 to become an emulsion. Each 0.1 ml of the obtained emulsion was injected to the tail of each mouse intradermally for immunization. After 21 days, Bovine type 2 collagen (Chondrex) and incomplete Freund's adjuvant (Chondrex) are mixed in 1:1 to become an emulsion. Each 0.1 ml of the obtained emulsion was injected to the hips of each mouse intradermally for boosting. After boosting, the mice were divided in each groups, and administered with compound 237 or only vehicle according to the administration schedule. Their body weights and clinical scores were evaluated three times per week.

### 5. Determination of Clinical Score

From each four feet of a mouse, the clinical scores were evaluated according to the following criterion and added together thereby to determine the clinical score of the mouse (normal, 0; and the most severe edema, 16).

0: normal;

1: observing edema in company with erythema in ankle joint or in tarsal bone joint, but no severe;

2: observing edema in company with erythema connected from ankle joint to tarsal bone joint, but no severe;

3: observing edema in company with erythema connected from ankle joint to metatarsal joint; and

4: observing severe edema in company with erythema connected from ankle joint to toe, or observing sclerosis of leg joint.

### 6. Statistical Processing

All the results were expressed as mean±SEM, and the experimented groups were compared with each other using one-way ANOVA test (Dunnett's test, p<0.001).

### 7. Experimental Result

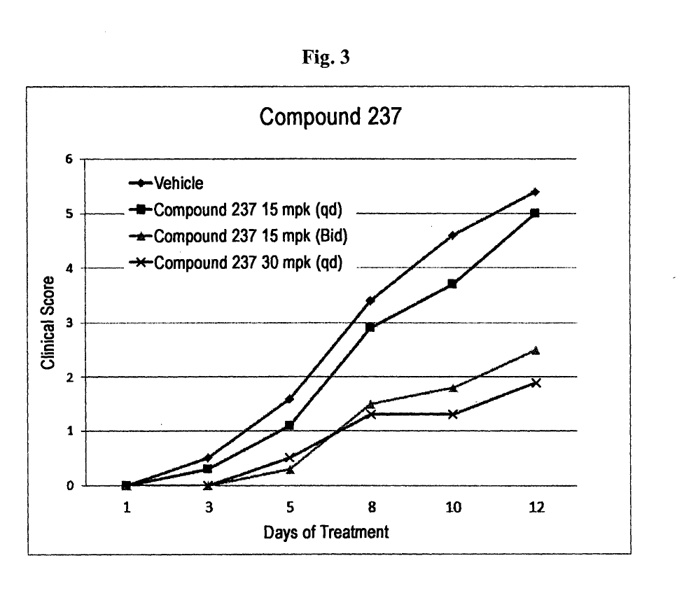

The results are shown in FIG. 3. The higher clinical score means more severe level of the arthritis symptom. In the case of administration of compound 237 of 15 mpk (q.d.), the clinical score was decreased by 7%, compared with the control group (i.e., the group administered with only vehicle). In the case of compound 237 of 15 mpk (b.i.d.), it was decreased by 53%, compared with the control group. In the case of compound 237 of 30 mpk (q.d.), it was decreased by 65%, compared with the control group. Therefore, it is clear that compound 237 has the dose-dependant and excellent anti-arthritis effect. During the administration of drug, body-weight loss or toxic side effect was not observed, so it is clear that compound 237 is safe.

1. A hydroxamate derivative of the following formula 1, isomers thereof, pharmaceutically acceptable salts thereof, hydrates or solvates thereof: 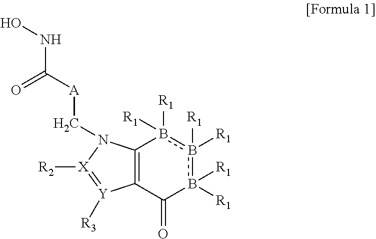
wherein
B is independently C or N;
R₁ is independently absent, -hydrogen, —C₁₋₆ alkyl, —C₂₋₆ alkenyl, —C₃₋₆ cycloalkyl, or 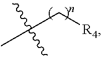
wherein n is 1, 2, 3 or 4, and R₄ is -halogen, —NH(C₁-C₆alkyl), —N(C₁-C₆alkyl)₂, —OH, —O(C₁-C₆alkyl), —S(C₁-C₆alkyl), —N[(C₁-C₆alkyl)(C₁-C₆alcohol)] or 4, 5 or 6 membered heteroaryl or hetero cyclo alkyl compound having 1 to 3 hetero atoms selected independently from the group consisting of O, N, and S, wherein heteroaryl or hetero cyclo alkyl compound optionally substituted with -hydrogen, -halogen, 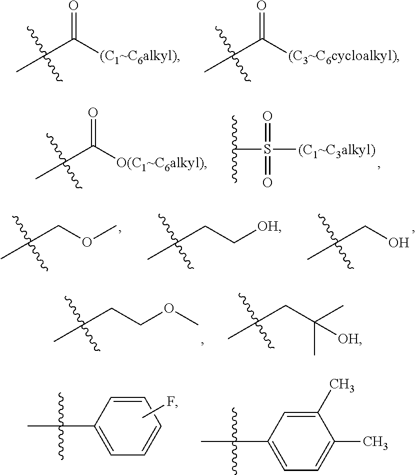
or —C₁-C₆ alkyl;
X and Y are independently C or N;
R₂ and R₃ are independently absent, -hydrogen, -halogen, —CF₃, —CHF₂, —CH₂F, -cyano, -nitro, —C₁-C₆ alkyl, —O(C₁-C₆ alkyl), —NH(C₁-C₆ alkyl), —N(C₁-C₆ alkyl)₂ or -(C₃-C₆ cycloalkyl), or
R₂ and R₃ together with X and Y to which they are bonded may form a 5 or 6 membered aryl or heteroaryl, wherein aryl or heteroaryl has substituent selected independently from -hydrogen, —CF₃, —C₁-C₆ alkyl, —O(C₁-C₆ alkyl), -halogen, —OH, —NH(C₁-C₆ alkyl), —N(C₁-C₆ alkyl)₂ or -nitro; and
A is C₁-C₅ alkyl, -cycloalkyl, -aryl or -heteroaryl, wherein, alkyl, cycloalkyl, aryl and heteroaryl has substituent selected from -hydrogen, —CF₃, —C₁-C₆ alkyl, —O(C₁-C₆ alkyl), -halogen, —OH, —NH(C₁-C₆ alkyl), —N(C₁-C₆ alkyl)₂ or -nitro.

2. The hydroxamate derivative, isomers thereof, pharmaceutically acceptable salts thereof, hydrates or solvates thereof according to claim 1, wherein A is

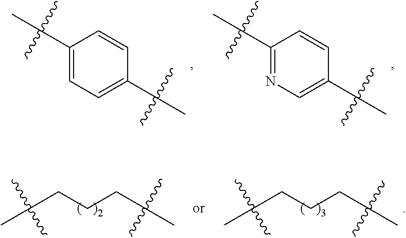

3. The hydroxamate derivative, isomers thereof, pharmaceutically acceptable salts thereof, hydrates or solvates thereof according to claim 1, wherein
B is independently C,
R₁ is independently -hydrogen, methyl or 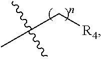
wherein n is 1, and R₄ is -halogen, —NH(C₁-C₆ alkyl), —N(C₁-C₆ alkyl)₂, —OH, —O(C₁-C₆ alkyl), —S(C₁-C₆ alkyl), —N[(C₁-C₆ alkyl)(C₁-C₆ alcohol)] or 4, 5 or 6 membered heteroaryl or hetero cyclo alkyl compound having 1 to 3 atoms selected independently from the group consisting of O, N, and S, wherein heteroaryl or hetero cyclo alkyl compound optionally substituted with -hydrogen, -halogen, 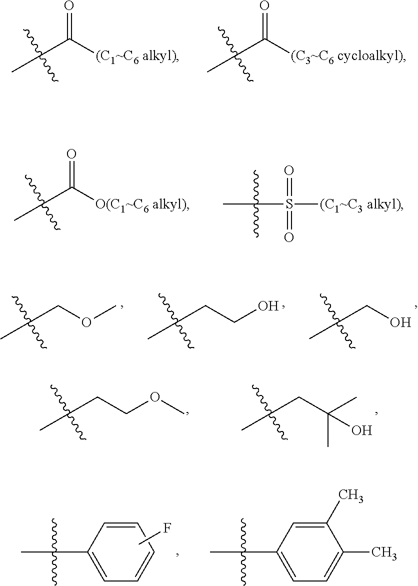
or —C₁-C₆ alkyl); and
A is

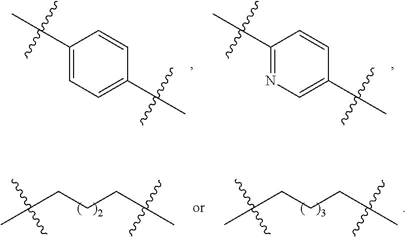

4. The hydroxamate derivative, isomers thereof, pharmaceutically acceptable salts thereof, hydrates or solvates thereof according to claim 1, wherein the hydroxamate derivative of the formula 1 is selected from the group consisting of:
6-(2,2-dimethyl-4-oxo-1,2,3,4-tetrahydrocarbazol-9-yl)-N-hydroxyhexanamide;
4-((2,2-dimethyl-4-oxo-1,2,3,4-tetrahydrocarbazol-9-yl)methyl)-N-hydroxybenzamide;
7-(2,2-dimethyl-4-oxo-1,2,3,4-tetrahydrocarbazol-9-yl)-N-hydroxyheptanamide;
6-(6-bromo-2,2-dimethyl-4-oxo-1,2,3,4-tetrahydrocarbazol-9-yl)-N-ydroxyhexanamide;
6-(7-bromo-2,2-dimethyl-4-oxo-1,2,3,4-tetrahydrocarbazol-9-yl)-N-ydroxyhexanamide;
4-((6-bromo-2,2-dimethyl-4-oxo-1,2,3,4-tetrahydrocarbazol-9-yl)methyl)-N-hydroxybenzamide;
6-(1-bromo-7,7-dimethyl-5-oxo-5,6,7,8-tetrahydrocarbazol-9-yl)-N-hydroxyhexanamide;
6-(1,3-dichloro-7,7-dimethyl-5-oxo-5,6,7,8-tetrahydrocarbazol-9-yl)-N-hydroxyhexanamide;
6-(1,4-dichloro-7,7-dimethyl-5-oxo-5,6,7,8-tetrahydrocarbazol-9-yl)-N-hydroxyhexanamide;
4-((1,4-dichloro-7,7-dimethyl-5-oxo-5,6,7,8-tetrahydrocarbazol-9-yl)methyl)-N-hydroxybenzamide;
7-(6-bromo-2,2-dimethyl-4-oxo-1,2,3,4-tetrahydrocarbazol-9-yl)-N-hydroxyheptanamide;
7-(1,3-dichloro-7,7-dimethyl-5-oxo-5,6,7,8-tetrahydrocarbazol-9-yl)-N-hydroxyheptanamide;
7-(1-bromo-7,7-dimethyl-5-oxo-5,6,7,8-tetrahydrocarbazol-9-yl)-N-hydroxyheptanamide;
4-((5-bromo-2,2-dimethyl-4-oxo-1,2,3,4-tetrahydrocarbazol-9-yl)methyl)-N-hydroxybenzamide;
4-((7-bromo-2,2-dimethyl-4-oxo-1,2,3,4-tetrahydrocarbazol-9-yl)methyl)-N-hydroxybenzamide;
6-(6-fluoro-2,2-dimethyl-4-oxo-1,2,3,4-tetrahydrocarbazol-9-yl)-N-hydroxyhexanamide;
7-(6-fluoro-2,2-dimethyl-4-oxo-1,2,3,4-tetrahydrocarbazol-9-yl)-N-hydroxyheptanamide;
4-((6-fluoro-2,2-dimethyl-4-oxo-1,2,3,4-tetrahydrocarbazol-9-yl)methyl)-N-hydroxybenz amide;
6-(5-fluoro-2,2-dimethyl-4-oxo-2,3,4,4a-tetrahydro-1H-carbazol-9(9aH)-yl)-N-hydroxyhexanamide;
4-((6,7-difluoro-2,2-dimethyl-4-oxo-1,2,3,4-tetrahydrocarbazol-9-yl)methyl)-N-hydroxybenzamide;
6-(6,7-difluoro-2,2-dimethyl-4-oxo-1,2,3,4-tetrahydrocarbazol-9-yl)-N-hydroxyhexanamide;
4-((5,6-difluoro-2,2-dimethyl-4-oxo-1,2,3,4-tetrahydrocarbazol-9-yl)methyl)-N-hydroxybenzamide
6-(5,6-difluoro-2,2-dimethyl-4-oxo-1,2,3,4-tetrahydrocarbazol-9-yl)-N-hydroxyhexanamide;
6-(7-fluoro-2,2-dimethyl-4-oxo-1,2,3,4-tetrahydrocarbazol-9-yl)-N-hydroxyhexanamide;
N-hydroxy-4-((3-((2-methyl-1H-imidazol-1-yl)methyl)-4-oxo-1,2,3,4-tetrahydrocarbazol-9-yl)methyl)benzamide;
N-hydroxy-4-((4-oxo-3-(piperidin-1-ylmethyl)-1,2,3,4-tetrahydrocarbazol-9-yl)methyl)benzamide;
N-hydroxy-4-((3-(morpholinomethyl)-4-oxo-1,2,3,4-tetrahydrocarbazol-9-yl)methyl)benzamide;
N-hydroxy-4-((4-oxo-3-(pyrrolidin-1-ylmethyl)-1,2,3,4-tetrahydrocarbazol-9-yl)methyl)benzamide;
N-hydroxy-4-((4-oxo-1,2,3,4-tetrahydrocarbazol-9-yl)methyl)benzamide;
N-hydroxy-4-((3,6,6-trimethyl-4-oxo-4,5,6,7-tetrahydroindazol-1-yl)methyl)benzamide;
4-((3-((2,6-dimethylmorpholino)methyl)-4-oxo-1,2,3,4-tetrahydrocarbazol-9-yl)methyl)-N-hydroxybenzamide;
N-hydroxy-4-((3-((4-methylpiperazin-1-yl)methyl)-4-oxo-1,2,3,4-tetrahydrocarbazol-9-yl)methyl)benzamide;
4-((3-((4-ethylpiperazin-1-yl)methyl)-4-oxo-1,2,3,4-tetrahydrocarbazol-9-yl)methyl)-N-hydroxybenzamide;
N-hydroxy-4-((3-((4-isopropylpiperazin-1-yl)methyl)-4-oxo-1,2,3,4-tetrahydrocarbazol-9-yl)methyl)benzamide;
N-hydroxy-4-((3-((4-(2-methoxyethyl)piperazin-1-yl)methyl)-4-oxo-1,2,3,4-tetrahydrocarbazol-9-yl)methyl)benzamide;
4-((3-((3,3-difluoroazetidin-1-yl)methyl)-4-oxo-1,2,3,4-tetrahydrocarbazol-9-yl)methyl)-N-hydroxybenzamide;
4-((2,2-dimethyl-3-(morpholinomethyl)-4-oxo-1,2,3,4-tetrahydrocarbazol-9-yl)methyl)-N-hydroxybenzamide;
4-((3-((3,3-difluoropyrrolidin-1-yl)methyl)-4-oxo-1,2,3,4-tetrahydrocarbazol-9-yl)methyl)-N-hydroxybenzamide;
4-((2,2-dimethyl-3-((2-methyl-1H-imidazol-1-yl)methyl)-4-oxo-1,2,3,4-tetrahydrocarbazol-9-yl)methyl)-N-hydroxybenzamide;
4-((3-((4-(4-fluorophenyl)piperazin-1-yl)methyl)-4-oxo-1,2,3,4-tetrahydrocarbazol-9-yl)methyl)-N-hydroxybenzamide;
4-((3-((4-(3,4-dimethylphenyl)piperazin-1-yl)methyl)-4-oxo-1,2,3,4-tetrahydrocarbazol-9-yl)methyl)-N-hydroxybenzamide;
4-((3-((dimethylamino)methyl)-2,2-dimethyl-4-oxo-1,2,3,4-tetrahydrocarbazol-9-yl)methyl)-N-hydroxybenzamide;
N-hydroxy-4-((3-((4-(methylsulfonyl)piperazin-1-yl)methyl)-4-oxo-1,2,3,4-tetrahydrocarbazol-9-yl)methyl)benzamide;
4-((3,3-dimethyl-4-oxo-1,2,3,4-tetrahydrocarbazol-9-yl)methyl)-N-hydroxybenzamide;
N-hydroxy-4-((3,6,6-trimethyl-4-oxo-4,5,6,7-tetrahydroindol-1-yl)methyl)benzamide;
tert-butyl 4-((9-(4-(hydroxycarbamoyl)benzyl)-4-oxo-2,3,4,9-tetrahydro-1H-carbazol-3-yl)methyl)piperazine-1-carboxylate;
4-((6-fluoro-3-(morpholinomethyl)-4-oxo-1,2,3,4-tetrahydrocarbazol-9-yl)methyl)-N-hydroxybenzamide;
4-((6-fluoro-3-((4-methylpiperazin-1-yl)methyl)-4-oxo-1,2,3,4-tetrahydrocarbazol-9-yl)methyl)-N-hydroxybenzamide;
tert-butyl 4-((6-fluoro-9-(4-(hydroxycarbamoyl)benzyl)-4-oxo-2,3,4,9-tetrahydro-1H-carbazol-3-yl)methyl)piperazine-1-carboxylate;
4-((3-((3,3-difluoroazetidin-1-yl)methyl)-6-fluoro-4-oxo-1,2,3,4-tetrahydrocarbazol-9-yl)methyl)-N-hydroxybenzamide;
4-((3-((4-(cyclopropanecarbonyl)piperazin-1-yl)methyl)-4-oxo-1,2,3,4-tetrahydrocarbazol-9-yl)methyl)-N-hydroxybenzamide;
4-((3-fluoro-5-oxo-5,6,7,8-tetrahydrocarbazol-9-yl)methyl)-N-hydroxybenzamide;
4-((6-fluoro-3-((2-methyl-1H-imidazol-1-yl)methyl)-4-oxo-1,2,3,4-tetrahydrocarbazol-9-yl)methyl)-N-hydroxybenzamide;
N-hydroxy-4-((3,6,6-trimethyl-4-oxo-4,5,6,7-tetrahydroindol-1-yl)methyl)benzamide;
N-hydroxy-4-((4-oxo-3,4-dihydropyrimido[4,5-b]indol-9-yl)methyl)benzamide;
4-((6-fluoro-3-((4-isopropylpiperazin-1-yl)methyl)-4-oxo-1,2,3,4-tetrahydrocarbazol-9-yl)methyl)-N-hydroxybenzamide;
4-((3-((4-ethylpiperazin-1-yl)methyl)-6-fluoro-4-oxo-1,2,3,4-tetrahydrocarbazol-9-yl)methyl)-N-hydroxybenzamide;
4-((3-((4-butylpiperazin-1-yl)methyl)-6-fluoro-4-oxo-1,2,3,4-tetrahydrocarbazol-9-yl)methyl)-N-hydroxybenzamide;
4-((6-fluoro-3-((4-(2-hydroxy-2-methylpropyl)piperazin-1-yl)methyl)-4-oxo-1,2,3,4-tetrahydrocarbazol-9-yl)methyl)-N-hydroxybenzamide;
4-((6-fluoro-2,2-dimethyl-3-((2-methyl-1-imidazol-1-yl)methyl)-4-oxo-1,2,3,4-tetrahydrocarbazol-9-yl)methyl)-N-hydroxybenzamide;
N-hydroxy-6-(3-(morpholinomethyl)-4-oxo-1,2,3,4-tetrahydrocarbazol-9-yl)hexanamide;
N-hydroxy-6-(3-((2-methyl-1H-imidazol-1-yl)methyl)-4-oxo-1,2,3,4-tetrahydrocarbazol-9-yl)hexanamide;
N-hydroxy-4-((3-(3-methylbut-2-enyl)-4-oxo-3,4,5,6-tetrahydropyrrolo[2,3-d]pyrimidin-7-yl)methyl)benzamide;
6-(3-((3,3-difluoropyrrolidin-1-yl)methyl)-4-oxo-1,2,3,4-tetrahydrocarbazol-9-yl)-N-hydroxyhexanamide;
N-hydroxy-6-(3-((4-methylpiperazin-1-yl)methyl)-4-oxo-1,2,3,4-tetrahydrocarbazol-9-yl)hexanamide;
6-(3-((4-ethylpiperazin-1-yl)methyl)-4-oxo-1,2,3,4-tetrahydrocarbazol-9-yl)-N-hydroxyhexanamide;
4-((6-fluoro-2,2-dimethyl-3-(morpholinomethyl)-4-oxo-1,2,3,4-tetrahydrocarbazol-9-yl)methyl)-N-hydroxybenzamide;
6-(3-((4-butylpiperazin-1-yl)methyl)-4-oxo-1,2,3,4-tetrahydrocarbazol-9-yl)-N-hydroxyhexanamide;
4-((6-fluoro-2,2-dimethyl-3-((4-methylpiperazin-1-yl)methyl)-4-oxo-1,2,3,4-tetrahydrocarbazol-9-yl)methyl)-N-hydroxybenzamide;
4-((2,3-dimethyl-4-oxo-4,5,6,7-tetrahydroindol-1-yl)methyl)-N-hydroxybenzamide;
N-hydroxy-4-((l-oxo-1,2,3,4-tetrahydropyrido[4,3-b]indol-5-yl)methyl)benzamide;
N-hydroxy-4-((3-methyl-4-oxo-4,5,6,7-tetrahydropyrrolo[3,2-c]pyridin-1-yl)methyl)benzamide;
6-(2,3-dimethyl-4-oxo-4,5,6,7-tetrahydroindol-1-yl)-N-hydroxyhexanamide;
N-hydroxy-4-((2,3,6,6-tetramethyl-4-oxo-4,5,6,7-tetrahydroindol-1-yl)methyl)benzamide;
4-((6,6-dimethyl-4-oxo-3-(trifluoromethyl)-4,5,6,7-tetrahydroindazol-1-yl)methyl)-N-hydroxybenzamide;
N-hydroxy-4-((2-(2-morpholinoethyl)-1-oxo-1,2,3,4-tetrahydropyrido[4,3-b]indol-5-yl)methyl)benzamide;
N-hydroxy-4-((3-methyl-5-(2-morpholinoethyl)-4-oxo-4,5,6,7-tetrahydropyrrolo[3,2-c]pyridin-1-yl)methyl)benzamide;
N-hydroxy-4-((2-(2-(4-methylpiperazin-1-yl)ethyl)-1-oxo-1,2,3,4-tetrahydropyrido[4,3-b]indol-5-yl)methyl)benzamide;
4-((8-fluoro-2-(2-morpholinoethyl)-1-oxo-1,2,3,4-tetrahydropyrido[4,3-b]indol-5-yl)methyl)-N-hydroxybenzamide;
(S)—N-hydroxy-4-((2-(2-(2-(hydroxymethyl)pyrrolidin-1-yl)ethyl)-1-oxo-1,2,3,4-tetrahydropyrido[4,3-b]indol-5-yl)methyl)benzamide;
4-((2,3-dimethyl-5-(morpholinomethyl)-4-oxo-4,5,6,7-tetrahydroindol-1-yl)methyl)-N-hydroxybenzamide;
4-((6-fluoro-3-(((R)-3-fluoropyrrolidin-1-yl)methyl)-4-oxo-1,2,3,4-tetrahydrocarbazol-9-yl)methyl)-N-hydroxybenzamide;
4-((6-fluoro-3-((4-(2-methoxyethyl)piperazin-1-yl)methyl)-4-oxo-1,2,3,4-tetrahydrocarbazol-9-yl)methyl)-N-hydroxybenzamide;
(S)-4-((8-fluoro-2-(2-(2-(methoxymethyl)pyrrolidin-1-yl)ethyl)-1-oxo-1,2,3,4-tetrahydropyrido[4,3-b]indol-5-yl)methyl)-N-hydroxybenzamide;
4-((8-fluoro-2-(2-(4-(hydroxymethyl)piperidin-1-yl)ethyl)-1-oxo-1,2,3,4-tetrahydropyrido[4,3-b]indol-5-yl)methyl)-N-hydroxybenzamide;
4-((8-fluoro-2-(2-(4-methylpiperazin-1-yl)ethyl)-1-oxo-1,2,3,4-tetrahydropyrido[4,3-b]indol-5-yl)methyl)-N-hydroxybenzamide;
4-((8-fluoro-2-(2-(4-(2-hydroxyethyl)piperazin-1-yl)ethyl)-1-oxo-1,2,3,4-tetrahydropyrido[4,3-b]indol-5-yl)methyl)-N-hydroxybenzamide;
(R)-4-((8-fluoro-2-(2-(2-(hydroxymethyl)pyrrolidin-1-yl)ethyl)-1-oxo-1,2,3,4-tetrahydropyrido[4,3-b]indol-5-yl)methyl)-N-hydroxybenzamide;
(S)-4-((8-fluoro-2-(2-(2-(hydroxymethyl)pyrrolidin-1-yl)ethyl)-1-oxo-1,2,3,4-tetrahydropyrido[4,3-b]indol-5-yl)methyl)-N-hydroxybenzamide;
4-((8-fluoro-2-(2-(4-methyl-1,4-diazepan-1-yl)ethyl)-1-oxo-1,2,3,4-tetrahydropyrido[4,3-b]indol-5-yl)methyl)-N-hydroxybenzamide;
4-((8-fluoro-2-(2-((2-hydroxyethyl)(methyl)amino)ethyl)-1-oxo-1,2,3,4-tetrahydropyrido[4,3-b]indol-5-yl)methyl)-N-hydroxybenzamide;
4-((8-fluoro-1-oxo-1,2-dihydropyrido[4,3-b]indol-5-yl)methyl)-N-hydroxybenzamide;
4-((8-fluoro-1-oxo-1,2,3,4-tetrahydropyrido[4,3-b]indol-5-yl)methyl)-N-hydroxybenzamide;
6-(6-fluoro-3-(morpholinomethyl)-4-oxo-1,2,3,4-tetrahydrocarbazol-9-yl)-N-hydroxyhexanamide;
6-(6-fluoro-3-((4-(4-fluorophenyl)piperazin-1-yl)methyl)-4-oxo-1,2,3,4-tetrahydrocarbazol-9-yl)-N-hydroxyhexanamide;
6-(3-((2,6-dimethylmorpholino)methyl)-6-fluoro-4-oxo-1,2,3,4-tetrahydrocarbazol-9-yl)-N-hydroxyhexanamide;
tert-butyl 4-((6-fluoro-9-(6-(hydroxyamino)-6-oxohexyl)-4-oxo-2,3,4,9-tetrahydro-1H-carbazol-3-yl)methyl)piperazine-1-carboxylate;
7-(3-fluoro-5-oxo-5,6,7,8-tetrahydrocarbazol-9-yl)-N-hydroxyheptanamide;
6-(3-((4-butylpiperazin-1-yl)methyl)-6-fluoro-4-oxo-1,2,3,4-tetrahydrocarbazol-9-yl)-N-hydroxyhexanamide;
7-(2,3-dimethyl-4-oxo-4,5,6,7-tetrahydroindol-1-yl)-N-hydroxyheptanamide;
7-(6-fluoro-3-(morpholinomethyl)-4-oxo-1,2,3,4-tetrahydrocarbazol-9-yl)-N-hydroxyheptanamide;
N-hydroxy-6-(2,6,6-trimethyl-4-oxo-4,5,6,7-tetrahydroindol-1-yl)hexanamide;
N-hydroxy-7-(2-methyl-4-oxo-4,5,6,7-tetrahydroindol-1-yl)heptanamide;
N-hydroxy-7-(2,6,6-trimethyl-4-oxo-4,5,6,7-tetrahydroindol-1-yl)heptanamide; and
N-hydroxy-6-(2,3,6,6-tetramethyl-4-oxo-4,5,6,7-tetrahydroindol-1-yl)hexanamide.

5. The hydroxamate derivative, isomers thereof, pharmaceutically acceptable salts thereof, hydrates or solvates thereof according to claim 4, wherein the hydroxamate derivative of the formula 1 is selected from the group consisting of:
N-hydroxy-4-((3-(morpholinomethyl)-4-oxo-1,2,3,4-tetrahydrocarbazol-9-yl)methyl)benzamide;
4-((6-fluoro-3-((4-methylpiperazin-1-yl)methyl)-4-oxo-1,2,3,4-tetrahydrocarbazol-9-yl)methyl)-N-hydroxybenzamide;
4-((6,6-dimethyl-4-oxo-3-(trifluoromethyl)-4,5,6,7-tetrahydroindazol-1-yl)methyl)-N-hydroxybenzamide; and
N-hydroxy-4-((2-(2-morpholinoethyl)-1-oxo-1,2,3,4-tetrahydropyrido[4,3-b]indol-5-yl)methyl)benzamide.

6. A pharmaceutical composition comprising the hydroxamate derivative of the formula 1, isomers thereof, pharmaceutically acceptable salts thereof, hydrates or solvates thereof according to claim 1, and pharmaceutically acceptable carriers.

7. The pharmaceutical composition according to claim 6, wherein the composition is used for prevention or treatment of a disease associated with HDAC activity.

8. The pharmaceutical composition according to claim 7, wherein said disease associated with HDAC activity is inflammatory disease, rheumatoid arthritis or neurodegenerative disease.

9. A pharmaceutical composition comprising the hydroxamate derivative according to claim 2, isomers thereof, pharmaceutically acceptable salts thereof, hydrates or solvates thereof, and pharmaceutically acceptable carriers.

10. A pharmaceutical composition comprising the hydroxamate derivative according to claim 3, isomers thereof, pharmaceutically acceptable salts thereof, hydrates or solvates thereof, and pharmaceutically acceptable carriers.
